(19) United States
(12) Patent Application Publication
(10) Pub. No.: US 2026/0209359 A1
(43) Pub. Date: Jul. 23, 2026

(54) ANTIGEN BINDING PROTEINS

(71) Applicant: NEXTERA AS, Oslo (NO)

(72) Inventors: Geir Åge LØSET, Oslo (NO); Gøril BERNTZEN, Oslo (NO); Vijay Kumar CHENNUPATI, Oslo (NO); Natalia Berges JIMENEZ, Oslo (NO); Terje FRIGSTAD, Oslo (NO); Mark Edward FIFE, Oslo (NO)

(21) Appl. No.: 19/141,018

(22) PCT Filed: Dec. 20, 2023
(86) PCT No.: PCT/EP2023/087120

§ 371 Date: Jun. 18, 2025

(30) Foreign Application Priority Data
Dec. 20, 2022 (IN) 202211073947

(51) Int. Cl. C07K 16/28; A61K 39/00; A61P 35/00; C07K 16/30

(52) U.S. Cl. CPC C07K 16/2833; A61P 35/00; C07K 16/30; A61K 2039/505; C07K 2317/31; C07K 2317/565; C07K 2317/569; C07K 2317/622; C07K 2317/73; C07K 2317/92; C07K 2317/94

(57) ABSTRACT

An antigen binding protein (e.g. antibody) comprising an antigen binding domain that binds to HLA-A*02:01/MAGE-A⁴²³⁰⁻²³⁹, said antigen binding domain comprising a heavy chain variable domain that comprises three complementarity determining regions (CDRs), and a light chain variable domain that comprises three CDRs. Antigen binding protein-based (e.g. antibody-based) compositions, immunoconjugates, therapeutic and diagnostic methods, and kits are also provided.

The present invention relates generally to the field of antigen binding proteins, in particular to antibodies which bind to, or specifically bind to, the pMHC (peptide-Major Histocompatibility Complex) HLA-A*02:01/MAGE-A4²³⁰⁻²³⁹. The invention further relates to compositions and immunoconjugates comprising such antibodies and to methods of producing such antibodies. The invention also relates to methods and uses which employ such antibodies, for example in the treatment of cancer.

The treatment of cancer is still one of the biggest unmet medical needs to date. While there have been advances in cancer therapy during the last decades, cancer remains one of the leading causes of death. As populations in industrialized countries are benefitting from longer average life expectancies, the urgency for improved or new cancer therapies is increasing.

Human leukocyte antigen (HLA) class I molecules are expressed on the surface of all nucleated cells presenting peptides for T-cell recognition. The peptides presented in HLA class I molecules are protein fragments of intracellular origin, which are degraded by an array of proteases. The protein fragments are truncated to smaller peptides and translocated into the endoplasmic reticulum (ER). In the ER, the peptide-HLA class I molecule (pHLA), a type of peptide-MHC (pMHC) complex, is assembled from a peptide, a polymorphic heavy chain, and the monomorphic light chain β2-microglobulin (β2m). A peptide with adequate binding motif residues will bind into the peptide-binding groove of the HLA class I molecule, allowing the assembled molecule to leave the ER and be transported via the Golgi complex to the cell surface to display the peptides.

The Melanoma Antigen Gene (MAGE) protein family is a large group of proteins (more than 40 human proteins) sharing a common MAGE homology domain. A subset of these proteins is aberrantly expressed in a wide variety of cancer types (solid tumours and blood cancers). MAGE proteins were originally discovered as antigens on tumour cells and are of interest as cancer immunotherapy targets. In particular, type I MAGE proteins (MAGE-A, -B and -C subfamily members) are considered as potential cancer immunotherapy targets.

The MAGE-A subfamily contains 12 genes (MAGE-A1 to -A12). The biological function of the MAGE-A4 protein (Uniprot Accession No. P43358) is not well-understood, but is nevertheless a target for cancer therapy. High expression levels of MAGE-A4 peptide-MHC (pMHC) has been reported in various cancers (solid tumours and blood cancers).

The 10-mer peptide GVYDGREHTV (SEQ ID NO: 19) corresponds to amino acids 230-239 of the full length MAGE-A4 protein. This peptide binds to HLA-A*02:01 and the peptide-HLA complex has been shown to stimulate cytotoxic T cells leading to lysis of MAGE-A4 positive, HLA-A*02:01 positive, cancer cells².

MAGE-A4 pMHC is a target for TCR-based T-cell therapy, currently in clinical development. Autologous T cells engineered to target MAGE-A4 pMHC have been demonstrated to reduce the size of solid tumours with a manageable toxicity profile.

Although the T-cell receptor (TCR) is the endogenous binding partner for pMHC, the use of recombinant TCRs for the purpose of detecting peptide presentation is challenging. TCRs have low affinity for pMHC, and soluble TCRs are intrinsically unstable and production is demanding. Monoclonal antibodies against pMHC targets are therefore desirable, but are difficult to generate given the small epitope of the bound peptide in the human leukocyte antigen (HLA). Given the complex structure of the epitope targets, generating monoclonal antibodies against pMHC targets with high binding specificity and acceptably low levels of off-target activity, and further with good cytotoxicity profiles at low doses, is a particular difficulty.

What are needed in the art are new, preferably improved, agents, such as antibodies, that target MAGE-A4 pMHC, which will be useful in the treatment, prophylaxis and diagnosis of cancer.

The present invention provides one such alternative and improved therapeutic option in the form of antigen binding proteins (e.g. antibodies) directed to HLA-A*02:01/MAGE-A4²³⁰⁻²³⁹. The antibodies generated by the inventors have advantageous properties which make them ideal agents for the above-mentioned uses.

As will be described in more detail elsewhere herein, antigen binding proteins (e.g. antibodies) of the invention have been shown to be capable of binding to HLA-A*02:01/MAGE-A4²³⁰⁻²³⁹ with high specificity, and have excellent ability to induce T-cell mediated killing of cancer cells. Advantageously, the cell killing effects are observed at very low concentrations, notably in assays comprising an effector cell to target cell ratio (E:T) of only 1:1, and notably in disparate cancer types. Advantageously, the cell killing effects are observed whilst the antigen binding proteins (e.g. antibodies) bind with relatively low strength to target. The antigen binding proteins (e.g. antibodies) thus achieve advantageous cytotoxicity whilst maintaining an advantageous safety profile (reduced risk of off-target effects). Indeed, the antigen binding proteins (e.g. antibodies) of the invention have also been shown to have outstanding specificity, in particular in terms of their demonstrated lack of binding to/cross-reactivity with many HLA-A*02:01 restricted peptides with high and very high sequence identity to the target peptide MAGE-A4²³⁰⁻²³⁹. The antigen binding proteins (e.g. antibodies) of the invention have also been shown to redirect T-cell activity preferentially against HLA-A*02:01/MAGE-A4²³⁰⁻²³⁹ positive cells, to preferentially induce activation, differentiation and proliferation of T cells directed against HLA-A*02:01/MAGE-A4²³⁰⁻²³⁹ positive cells and to induce T-cell mediated cytotoxicity preferentially against HLA-A*02:01/MAGE-A4²³⁰⁻²³⁹ positive cells. The antigen binding proteins (e.g. antibodies) of the invention have also been shown to have advantageous thermal stability.

To the inventors' knowledge, no other anti-MAGE-A4 pMHC antibodies have been disclosed to have this advantageous combination of properties, and antigen binding proteins (e.g. antibodies) with one or more, preferably all, of these properties are preferred.

Such antigen binding proteins (e.g. antibodies) of the invention comprise a HLA-A*02:01/MAGE-A4²³⁰⁻²³⁹ antigen binding domain as described herein, and can conveniently and advantageously be used for the treatment of diseases associated with HLA-A*02:01/MAGE-A4²³⁰⁻²³⁹ expression, in particular for the treatment of cancer.

A1 In one aspect, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
(a) a variable heavy (VH) CDR1 that comprises the amino acid sequence of GYTLTELS (SEQ ID NO:5) or a sequence substantially homologous thereto,
(b) a VH CDR2 that comprises the amino acid sequence of FDPEDGET (SEQ ID NO:6) or a sequence substantially homologous thereto, and
(c) a VH CDR3 that comprises the amino acid sequence of ATDQGSSWGFY (SEQ ID NO:7) or a sequence substantially homologous thereto;
and/or (preferably “and”) wherein
said light chain variable domain comprises:
(d) a variable light (VL) CDR1 that comprises the amino acid sequence of QSISSY (SEQ ID NO:8) or a sequence substantially homologous thereto,
(e) a VL CDR2 that comprises the amino acid sequence of AAS (SEQ ID NO:9) or a sequence substantially homologous thereto, and
(f) a VL CDR3 that comprises the amino acid sequence of QQSYSTPYT (SEQ ID NO:10) or a sequence substantially homologous thereto.

It is the antigen binding domain that confers the binding ability of the antigen binding protein. Thus, alternatively viewed, the present invention provides an antigen binding protein comprising at least one antigen binding domain which binds to, or specifically binds to, HLA-A*02:01/MAGE-A4²³⁰⁻²³⁹, said antigen binding domain comprising a heavy chain variable domain (VH domain) that comprises three complementarity determining regions (CDRs), and a light chain variable domain (VL domain) that comprises three CDRs, wherein said VH and VL domains are as defined above.

By “at least one” antigen binding domain means “at least a first antigen binding domain”. As discussed elsewhere herein, further (i.e. second, third) etc. antigen binding domains may be present. However, it is the “first” antigen binding domain referred to herein that binds to, or specifically binds to, HLA-A*02:01/MAGE-A4²³⁰⁻²³⁹.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3 or a sequence substantially homologous thereto, and/or (preferably “and”) a VL domain that comprises the amino acid sequence of SEQ ID NO: 4 or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3 or a sequence substantially homologous thereto, and a VL domain that comprises the amino acid sequence of SEQ ID NO: 4 or a sequence substantially homologous thereto.

### Substantially Homologous Sequences

The invention provides the specific antigen binding proteins (e.g. antibodies) disclosed in Table A. Table A provides the specific amino acid and nucleotide sequences of these antigen binding proteins (e.g. antibodies) and the regions/domains thereof, including the CDR sequences, the framework region sequences, and the VH and VL domain sequences. These sequences of the specific antibodies of the invention disclosed in Table A, i.e. those having a given SEQ ID NO., are referred to herein as “specific”, “reference”, “given” or “disclosed” sequences.

Throughout this application, reference is also made to “substantially homologous” sequences, i.e. sequences that are “substantially homologous” to a specific sequence disclosed in Table A. The invention also encompasses antigen binding proteins (e.g. antibodies) that comprise i) one or more CDRs, and/or ii) one or more framework regions, and/or iii) a VH domain and/or iv) a VL domain that have a sequence substantially homologous to a specific sequence disclosed in Table A. Substantially homologous sequences are defined by reference to a “given”, “reference”, “disclosed” or “specific” sequence, which is a sequence disclosed in Table A, having a specific SEQ ID NO. Alternatively viewed, the substantially homologous sequences are “based on” a specific sequence of Table A.

The discussion below relating to substantially homologous sequences applies to all aspects and embodiments of the invention described elsewhere herein, wherever the terms “substantially homologous”, “or a sequence substantially homologous thereto”, or similar terms, are used.

In all aspects and embodiments, the specific antigen binding protein (e.g. antibody), which provides the “specific”, “given”, “disclosed”, or “reference” sequence(s) on which the substantially homologous sequence(s) is/are based is an antibody disclosed in Table A, i.e. the 1-H02 antibody, the AM15 antibody, the AM2 antibody, the AMC9 antibody, the AM1 antibody, the AM3 antibody, the AM4 antibody, the AM5 antibody, the AM6 antibody, the AM7 antibody, the AM9 antibody, the AM10 antibody, the AM11 antibody, the AM12 antibody, the AM13 antibody, the AM14 antibody, the AM16 antibody, the AM17 antibody, the AM18 antibody, the AMC1 antibody, the AMC3 antibody, the AMC4 antibody, the AMC5 antibody, the AMC6 antibody, the AMC7 antibody, the AMC8 antibody, the AMC10 antibody, the AMC11 antibody, the AMC12 antibody, or the AMC14 antibody. Preferably, the “specific”, “given”, “disclosed” or “reference” antigen binding protein (e.g. antibody) is the 1-H02 antibody, the AM15 antibody, the AM2 antibody, the AMC9 antibody, the AM6 antibody, the AMC8 antibody, or the AMC11 antibody. More preferably, the “specific”, “given”, “disclosed” or “reference” antigen binding protein (e.g. antibody) is the 1-H02 antibody, the AM15 antibody, the AM2 antibody, or the AMC9 antibody, still more preferably the 1-H02 antibody or the AM15 antibody.

Antigen binding proteins (e.g. antibodies) of the invention comprising one or more CDR sequences that are “substantially homologous” to one or more of the specific CDRs disclosed in Table A, e.g. to SEQ ID Nos: 5 to 7 and/or to SEQ ID Nos: 8 to 10, and antigen binding proteins (e.g. antibodies) of the invention comprising heavy and/or light chain variable domains that comprise an amino acid sequence that is substantially homologous to a specific heavy and/or light chain variable domain disclosed in Table A, e.g. to SEQ ID NO:3 and/or 4, respectively, are termed “substantially homologous antigen binding proteins” (e.g. substantially homologous antibodies”) herein. Thus, “substantially homologous antigen binding proteins” (e.g. substantially homologous antibodies”) are any such antigen binding protein/antibody comprising one or more CDR or variable domain sequence that is substantially homologous to the sequence of a specific CDR or variable domain sequence with a given SEQ ID NO herein.

In all aspects and embodiments, antigen binding proteins, e.g. antibodies, containing substantially homologous sequences retain the ability to bind to HLA-A*02:01/MAGE-A4²³⁰⁻²³⁹. Preferably, antigen binding proteins, e.g. antibodies, containing substantially homologous sequences retain one or more (preferably all) of the other properties of a specific antigen binding protein (e.g. antibody) of the invention, i.e. an antigen binding protein (e.g. antibody) disclosed in Table A, e.g. the 1-H02 antibody, the AM15 antibody, the AM2 antibody, the AMC9 antibody, the AM6 antibody, the AMC8 antibody, or the AMC11 antibody, preferably the 1-H02 antibody, the AM15 antibody, the AM2 antibody, or the AMC9 antibody, more preferably the 1-H02 antibody or the AM15 antibody.

The term “substantially homologous” as used herein in connection with an amino acid or nucleic acid sequence includes sequences having at least 30%, 40%, 50%, 55%, 60%, 62%, 65%, 70% or 75%, preferably at least 80%, and even more preferably at least 85%, 90%, 95%, 96%, 97%, 98% or 99%, sequence identity to the specific amino acid or nucleic acid sequence disclosed. Substantially homologous sequences of the invention thus include single or multiple base or amino acid alterations (additions, substitutions, insertions or deletions) to the specific sequences of the invention.

Other preferred examples of substantially homologous sequences are sequences containing conservative amino acid substitutions of the specific amino acid sequences disclosed.

The antigen binding proteins (e.g. antibodies) of the invention preferably comprise at least one heavy chain variable domain (region) that includes an amino acid sequence region of at least 60%, 62%, 65%, 70% or 75%, more preferably at least 80%, more preferably at least 85%, more preferably at least 90% or 95% and most preferably at least 96%, 97%, 98% or 99% amino acid sequence identity to the amino acid sequence of a heavy chain variable domain of a specific antigen binding protein (e.g. antibody) of the invention (disclosed in Table A), e.g. SEQ ID NO: 3, 118, or 130, preferably SEQ ID NO: 3 or 118, more preferably SEQ ID NO: 3; and/or (preferably “and”) at least one light chain variable domain (region) that includes an amino acid sequence region of at least 60%, 62%, 65%, 70% or 75%, more preferably at least 80%, more preferably at least 85%, more preferably at least 90% or 95% and most preferably at least 96%, 97%, 98% or 99% amino acid sequence identity to the amino acid sequence of a light chain variable domain of a specific antigen binding protein (e.g. antibody) of the invention (disclosed in Table A), e.g. SEQ ID NO: 4, 114, 116, 142 or 147, preferably SEQ ID NO: 4, 114 or 116, more preferably SEQ ID NO: 4 or 114.

Preferably, the antigen binding proteins (e.g. antibodies) of the invention comprise at least one heavy chain variable domain that includes an amino acid sequence region of at least 95% and most preferably at least 96%, 97%, 98% or 99% amino acid sequence identity to the amino acid sequence of a heavy chain variable domain of a specific antigen binding protein (e.g. antibody) of the invention (disclosed in Table A, e.g. SEQ ID NO: 3, 118, or 130, preferably SEQ ID NO: 3 or 118, more preferably SEQ ID NO: 3); and/or (preferably “and”) at least one light chain variable domain that includes an amino acid sequence region of at least 75%, more preferably at least 80%, more preferably at least 90% amino acid sequence identity to the amino acid sequence of a light chain variable domain of a specific antigen binding protein (e.g. antibody) of the invention (disclosed in Table A, e.g. SEQ ID NO: 4, 114, 116, 142 or 147, preferably SEQ ID NO: 4, 114 or 116, more preferably SEQ ID NO: 4 or 114).

Preferably, sequences that are substantially homologous to a given VH domain sequence have at least 90% identity to said given sequence; and/or (preferably “and”) sequences that are substantially homologous to a given VL domain sequence have at least 80% identity (preferably at least 90% identity) to said given sequence.

Preferably, sequences that are substantially homologous to a given VH domain sequence have at least 95% identity to said given sequence; and/or (preferably “and”) sequences that are substantially homologous to a given VL domain sequence have at least 80% identity (preferably at least 90% identity) to said given sequence.

Alternatively, or in addition, at the amino acid level, preferred substantially homologous antigen binding proteins (e.g. antibodies) contain up to 21, more preferably up to 10, e.g. only 1, 2, 3, 4, 5, 6, 7, 8, 9 or 10, more preferably up to 7, e.g. only 1, 2, 3, 4, 5, 6, or 7, more preferably up to 6, e.g. only 1, 2, 3, 4, 5 or 6, more preferably up to 5, for example only 1, 2, 3, 4 or 5, more preferably up to 4, e.g. only 1, 2, 3 or 4, more preferably up to 3, e.g. only 1, 2 or 3, more preferably up to 2, e.g. only 1 or 2, more preferably only 1, or 0, altered amino acids, in the VH domain and/or the VL domain as compared to the VH and/or VL domain of a specific antigen binding protein (e.g. antibody) of the invention (disclosed in Table A, e.g. 1-H02).

Preferred substantially homologous antigen binding proteins (e.g. antibodies) contain up to 5, for example only 1, 2, 3, 4 or 5, more preferably up to 4, e.g. only 1, 2, 3 or 4, more preferably up to 3, e.g. only 1, 2 or 3, more preferably up to 2, e.g. only 1 or 2, more preferably only 1, or 0, altered amino acids, in the VH domain as compared to the VH domain of a specific antigen binding protein (e.g. antibody) of the invention (disclosed in Table A e.g. 1-H02, i.e. SEQ ID NO: 3);
and/or, (preferably “and”) contain up to 21, more preferably up to 10, e.g. only 1, 2, 3, 4, 5, 6, 7, 8, 9, 10, more preferably up to 7, e.g. only 1, 2, 3, 4, 5, 6, or 7, more preferably up to 6, e.g. only 1, 2, 3, 4, 5 or 6, more preferably up to 5, for example only 1, 2, 3, 4 or 5, more preferably up to 4, e.g. only 1, 2, 3 or 4, more preferably up to 3, e.g. only 1, 2 or 3, more preferably up to 2, e.g. only 1 or 2, more preferably only 1, or 0, altered amino acids in the VL domain as compared to the VL domain of a specific antigen binding protein (e.g. antibody) of the invention (disclosed in Table A e.g. 1-H02, i.e. SEQ ID NO: 4.

Preferred substantially homologous antigen binding proteins (e.g. antibodies) contain up to 4, e.g. only 1, 2, 3 or 4, more preferably only 1, or 0, altered amino acids in the VH domain as compared to the VH domain of a specific antigen binding protein (e.g. antibody) of the invention (disclosed in Table A e.g. 1-H02, i.e. SEQ ID NO: 3);
and/or, preferably “and” contain up to 21, more preferably up to 7, e.g. only 1, 2, 3, 4, 5, 6, or 7, more preferably up to 6, e.g. only 1, 2, 3, 4, 5 or 6, more preferably up to 3, e.g. only 1, 2 or 3, or 0, altered amino acids in the VL domain as compared to the VL domain of a specific antigen binding protein (e.g. antibody) of the invention (disclosed in Table A e.g. 1-H02, i.e. SEQ ID NO: 4.

Preferably, the antigen binding proteins (e.g. antibodies) of the invention comprise at least one heavy chain variable domain and/or (preferably “and”) at least one light chain variable domain as defined above, and further wherein
said heavy chain variable domain comprises three CDRs comprising the amino acid sequences of the VH CDR1, VH CDR2, VH CDR3 of a specific antibody of the invention (disclosed in Table A e.g. 1-H02), or sequences substantially homologous thereto; and/or (preferably “and”) said light chain variable domain comprises three CDRs comprising the amino acid sequences of the VL CDR1, VL CDR2, VL CDR3 of a (preferably said) specific antibody of the invention (disclosed in Table A e.g. 1-H02), or sequences substantially homologous thereto.

Sequences substantially homologous to a given CDR sequence are preferably as described below.

Other preferred examples of “substantially homologous” sequences are sequences having at least 30%, at least 40%, at least 50%, at least 55%, at least 60%, at least 62%, at least 65%, at least 70%, at least 75%, at least 80%, at least 85%, at least 90%, or at least 95% amino acid sequence identity to the amino acid sequence of one or more of the CDR regions or one or more of the framework (FR) regions disclosed in Table A. Thus, in some embodiments, a “substantially homologous” CDR sequence may be a sequence having at least 30%, at least 40%, at least 50%, at least 55%, at least 60%, at least 62%, at least 65%, at least 70%, at least 75%, at least 80%, at least 85%, at least 90% or at least 95% sequence identity to a given CDR sequence described herein.

In some embodiments, in antigen binding proteins (e.g. antibodies) having a “substantially homologous” sequence as compared to a given sequence, or having a certain degree of sequence identity as compared to a given sequence, the altered amino acid residues(s) are not in a CDR region. For example, in some embodiments, in antigen binding proteins (e.g. antibodies) having a VH domain that has a certain degree of sequence identity to a given VH domain sequence of a particular antigen binding protein (e.g. antibody) of the invention (disclosed in Table A, e.g. 1-H02, AM15, AM2, AMC9, AM6, AMC8 or AMC11, preferably 1-H02, AM15, AM2 or AMC9, more preferably 1-H02 or AM15), the altered (or variant) residue(s) are not in a CDR region. Thus, in some embodiments, in antigen binding proteins (e.g. antibodies) having a “substantially homologous” sequence as compared to a given sequence, or having a certain degree of sequence identity as compared to a given sequence, the altered amino acid residues(s) are in one or more framework regions.

As is evident from elsewhere herein, in other embodiments, in antigen binding proteins (e.g. antibodies) having a “substantially homologous” sequence as compared to a given sequence, or having a certain degree of sequence identity as compared to a given sequence, the altered amino acid residues(s) may be in one or more CDR regions.

At the amino acid level preferred substantially homologous sequences contain up to 4, e.g. only 1, 2, 3, or 4, preferably up to 3, e.g. only 1, 2 or 3, more preferably up to 2, e.g. only 1 or 2, more preferably only 1 altered amino acids, in one or more of the framework regions and/or one or more of the CDRs making up the sequences of the invention (disclosed in Table A). Such alterations might be amino acid substitutions, e.g. conserved or non-conserved amino acid substitutions, or a mixture thereof.

Thus, a sequence substantially homologous to a given CDR sequence preferably comprises up to 4, e.g. only 1, 2, 3, or 4, preferably up to 3, e.g. only 1, 2 or 3, more preferably up to 2, e.g. only 1 or 2, more preferably only 1 altered amino acids (preferably substitutions) as compared to the given CDR sequence. This is particularly the case for sequences substantially homologous to a given VH CDR1, VH CDR2, VH CDR3, VL CDR1 and/or VL CDR3 sequence herein (e.g. disclosed in Table A).

Sequences substantially homologous to a given VL CDR2 herein (e.g. disclosed in Table A) preferably comprise up to 2, e.g. only 1 or 2, more preferably only 1 altered amino acids (preferably substitutions) as compared to the given CDR sequence.

In certain embodiments, if a given starting (reference) sequence is relatively short (e.g. three amino acids in length), then fewer amino acid substitutions may be present in sequences substantially homologous thereto as compared with the number of amino acid substitutions that might optionally be made in a sequence substantially homologous to a longer starting (reference) sequence. For example, in certain preferred embodiments the VL CDR2 sequence of antigen binding proteins of the invention is three amino acids in length. A sequence substantially homologous to a starting (reference) VL CDR2 sequence in accordance with the present invention, e.g. a starting (reference) VL CDR2 sequence which is three amino acid residues in length, preferably has only 1 or 2, more preferably only 1 altered amino acid in comparison with the starting sequence. A sequence substantially homologous to a starting (reference) VH CDR3 sequence in accordance with the present invention, e.g. a starting (reference) VH CDR3 sequence which is 11 amino acid residues in length, preferably has up to 3 (e.g. only 1, 2 or 3) altered amino acid in comparison with the starting sequence. A sequence substantially homologous to a starting VL CDR1 sequence in accordance with the present invention, e.g. a starting VL CDR1 sequence which is six amino acid residues in length, preferably has up to 4 (e.g. only 1, 2, 3, or 4), more preferably up to 3 (e.g. only 1, 2 or 3), more preferably up to 2 (e.g. only 1 or 2) altered amino acid in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 30%, 40%, 50%, 55%, 60%, 62%, 65%, 70%, 75%, 80%, 85%, 90%, 95%, 96%, 97%, 98% or 99%.

Thus, in all aspects and embodiments herein:

A sequence substantially homologous to a given VH CDR1 sequence (i.e. a VH CDR1 sequence of the invention (shown in Table A) is preferably a sequence containing up to 2 (e.g. only 1 or 2), preferably only 1 amino acid substitutions as compared to the given CDR sequence.

Alternatively or in addition, a sequence substantially homologous to a given VH CDR2 sequence is preferably a sequence containing up to 2 (e.g. only 1 or 2), preferably only 1 amino acid substitutions as compared to the given CDR sequence.

Alternatively or in addition, a sequence substantially homologous to a given VH CDR3 sequence is preferably a sequence containing up to 3 (e.g. only 1, 2, or 3), preferably up to 2 (e.g. only 1 or 2), preferably only 1 amino acid substitutions as compared to the given CDR sequence,

Alternatively or in addition, a sequence substantially homologous to a given VL CDR1 sequence is preferably a sequence containing up to 2 (e.g. only 1 or 2), preferably only 1 amino acid substitutions as compared to the given CDR sequence.

Alternatively or in addition, a sequence substantially homologous to a given VL CDR2 sequence is preferably a sequence containing only 1 amino acid substitution as compared to the given CDR sequence.

Alternatively or in addition, a sequence substantially homologous to a given VL CDR3 sequence is preferably a sequence containing up to 3 (e.g. only 1, 2 or 3), more preferably only 1 or 2, more preferably only 1 amino acid substitutions as compared to the given CDR sequence.

Preferably, each of the sequences substantially homologous to a given CDR sequence comprises only 1 amino acid substitution as compared to the given CDR sequence.

Preferably, in all aspects and embodiments, the antigen binding protein (e.g. antibody) of the invention comprises
a VH CDR1 comprising the amino acid sequence of the given VH CDR1 of a specific antigen binding protein (e.g. antibody) of the invention (disclosed in Table A), or a sequence substantially homologous thereto, wherein said substantially homologous sequence comprises up to 2 (e.g. only 1 or 2) amino acid substitutions as compared to the given VH CDR1 sequence;
a VH CDR2 comprising the amino acid sequence of the given VH CDR2 of a (preferably said) specific antigen binding protein (e.g. antibody) of the invention (disclosed in Table A) or a sequence substantially homologous thereto, wherein said substantially homologous sequence comprises up to 2 (e.g. only 1 or 2), amino acid substitutions as compared to the given VH CDR1 sequence;
a VH CDR3 comprising the amino acid sequence of the given VH CDR3 of a (preferably said) specific antigen binding protein (e.g. antibody) of the invention (disclosed in Table A), or a sequence substantially homologous thereto, wherein said substantially homologous sequence comprises up to 3 (e.g. only 1, 2, or 3), amino acid substitutions as compared to the given CDR sequence;
a VL CDR1 comprising the specific amino acid sequence of the given VL CDR1 of a (preferably said) specific antigen binding protein (e.g. antibody) of the invention (disclosed in Table A), or a sequence substantially homologous thereto, wherein said substantially homologous sequence comprises up to 2 (e.g. only 1 or 2) amino acid substitutions as compared to the given VL CDR1 sequence;
a VL CDR2 comprising the specific amino acid sequence of the given VL CDR2 of a (preferably said) specific antigen binding protein (e.g. antibody) of the invention (disclosed in Table A), or a sequence substantially homologous thereto, wherein said substantially homologous sequence comprises only 1 amino acid substitutions as compared to the given VL CDR2 sequence; and
a VL CDR3 comprising the specific amino acid sequence of the given VL CDR3 of a (preferably said) specific antigen binding protein (e.g. antibody) of the invention (disclosed in Table A), or a sequence substantially homologous thereto, wherein said substantially homologous sequence comprises up to 3 (e.g. only 1, 2 or 3), amino acid substitutions as compared to the given VL CDR3 sequence.

In all aspects and embodiments, preferably, each sequence substantially homologous to a given CDR sequence comprises only 1 amino acid substitution as compared to the given CDR sequence.

In all aspects and embodiments, in a substantially homologous antigen binding protein (e.g. antibody) of the invention, each (i.e. all) of the substantially homologous sequences are substantially homologous to a given sequence derived from the same specific antigen binding protein (e.g. antibody) of the invention (disclosed in Table A). Preferred specific antigen binding proteins (e.g. antibodies) of the invention (disclosed in Table A) are described elsewhere herein.

In all aspects and embodiments herein a sequence substantially homologous to a given VH CDR1 sequence (i.e. a VH CDR1 sequence of the invention (shown in Table A, e.g. SEQ ID NO: 5)) is preferably a sequence containing up to 4 (e.g. only 1, 2, 3 or 4) amino acid substitutions as compared to the given CDR sequence.

In all aspects and embodiments herein a sequence substantially homologous to a given VH CDR2 sequence (e.g. to SEQ ID NO: 6) is preferably a sequence containing up to 4 (e.g. only 1, 2, 3 or 4) amino acid substitutions as compared to the given CDR sequence.

In all aspects and embodiments herein a sequence substantially homologous to a given VH CDR3 (e.g. to SEQ ID NO: 7) sequence is preferably a sequence containing only 1 amino acid substitution as compared to the given CDR sequence,

In all aspects and embodiments herein a sequence substantially homologous to a given VL CDR1 (e.g. to SEQ ID NO: 8) sequence is preferably a sequence containing up to 4 (e.g. only 1, 2, 3 or 4), preferably up to 3 (e.g. only 1, 2 or 3), amino acid substitutions as compared to the given CDR sequence.

In all aspects and embodiments herein, a sequence substantially homologous to a given VL CDR2 sequence (e.g. to SEQ ID NO: 9) is preferably a sequence containing up to 2 (e.g. only 1 or 2), preferably only 1, amino acid substitutions as compared to the given CDR sequence.

In all aspects and embodiments herein a sequence substantially homologous to a given VL CDR3 sequence (e.g. to SEQ ID NO: 10) is preferably a sequence containing up to 4 (e.g. only 1, 2, 3 or 4), preferably up to 3 (e.g. only 1, 2 or 3), more preferably only 1 or 2, more preferably only 1, amino acid substitutions as compared to the given CDR sequence.

Preferably, in all aspects and embodiments, the antigen binding protein (e.g. antibody) of the invention comprises
a VH CDR1 comprising the amino acid sequence of the given VH CDR1 of a specific antigen binding protein (e.g. antibody) of the invention (disclosed in Table A, e.g. SEQ ID NO: 5, or a sequence substantially homologous thereto, wherein said substantially homologous sequence comprises up to 4 (e.g. only 1, 2, 3 or 4) amino acid substitutions as compared to the given VH CDR1 sequence;
a VH CDR2 comprising the amino acid sequence of the given VH CDR2 of a (preferably said) specific antigen binding protein (e.g. antibody) of the invention (disclosed in Table A, e.g. SEQ ID NO: 6);
a VH CDR3 comprising the amino acid sequence of the given VH CDR3 of a (preferably said) specific antigen binding protein (e.g. antibody) of the invention (disclosed in Table A, e.g. SEQ ID NO: 7), or a sequence substantially homologous thereto, wherein said substantially homologous sequence comprises only 1 amino acid substitution as compared to the given VH CDR3 sequence;
a VL CDR1 comprising the specific amino acid sequence of the given VL CDR1 of a (preferably said) specific antigen binding protein (e.g. antibody) of the invention (disclosed in Table A, e.g. SEQ ID NO: 8), or a sequence substantially homologous thereto, wherein said substantially homologous sequence comprises up to 4 (e.g. only 1, 2, 3, or 4), preferably up to 3 (e.g. only 1, 2, or 3) amino acid substitutions as compared to the given VL CDR1 sequence;
a VL CDR2 comprising the specific amino acid sequence of the given VL CDR2 of a (preferably said) specific antigen binding protein (e.g. antibody) of the invention (disclosed in Table A, e.g. SEQ ID NO: 9), or a sequence substantially homologous thereto, wherein said substantially homologous sequence comprises up to 2 (e.g. only 1 or 2) amino acid substitutions as compared to the given VL CDR2 sequence; and
a VL CDR3 comprising the specific amino acid sequence of the given VL CDR3 of a (preferably said) specific antigen binding protein (e.g. antibody) of the invention (disclosed in Table A, e.g. SEQ ID NO: 10), or a sequence substantially homologous thereto, wherein said substantially homologous sequence comprises up to 3 (e.g. only 1, 2 or 3), preferably only 1, amino acid substitutions as compared to the given VL CDR3 sequence.

In a substantially homologous antigen binding protein (e.g. antibody) of the invention, each (i.e. all) of the substantially homologous sequences are substantially homologous to a given sequence derived from the same specific antigen binding protein (e.g. antibody) of the invention (disclosed in Table A).

Preferably, the antigen binding protein (e.g. antibody) of the invention comprises
a VH CDR1, a VH CDR2, and a VL CDR2 comprising, respectively, the amino acid sequence of the given VH CDR1, VH CDR2, and VL CDR2 of a specific antigen binding protein (e.g. antibody) of the invention (disclosed in Table A, e.g. SEQ ID NOs: 5, 6 and 9);
a VH CDR3 comprising the amino acid sequence of the given VH CDR3 of a (preferably said) specific antigen binding protein (e.g. antibody) of the invention (disclosed in Table A, e.g. SEQ ID NO: 7), or a sequence substantially homologous thereto, wherein said substantially homologous sequence comprises only 1 amino acid substitution as compared to the given VH CDR3 sequence;
a VL CDR1 comprising the specific amino acid sequence of the given VL CDR1 of a (preferably said) specific antigen binding protein (e.g. antibody) of the invention (disclosed in Table A, e.g. SEQ ID NO: 8), or a sequence substantially homologous thereto, wherein said substantially homologous sequence comprises up to 3 (e.g. only 1, 2, or 3) amino acid substitutions as compared to the given VL CDR1 sequence; and
a VL CDR3 comprising the amino acid sequence of the given VL CDR3 of a (preferably said) specific antigen binding protein (e.g. antibody) of the invention (disclosed in Table A, e.g. SEQ ID NO: 10), or a sequence substantially homologous thereto, wherein said substantially homologous sequence comprises only 1 amino acid substitution compared to the given VL CDR3 sequence.

In a substantially homologous antigen binding protein (e.g. antibody) of the invention, each (i.e. all) of the substantially homologous sequences are substantially homologous to a given sequence derived from the same specific antigen binding protein (e.g. antibody) of the invention (disclosed in Table A).

In some embodiments, in an antigen binding protein (e.g. antibody) having a “substantially homologous” sequence as compared to a given sequence, or having a certain degree of sequence identity as compared to a given sequence, the three VH CDR amino acid sequences (i.e. all three VH CDR sequences taken together) and the three VL CDR amino acid sequences (i.e. all three VL CDR sequences taken together), to make up a set of six CDRs in total, are considered together to be the whole (or entire) CDR complement of the antigen binding protein/antibody, and the amino acid sequence of said whole CDR complement of said antigen binding protein/antibody is at least 70%, preferably at least 80%, or at least 85%, or at least 90%, or at least 95% identical to the corresponding whole (or entire) CDR complement of a given starting (or reference) antigen binding protein/antibody. The starting (or reference) antigen binding protein (e.g. antibody) may have the CDR sequences of a specific antibody of the present invention shown in Table A, e.g. the 1-H02 antibody.

Preferred substantially homologous antigen binding proteins (e.g. antibodies) contain up to 13, e.g. only 1, 2, 3, 4, 5, 6, 7, 8, 9, 10, 11, 12 or 13, preferably up to 9, e.g. only 1, 2, 3, 4, 5, 6, 7, 8 or 9, preferably up to 6, e.g. only 1, 2, 3, 4, 5 or 6, preferably up to 5, for example only 1, 2, 3, 4 or 5, preferably up to 4, for example only 1, 2, 3 or 4, preferably up to 3, e.g. only 1, 2 or 3, more preferably only 1 or 2, more preferably only 1 altered amino acids (preferably substitutions), in the whole CDR complement as compared to in the whole CDR complement of a specific antigen binding protein (e.g. antibody) of the invention (disclosed in Table A, e.g. 1-H02). Preferably, the antigen binding protein (e.g. antibody) contains up to 6, e.g. only 1, 2, 3, 4, 5 or 6 altered amino acids (preferably substitutions) as compared to the whole complement of the given CDR sequences.

At the amino acid level, preferred substantially homologous antigen binding proteins (e.g. antibodies) contain up to 8, e.g. only 1, 2, 3, 4, 5, 6, 7, or 8, preferably up to 6, e.g. only 1, 2, 3, 4, 5 or 6, preferably up to 5, for example only 1, 2, 3, 4 or 5, preferably up to 4, for example only 1, 2, 3 or 4, preferably up to 3, e.g. only 1, 2 or 3, more preferably only 1 or 2, more preferably only 1 altered amino acids, in the combined framework regions (e.g. the four framework regions), and/or the combined CDRs (e.g. the three CDR regions) making up the VL domain, or the VH domain, as compared to those of a specific antigen binding protein (e.g. antibody) of the invention (disclosed in Table A).

At the amino acid level, preferred substantially homologous antigen binding proteins (e.g. antibodies) contain up to 5, for example only 1, 2, 3, 4 or 5, preferably up to 4, e.g. only 1, 2, 3 or 4, preferably up to 3, e.g. only 1, 2 or 3, more preferably only 1 or 2, more preferably only 1 altered amino acids, more preferably no amino altered amino acids, in the combined framework regions (e.g. the four framework regions), and/or the combined CDRs (e.g. the three CDR regions) making up the VH domain as compared to those of a specific antigen binding protein (e.g. antibody) of the invention (disclosed in Table A);
and/or (preferably “and”), contain up to 8, e.g. only 1, 2, 3, 4, 5, 6, 7, or 8, preferably up to 6, e.g. only 1, 2, 3, 4, 5 or 6, preferably up to 5, for example only 1, 2, 3, 4 or 5, preferably up to 4, e.g. only 1, 2, 3 or 4, preferably up to 3, e.g. only 1, 2 or 3, more preferably only 1 or 2, more preferably only 1 altered amino acids, more preferably no altered amino acids, in the combined framework regions (e.g. the four framework regions), and/or the combined CDRs (e.g. the three CDR regions) making up the VL domain of as compared to those of a specific antigen binding protein (e.g. antibody) of the invention (disclosed in Table A).

At the amino acid level, preferred substantially homologous antigen binding proteins (e.g. antibodies) contain up to 5, for example only 1, 2, 3, 4 or 5, preferably up to 4, e.g. only 1, 2, 3 or 4, preferably up to 3, e.g. only 1, 2 or 3, more preferably only 1 or 2, more preferably only 1 altered amino acids, more preferably no amino altered amino acids, in the combined CDRs (e.g. the three CDR regions) making up the VH domain as compared to those of a specific antigen binding protein (e.g. antibody) of the invention (disclosed in Table A, e.g. 1-H02);
and/or (preferably “and”), contain up to 8, e.g. only 1, 2, 3, 4, 5, 6, 7, or 8, preferably up to 6, e.g. only 1, 2, 3, 4, 5 or 6, preferably up to 5, for example only 1, 2, 3, 4 or 5, preferably up to 4, e.g. only 1, 2, 3 or 4, preferably up to 3, e.g. only 1, 2 or 3, more preferably only 1 or 2, more preferably only 1 altered amino acids, more preferably no amino altered amino acids, in the combined CDRs (e.g. the three CDR regions) making up the VL domain as compared to those of a specific antigen binding protein (e.g. antibody) of the invention (disclosed in Table A, e.g. 1-H02).

At the amino acid level, preferred substantially homologous antigen binding proteins (e.g. antibodies) contain up to 4 (e.g. only 1, 2, 3, or 4), preferably only 1 altered amino acid, more preferably no amino altered amino acids, in the combined CDRs (e.g. the three CDR regions) making up the VH domain as compared to those of a specific antigen binding protein (e.g. antibody) of the invention (disclosed in Table A, e.g. 1-H02);

and/or (preferably “and”),
contain up 8 (e.g. only 1, 2, 3, 4, 5, 6, 7, or 8), preferably up to 6 (e.g. only 1, 2, 3, 4, 5, 6), preferably up to 4, e.g. only 1, 2, 3, or 4, preferably up to 3, e.g. only 1, 2 or 3, more preferably only 1 or 2, more preferably only 1 altered amino acids, more preferably no amino altered amino acids, in the combined CDRs (e.g. the three CDR regions) making up the VL domain as compared to those of a specific antigen binding protein (e.g. antibody) of the invention (disclosed in Table A e.g. 1-H02).

At the amino acid level, preferred substantially homologous antigen binding proteins (e.g. antibodies) contain only 1 altered amino acid, more preferably no amino altered amino acids, in the combined CDRs (e.g. the three CDR regions) making up the VH domain as compared to those of a specific antigen binding protein (e.g. antibody) of the invention (disclosed in Table A);

and/or (preferably “and”),
contain up 4, e.g. only 1, 2, 3, or 4, preferably up to 3, e.g. only 1, 2 or 3, more preferably only 1 or 2, more preferably only 1 altered amino acids, more preferably no amino altered amino acids, in the combined CDRs (e.g. the three CDR regions) making up the VL domain as compared to those of a specific antigen binding protein (e.g. antibody) of the invention (disclosed in Table A).

Optionally, the antigen binding protein (e.g. antibody) comprises a VH CDR1, a VH CDR2, and a VH CDR3 comprising, respectively, the amino acid sequence of the given VH CDR1, VH CDR2, and VH CDR3 of a specific antigen binding protein (e.g. antibody) of the invention (disclosed in Table A), i.e. rather than sequences substantially homologous thereto; and/or the antigen binding protein (e.g. antibody) comprises a VL CDR1, a VL CDR2, and a VL CDR3 comprising, respectively, the amino acid sequence of the given VL CDR1, VL CDR2, and VL CDR3 of a specific antigen binding protein (e.g. antibody) of the invention (disclosed in Table A), i.e. rather than sequences substantially homologous thereto. In other words, optionally the variant amino acids lie only in either the VH CDRs, or only in the VL CDRs.

Preferably, the antigen binding proteins (e.g. antibodies) of the invention comprise at least one heavy chain variable domain that includes an amino acid sequence region of at least 95% and most preferably at least 96%, 97%, 98% or 99% amino acid sequence identity to the amino acid sequence of a heavy chain variable domain of a specific antigen binding protein (e.g. antibody) of the invention (disclosed in Table A, e.g. SEQ ID NO: 3),
further wherein
   said heavy chain variable domain comprises a VH CDR1, a VH CDR2, and a VH CDR3 comprising the amino acid sequences of the specific VH CDR1, VH CDR2, and VH CDR3 of said specific antigen binding protein (e.g. antibody) of the invention (shown in Table A, e.g. SEQ ID NOs: 5, 6, and 7), or sequences substantially homologous thereto,
   and/or (preferably “and”)
at least one light chain variable domain that includes an amino acid sequence region of at least 75%, more preferably at least 80%, more preferably at least 90% amino acid sequence identity to the amino acid sequence of a specific light chain variable domain of said specific antigen binding protein (e.g. antibody) of the invention (shown in Table A, e.g. SEQ ID NO: 4),
   further wherein
   said light chain variable domain comprises a VL CDR1, a VL CDR2, and a VL CDR3, comprising the amino acid sequences of the specific VL CDR1, VL CDR2, and VL CDR3 of said specific antigen binding protein (e.g. antibody) of the invention (shown in Table A, e.g. SEQ ID NOs: 8, 9 and 10), or sequences substantially homologous thereto,
wherein said sequences substantially homologous to a given CDR sequence are as defined anywhere else herein.

In all embodiments, said alterations can be with conservative or non-conservative amino acids. Preferably said alterations are conservative amino acid substitutions.

Altered residues might be conserved or non-conserved amino acid substitutions, or a mixture thereof. In such embodiments, preferred alterations are conservative amino acid substitutions.

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). In other examples, families of amino acid residues can be grouped based on hydrophobic side groups or hydrophilic side groups.

Routine methods in the art such as alanine scanning mutagenesis and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

Once identified, the addition, deletion, substitution or insertion of one or more amino acids in the amino acid sequence of a parent antigen binding protein (e.g. antibody) to form a new antigen binding protein (e.g. antibody), wherein said parent antigen binding protein (e.g. antibody) is one of the antigen binding proteins (e.g. antibodies) of the invention as defined elsewhere herein (e.g. disclosed in Table A), and testing the resulting new antigen binding protein (e.g. antibody) to identify antigen binding proteins (e.g. antibodies) that bind to HLA-A*02:01/MAGE-A4²³⁰⁻²³⁹ in accordance with the invention can be carried out using techniques which are routine in the art. Such methods can be used to form multiple new antigen binding proteins (e.g. antibodies) that can all be tested for their ability to bind HLA-A*02:01/MAGE-A4²³⁰⁻²³⁹. Preferably said addition, deletion, substitution or insertion of one or more amino acids takes place in one or more of the CDR domains.

For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way. Testing the ability of one or more of the modified antigen binding proteins (e.g. antibodies) to bind to HLA-A*02:01/MAGE-A4²³⁰⁻²³⁹ can be carried out by any appropriate method, which are well known and described in the art. Suitable methods are also described elsewhere herein and in the Examples section.

New antigen binding proteins (e.g. antibodies) produced, obtained or obtainable by these methods form a yet further aspect of the invention.

The term “substantially homologous” also includes modifications or chemical equivalents of the amino acid and nucleotide sequences of the present invention that perform substantially the same function as the proteins or nucleic acid molecules of the invention in substantially the same way. For example, any substantially homologous antigen binding protein (e.g. antibody) should retain the ability to bind to the antigen (pMHC antigen) as described herein. Preferably, any substantially homologous antigen binding protein (e.g. antibody) should retain one or more (or all) of the functional capabilities of the starting antigen binding protein (e.g. antibody).

Substantially homologous sequences of proteins of the invention include, without limitation, conservative amino acid substitutions, or for example alterations that do not affect the VH, VL or CDR domains of the antigen binding proteins (e.g. antibodies), e.g. antigen binding proteins (e.g. antibodies) where tag sequences, toxins or other components are added that do not contribute to the binding of antigen, or alterations to convert one type or format of antibody molecule or fragment to another type or format of antibody molecule or fragment (e.g. conversion from Fab to scFv or whole antibody or vice versa), or the conversion of an antibody molecule to a particular class or subclass of antibody molecule (e.g. the conversion of an antibody molecule to IgG or a subclass thereof, e.g. IgG₂).

Homology or sequence identity may be assessed by any convenient method. However, for determining the degree of homology between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having at least 30%, 40%, 50%, 55%, 60%, 62%, 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

Further examples of substantially homologous amino acid sequences in accordance with the present invention are described elsewhere herein.

In all aspects and embodiments, the antigen binding proteins (e.g. antibodies) of the invention preferably comprise the specific sequences disclosed, and not sequences substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of

(SEQ ID NO: 5)
GYTLTELS;
or
(SEQ ID NO: 81)
GPKLYEVS,

or a sequence substantially homologous thereto,
(b) a VH CDR 2 that comprises the amino acid sequence of

(SEQ ID NO: 6)
FDPEDGET;
(SEQ ID NO: 82)
FDPYMSRT;
(SEQ ID NO: 83)
FDPYLART;
or
(SEQ ID NO: 84)
FDPEQGET,

or a sequence substantially homologous thereto, and
(c) a VH CDR 3 that comprises the amino acid sequence of

(SEQ ID NO: 7)
ATDQGSSWGFY;
(SEQ ID NO: 85)
ATDQGASWGFY;
or
(SEQ ID NO: 86)
AADQGSSWGFY,

or a sequence substantially homologous thereto;
   and/or (preferably “and”) wherein
said light chain variable domain comprises:
   (d) a variable light (VL) CDR1 that comprises the amino acid sequence of

(SEQ ID NO: 8)
QSISSY;
(SEQ ID NO: 87)
QNIMWY;
(SEQ ID NO: 88)
VHIYWY;
(SEQ ID NO: 89)
HHIFWY;
(SEQ ID NO: 90)
IDIRWY;
(SEQ ID NO: 91)
QSIMTY;
(SEQ ID NO: 92)
QTVATY;
or
(SEQ ID NO: 93)
EDIRYY,

or a sequence substantially homologous thereto,
(e) a variable light VL CDR2 that comprises the amino acid sequence of

(SEQ ID NO: 9)
AAS;
(SEQ ID NO: 94)
SAS;
or
(SEQ ID NO: 95)
VTS,

or a sequence substantially homologous thereto, and
(f) a VL CDR3 that comprises the amino acid sequence of

(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of

(SEQ ID NO: 5)
GYTLTELS;
or
(SEQ ID NO: 81)
GPKLYEVS,

or a sequence substantially homologous thereto;
preferably GYTLTELS (SEQ ID NO: 5); or a sequence substantially homologous thereto,
(b) a VH CDR 2 that comprises the amino acid sequence of

(SEQ ID NO: 6)
FDPEDGET;

or a sequence substantially homologous thereto, and
(c) a VH CDR 3 that comprises the amino acid sequence of

(SEQ ID NO: 7)
ATDQGSSWGFY;
(SEQ ID NO: 85)
ATDQGASWGFY;

or a sequence substantially homologous thereto;
   and/or (preferably “and”) wherein
said light chain variable domain comprises:
   (d) a variable light (VL) CDR1 that comprises the amino acid sequence of

(SEQ ID NO: 8);
QSISSY
(SEQ ID NO: 87)
QNIMWY;
(SEQ ID NO: 88)
VHIYWY;
(SEQ ID NO: 89)
HHIFWY;
(SEQ ID NO: 90)
IDIRWY;
(SEQ ID NO: 91)
QSIMTY;
(SEQ ID NO: 92)
QTVATY;
or
(SEQ ID NO: 93)
EDIRYY,

or a sequence substantially homologous thereto,
(e) a variable light VL CDR2 that comprises the amino acid sequence of

(SEQ ID NO: 9)
AAS;
(SEQ ID NO: 94)
SAS;
or
(SEQ ID NO: 95)
VTS,

or a sequence substantially homologous thereto, and
(f) a VL CDR3 that comprises the amino acid sequence of

(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of

(SEQ ID NO: 5)
GYTLTELS;
or
(SEQ ID NO: 81)
GPKLYEVS,

or a sequence substantially homologous thereto,
(b) a VH CDR 2 that comprises the amino acid sequence of

(SEQ ID NO: 6)
FDPEDGET;
(SEQ ID NO: 82)
FDPYMSRT;
(SEQ ID NO: 83)
FDPYLART;
or
(SEQ ID NO: 84)
FDPEQGET,

or a sequence substantially homologous thereto, and
(c) a VH CDR 3 that comprises the amino acid sequence of

(SEQ ID NO: 7)
ATDQGSSWGFY;
(SEQ ID NO: 85)
ATDQGASWGFY;
or
(SEQ ID NO: 86)
AADQGSSWGFY,

or a sequence substantially homologous thereto;
   and/or (preferably “and”) wherein
said light chain variable domain comprises:
   (d) a variable light (VL) CDR1 that comprises the amino acid sequence of

(SEQ ID NO: 8)
QSISSY;
(SEQ ID NO: 87)
QNIMWY;
or
(SEQ ID NO: 92)
QTVATY,

or a sequence substantially homologous thereto,
(e) a variable light VL CDR2 that comprises the amino acid sequence of

(SEQ ID NO: 9)
AAS;
or
(SEQ ID NO: 95)
VTS,

or a sequence substantially homologous thereto, and
(f) a VL CDR3 that comprises the amino acid sequence of

(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of

(SEQ ID NO: 5)
GYTLTELS;
or
(SEQ ID NO: 81)
GPKLYEVS,

or a sequence substantially homologous thereto,
(b) a VH CDR 2 that comprises the amino acid sequence of

(SEQ ID NO: 6)
FDPEDGET;
(SEQ ID NO: 82)
FDPYMSRT;
(SEQ ID NO: 83)
FDPYLART;
or
(SEQ ID NO: 84)
FDPEQGET,

or a sequence substantially homologous thereto, and
(c) a VH CDR 3 that comprises the amino acid sequence of

(SEQ ID NO: 7)
ATDQGSSWGFY;
(SEQ ID NO: 85)
ATDQGASWGFY;
or
(SEQ ID NO: 86)
AADQGSSWGFY,

or a sequence substantially homologous thereto;
   and/or (preferably “and”) wherein
said light chain variable domain comprises:
   (d) a variable light (VL) CDR1 that comprises the amino acid sequence of

(SEQ ID NO: 8)
QSISSY;
or
(SEQ ID NO: 87)
QNIMWY;

or a sequence substantially homologous thereto,
(e) a variable light VL CDR2 that comprises the amino acid sequence of

(SEQ ID NO: 9)
AAS;

or a sequence substantially homologous thereto, and
(f) a VL CDR3 that comprises the amino acid sequence of

(SEQ ID NO: 10)
QQSYSTPYT;
(SEQ ID NO: 96)
QQSYSTPFT;

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of

(SEQ ID NO: 5)
GYTLTELS;
or
(SEQ ID NO: 81)
GPKLYEVS,

or a sequence substantially homologous thereto,
(b) a VH CDR 2 that comprises the amino acid sequence of

(SEQ ID NO: 6)
FDPEDGET,

or a sequence substantially homologous thereto, and
(c) a VH CDR 3 that comprises the amino acid sequence of

(SEQ ID NO: 7)
ATDQGSSWGFY;
or
(SEQ ID NO: 85)
ATDQGASWGFY,

or a sequence substantially homologous thereto;
   and/or (preferably “and”) wherein
said light chain variable domain comprises:
   (d) a variable light (VL) CDR1 that comprises the amino acid sequence of

(SEQ ID NO: 8)
QSISSY;
(SEQ ID NO: 87)
QNIMWY;
or
(SEQ ID NO: 92)
QTVATY,

or a sequence substantially homologous thereto,
(e) a variable light VL CDR2 that comprises the amino acid sequence of

(SEQ ID NO: 9)
AAS;
or
(SEQ ID NO: 95)
VTS,

or a sequence substantially homologous thereto, and
(f) a VL CDR3 that comprises the amino acid sequence of

(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO: 5) or a sequence substantially homologous thereto,
   (b) a VH CDR 2 that comprises the amino acid sequence of FDPEDGET (SEQ ID NO:6) or a sequence substantially homologous thereto, and
   (c) a VH CDR 3 that comprises the amino acid sequence of

(SEQ ID NO: 7)
ATDQGSSWGFY;
or
(SEQ ID NO: 85)
ATDQGASWGFY,

or a sequence substantially homologous thereto;
   and/or (preferably “and”) wherein
said light chain variable domain comprises:
   (d) a variable light (VL) CDR1 that comprises the amino acid sequence of

(SEQ ID NO: 8)
QSISSY;
or
(SEQ ID NO: 87)
QNIMWY,

or a sequence substantially homologous thereto,
(e) a variable light VL CDR2 that comprises the amino acid sequence of AAS (SEQ ID NO:9), or a sequence substantially homologous thereto, and
(f) a VL CDR3 that comprises the amino acid sequence of

(SEQ ID NO: 10)
QQSYSTPYT;
or
(SEQ ID NO: 96)
QQSYSTPFT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO: 5), or a sequence substantially homologous thereto,
   (b) a VH CDR 2 that comprises the amino acid sequence of FDPEDGET (SEQ ID NO:6), or a sequence substantially homologous thereto, and
   (c) a VH CDR 3 that comprises the amino acid sequence of ATDQGSSWGFY (SEQ ID NO:7), or a sequence substantially homologous thereto;
      and/or (preferably “and”) wherein
said light chain variable domain comprises:
   (d) a variable light (VL) CDR1 that comprises the amino acid sequence of

(SEQ ID NO: 8)
QSISSY;
(SEQ ID NO: 87)
QNIMWY;
(SEQ ID NO: 88)
VHIYWY;
(SEQ ID NO: 89)
HHIFWY;
(SEQ ID NO: 90)
IDIRWY;
(SEQ ID NO: 91)
QSIMTY;
(SEQ ID NO: 92)
QTVATY;
or
(SEQ ID NO: 93)
EDIRYY,

or a sequence substantially homologous thereto,
(e) a variable light VL CDR2 that comprises the amino acid sequence of

(SEQ ID NO: 9)
AAS;
(SEQ ID NO: 94)
SAS;
or
(SEQ ID NO: 95)
VTS,

or a sequence substantially homologous thereto, and
(f) a VL CDR3 that comprises the amino acid sequence of

(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO: 5) or a sequence substantially homologous thereto,
   (b) a VH CDR 2 that comprises the amino acid sequence of FDPEDGET (SEQ ID NO:6) or a sequence substantially homologous thereto, and
   (c) a VH CDR 3 that comprises the amino acid sequence of

(SEQ ID NO: 7)
ATDQGSSWGFY;

or a sequence substantially homologous thereto;
   and/or (preferably “and”) wherein
said light chain variable domain comprises:
   (d) a variable light (VL) CDR1 that comprises the amino acid sequence of

(SEQ ID NO: 8)
QSISSY

or a sequence substantially homologous thereto,
(e) a variable light VL CDR2 that comprises the amino acid sequence of AAS (SEQ ID NO:9), or a sequence substantially homologous thereto, and
(f) a VL CDR3 that comprises the amino acid sequence of

(SEQ ID NO: 10)
QQSYSTPYT;
or
(SEQ ID NO: 96)
QQSYSTPFT,

or a sequence substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises a VH CDR1, a VH CDR2 and a VH CDR3 comprising the following amino acid sequences or sequences substantially homologous thereto:

| | VH CDR1 | VH CDR2 | VH CDR3 |
|---|---|---|---|
| a) | GYTLT | FDPED | ATDQG |
| | ELS | GET | SSWGF |
| | (SEQ ID | (SEQ ID | Y; or |
| | NO: 5) | NO: 6) | (SEQ ID |
| | | | NO: 7) |
| b) | GYTLT | FDPED | ATDQG |
| | ELS | GET | ASWGF |
| | (SEQ ID | (SEQ ID | Y; or |
| | NO: 5) | NO: 6) | (SEQ ID |
| | | | NO: 85) |
| c) | GYTLT | FDPED | AADQG |
| | ELS | GET | SSWGF |
| | (SEQ ID | (SEQ ID | Y; or |
| | NO: 5) | NO: 6) | (SEQ ID |
| | | | NO: 86) |
| d) | GPKLY | FDPED | ATDQG |
| | EVS | GET | SSWGF |
| | (SEQ ID | (SEQ ID | Y |
| | NO: 81) | NO: 6) | (SEQ ID |
| | | | NO: 7); or |
| e) | GYTLT | FDPYM | ATDQG |
| | ELS | SRT | SSWGF |
| | (SEQ ID | (SEQ ID | Y |
| | NO: 5) | NO: 82) | (SEQ ID |
| | | | NO: 7); or |
| f) | GYTLT | FDPYL | ATDQG |
| | ELS | ART | SSWGF |
| | (SEQ ID | (SEQ ID | Y |
| | NO: 5) | NO: 83) | (SEQ ID |
| | | | NO: 7); or |
| g) | GYTLT | FDPEQ | ATDQG |
| | ELS | GET | ASWGF |
| | (SEQ ID | (SEQ ID | Y |
| | NO: 5) | NO: 84) | (SEQ ID |
| | | | NO: 85); or |
| h) | GYTLT | FDPYL | ATDQG |
| | ELS | ART | ASWGF |
| | (SEQ ID | (SEQ ID | Y |
| | NO: 5) | NO: 83) | (SEQ ID |
| | | | NO: 85) |
| i) | GPKLY | FDPED | ATDQG |
| | EVS | GET | ASWGF |
| | (SEQ ID | (SEQ ID | Y |
| | NO: 81) | NO: 6) | (SEQ ID |
| | | | NO: 85) |

and/or (preferably “and”) a light chain variable domain (VL domain) that comprises a VL CDR1, a VL CDR2 and a VL CDR3 comprising the following amino acid sequences or sequences substantially homologous thereto:

| | VL CDR1 | VL CDR2 | VL CDR3 |
|---|---|---|---|
| a) | QSISS | AAS | QQSYS |
| | Y | (SEQ ID | TPYT; or |
| | (SEQ ID | NO: 9) | (SEQ ID |
| | NO: 8) | | NO: 10) |
| b) | QSISS | AAS | QQSYS |
| | Y | (SEQ ID | TPFT; or |
| | (SEQ ID | NO: 9) | (SEQ ID |
| | NO: 8) | | NO: 96) |
| c) | QNIMW | AAS | QQSYS |
| | Y | (SEQ ID | TPYT; or |
| | (SEQ ID | NO: 9) | (SEQ ID |
| | NO: 87) | AAS | NO: 10) |
| d) | VHIYW | AAS | QQSYS |
| | Y | (SEQ ID | TPYT; or |
| | (SEQ ID | NO: 9) | (SEQ ID |
| | NO: 88) | AAS | NO: 10) |
| e) | HHIFW | AAS | QQSYS |
| | Y | (SEQ ID | TPYT; or |
| | (SEQ ID | NO: 9) | (SEQ ID |
| | NO: 89) | AAS | NO: 10) |
| f) | IDIRW | AAS | QQSYS |
| | Y | (SEQ ID | TPYT; or |
| | (SEQ ID | NO: 9) | (SEQ ID |
| | NO: 90) | AAS | NO: 10) |
| g) | QSISS | AAS | QQAYR |
| | Y | (SEQ ID | IPYT; or |
| | (SEQ ID | NO: 9) | (SEQ ID |
| | NO: 8) | AAS | NO: 97) |
| h) | QSISS | AAS | QQSYS |
| | Y | (SEQ ID | TPYT; or |
| | (SEQ ID | NO: 9) | (SEQ ID |
| | NO: 8) | AAS | NO: 10) |
| i) | IDIRW | AAS | QQSYS |
| | Y | (SEQ ID | TPFT; or |
| | (SEQ ID | NO: 9) | (SEQ ID |
| | NO: 90) | | NO: 96) |
| j) | QSIMT | SAS | QQSYS |
| | Y | (SEQ ID | TPFT; or |
| | (SEQ ID | NO: 94) | (SEQ ID |
| | NO: 91) | | NO: 96) |
| k) | QTVAT | VTS | QQAYS |
| | Y | (SEQ ID | TPVT; or |
| | (SEQ ID | NO: 95) | (SEQ ID |
| | NO: 92) | | NO: 98) |
| l) | EDIRY | AAS | QQSYS |
| | Y | (SEQ ID | TPYT; or |
| | (SEQ ID | NO: 9) | (SEQ ID |
| | NO: 93) | | NO: 10) |
| m) | QNIMW | AAS | QQSYS |
| | Y | (SEQ ID | TPFT; or |
| | (SEQ ID | NO: 9) | (SEQ ID |
| | NO: 87) | AAS | NO: 96) |
| n) | QNIMW | AAS | QQAYR |
| | Y | (SEQ ID | IPYT. |
| | (SEQ ID | NO: 9) | (SEQ ID |
| | NO: 87) | AAS | NO: 97) |

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises a VH CDR1, a VH CDR2 and a VH CDR3 comprising the following amino acid sequences or sequences substantially homologous thereto:

| | VH CDR1 | VH CDR2 | VH CDR3 |
|---|---|---|---|
| a) | GYTLT | FDPED | ATDQG |
| | ELS | GET | SSWGF |
| | (SEQ ID | (SEQ ID | Y; or |
| | NO: 5) | NO: 6) | (SEQ ID |
| | | | NO: 7) |
| b) | GYTLT | FDPED | ATDQG |
| | ELS | GET | ASWGF |
| | (SEQ ID | (SEQ ID | Y; or |
| | NO: 5) | NO: 6) | (SEQ ID |
| | | | NO: 85) |
| c) | GPKLY | FDPED | ATDQG |
| | EVS | GET | SSWGF |
| | (SEQ ID | (SEQ ID | Y |
| | NO: 81) | NO: 6) | (SEQ ID |
| | | | NO: 7) |

and/or (preferably “and”) a light chain variable domain (VL domain) that comprises a VL CDR1, a VL CDR2 and a VL CDR3 comprising the following amino acid sequences or sequences substantially homologous thereto:

| | VL CDR1 | VL CDR2 | VL CDR3 |
|---|---|---|---|
| a) | QSISS | AAS | QQSYS |
| | Y | (SEQ ID | TPYT; or |
| | (SEQ ID | NO: 9) | (SEQ ID |
| | NO: 8) | | NO: 10) |
| b) | QSISS | AAS | QQSYS |
| | Y | (SEQ ID | TPFT; or |
| | (SEQ ID | NO: 9) | (SEQ ID |
| | NO: 8) | | NO: 96) |
| c) | QNIMW | AAS | QQSYS |
| | Y | (SEQ ID | TPYT; or |
| | (SEQ ID | NO: 9) | (SEQ ID |
| | NO: 87) | | NO: 10) |
| d) | QTVAT | VTS | QQAYS |
| | Y | (SEQ ID | TPVT; or |
| | (SEQ ID | NO: 95) | (SEQ ID |
| | NO: 92) | | NO: 98) |
| e) | QNIMW | AAS | QQAYR |
| | Y | (SEQ ID | IPYT. |
| | (SEQ ID | NO: 9) | (SEQ ID |
| | NO: 87) | | NO: 97) |

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises a VH CDR1, a VH CDR2 and a VH CDR3 comprising the following amino acid sequences or sequences substantially homologous thereto:

| | VH CDR1 | VH CDR2 | VH CDR3 |
|---|---|---|---|
| a) | GYTLT | FDPED | ATDQG |
| | ELS | GET | SSWGF |
| | (SEQ ID | (SEQ ID | Y; or |
| | NO: 5) | NO: 6) | (SEQ ID |
| | | | NO: 7) |
| b) | GYTLT | FDPED | ATDQG |
| | ELS | GET | ASWGF |
| | (SEQ ID | (SEQ ID | Y |
| | NO: 5) | NO: 6) | (SEQ ID |
| | | | NO: 85) |

and/or (preferably “and”) a light chain variable domain (VL domain) that comprises a VL CDR1, a VL CDR2 and a VL CDR3 comprising the following amino acid sequences or sequences substantially homologous thereto:

| | VL CDR1 | VL CDR2 | VL CDR3 |
|---|---|---|---|
| a) | QSISS | AAS | QQSYS |
| | Y | (SEQ ID | TPYT; |
| | (SEQ ID | NO: 9) | or |
| | NO: 8) | | (SEQ ID |
| | | | NO: 10) |
| b) | QSISS | AAS | QQSYS |
| | Y | (SEQ ID | TPFT; |
| | (SEQ ID | NO: 9) | or |
| | NO: 8) | | (SEQ ID |
| | | | NO: 96) |
| c) | QNIMW | AAS | QQSYS |
| | Y | (SEQ ID | TPYT. |
| | (SEQ ID | NO: 9) | (SEQ ID |
| | NO: 87) | | NO: 10) |

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises a variable heavy (VH) CDR1, a VH CDR2 and a VH CDR3 that comprise, respectively, the amino acid sequences of the VH CDR1, VH CDR2 and VH CDR3 of a specific antibody of the invention (disclosed in Table A), or sequences substantially homologous thereto;
   and/or (preferably “and”) wherein
said light chain variable domain comprises a variable light (VL) CDR1, a VL CDR2 and a VL CDR3 that comprise, respectively, the amino acid sequences of the VL CDR1, VL CDR2 and VL CDR3 of a (preferably said) specific antibody of the invention (disclosed in Table A), or sequences substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of the VH domain of a specific antibody of the invention (disclosed in Table A) or a sequence substantially homologous thereto, and/or (preferably “and”) a VL domain that comprises the amino acid sequence of the VL domain of a (preferably said) specific antibody of the invention (disclosed in Table A).

In all aspects and embodiments, the specific antibody of the invention (disclosed in Table A) may be the 1-H02 antibody, the AM15 antibody, the AM2 antibody, the AMC9 antibody, the AM1 antibody, the AM3 antibody, the AM4 antibody, the AM5 antibody, the AM6 antibody, the AM7 antibody, the AM9 antibody, the AM10 antibody, the AM11 antibody, the AM12 antibody, the AM13 antibody, the AM14 antibody, the AM16 antibody, the AM17 antibody, the AM18 antibody, the AMC1 antibody, the AMC3 antibody, the AMC4 antibody, the AMC5 antibody, the AMC6 antibody, the AMC7 antibody, the AMC8 antibody, the AMC10 antibody, the AMC11 antibody, the AMC12 antibody, or the AMC14 antibody. Preferably, the specific antibody is the 1-H02 antibody, the AM15 antibody, the AM2 antibody, the AMC9 antibody, the AM6 antibody, the AMC8 antibody, or the AMC11 antibody. More preferably, the specific antibody is the 1-H02 antibody, the AM15 antibody, the AM2 antibody, or the AMC9 antibody, still more preferably the 1-H02 antibody or the AM15 antibody.

Alternatively viewed, in another aspect and in certain embodiments, the present invention provides an antigen binding protein (e.g. antibody) that binds to, or specifically binds to, HLA-A*02:01/MAGE-A4²³⁰⁻²³⁹, and comprises
i) the three VH CDR sequences of a specific antibody of the invention (disclosed in Table A) and/or (preferably “and”) the three VL CDR sequences of said specific antibody, or sequences substantially homologous thereto;
and/or (preferably “and”)
ii) the VH domain sequence and/or (preferably “and”) the VL domain sequence) of said specific antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the 1-H02 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM15 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM2 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC9 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM1 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM3 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM4 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM5 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM6 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM7 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM9 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM10 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM11 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM12 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM13 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM14 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM16 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM17 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AM18 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC1 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC3 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC4 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC5 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC6 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC7 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC8 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC10 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC11 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC12 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein (e.g. antibody) that binds to, or specifically binds to, HLA-A*02:01/MAGE-A4²³⁰⁻²³⁹, and comprises i) the three VH CDR sequences of the AMC14 antibody (disclosed in Table A) and/or (preferably “and”) the three VL CDR sequences of said antibody, or sequences substantially homologous thereto; and/or (preferably “and”) ii) the VH domain sequence and/or (preferably “and”) the VL domain sequence) of said antibody, or sequences substantially homologous thereto.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO:5) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGSSWGFY (SEQ ID NO:7) or a sequence substantially homologous thereto;
      and/or (preferably “and”) wherein
said light chain variable domain comprises:
   (d) a variable light (VL) CDR1 that comprises the amino acid sequence of QSISSY (SEQ ID NO:8) or a sequence substantially homologous thereto,
   (e) a VL CDR2 that comprises the amino acid sequence of AAS (SEQ ID NO:9) or a sequence substantially homologous thereto, and
   (f) a VL CDR3 that comprises the amino acid sequence of QQSYSTPYT (SEQ ID NO:10) or a sequence substantially homologous thereto,
      wherein each of said substantially homologous sequences is separately a sequence containing 1, 2, 3 or 4, (preferably 1, 2 or 3) amino acid substitutions compared to the given CDR sequence.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO:5) or a sequence substantially homologous thereto, wherein said substantially homologous sequence is a sequence containing 1, 2, 3 or 4, (preferably 1, 2 or 3) amino acid substitutions compared to the given CDR sequence,
   (b) a VH CDR2 that comprises the amino acid sequence of FDPEDGET (SEQ ID NO:6) or a sequence substantially homologous thereto, wherein said substantially homologous sequence is a sequence containing 1, 2, 3 or 4, (preferably 1, 2 or 3) amino acid substitutions compared to the given CDR sequence, and
   (c) a VH CDR3 that comprises the amino acid sequence of ATDQGSSWGFY (SEQ ID NO:7) or a sequence substantially homologous thereto, wherein said substantially homologous sequence is a sequence containing 1, 2, 3 or 4 (preferably 1) amino acid substitutions compared to the given CDR sequence;
      and/or (preferably “and”) wherein
said light chain variable domain comprises:
   (d) a variable light (VL) CDR1 that comprises the amino acid sequence of QSISSY (SEQ ID NO:8) or a sequence substantially homologous thereto, wherein said substantially homologous sequence is a sequence containing 1, 2, 3 or 4 (preferably 1, 2, or 3, more preferably 1 or 2) amino acid substitutions compared to the given CDR sequence,
   (e) a VL CDR2 that comprises the amino acid sequence of AAS (SEQ ID NO:9) or a sequence substantially homologous thereto, wherein said substantially homologous sequence is a sequence containing 1 or 2 (preferably 1) amino acid substitutions compared to the given CDR sequence, and
   (f) a VL CDR3 that comprises the amino acid sequence of QQSYSTPYT (SEQ ID NO:10) or a sequence substantially homologous thereto, wherein said substantially homologous sequence is a sequence containing 1, 2 or 3 (preferably 1 or 2, more preferably 1) amino acid substitutions compared to the given CDR sequence.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain that comprises three complementarity determining regions (CDRs), and a light chain variable domain that comprises three CDRs,
wherein said heavy chain variable domain comprises:
   (a) a variable heavy (VH) CDR1 that comprises the amino acid sequence of GYTLTELS (SEQ ID NO:5),
   (b) a VH CDR2 that comprises the amino acid sequence of FDPEDGET (SEQ ID NO:6), and
   (c) a VH CDR3 that comprises the amino acid sequence of ATDQGSSWGFY (SEQ ID NO:7);
      and/or (preferably “and”)
wherein said light chain variable domain comprises:
   (d) a variable light (VL) CDR1 that comprises the amino acid sequence of QSISSY (SEQ ID NO:8),
   (e) a VL CDR2 that comprises the amino acid sequence of AAS (SEQ ID NO:9), and
   (f) a VL CDR3 that comprises the amino acid sequence of QQSYSTPYT (SEQ ID NO:10).

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain that comprises three complementarity determining regions (CDRs), and a light chain variable domain that comprises three CDRs,
wherein said heavy chain variable domain comprises:
   (a) a variable heavy (VH) CDR1 that comprises the amino acid sequence of GYTLTELS (SEQ ID NO:5),
   (b) a VH CDR2 that comprises the amino acid sequence of FDPEDGET (SEQ ID NO:6), and
   (c) a VH CDR3 that comprises the amino acid sequence of ATDQGSSWGFY (SEQ ID NO:7);
      and wherein said light chain variable domain comprises:
(d) a variable light (VL) CDR1 that comprises the amino acid sequence of QSISSY (SEQ ID NO:8),
(e) a VL CDR2 that comprises the amino acid sequence of AAS (SEQ ID NO:9), and
(f) a VL CDR3 that comprises the amino acid sequence of QQSYSTPYT (SEQ ID NO:10).

A2 In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO:5) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGSSWGFY (SEQ ID NO:7) or a sequence substantially homologous thereto;
      and/or (preferably “and”) wherein
said light chain variable domain comprises:
   (d) a variable light (VL) CDR1 that comprises the amino acid sequence of QSISSY (SEQ ID NO:8) or a sequence substantially homologous thereto,
   (e) a VL CDR2 that comprises the amino acid sequence of AAS (SEQ ID NO:9) or a sequence substantially homologous thereto, and
   (f) a VL CDR3 that comprises the amino acid sequence of QQSYSTPFT (SEQ ID NO:96) or a sequence substantially homologous thereto.

A3 In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO:5) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGSSWGFY (SEQ ID NO:7) or a sequence substantially homologous thereto;
      and/or (preferably “and”) wherein
said light chain variable domain comprises:
   (d) a variable light (VL) CDR1 that comprises the amino acid sequence of QNIMWY (SEQ ID NO:87) or a sequence substantially homologous thereto,
   (e) a VL CDR2 that comprises the amino acid sequence of AAS (SEQ ID NO:9) or a sequence substantially homologous thereto, and
   (f) a VL CDR3 that comprises the amino acid sequence of QQSYSTPYT (SEQ ID NO:10) or a sequence substantially homologous thereto.

A4 In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO:5) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGASWGFY (SEQ ID NO:85) or a sequence substantially homologous thereto;
      and/or (preferably “and”) wherein
said light chain variable domain comprises:
   (d) a variable light (VL) CDR1 that comprises the amino acid sequence of QNIMWY (SEQ ID NO:87) or a sequence substantially homologous thereto,
   (e) a VL CDR2 that comprises the amino acid sequence of AAS (SEQ ID NO:9) or a sequence substantially homologous thereto, and
   (f) a VL CDR3 that comprises the amino acid sequence of QQSYSTPYT (SEQ ID NO:10) or a sequence substantially homologous thereto.

A5 In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GPKLYEVS (SEQ ID NO:81) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGSSWGFY (SEQ ID NO:7) or a sequence substantially homologous thereto;
      and/or (preferably “and”) wherein
said light chain variable domain comprises:
   (d) a variable light (VL) CDR1 that comprises the amino acid sequence of QSISSY (SEQ ID NO:8) or a sequence substantially homologous thereto,
   (e) a VL CDR2 that comprises the amino acid sequence of AAS (SEQ ID NO:9) or a sequence substantially homologous thereto, and
   (f) a VL CDR3 that comprises the amino acid sequence of QQSYSTPYT (SEQ ID NO:10) or a sequence substantially homologous thereto.

A6 In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that
comprises three CDRs, wherein said heavy chain variable domain comprises:
   (a) a variable heavy (VH) CDR1 that comprises the amino acid sequence of GYTLTELS (SEQ ID NO:5) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGASWGFY (SEQ ID NO:85) or a sequence substantially homologous thereto;
      and/or (preferably “and”) wherein
said light chain variable domain comprises:
   (d) a variable light (VL) CDR1 that comprises the amino acid sequence of QTVATY (SEQ ID NO:92) or a sequence substantially homologous thereto,
   (e) a VL CDR2 that comprises the amino acid sequence of VTS (SEQ ID NO:95) or a sequence substantially homologous thereto, and
   (f) a VL CDR3 that comprises the amino acid sequence of QQAYSTPVT (SEQ ID NO:98) or a sequence substantially homologous thereto.

A7 In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
   (a) a variable heavy (VH) CDR1 that comprises the amino acid sequence of GYTLTELS (SEQ ID NO:5) or a sequence substantially homologous thereto,
   (b) a VH CDR2 that comprises the amino acid sequence of FDPEDGET (SEQ ID NO:6) or a sequence substantially homologous thereto, and
   (c) a VH CDR3 that comprises the amino acid sequence of ATDQGSSWGFY (SEQ ID NO:7) or a sequence substantially homologous thereto;
      and/or (preferably “and”) wherein
said light chain variable domain comprises:
   (d) a variable light (VL) CDR1 that comprises the amino acid sequence of QNIMWY (SEQ ID NO:87) or a sequence substantially homologous thereto,
   (e) a VL CDR2 that comprises the amino acid sequence of AAS (SEQ ID NO:9) or a sequence substantially homologous thereto, and
   (f) a VL CDR3 that comprises the amino acid sequence of QQAYRIPYT (SEQ ID NO:97) or a sequence substantially homologous thereto.

It is expressly disclosed that in each of the aspects/embodiments described in paragraphs A1, A2, A3, A4, A5, A6 and A7 above, the substantially homologous sequences may be as described anywhere else herein, and are preferably as follows: a sequence substantially homologous to a given VH CDR1 sequence (i.e. a VH CDR1 is preferably a sequence containing up to 2 (e.g. only 1 or 2), preferably only 1 amino acid substitutions as compared to the given CDR sequence;
a sequence substantially homologous to a given VH CDR2 sequence is preferably a sequence containing up to 2 (e.g. only 1 or 2), preferably only 1 amino acid substitutions as compared to the given CDR sequence;
a sequence substantially homologous to a given VH CDR3 sequence is preferably a sequence containing up to 3 (e.g. only 1, 2, or 3), preferably up to 2 (e.g. only 1 or 2), preferably only 1 amino acid substitutions as compared to the given CDR sequence;
a sequence substantially homologous to a given VL CDR1 sequence is preferably a sequence containing up to 2 (e.g. only 1 or 2), preferably only 1 amino acid substitutions as compared to the given CDR sequence;
a sequence substantially homologous to a given VL CDR2 sequence is preferably a sequence containing only 1 amino acid substitution as compared to the given CDR sequence; and
a sequence substantially homologous to a given VL CDR3 sequence is preferably a sequence containing up to 3 (e.g. only 1, 2 or 3), more preferably only 1 or 2, more preferably only 1 amino acid substitutions as compared to the given CDR sequence.

Preferably, each of the sequences substantially homologous to a given CDR sequence comprises only 1 amino acid substitution as compared to the given CDR sequence.

Preferably, the antigen binding protein comprises the given VL CDR2 sequence (i.e. rather than a sequence substantially homologous thereto).

Optionally, the heavy chain variable domain comprises the given CDR sequences (i.e. rather than sequences substantially homologous thereto), and/or the light chain variable domain comprises the given CDR sequences (i.e. rather than sequences substantially homologous thereto).

Preferably, the antigen binding protein (e.g. antibody) contains up to 6, e.g. only 1, 2, 3, 4, 5 or 6, preferably up to 5, for example only 1, 2, 3, 4 or 5, preferably up to 4, for example only 1, 2, 3 or 4, preferably up to 3, e.g. only 1, 2 or 3, more preferably only 1 or 2, more preferably only 1 altered amino acids (preferably substitutions) as compared to the whole complement of the given CDR sequences.

CDR sequences of certain antibodies of the invention are set forth herein in Table A. In some other embodiments, CDR sequences of antibodies of the invention may be CDR sequences in the VH domains and VL domains of antibodies of the invention as identified using any suitable method (or tool), for example as identified using the well-known ImMunoGeneTics information system method, i.e. the IMGT numbering scheme (e.g. Lefranc, M.-P., The Immunologist, 7, 132-136 (1999); www.imqt.org), e.g. as shown in Table A, or as identified according to the well-known methods of Kabat (e.g. Kabat et al., “Sequences of Proteins of Immunological Interest”, 5th Ed. Public Health Service, National Institutes of Health, Bethesda, MD, 647-669, 1991) or Chothia (e.g. Chothia C, et al. (1989) Nature, 342:877-883, or Al-Lazikani et al., (1997) JMB 273, 927-948), or by AbM numbering (e.g. Abhinandan and Martin, 2008, Mol. Immunol. 45:3832-3839).

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3, 118, 126, 130, 132, 134, 136, 137, or 145, or a sequence substantially homologous thereto, and/or (preferably “and”) a VL domain that comprises the amino acid sequence of SEQ ID NO: 4, 114, 116, 120, 122, 124, 128, 138, 140, 142, 144, 146 or 147, or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3, 118, or 130 or a sequence substantially homologous thereto, and/or (preferably “and”) a VL domain that comprises the amino acid sequence of SEQ ID NO: 4, 114, 116, 142 or 147, or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3 or 118, or a sequence substantially homologous thereto, and/or (preferably “and”) a VL domain that comprises the amino acid sequence of SEQ ID NO: 4, 114, or 116, or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3, or a sequence substantially homologous thereto, and/or (preferably “and”) a VL domain that comprises the amino acid sequence of SEQ ID NO: 4 or 114, or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain and/or (preferably “and”) a VL domain comprising the following amino acid sequences or sequences substantially homologous thereto:

| | VH domain | VL domain | |
|---|---|---|---|
| i) | SEQ ID NO: 3 | SEQ ID NO: 4 | ; or |
| ii) | SEQ ID NO: 3 | SEQ ID NO: 114 | ; or |
| iii) | SEQ ID NO: 3 | SEQ ID NO: 116 | ; or |
| iv) | SEQ ID NO: 118 | SEQ ID NO: 116 | ; or |
| v) | SEQ ID NO: 3 | SEQ ID NO: 120 | ; or |
| vi) | SEQ ID NO: 3 | SEQ ID NO: 122 | ; or |
| vii) | SEQ ID NO: 3 | SEQ ID NO: 124 | ; or |
| viii) | SEQ ID NO: 126 | SEQ ID NO: 128 | ; or |
| ix) | SEQ ID NO: 130 | SEQ ID NO: 4 | ; or |
| x) | SEQ ID NO: 132 | SEQ ID NO: 4 | ; or |
| xi) | SEQ ID NO: 134 | SEQ ID NO: 4 | ; or |
| xii) | SEQ ID NO: 118 | SEQ ID NO: 4 | ; or |
| xiii) | SEQ ID NO: 134 | SEQ ID NO: 124 | ; or |
| xiv) | SEQ ID NO: 136 | SEQ ID NO: 116 | ; or |
| xv) | SEQ ID NO: 137 | SEQ ID NO: 114 | ; or |
| xvi) | SEQ ID NO: 3 | SEQ ID NO: 138 | ; or |
| xvii) | SEQ ID NO: 3 | SEQ ID NO: 140 | ; or |
| xviii) | SEQ ID NO: 3 | SEQ ID NO: 142 | ; or |
| xix) | SEQ ID NO: 3 | SEQ ID NO: 144 | ; or |
| xx) | SEQ ID NO: 145 | SEQ ID NO: 142 | ; or |
| xxi) | SEQ ID NO: 145 | SEQ ID NO: 116 | ; or |
| xxii) | SEQ ID NO: 145 | SEQ ID NO: 122 | ; or |
| xxiii) | SEQ ID NO: 145 | SEQ ID NO: 124 | ; or |
| xxiv) | SEQ ID NO: 130 | SEQ ID NO: 142 | ; or |
| xxv) | SEQ ID NO: 130 | SEQ ID NO: 116 | ; or |
| xxvi) | SEQ ID NO: 118 | SEQ ID NO: 142 | ; or |
| xxvii) | SEQ ID NO: 3 | SEQ ID NO: 146 | ; or |
| xxviii) | SEQ ID NO: 3 | SEQ ID NO: 147 | ; or |
| xxix) | SEQ ID NO: 145 | SEQ ID NO: 147 | ; or |
| xxx) | SEQ ID NO: 130 | SEQ ID NO: 147 | . |

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain and/or (preferably “and”) a VL domain comprising the following amino acid sequences or sequences substantially homologous thereto:

| | VH domain | VL domain | |
|---|---|---|---|
| i) | SEQ ID NO: 3 | SEQ ID NO: 4 | ; or |
| ii) | SEQ ID NO: 3 | SEQ ID NO: 114 | ; or |
| iii) | SEQ ID NO: 3 | SEQ ID NO: 116 | ; or |
| iv) | SEQ ID NO: 118 | SEQ ID NO: 116 | ; or |
| v) | SEQ ID NO: 130 | SEQ ID NO: 4 | ; or |
| vi) | SEQ ID NO: 118 | SEQ ID NO: 142 | ; or |
| vii) | SEQ ID NO: 3 | SEQ ID NO: 147 | . |

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain and/or (preferably “and”) a VL domain comprising the following amino acid sequences or sequences substantially homologous thereto:

| | VH domain | VL domain | |
|---|---|---|---|
| i) | SEQ ID NO: 3 | SEQ ID NO: 4 | ; or |
| ii) | SEQ ID NO: 3 | SEQ ID NO: 114 | ; or |
| iii) | SEQ ID NO: 3 | SEQ ID NO: 116 | ; or |
| iv) | SEQ ID NO: 118 | SEQ ID NO: 116 | . |

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3 or a sequence substantially homologous thereto, and/or (preferably “and”) a VL domain that comprises the amino acid sequence of SEQ ID NO: 114 or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3 or a sequence substantially homologous thereto, and/or (preferably “and”) a VL domain that comprises the amino acid sequence of SEQ ID NO: 116 or a sequence substantially homologous thereto.

In another aspect and in certain embodiments, the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 118 or a sequence substantially homologous thereto, and/or (preferably “and”) a VL domain that comprises the amino acid sequence of SEQ ID NO: 116 or a sequence substantially homologous thereto.

Preferably, as described elsewhere herein, sequences that are substantially homologous to a given VH domain sequence have at least 90% identity to said given sequence; and/or (preferably “and”) sequences that are substantially homologous to a given VL domain sequence have at least 80% identity (preferably at least 90% identity) to said given sequence.

Preferably, as described elsewhere herein, sequences that are substantially homologous to a given VH domain sequence have at least 95% identity to said given sequence; and/or (preferably “and”) sequences that are substantially homologous to a given VL domain sequence have at least 80% identity (preferably at least 90% identity) to said given sequence.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO:3, or a sequence having at least 80% sequence identity thereto (e.g. at least 85%, 90%, 95% or 98%) and/or (preferably “and”) a VL domain that comprises the amino acid sequence of SEQ ID NO:4, 114, 116 or 147, or a sequence having at least 80% sequence identity thereto (e.g. at least 85%, 90%, 95% or 98%).

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO:118, or a sequence having at least 80% sequence identity thereto (e.g. at least 85%, 90%, 95% or 98%) and a VL domain that comprises the amino acid sequence of SEQ ID NO: 116 or 142, or a sequence having at least 80% sequence identity thereto (e.g. at least 85%, 90%, 95% or 98%).

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO:130, or a sequence having at least 80% sequence identity thereto (e.g. at least 85%, 90%, 95% or 98%) and a VL domain that comprises the amino acid sequence of SEQ ID NO:4, or a sequence having at least 80% sequence identity thereto (e.g. at least 85%, 90%, 95% or 98%).

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising
a heavy chain variable domain that comprises the amino acid sequence of SEQ ID NO:3, or a sequence having at least 80% sequence identity thereto (e.g. at least 85%, 90%, 95% or 98%), further wherein said heavy chain variable domain comprises three CDRs, preferably comprising the amino acid sequences of SEQ ID NO:5, 6 and 7, or sequences substantially homologous thereto, as defined elsewhere herein; and/or (preferably “and”)
a light chain variable domain that comprises the amino acid sequence of SEQ ID NO:4, or a sequence having at least 80% sequence identity thereto (e.g. at least 85%, 90%, 95% or 98%), further wherein said light chain variable domain comprises three CDRs, preferably comprising the amino acid sequences of SEQ ID NO:8, 9 and 10, or sequences substantially homologous thereto, as defined elsewhere herein.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising
a heavy chain variable domain that comprises the amino acid sequence of SEQ ID NO:3, or a sequence having at least 80% sequence identity thereto (e.g. at least 85%, 90%, 95% or 98%), further wherein said heavy chain variable domain comprises three CDRs comprising the amino acid sequences of SEQ ID NO:5, 6 and 7, or sequences substantially homologous thereto, as defined elsewhere herein; and/or (preferably “and”)
a light chain variable domain that comprises the amino acid sequence of SEQ ID NO: 114, or a sequence having at least 80% sequence identity thereto (e.g. at least 85%, 90%, 95% or 98%), further wherein said light chain variable domain comprises three CDRs comprising the amino acid sequences of SEQ ID NO:8, 9 and 96, or sequences substantially homologous thereto, as defined elsewhere herein.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising
a heavy chain variable domain that comprises the amino acid sequence of SEQ ID NO:3, or a sequence having at least 80% sequence identity thereto (e.g. at least 85%, 90%, 95% or 98%), further wherein said heavy chain variable domain comprises three CDRs comprising the amino acid sequences of SEQ ID NO:5, 6 and 7, or sequences substantially homologous thereto, as defined elsewhere herein; and/or (preferably “and”)
a light chain variable domain that comprises the amino acid sequence of SEQ ID NO:116, or a sequence having at least 80% sequence identity thereto (e.g. at least 85%, 90%, 95% or 98%), further wherein said light chain variable domain comprises three CDRs comprising the amino acid sequences of SEQ ID NO:87, 9 and 10, or sequences substantially homologous thereto, as defined elsewhere herein.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising
a heavy chain variable domain that comprises the amino acid sequence of SEQ ID NO: 118, or a sequence having at least 80% sequence identity thereto (e.g. at least 85%, 90%, 95% or 98%), further wherein said heavy chain variable domain comprises three CDRs comprising the amino acid sequences of SEQ ID NO:5, 6 and 85, or sequences substantially homologous thereto, as defined elsewhere herein; and/or (preferably “and”)
a light chain variable domain that comprises the amino acid sequence of SEQ ID NO: 116, or a sequence having at least 80% sequence identity thereto (e.g. at least 85%, 90%, 95% or 98%), further wherein said light chain variable domain comprises three CDRs comprising the amino acid sequences of SEQ ID NO:87, 9 and 10, or sequences substantially homologous thereto, as defined elsewhere herein.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising
a heavy chain variable domain that comprises the amino acid sequence of SEQ ID NO:3, or a sequence having at least 80% sequence identity thereto (e.g. at least 85%, 90%, 95% or 98%), further wherein said heavy chain variable domain comprises three CDRs comprising the amino acid sequences of SEQ ID NO:5, 6 and 7; and/or (preferably “and”)
i) a light chain variable domain that comprises the amino acid sequence of SEQ ID NO:4, or a sequence having at least 80% sequence identity thereto (e.g. at least 85%, 90%, 95% or 98%), further wherein said light chain variable domain comprises three CDRs comprising the amino acid sequences of SEQ ID NO:8, 9 and 10; or
ii) a light chain variable domain that comprises the amino acid sequence of SEQ ID NO: 114, or a sequence having at least 80% sequence identity thereto (e.g. at least 85%, 90%, 95% or 98%), further wherein said light chain variable domain comprises three CDRs comprising the amino acid sequences of SEQ ID NO:8, 9 and 96; or
iii) a light chain variable domain that comprises the amino acid sequence of SEQ ID NO: 116, or a sequence having at least 80% sequence identity thereto (e.g. at least 85%, 90%, 95% or 98%), further wherein said light chain variable domain comprises three CDRs comprising the amino acid sequences of SEQ ID NO:87, 9 and 10.

In another aspect and in certain embodiments, the present invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising
a heavy chain variable domain that comprises the amino acid sequence of SEQ ID NO:118, or a sequence having at least 80% sequence identity thereto (e.g. at least 85%, 90%, 95% or 98%), further wherein said heavy chain variable domain comprises three CDRs comprising the amino acid sequences of SEQ ID NO:5, 6 and 85; and
a light chain variable domain that comprises the amino acid sequence of SEQ ID NO:116, or a sequence having at least 80% sequence identity thereto (e.g. at least 85%, 90%, 95% or 98%), further wherein said light chain variable domain comprises three CDRs comprising the amino acid sequences of SEQ ID NO:87, 9 and 10.

In alternative embodiments of the invention, sequences indicated as having sequence identity to a sequence with a given SEQ ID NO: can have at least 60%, 65%, 70% or 75% identity to the sequence with the given SEQ ID NO.

In another aspect and in certain embodiments the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 3 and/or (preferably “and”) a VL domain that comprises the amino acid sequence of SEQ ID NO: 4, 114 or 116.

In another aspect and in certain embodiments the invention provides an antigen binding protein, for example an antibody, which binds to, or specifically binds to, HLA-A*02:01/MAGE-A4²³⁰⁻²³⁹, said antigen binding protein comprising at least one antigen binding domain, said antigen binding domain comprising a VH domain that comprises the amino acid sequence of SEQ ID NO: 118 and/or (preferably “and”) a VL domain that comprises the amino acid sequence of SEQ ID NO: 116.

A preferred antigen binding protein of the invention is or comprises an antibody defined herein (in Table A) selected from the group consisting of the 1-H02 antibody, the the AM15 antibody, the AM2 antibody, the AMC9 antibody, the AM1 antibody, the AM3 antibody, the AM4 antibody, the AM5 antibody, the AM6 antibody, the AM7 antibody, the AM9 antibody, the AM10 antibody, the AM11 antibody, the AM12 antibody, the AM13 antibody, the AM14 antibody, the AM16 antibody, the AM17 antibody, the AM18 antibody, the AMC1 antibody, the AMC3 antibody, the AMC4 antibody, the AMC5 antibody, the AMC6 antibody, the AMC7 antibody, the AMC8 antibody, the AMC10 antibody, the AMC11 antibody, the AMC12 antibody, and the AMC14 antibody.

A preferred antigen binding protein of the invention is or comprises an antibody defined herein (in Table A) selected from the group consisting of the 1-H02 antibody, the the AM15 antibody, the AM2 antibody, the AMC9, the AM6 antibody, the AMC8 antibody and the AMC11 antibody.

A preferred antigen binding protein of the invention is or comprises an antibody defined herein (in Table A) selected from the group consisting of the 1-H02 antibody, the the AM15 antibody, the AM2 antibody, and the AMC9 antibody.

A preferred antigen binding protein of the invention is or comprises the AM15 antibody, the AM2 antibody or the AMC9 antibody defined herein (in Table A).

A particularly preferred antigen binding protein of the invention is or comprises the 1-H02 antibody or the AM15 antibody defined herein (in Table A).

An antigen binding protein according to any aspect of the present invention and disclosure may be defined as a binding protein comprising an antigen-binding domain obtained or derived from an antibody, or based on an antigen binding domain of an antibody. Thus, for example, light and heavy chain variable domains (i.e. light and heavy chain variable regions) as described herein are those obtained or derived from an antibody, or based on an antigen binding domain of an antibody.

As described above, the present invention provides antigen binding proteins, for example antibodies, or antigen binding proteins comprising antibodies or the antigen binding domain of an antibody, which bind to (or specifically recognise or specifically bind to) HLA-A*02:01/MAGE-A4²³⁰⁻²³⁹. Preferred antigen binding proteins of the invention are antibodies. However, embodiments as described herein which relate to antibodies, apply equally, mutatis mutandis, to other types of antigen binding proteins, or vice versa. Thus, other antigen binding proteins can comprise the antibodies of the invention or can comprise the antigen binding domains of the antibodies of the invention, e.g. the three VL CDR regions and the three VH CDR regions of the antibodies of the invention, or a VL and VH domain of the antibodies of the invention (a domain typically comprising the three VH or the three VL CDR regions (CDR1, CDR2 and CDR3) and the four VH or the four VL framework (“FR”) regions (FR1, FR2, FR3 and FR4)).

Appropriate types of antigen binding protein which could be used in the invention are known in the art. For example, in some embodiments immunoglobulin based polypeptides are used, which generally comprise CDR regions (and optionally FR regions or an immunoglobulin based scaffold), such that the CDR regions (and optionally FR regions) of the antibodies of the invention can be grafted onto an appropriate scaffold or framework, e.g. an immunoglobulin scaffold. Alternatively, the antigen binding domains or the antibodies of the invention can be incorporated into any appropriate antigen binding fragment or antibody containing format, e.g. can be incorporated into a chimeric antigen receptor (CAR) format or a CAR-T cell format.

For the avoidance of doubt, in accordance with the present invention the antigen binding domain that binds to (or specifically binds to) HLA-A*02:01/MAGE-A4²³⁰⁻²³⁹ is not from, or does not correspond to, a T-cell receptor (TCR). Thus, for the avoidance of doubt, antigen binding proteins in accordance with the present invention do not include TCRs as an antigen binding domain specific for HLA-A*02:01/MAGE-A4²³⁰⁻²³⁹. Thus, the antigen binding proteins of the present invention are not TCRs, i.e. do not comprise a T-cell receptor α-chain, and/or preferably (“and”) do not comprise a T-cell receptor β-chain.

Preferably, the antigen binding protein (i.e. the protein having an antigen binding domain) is an antibody or an antigen binding fragment thereof (an antibody fragment). The term “antibody” is used as shorthand to refer to “antibody or an antigen binding fragment thereof” unless otherwise clear from context.

The terms “antibody” and “immunoglobulin”, as used herein, refer broadly to any immunological binding agent that comprises an antigen binding domain (e.g. a human antigen binding domain), including polyclonal and monoclonal antibodies. Depending on the type of constant domain in the heavy chains, whole antibodies are assigned to one of five major classes: IgA, IgD, IgE, IgG, and IgM and the antibodies of the invention may be in any one of these classes. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

The “light chains” of mammalian antibodies are assigned to one of two clearly distinct types: kappa (κ) and lambda (λ), based on the amino acid sequences of their constant domains and some amino acids in the framework regions of their variable domains.

The term “heavy chain complementarity determining region” (“heavy chain CDR”) as used herein refers to regions of hypervariability within the heavy chain variable region (V_{H} domain) of an antibody molecule. The heavy chain variable region (i.e. domain) has three CDRs termed heavy chain CDR1, heavy chain CDR2 and heavy chain CDR3 from the amino terminus to carboxy terminus. The heavy chain variable region (domain) also has four framework regions (FR1, FR2, FR3 and FR4 from the amino terminus to carboxy terminus). These framework regions separate the CDRs.

The term “heavy chain variable region” (V_{H} domain) as used herein refers to the variable region of a heavy chain of an antibody molecule.

The term “light chain complementarity determining region” (“light chain CDR”) as used herein refers to regions of hypervariability within the light chain variable region (V_{L} domain) of an antibody molecule. Light chain variable regions (domains) have three CDRs termed light chain CDR1, light chain CDR2 and light chain CDR3 from the amino terminus to the carboxy terminus. The light chain variable region (domain) also has four framework regions (FR1, FR2, FR3 and FR4 from the amino terminus to carboxy terminus). These framework regions separate the CDRs.

The term “light chain variable region” (V_{L} domain) as used herein refers to the variable region of a light chain of an antibody molecule.

As will be understood by those in the art, the immunological binding reagents encompassed by the term “antibody” includes or extends to all antibodies, antigen binding fragments thereof and antibody “formats”, including whole antibodies, dimeric, trimeric and multimeric antibodies; bispecific antibodies; trispecific antibodies; multispecific antibodies; chimeric antibodies; recombinant and engineered antibodies, and fragments thereof. The terms “antibody format” and “antibody construct” are used interchangeably herein. In the field, and herein, the term “antibody format” encompasses both antibody fragments, whole antibodies and multimeric antibodies.

The term “antibody fragment” as used herein refers to fragments of biological relevance, e.g. fragments that comprise the above-mentioned antigen binding domain, i.e. contribute to antigen binding, e.g. form part of the antigen binding domain. Certain preferred fragments comprise a heavy chain variable region (V_{H} domain) and a light chain variable region (V_{L} domain) of the antibodies of the invention.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

The term “antibody” is thus used to refer to any antibody-like molecule that has an immunoglobulin (Ig) antigen binding domain, and this term includes antibody fragments and formats that comprise an antigen binding domain including but not limited to Fab′, Fab, F(ab′)₂, single domain antibodies (DABs), TandAbs dimer, Fv, scFv (single chain Fv), dsFv, ds-scFv, Fd, scFv/Fc^{KIH}, linear antibodies, minibodies, diabodies, bispecific antibody fragments, bibody, tribody (scFv-Fab fusions, bispecific or trispecific, respectively, including scFv/Fab-Fc, and scFv/Fab-Fc^{KIH}); sc-diabody; single chain bispecific diabody (scDb); kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”), DART-Fc; Duabody, CrossMab, DuetMab, DNL, small antibody mimetics.

In embodiments, the antigen binding protein (e.g. antibody) of the present invention may be in a single chain format, e.g. a single chain antibody format. In other embodiments, the antigen binding protein (e.g. antibody) of the present invention may be in a multichain format.

Antibody formats, and their preparation, is within the general competencies of the person of ordinary skill in the antibody field.

In certain embodiments, the antigen binding protein (e.g. antibody) of the present invention comprises all or a portion of a heavy chain constant region, such as an IgG1, IgG2, IgG3, IgG4, IgA1, IgA2, IgE, IgM, or IgD constant region. Preferably, the heavy chain constant region is an IgG heavy chain constant region or a portion thereof. IgG1 and IgG4 are examples of appropriate formats for the antibodies of the invention. Antibodies of the invention may comprise or contain human heavy-chain constant regions.

Furthermore, the antigen binding protein (e.g. antibody) of the invention can comprise all or a portion of a kappa light chain constant region or a lambda light chain constant region, or a portion thereof. Antibodies of the invention may comprise or contain human light-chain constant regions.

All or part of such constant regions may be produced naturally or may be wholly or partially synthetic. Appropriate sequences for such constant regions are well known and documented in the art. In embodiments, full length heavy chain sequences of the antigen binding proteins (e.g. antibodies) of the invention comprise an amino acid sequence comprising or consisting of SEQ ID NOs: 77, 150, 154 or 158, or a sequence substantially homologous thereto (e.g. having at least 80% sequence identity thereto (e.g. at least 85%, 90%, 95% or 98%)).

In embodiments, full length light chain sequences of the antigen binding proteins (e.g. antibodies) of the invention comprise an amino acid sequence comprising or consisting of SEQ ID NOs: 78, 151, 155 or 159, or a sequence substantially homologous thereto (e.g. having at least 60%, preferably at least 70%, more preferably at least 80% sequence identity thereto (e.g. at least 85%, 90%, 95% or 98%)).

Preferably, the CDR, VH and/or VL sequences in said full heavy and light chain sequences are as defined anywhere else herein.

Thus, for instance in embodiments, the antigen binding proteins (e.g. antibodies) of the invention may comprise:
a heavy chain comprising or consisting of the amino acid sequence of SEQ ID NO: 77, or a sequence substantially homologous thereto (e.g. having at least 80% sequence identity thereto (e.g. at least 85%, 90%, 95% or 98%), and in which said heavy chain comprises a heavy chain variable domain that comprises three CDRs comprising the amino acid sequences of SEQ ID NO:5, 6 and 7, or sequences substantially homologous thereto, as defined elsewhere herein; and/or
a light chain comprising or consisting of the amino acid sequence of SEQ ID NO: 78, or a sequence substantially homologous thereto (e.g. having at least 80% sequence identity thereto (e.g. at least 85%, 90%, 95% or 98%), and in which said light chain comprises a light chain variable domain that comprises three CDRs comprising the amino acid sequences of SEQ ID NO:8, 9 and 10, or sequences substantially homologous thereto, as defined elsewhere herein.

In embodiments, the antigen binding proteins (e.g. antibodies) of the invention may comprise:
a heavy chain comprising or consisting of the amino acid sequence of SEQ ID NO: 150, or a sequence substantially homologous thereto (e.g. having at least 80% sequence identity thereto (e.g. at least 85%, 90%, 95% or 98%), and in which said heavy chain comprises a heavy chain variable domain that comprises three CDRs comprising the amino acid sequences of SEQ ID NO:5, 6 and 7, or sequences substantially homologous thereto, as defined elsewhere herein; and/or
a light chain comprising or consisting of the amino acid sequence of SEQ ID NO: 151, or a sequence substantially homologous thereto (e.g. having at least 80% sequence identity thereto (e.g. at least 85%, 90%, 95% or 98%), and in which said light chain comprises a light chain variable domain that comprises three CDRs comprising the amino acid sequences of SEQ ID NO:8, 9 and 96, or sequences substantially homologous thereto, as defined elsewhere herein.

When a full complement of constant regions from the heavy and light chains are included in the antigen binding proteins (e.g. antibodies) of the invention, such antigen binding proteins (e.g. antibodies) are typically referred to herein as “full length” antibodies or “whole” antibodies. In some embodiments such full length or whole antibodies are provided.

The antigen binding proteins (e.g. antibodies) can be produced naturally or can be wholly or partially synthetically produced.

The antigen binding proteins (e.g. antibodies) may be from any appropriate source, for example recombinant sources and/or produced in transgenic animals or transgenic plants, or in eggs using the IgY technology. Thus, the antigen binding protein (e.g. antibody) molecules can be produced in vitro or in vivo.

The antigen binding domains of the antigen binding proteins (e.g. antibodies) of the invention generally comprise an antibody light chain variable region (domain) (V_{L}) that comprises three CDR domains and an antibody heavy chain variable region (domain) (V_{H}) that comprises three CDR domains.

However, it is well documented in the art that the presence of three CDRs from the light chain variable domain and three CDRs from the heavy chain variable domain of an antigen binding protein (e.g. antibody) is not always necessary for antigen binding. Thus, constructs smaller than the above classical antigen binding domain are known to be effective.

For example, camelid VHH antibodies and other single domain antibodies comprising VH domains alone show that these domains can bind to antigen with acceptably high affinities. Thus, three CDRs (or even a single CDR) can effectively bind antigen and form an antigen binding domain.

Thus, although preferred antigen binding domains in the antigen binding proteins (e.g. antibodies) of the invention might comprise six CDR regions (three from a light chain and three from a heavy chain), antigen binding proteins (e.g. antibodies) with antigen binding domains with fewer than six CDR regions (e.g. 3 CDR regions) are encompassed by the invention. Antigen binding proteins (e.g. antibodies) with antigen binding domains with CDRs from only the heavy chain or light chain are also contemplated.

Preferred light chain CDR regions (domains) for use in conjunction with the specified heavy chain CDR regions to form the antigen binding domain are described elsewhere herein. However, other light chain variable regions (domains) that comprise three CDRs for use in conjunction with the heavy chain variable regions (domains) of the invention are also contemplated. Appropriate light chain variable regions (domains) which can be used in combination with the heavy chain variable regions (domains) of the invention and which give rise to an antibody which binds to HLA-A*02:01/MAGE-A4²³⁰⁻²³⁹ in accordance with the invention can be readily identified by a person skilled in the art.

For example, a heavy chain variable region (domain) of the invention can be combined with a single light chain variable region (domain) or a repertoire of light chain variable regions (domains) and the resulting antigen binding proteins (e.g. antibodies) tested for binding to HLA-A*02:01/MAGE-A4²³⁰⁻²³⁹.

If desired, similar methods could be used to identify alternative heavy chain variable regions (domains) for use in combination with preferred light chain variable regions (domains) of the invention.

The techniques for preparing and using various antigen binding protein-based constructs (e.g. antibody-based constructs) and fragments are well known in the art. Diabodies, in particular, are further described in EP404097 and WO 93/11161; whereas linear antibodies are further described in the art.

For convenience, an antigen binding protein (e.g. antibody) of the invention that binds to HLA-A*02:01/MAGE-A4²³⁰⁻²³⁹, may be referred to elsewhere herein simply as a MAGE-A4 pMHC antigen binding protein, or a MAGE-A4 antigen binding protein, e.g. a MAGE-A4 pMHC antibody, or a MAGE-A4 antibody.

As described above, the present invention provides antigen binding proteins (e.g. antibodies), for example isolated antigen binding proteins (e.g. isolated antibodies), which bind to (or specifically recognise or specifically bind to) HLA-A*02:01/MAGE-A4²³⁰⁻²³⁹. This is the “target” antigen of the antigen binding proteins (e.g. antibodies) of the invention. The antigen binding proteins (e.g. antibodies) of the invention are described as having a “specificity” for this target antigen. Due to their binding to (or specifically recognising or specifically binding to) a HLA-restricted peptide, the antigen binding proteins (e.g. antibodies) of the invention are termed “TCR-like” antigen binding proteins (e.g. TCR-like antibodies).

For the avoidance of doubt, the antigen binding proteins, e.g. antibodies, of the invention comprise at least one antigen binding domain that binds to, or specifically binds to, HLA-A*02:01/MAGE-A4²³⁰⁻²³⁹, i.e. it is this antigen binding domain that confers onto the antigen binding proteins of the invention the ability to bind to (or specifically recognise or specifically bind to) this antigen. The antigen binding proteins (e.g. antibodies) of the invention thus comprise a “first” antigen binding domain, with a “first” binding specificity, also termed “specificity A”, which is for HLA-A*02:01/MAGE-A4²³⁰⁻²³⁹. The presence of an antigen binding domain described herein as a “first” antigen binding domain does not imply that the presence of one or more further antigen binding domains is essential; such further antigen binding domains are optional.

Within an antigen binding domain, the heavy chain variable domain and the light chain variable domain can also each be described as having a “specificity” for the antigen to which the antigen binding domain binds. Thus, the antigen binding proteins, e.g. antibodies, of the invention, comprise at least one antigen binding domain that comprises a heavy chain variable domain with specificity for HLA-A*02:01/MAGE-A4²³⁰⁻²³⁹, and a light chain variable domain with specificity for HLA-A*02:01/MAGE-A4²³⁰⁻²³⁹. As above, in the present disclosure, such specificity for HLA-A*02:01/MAGE-A4²³⁰⁻²³⁹ is termed the “first” specificity, or “specificity A”.

HLA-A*02 (also termed “HLA-A2”, “HLA-A02”, and “HLA-A*2”) is a human class I major histocompatibility complex (MHC) allele group at the HLA-A locus. HLA-A*02 is a human leukocyte antigen serotype within the HLA-A serotype group.

The HLA-A*02 protein (encoded by the respective HLA gene) constitutes the α chain of the respective class I MHC (major histocompatibility complex) protein, which further comprises a β2 microglobulin subunit, also termed the “β chain”, which is encoded by the β2 microglobulin (β2M) locus.

A specific HLA-A2 protein is HLA-A*02:01 (also referred to as HLA-A02:01, HLA-A0201, or HLA-A*02.01), which is a common MHC class I allele in the HLA-A*02 group. In the present invention, the HLA-A2 protein described is HLA-A*02:01 (IPD Accession: HLA00005 and HLA00006).

The amino acid sequence of HLA-A*02:01 encoded at these loci is set forth herein in SEQ ID NO: 21. This protein comprises a N-terminal leader peptide, an extracellular domain (ECD), a transmembrane domain and a C-terminal intracellular domain. In the pHLA complexes to which the antigen binding proteins (e.g. antibodies) of the invention bind, it is the extracellular domain of the HLA that is bound, the amino acid sequence of which is set forth in SEQ ID NO: 22. Recombinant forms of the protein typically comprise only the extracellular domain.

The antigen binding proteins (e.g. antibodies) of the invention may bind to functionally equivalent pHLA-A2 complexes in which MAGE-A4²³⁰⁻²³⁹ is displayed, and in which the HLA-A2 comprises an amino acid sequence substantially homologous to SEQ ID NO: 21.

The amino acid sequence of human P2 microglobulin is set forth herein in SEQ ID NO: 23.

HLA-A*02:01 can present peptides that are fragments of intracellular proteins, including MAGE-derived peptides, i.e. peptides derived from MAGE proteins.

“MAGE-A4” stands for “Melanoma-associated antigen 4”, which is a member of the MAGE family of Cancer Testis Antigens (CTAs). The MAGE-A family of proteins encompasses 12 highly homologous genes clustered at Xq26-28 and characterized by the presence of a conserved domain (MAGE Homology Domain, MHD).

“MAGE-A4” as used herein, refers to any native MAGE-A4 from any vertebrate source, including mammals such as primates (e.g. humans), non-human primates (e.g. cynomolgus monkeys) and rodents (e.g. mice and rats), unless otherwise indicated. The term encompasses “full-length,” unprocessed MAGE-A4 as well as any form of MAGE-A4 that results from processing in the cell. The term also encompasses naturally occurring variants of MAGE-A4, e.g., splice variants or allelic variants.

Preferably, MAGE-A4 is human MAGE-A4, which is described in UniProt (www.uniprot.org) accession no. P43358 (entry version 163). An amino acid sequence of human MAGE-A4 is set forth in SEQ ID NO: 20 herein. Recombinant human MAGE-A4 is commercially available.

The antigen binding proteins (e.g. antibodies) of the present invention bind to (or specifically bind to) the HLA-A*02:01 restricted MAGE-A4 derived peptide MAGE-A4²³⁰⁻²³⁹. By “MAGE-A4²³⁰⁻²³⁹” is meant the MAGE-A4 derived peptide having the amino acid sequence GVYDGREHTV (SEQ ID NO: 19; found at positions 230-239 of the MAGE-A4 protein of SEQ ID NO: 20).

Thus, HLA-A*02:01/MAGE-A4²³⁰⁻²³⁹ refers to a complex of MAGE-A4²³⁰⁻²³⁹ presented/displayed on the class I MHC molecule comprising HLA-A*02:01, i.e. HLA-A*02:01 restricted MAGE-A4²³⁰⁻²³⁹. In other words, HLA-A*02:01/MAGE-A4²³⁰⁻²³⁹ means a HLA-A*02:01 molecule that is presenting (or “loaded” with) the MAGE-A4²³⁰⁻²³⁹ peptide. Put another way, HLA-A*02:01/MAGE-A4²³⁰⁻²³⁹ means a HLA-A*02:01-peptide complex (pMHC) in which the MAGE-A4²³⁰⁻²³⁹ epitope is presented in the antigen binding groove (or accommodated in the antigen binding groove) of the MHC.

Preferred and convenient forms of HLA-A*02:01/MAGE-A4²³⁰⁻²³⁹ to which the antigen binding proteins (e.g. antibodies) of the invention can bind may comprise recombinant MAGE-A4²³⁰⁻²³⁹, e.g. a recombinant human MAGE-A4²³⁰⁻²³⁹, or a native or natural form of MAGE-A4²³⁰⁻²³⁹, for example MAGE-A4²³⁰⁻²³⁹ when presented via HLA-A*02:01 on the cell surface, e.g. on the surface of a cancer cell.

The antigen binding proteins (e.g. antibodies) of the invention do not bind to (or do not cross-react with), e.g. do not significantly bind to (or do not significantly cross-react with) the HLA-A*02:01 molecule itself/alone (i.e. the MHC molecule itself), i.e. in the absence of a presented MAGE-A4²³⁰⁻²³⁹ peptide, i.e. without the MAGE-A4²³⁰⁻²³⁹ peptide presented or complexed thereto, i.e. to an unloaded HLA-A*02:01 molecule.

The antigen binding proteins (e.g. antibodies) of the invention do not bind to (or do not cross-react with), e.g. do not significantly bind to (or do not significantly cross-react with) the soluble form of MAGE-A4²³⁰⁻²³⁹, i.e. they do not bind (or do not significantly bind) to MAGE-A4²³⁰⁻²³⁹ unless it is presented by a HLA-A*02:01 complex, i.e. unless it is HLA-A*02:01 restricted.

Thus, the antigen binding proteins (e.g. antibodies) of the invention typically bind to, or specifically bind to, the MAGE-A4²³⁰⁻²³⁹ epitope (or peptide), solely (or strictly) in the context of the MHC, i.e. HLA-A*02:01.

A convenient and appropriate method for assessing binding would include in vitro binding assays such as ELISA assays to assess binding of antigen binding proteins (e.g. antibodies) to immobilised antigen, such as immobilised forms of HLA-A*02:01/MAGE-A4²³⁰⁻²³⁹ as described elsewhere herein.

Thus, in certain embodiments, antigen binding proteins (e.g. antibodies) of the present invention can bind to HLA-A*02:01/MAGE-A4²³⁰⁻²³⁹ in an ELISA assay. The skilled person will be familiar with ELISA assays and readily able to establish suitable conditions to assess the ability of an antibody to bind to HLA-A*02:01/MAGE-A4²³⁰⁻²³⁹ in such an assay. Suitable assays are discussed elsewhere herein. Particularly preferred ELISA assays are described in the Examples section herein

In certain embodiments, antigen binding proteins (e.g. antibodies) of the present invention bind to HLA-A*02:01/MAGE-A4²³⁰⁻²³⁹ in (as determined in) a Surface Plasmon Resonance (SPR) assay (e.g. a BIACore assay, e.g. using a BIAcore S200 instrument). Suitable SPR assays are known in the art and are discussed elsewhere herein. Particularly preferred SPR assays are described in the Examples section herein.

Thus, in some embodiments, antibodies (or binding proteins) of the invention are able to bind to HLA-A*02:01/MAGE-A4²³⁰⁻²³⁹ in an SPR assay, or in an ELISA assay, preferably both.

Any substantially homologous antigen binding protein (e.g. antibody) of the invention preferably exhibits such binding.

In preferred embodiments, an antigen binding protein (e.g. antibody) of the invention binds to, or specifically binds to, the HLA-A*02:01/MAGE-A4²³⁰⁻²³⁹ complex when said complex is present on cells, e.g. cancer cells.

In other words, the antigen binding proteins (e.g. antibodies) of the invention are capable of binding to HLA-A*02:01/MAGE-A4²³⁰⁻²³⁹ positive cells, i.e. HLA-A*02:01 positive, MAGE-A4²³⁰⁻²³⁹ positive cells. Such cells are preferred “target cells” herein; target cells being cells displaying an antigen to which the antibodies bind, or specifically bind.

Preferred target cells are cancer cells, which includes tumour cells and blood cancer cells. Cancer cells may be any HLA-A*02:01/MAGE-A4²³⁰⁻²³⁹ positive cancer cell, i.e. any cancer cell on the surface of which is displayed HLA-A*02:01/MAGE-A4²³⁰⁻²³⁹. In certain embodiments, the cancer cells are selected from the group consisting of lung cancer cells (e.g. NCI-H1703 cells), melanoma cells (e.g. A-375 cells), and monocytes (e.g. THP-1 cells). Preferably the cancer cells are cancer cells in or from a subject suffering from cancer. In certain embodiments, the cancer is lung cancer, skin cancer or leukaemia. Cancer cells may be cancer cell line cells, which may be used in the assays described herein.

Alternatively, the cells may be T2 cells, which are well-known and widely used in the field. T2 cells can be induced (“pulsed”) to display MHC class I molecules complexed with exogenously administered peptides. T2 cells are deficient in a peptide transporter involved in endogenous antigen processing (TAP) and therefore have markedly reduced ability to display MHC class I molecules complexed with endogenous peptides. Thus, the antigen binding proteins (e.g. antibodies) of the invention are capable of binding to T2 cells induced to display exogenously administered MAGE-A4²³⁰⁻²³⁹ in a HLA-A*02:01 restricted manner.

Methods of assessing binding to HLA-A*02:01/MAGE-A4²³⁰⁻²³⁹ on or in cells (e.g. cancer cells or peptide pulsed T2 cells) would be well-known to a person skilled in the art and any appropriate method can be used.

Any substantially homologous antigen binding protein (e.g. antibody) of the invention preferably exhibits such binding.

As described above, the antigen binding proteins (e.g. antibodies) of the invention comprise a first antigen binding domain which binds to (or specifically binds to) HLA-A*02:01/MAGE-A4²³⁰⁻²³⁹.

The antigen binding proteins (e.g. antibodies) of the invention may comprise further antigen binding domains. The number of antigen binding domains possessed by an antigen binding protein (e.g. antibody) determines its valency. Thus, in certain embodiments, the antigen binding proteins (e.g. antibodies) of the invention comprise a second antigen binding domain (i.e. are bivalent), and optionally a third antigen binding domain (i.e. are trivalent), and optionally further antigen binding domains (i.e. are multivalent).

In certain embodiments some or all of the further antigen binding domains may also bind to (or specifically bind to) HLA-A*02:01/MAGE-A4²³⁰⁻²³⁹. In these embodiments, any further antigen binding domain(s) may incorporate, singly or in combination, any of the features described herein in relation to the first antigen binding domain.

However, in preferred embodiments, some or all of the further antigen binding domains bind to (or specifically bind to) an antigen other than HLA-A*02:01/MAGE-A4²³⁰⁻²³⁹. Thus, the antigen binding proteins (e.g. antibodies) of the invention may be bispecific, trispecific or multispecific. The term “-specific” or “-specificity” in the context of bispecific, trispecific, multispecific, etc. as used herein refers to the ability of a domain (or antigen binding domain) to recognize a particular target antigen and refers to the capability of binding that target antigen. Bispecific antibodies are therefore capable of recognizing and binding to (or specifically binding to) two different target antigens.

Thus, in addition to a first antigen binding domain that binds to, or specifically binds to, HLA-A*02:01/MAGE-A4²³⁰⁻²³⁹, bispecific antigen binding proteins (e.g. antibodies) of the invention comprise a “second antigen binding domain” having a “second antigen binding specificity” (or simply a “second specificity” or “specificity B”) which is for an antigen other than HLA-A*02:01/MAGE-A4²³⁰⁻²³⁹. Trispecific antigen binding proteins (e.g. antibodies) of the invention comprise a “third antigen binding domain” having a “third antigen binding specificity” (or simply a “third specificity”, or “specificity C”), which is for an antigen other than HLA-A*02:01/MAGE-A4²³⁰⁻²³⁹ and which is different from the specificity of the second antigen binding domain (i.e. the second antigen binding specificity). Multispecific antigen binding proteins (e.g. antibodies) comprise four or more antigen binding domains each of which have a different antigen binding specificity.

The bivalent, trivalent, multivalent, bispecific, trispecific and multispecific antigen binding proteins (e.g. antibodies) of the invention may comprise antigen binding domains fused to each other, optionally via peptide linkers. The production of bivalent, trivalent, multivalent, bispecific, trispecific and multispecific antigen binding proteins (e.g. antibodies), the selection of (e.g. length and sequence of) peptide linkers, and the orientation of domains and peptide linkers, are well-known in the field, and would be within the competencies of the person of ordinary skill in the art.

For instance, in a bispecific antigen binding protein (e.g. antibody) of the invention, the first and second antigen binding domains may be fused to each other, optionally via a peptide linker. The first and the second antigen binding domain may be fused in any orientation, for instance (i) the C-terminus of the first antigen binding domain may be fused, optionally via a peptide linker, to the N-terminus of the second antigen binding domain, or (ii) the C-terminus of the second antigen binding domain may be fused, optionally via a peptide linker, to the N-terminus of the first antigen binding domain.

In certain antibody formats, particularly single chain bispecific antibody formats (e.g. BiTE and scDb formats), the variable light (VL) and variable heavy (VH) domains from the first (A) and second (B) antigen binding domains may be fused (optionally via peptide linkers) in any sequence/orientation (i.e. order), provided that upon proper folding of the resulting polypeptide, two functional antigen binding domains are formed. Again, the production of such antigen binding proteins (e.g. antibodies), the selection of (e.g. length and sequence of) peptide linkers, and the orientation of domains and peptide linkers, are well-known in the field, and would be within the competencies of the person of ordinary skill in the art.

Preferred orientations are, in the N to C direction, i) VLA-VHB-VLB-VHA, ii) VLB-VHA-VLA-VHB; iii) VHA-VLB-VHB-VLA and iv) VHB-VLA-VHA-VLB, most preferred are i) VLA-VHB-VLB-VHA, and ii) VLB-VHA-VLA-VHB, particularly i) VLA-VHB-VLB-VHA.

In a preferred embodiment, the antigen binding protein (e.g. antibody) is at least bispecific, e.g. is a bispecific antibody. In other words, the antigen binding protein (e.g. antibody) of the invention preferably comprises a second antigen binding domain with a second specificity, i.e. which binds to, or specifically binds to, a second antigen, i.e. to an antigen other than HLA-A*02:01/MAGE-A4²³⁰⁻²³⁹.

The (at least) bispecific antigen binding protein (e.g. antibody) of the present invention may be of any antibody fragment, format or construct described herein, including single chain formats and multichain formats.

Preferred antigen binding proteins (e.g. antibodies) of the invention are in a T-cell engager format, i.e. are T-cell engagers. This is a well-understood term in the art, which refers to antigen binding proteins (e.g. antibodies) that bind to (or specifically bind to) HLA-A*02:01/MAGE-A4²³⁰⁻²³⁹ and to an antigen present on T cells (T lymphocytes) via different antigen binding domains. Such T-cell engagers (TCE) are also termed T-cell engaging antibodies (or antigen binding proteins). TCEs physically recruit T cells to target cells (e.g. cancer cells) by binding simultaneously via separate antigen binding domains to both the target cell antigen HLA-A*02:01/MAGE-A4²³⁰⁻²³⁹ and to a T-cell surface antigen (referred to herein as simply a T-cell antigen). A T-cell surface antigen is an antigen present on the surface of T cells. A T-cell specific antigen is an antigen present in or on T-cells specifically. This dual binding and recruitment of T cells to target cells leads to T-cell activation, proliferation and T-cell mediated target cell killing. T-cell engagers may be trispecific or multispecific, but they are at least bispecific, as described above.

Thus, in a preferred embodiment the antigen binding protein (e,g. antibody) of the invention further comprises a second antigen binding domain that binds to (or specifically binds to) a T-cell surface antigen.

The T-cell surface antigen is preferably a T-cell specific antigen, particularly CD3, most particularly CD3ε. Alternatively, the second antigen binding domain may be specific for another T-cell surface antigen, e.g. the T-cell receptor (i.e. a polypeptide comprised in the T-cell receptor). Preferably the T-cell surface antigen, e.g. CD3, is a human T-cell antigen, e.g. human CD3.

Such (at least) bispecific antibodies trigger T-cell activation in a target specific manner, i.e. they redirect T cell activity (e.g. T-cell mediated lysis) against target cells (e.g. cancer cells and tumours) displaying the HLA-A*02:01/MAGE-A4²³⁰⁻²³⁹ antigen.

Antibodies against suitable T-cell surface antigens are numerous and widely available, for instance OKT3 and UCHT1. The second antigen binding domain which is specific for a T-cell surface antigen may be the antigen binding domain of any such suitable antibody against a T-cell surface antigen, preferably CD3. The skilled person will readily be able to identify suitable antibodies and binding domains for their purposes.

A preferred anti-CD3 antibody in this regard is UCHT1, the heavy chain and light chain variable domain amino acid sequences of which are well-known (SEQ ID NOs: 24 and 25 herein, respectively). These VH and VL domain sequences represent preferred VH and VL domains of the second antigen binding domain of the (at least) bispecific antigen binding proteins (e.g. antibodies) of the present invention.

Thus, in embodiments, the antigen binding proteins (e.g. antibodies) of the invention preferably comprise a second antigen binding domain that binds to (or specifically binds to) CD3, said antigen binding domain comprising a heavy chain variable region (domain) that comprises the amino acid sequence of SEQ ID NO:24, or a sequence substantially homologous thereto (e.g. having at least 80% sequence identity thereto), and/or (preferably “and”) a light chain variable region (domain) that comprises the amino acid sequence of SEQ ID NO:25, or a sequence substantially homologous thereto (e.g. having at least 80% sequence identity thereto). The meaning of “substantially homologous” is as defined elsewhere herein.

Thus, antigen binding proteins (e.g. antibodies) of the invention that are in the T-cell engager format (i.e. that comprise a second antigen binding domain that is specific for a T-cell surface antigen) redirect T cells displaying said T-cell surface antigen to the site of the target antigen HLA-A*02:01/MAGE-A4²³⁰⁻²³⁹, e.g. to cancer cells or tumours, and induce activation, differentiation, proliferation of T-cells directed to said target antigen, and thus T-cell mediated cytotoxicity against said target cells.

The T cells redirected in this manner are termed “effector T cells” herein, and may be CD8⁺ T cells, CD4⁺ T cells, CD4⁺CD8⁺ T cells, regulatory T cells, gamma-delta T cells, memory T cells, natural killer T cells (NKTs) or any combination thereof.

The T cells may be comprised within an effector cell population, which may also comprise other effector cells. “Effector cell” is a term well-known in the art to mean any immune cell that can effect or enhance an immune response. Preferred effector cells are peripheral blood mononuclear cells (PBMCs), and pan-T cells derived therefrom. PBMCs are a mixed composition of various effector cell types including lymphocytes (T cells, B cells, and NK cells) and monocytes. These cells can be extracted from whole blood or buffy coat samples, e.g. by using a hydrophilic colloid and density gradient centrifugation, which separates the blood into a top layer of plasma, followed by a layer of PBMCs and a bottom fraction of polymorphonuclear cells (such as neutrophils and eosinophils) and erythrocytes. Pan T-cells are a population of CD3+ cells that be isolated from PBMCs. Obtaining PBMCs, pan-T cells and other effector cell populations is within the competencies of the person of ordinary skill in the art.

CD3 is a preferred T cell surface antigen, and is a T cell specific antigen, i.e. is displayed specifically on T cells, including on CD8⁺ T cells, CD4⁺ T cells, CD4⁺ CD8⁺ T cells, regulatory T cells, gamma-delta T cells, memory T Cells, and natural killer T cells (NKTs).

It is possible to recruit and activate specific subsets of T cells to the site of the target antigen HLA-A*02:01/MAGE-A4²³⁰⁻²³⁹, e.g. to cancer cells or tumours. In embodiments the second antigen binding domain may bind to (or specifically bind to) a T-cell surface antigen specific to the desired subset of T cells.

For instance, in certain embodiments the second antigen binding domain may bind to (or specifically bind to) a “public” T-cell receptor (i.e. polypeptides thereof), i.e. TCRs of a sequence known to be shared by a large number of individuals within a population. Antigen binding proteins (e.g. antibodies) comprising such a second antigen binding domain would have likely therapeutic utility in a wide range of patients, given that the mechanism of action would be to redirect T cells known to be present in a large number of individuals against the target antigen HLA-A*02:01/MAGE-A4²³⁰⁻²³⁹. Such public T-cell receptors, and the sequences of their TCRs, are well documented in the field. Such public TCRs typically arise in response to a common infectious agent, e.g. a virus. Thus, preferred public TCR to which the second antigen binding domain of the antigen binding proteins (e.g. antibodies) binds (or specifically binds), are virus specific public TCRs, preferably human virus specific public TCRs.

Alternatively, gamma-delta T cells may be redirected to the site of the target antigen HLA-A*02:01/MAGE-A4²³⁰⁻²³⁹, e.g. to cancer cells or tumours, and activated, in embodiments wherein the second antigen binding domain of the antigen binding proteins (e.g. antibodies) of the invention binds to (or specifically binds to) an antigen present on the surface of the gamma-delta T cells. For instance, the second antigen binding domain may be bind to (or specifically bind to) the public Vγ9Vδ2 TCR, preferably to human TCRs.

Alternatively, NKT cells may be redirected to the site of the target antigen HLA-A*02:01/MAGE-A4²³⁰⁻²³⁹, e.g. to cancer cells or tumours, and activated, in embodiments wherein the second antigen binding domain of the antigen binding proteins (e.g. antibodies) of the invention binds to (or specifically binds to) an antigen present on the surface of the NKT cells. For instance, the second antigen binding domain may bind to (or specifically bind to) the public Vα24 TCR (Vα14 in mice), preferably to human TCRs.

Furthermore, other effector cells (non-T cells) may be redirected to the site of the target antigen HLA-A*02:01/MAGE-A4²³⁰⁻²³⁹ antigen, e.g. to cancer cells or tumours, and activated, in embodiments wherein the second antigen binding domain of the antigen binding proteins (e.g. antibodies) of the invention binds to (or specifically binds to) an antigen present on the surface of the effector cells. For instance, the second antigen binding domain may bind to (or specifically bind to) CD16, NKG2D, NKp30, or NKp46 receptors, in which case natural killer (NK) cells may be redirected and activated. Alternatively, the second antigen binding domain may be bind to (e.g. specifically bind to) SIRPα, in which case macrophages may be recruited and activated.

In an embodiment, the antigen binding protein (e.g. antibody) of the invention is bispecific, e.g. is a bispecific antibody, which comprises a third, and optionally further, antigen binding domain(s). In this embodiment, the second antigen binding domain binds to, or specifically binds to, a second antigen, i.e. to an antigen other than HLA-A*02:01/MAGE-A4²³⁰⁻²³⁹, preferably as described elsewhere herein, and the third antigen binding domain binds to, or specifically binds to HLA-A*02:01/MAGE-A4²³⁰⁻²³⁹. The third antigen binding domain may incorporate, singly or in combination, any of the features described herein in relation to the first antigen binding domain. In one embodiment, the third antigen domain is identical to the first antigen binding domain. Further antigen binding domains may bind to, i.e. specifically bind to HLA-A*02:01/MAGE-A4²³⁰⁻²³⁹ or to the same antigen as the second binding domain.

In an embodiment, the antigen binding protein (e.g. antibody) of the invention is trispecific, e.g. is a trispecific antibody. In other words, the antigen binding protein (e.g. antibody) of the invention preferably comprises a second antigen binding domain which binds to, or specifically binds to, a second antigen, preferably as described elsewhere herein, and a third antigen binding domain which binds to, or specifically binds to, a third antigen, wherein said first, second and third antigens are different from each other. The trispecific antigen binding proteins (e.g. antibodies) of the invention are preferably also T-cell engagers (as defined and described above).

The bispecific, trispecific or multispecific antigen binding protein (e.g. antibody) may be of any known format, including one selected from the group consisting of single-chain diabody (scDb), a tandem scDb (Tandab), a linear dimeric scDb (LD-scDb), a circular dimeric scDb (CD-scDb), a bispecific T-cell engager (BiTE; tandem di-scFv), a tandem tri-scFv, a tribody (Fab-(scFv)2) or bibody (Fab-(scFv)1), triabody, scDb-scFv, bispecific Fab2, di-miniantibody, tetrabody, scFv-Fc-scFv fusion, di-diabody, DVD-Ig, COVD, IgG-scFab, scFab-dsscFv, Fv2-Fc, IgG-scFv fusions, such as bsAb (scFv linked to C-terminus of light chain), Bs1Ab (scFv linked to N-terminus of light chain), Bs2Ab (scFv linked to N-terminus of heavy chain), Bs3Ab (scFv linked to C-terminus of heavy chain), Ts1Ab (scFv linked to N-terminus of both heavy chain and light chain), Ts2Ab (dsscFv linked to C-terminus of heavy chain), Knob-into-Hole antibodies (KiHs), a MATCH and DuoBodies.

As mentioned above, the antigen binding proteins (e.g. antibodies) of the present invention are preferably T-cell engaging antibodies (or antigen binding proteins), also termed “T-cell engagers” (TCEs). These are (at least) bispecific molecules, and in a preferred embodiment are T-cell engaging bispecific antigen binding proteins (e.g. antibodies).

In a preferred embodiment, the antigen binding protein (e.g. antibody) of the invention is a single-chain T-cell engaging bispecific antigen binding protein (e.g. antibody).

As mentioned above, in such antigen binding proteins (e.g. antibodies), the second antigen binding preferably binds to (or specifically binds to) CD3. The antigen binding domains in such bispecific molecules are as defined elsewhere herein.

In a preferred embodiment, the antigen binding protein of the invention is a single-chain bispecific diabody, i.e. a bispecific antibody in the single-chain bispecific diabody (scDb) format.

Preferably, the scDb has the capability of binding to (or specifically binding to) both HLA-A*02:01/MAGE-A4²³⁰⁻²³⁹ via a first antigen binding domain, as described above, and to a T-cell surface antigen, preferably CD3, via a second antigen binding domain as described above. The antigen binding domains in such bispecific molecules are as defined elsewhere herein.

The scDb format has a more compact form in comparison to other bispecific antibody formats and allows the formation of immunocytolytic synapses.

Thus, in a further aspect and in certain embodiments, the present invention provides a single-chain bispecific diabody comprising:
i) a first heavy chain variable domain of an immunoglobulin with a first specificity (VHA), wherein said first specificity is for HLA-A*02:01/MAGE-A4²³⁰⁻²³⁹ wherein said VHA comprises:
   a variable heavy (VH) CDR1, a VH CDR2 and a VH CD3 comprising, respectively, the amino acid sequences of the VH CDR1, VH CDR2 and VH CDR3 of a specific antigen binding protein (e.g. antibody) of the invention (disclosed in Table A), or sequences substantially homologous thereto;
ii) a first light chain variable domain of an immunoglobulin with said first specificity (VLA), wherein said VLA comprises:
   a variable light (VL) CDR1, a VL CDR2 and a VL CD3 comprising, respectively, the amino acid sequences of the VL CDR1, VL CDR2 and VL CDR3 of a (preferably said) specific antigen binding protein (e.g. antibody) of the invention (disclosed in Table A), or sequences substantially homologous thereto;
iii) a second heavy chain variable domain of an immunoglobulin with a second specificity (VHB), wherein said second specificity is for a T cell CD3; and
iv) a second light chain variable domain of an immunoglobulin with said second specificity (VLB);
   wherein the VLA, VHB, VLB and VHA domains are connected within a single polypeptide chain, and wherein each domain is connected to the adjacent domain(s) via a peptide linker. Preferred specific antibodies of the invention and the CDR sequences thereof that are disclosed in Table A are as described above.

In a further aspect and in certain embodiments the present invention provides a single-chain bispecific diabody comprising:
i) a first heavy chain variable domain of an immunoglobulin with a first specificity (VHA), wherein said first specificity is for HLA-A*02:01/MAGE-A4²³⁰⁻²³⁹ wherein said VHA comprises the amino acid sequence of a heavy chain variable domain of a specific antigen binding protein (e.g. antibody) of the invention (disclosed in Table A), or a sequence substantially homologous thereto;
ii) a first light chain variable domain of an immunoglobulin with said first specificity (VLA), wherein said VLA comprises the amino acid sequence of a light chain variable domain of a (preferably said) specific antigen binding protein (e.g. antibody) of the invention (disclosed in Table A), or a sequence substantially homologous thereto;
iii) a second heavy chain variable domain of an immunoglobulin with a second specificity (VHB), wherein said second specificity is for a T cell CD3; and
iv) a second light chain variable domain of an immunoglobulin with said second specificity (VLB);
wherein the VLA, VHB, VLB and VHA domains are connected within a single polypeptide chain, and wherein each domain is connected to the adjacent domain(s) via a peptide linker. Preferred specific antibodies of the invention and the VH and VL domain sequences thereof that are disclosed in Table A are as described above.

In a further aspect and in certain embodiments, the present invention provides a single-chain bispecific diabody comprising:
i) a first heavy chain variable domain of an immunoglobulin with a first specificity (VHA), wherein said first specificity is for HLA-A*02:01/MAGE-A4²³⁰⁻²³⁹ wherein said VHA comprises:
   (a) a variable heavy (VH) CDR1 that comprises the amino acid sequence of GYTLTELS (SEQ ID NO:5) or a sequence substantially homologous thereto;
   (b) a VH CDR2 that comprises the amino acid sequence of FDPEDGET (SEQ ID NO:6) or a sequence substantially homologous thereto; and
   (c) a VH CDR3 that comprises the amino acid sequence of ATDQGSSWGFY (SEQ ID NO:7) or a sequence substantially homologous thereto;
ii) a first light chain variable domain of an immunoglobulin with said first specificity (VLA), wherein said VLA comprises:
   (d) a variable light (VL) CDR1 that comprises the amino acid sequence of QSISSY (SEQ ID NO:8) or a sequence substantially homologous thereto;
   (e) a VL CDR2 that comprises the amino acid sequence of AAS (SEQ ID NO:9) or a sequence substantially homologous thereto; and
   (f) a VL CDR3 that comprises the amino acid sequence of QQSYSTPYT (SEQ ID NO:10) or QQSYSTPFT (SEQ ID NO:96), or a sequence substantially homologous thereto;
iii) a second heavy chain variable domain of an immunoglobulin with a second specificity (VHB), wherein said second specificity is for a T cell CD3; and
iv) a second light chain variable domain of an immunoglobulin with said second specificity (VLB);
   wherein the VLA, VHB, VLB and VHA domains are connected within a single polypeptide chain, and wherein each domain is connected to the adjacent domain(s) via a peptide linker. Preferred CDRs and substantially homologous sequences are as described anywhere else herein.

In a further aspect and in certain embodiments, the present invention provides a single-chain bispecific diabody comprising:
ii) a first heavy chain variable domain of an immunoglobulin with a first specificity (VHA), wherein said first specificity is for HLA-A*02:01/MAGE-A4²³⁰⁻²³⁹ wherein said VHA comprises:
   (a) a variable heavy (VH) CDR1 that comprises the amino acid sequence of GYTLTELS (SEQ ID NO:5) or a sequence substantially homologous thereto;
   (b) a VH CDR2 that comprises the amino acid sequence of FDPEDGET (SEQ ID NO:6) or a sequence substantially homologous thereto; and
   (c) a VH CDR3 that comprises the amino acid sequence of ATDQGSSWGFY (SEQ ID NO:7) or ATDQGASWGFY (SEQ ID NO: 85), or a sequence substantially homologous thereto;
ii) a first light chain variable domain of an immunoglobulin with said first specificity (VLA), wherein said VLA comprises:
   (d) a variable light (VL) CDR1 that comprises the amino acid sequence of QNIMWY (SEQ ID NO:87) or a sequence substantially homologous thereto;
   (e) a VL CDR2 that comprises the amino acid sequence of AAS (SEQ ID NO:9) or a sequence substantially homologous thereto; and
   (f) a VL CDR3 that comprises the amino acid sequence of QQSYSTPYT (SEQ ID NO:10), or a sequence substantially homologous thereto;
v) a second heavy chain variable domain of an immunoglobulin with a second specificity (VHB), wherein said second specificity is for a T cell CD3; and
vi) a second light chain variable domain of an immunoglobulin with said second specificity (VLB);
   wherein the VLA, VHB, VLB and VHA domains are connected within a single polypeptide chain, and wherein each domain is connected to the adjacent domain(s) via a peptide linker.

In a further aspect and in certain embodiments, the present invention provides a single-chain bispecific diabody comprising:
i) a first heavy chain variable domain of an immunoglobulin with a first specificity (VHA), wherein said first specificity is for HLA-A*02:01/MAGE-A4²³⁰⁻²³⁹ wherein said VHA comprises the amino acid sequence of SEQ ID NO: 3 or a sequence substantially homologous thereto;
ii) a first light chain variable domain of an immunoglobulin with said first specificity (VLA), wherein said VLA comprises the amino acid sequence of SEQ ID NO: 4, 114, 116 or 147, or a sequence substantially homologous thereto;
iii) a second heavy chain variable domain of an immunoglobulin with a second specificity (VHB), wherein said second specificity is for a T cell CD3; and
iv) a second light chain variable domain of an immunoglobulin with said second specificity (VLB);
wherein the VLA, VHB, VLB and VHA domains are connected within a single polypeptide chain, and wherein each domain is connected to the adjacent domain(s) via a peptide linker. Preferred VH and VL domains and substantially homologous sequences are as described anywhere else herein.

In a further aspect and in certain embodiments, the present invention provides a single-chain bispecific diabody comprising:
ii) a first heavy chain variable domain of an immunoglobulin with a first specificity (VHA), wherein said first specificity is for HLA-A*02:01/MAGE-A4²³⁰⁻²³⁹ wherein said VHA comprises the amino acid sequence of SEQ ID NO: 118, or a sequence substantially homologous thereto;
ii) a first light chain variable domain of an immunoglobulin with said first specificity (VLA), wherein said VLA comprises the amino acid sequence of SEQ ID NO: 116 or 142, or a sequence substantially homologous thereto;
v) a second heavy chain variable domain of an immunoglobulin with a second specificity (VHB), wherein said second specificity is for a T cell CD3; and
vi) a second light chain variable domain of an immunoglobulin with said second specificity (VLB);
wherein the VLA, VHB, VLB and VHA domains are connected within a single polypeptide chain, and wherein each domain is connected to the adjacent domain(s) via a peptide linker.

In a further aspect and in certain embodiments, the present invention provides a single-chain bispecific diabody comprising:
iii) a first heavy chain variable domain of an immunoglobulin with a first specificity (VHA), wherein said first specificity is for HLA-A*02:01/MAGE-A4²³⁰⁻²³⁹ wherein said VHA comprises the amino acid sequence of SEQ ID NO: 130, or a sequence substantially homologous thereto;
ii) a first light chain variable domain of an immunoglobulin with said first specificity (VLA), wherein said VLA comprises the amino acid sequence of SEQ ID NO: 4, or a sequence substantially homologous thereto;
vii) a second heavy chain variable domain of an immunoglobulin with a second specificity (VHB), wherein said second specificity is for a T cell CD3; and
viii) a second light chain variable domain of an immunoglobulin with said second specificity (VLB);
wherein the VLA, VHB, VLB and VHA domains are connected within a single polypeptide chain, and wherein each domain is connected to the adjacent domain(s) via a peptide linker.

The VLA, VHB, VLB and VHA domains are connected within (i.e. in) a single polypeptide chain, i.e. are fused, i.e. connected linearly, via peptide linkers. There are thus three peptide linkers connecting the four domains. The domains may be connected in any order/orientation, provided that upon proper folding of the resulting polypeptide, two functional antigen binding domains are formed.

Preferred orders/orientations are, in the N to C direction: i) VLA-VHB-VLB-VHA, ii) VLB-VHA-VLA-VHB; iii) VHA-VLB-VHB-VLA and iv) VHB-VLA-VHA-VLB, most preferred are i) VLA-VHB-VLB-VHA, and ii) VLB-VHA-VLA-VHB, particularly i) VLA-VHB-VLB-VHA.

As stated above, the antigen binding proteins of the invention comprise at least one antigen binding domain, said antigen binding domain comprising a heavy chain variable domain (VH domain) that comprises three complementarity determining regions (CDRs), and a light chain variable domain (VL domain) that comprises three CDRs. The scDbs of the invention comprise two such antigen binding domains: one comprising VHA and VLA, and one comprising VHB and VLB.

In the scDbs of the invention, optional and preferred features and definitions of the first heavy and light chain variable domains, including the CDR and domain sequences, and the substantially homologous sequences, are as defined elsewhere herein. Optional and preferred features and definitions of the second heavy and light chain variable domains, including the CDR and domain sequences, and the substantially homologous sequences, are as defined elsewhere herein.

As mentioned above, the selection of (e.g. length and sequence of) peptide linkers, and the orientation of domains and peptide linkers, are well-known in the field, and would be within the competencies of the person of ordinary skill in the art. Linkers suitable for use have been studied in detail and are well known in the field, e.g. as described in Völkel et al., (2001) Protein Engineering, Design and Selection, Volume 14, Issue 10, Pages 815-823.

The scDbs comprise a peptide linker between each of:
i) the VLA and the VHB domains (or vice versa, depending on which orientation of domains is present); and
ii) the VLB and the VHA domains (or vice versa, depending on which orientation of domains is present); and
iii) the VHB and the VLB domains (or vice versa, depending on which orientation of domains is present); or
iv) the VHA and VLA domains (or vice versa) depending on which orientation of domains is present.

Thus, the sDbs comprise both linker i) and linker ii) and either linker iii) or linker iv), depending on the orientation of domains therein.

Typically, the peptide linker between the VLA and the VHB domains (or vice versa) (linker (i)) and the peptide linker between the VLB and the VHA domains (or vice versa) (linker (ii)), is relatively short, e.g. 3 to 7 amino acids, e.g. 4 to 7 amino acids or 3 to 5 amino acids, i.e. 3, 4 or preferably 5 amino acids. These are termed the “scDb short linkers” herein. In contrast, the peptide linker between the VHB and the VLB domains (or vice versa) (linker (iii)) or in other arrangements between the VHA and VLA domains (or vice versa) (linker (iv)), is relatively longer, e.g. 12 to 21, preferably 13-19, more preferably 14 to 17 amino acids, e.g. 14, 15, 16 or 17 amino acids. This is termed the “scDb short linker” herein.

Typically, the peptide linker between the VHB and the VLB domains (or vice versa) (linker (iii)) or in other arrangements between the VHA and VLA domains (or vice versa) (linker (iv)), is 2.5 to 3.5 times longer, e.g. about 3 times longer, than the peptide linker between the VLA and the VHB domains (or vice versa) (linker (i)), and the peptide linker between the VLB and the VHA domains (or vice versa) (linker (ii)). The presence of a longer peptide linker between the two central domains of the single polypeptide chain, and the presence of shorter peptide linkers between each of the N and C terminal domains and their respective adjacent domains, achieves correct folding and formation of the two antigen binding domains.

The short linker between the VLA and the VHB domains (or vice versa) (linker (i)) and the short linker between the VLB and the VHA domains (or vice versa) (linker (ii)), may be of identical length and/or (preferably “and”) amino acid sequence.

The amino acid sequences of the various peptide linkers is not particularly limited; suitable linker sequences could be identified and prepared by the person of ordinary skill in the art, e.g. with reference to Völkel et al., (2001) Protein Engineering, Design and Selection, Volume 14, Issue 10, Pages 815-823, and any suitable linker sequence may be used.

In some embodiments, the peptide linker sequences comprise only hydrophilic amino acids (e.g. arginine, asparagine, aspartic acid, glutamine, glutamic acid, histidine, lysine, serine and threonine) and aliphatic amino acids (e.g. alanine, glycine, isoleucine, leucine, proline, and valine).

In some embodiments, the peptide linker sequences comprise only amino acids selected from the group consisting of threonine, asparagine, serine, aspartic acid, glycine and alanine.

In certain embodiments relating to single-chain bispecific diabodies, said peptide linker between the VLA and the VHB domains (or vice versa) (linker (i)) and between the VLB and the VHA domains (or vice versa) (linker (ii)) consists of four Glycine amino acid residues and one Serine amino acid residue (GGGGS, SEQ ID NO: 26), and the peptide linker between the VHB and the VLB domains (or vice versa) (linker (iii)), or between the VHA and VLA domains (or vice versa) (linker (iv)) consists of three contiguous sequences consisting each of four Glycine amino acid residues and one Serine amino acid residues (GGGGS, SEQ ID NO: 26), i.e. GGGGSGGGGSGGGGS (SEQ ID NO: 27).

In one embodiment, the antigen binding proteins are chimeric antigen receptors (CARs). As used herein, the term “chimeric antigen receptor” or “CAR” refers to a receptor that is capable of activating an immune cell in response to antigen binding. CARs are recombinant membrane spanning molecules and are advantageously expressed on immune cells. Their structure typically comprises (i) an extracellular domain (“ectodomain” or “antibody domain”), (ii) a transmembrane domain and (iii) a cytoplasmic domain (endodomain or intracellular signalling domain).

The ectodomain (i.e., antibody domain) comprises an antigen binding domain that binds to, or specifically binds to, HLA-A*02:01/MAGE-A4²³⁰⁻²³⁹, as defined elsewhere herein. Typically, the ectodomain comprises an antigen binding protein in the scFv format, but other antibody fragments/formats may also be used. A spacer sequence generally connects the ectodomain and the transmembrane domain, which in turn is connected to an endodomain. Upon binding of the ectodomain to the antigen, the receptors cluster and an activation signal is transmitted to the immune cell which results in initiation of an immune response against the cell on which the target antigen (HLA-A*02:01/MAGE-A4²³⁰⁻²³⁹) is displayed.

First generation CARs have a simply structured endodomain comprising CD3-zeta. To increase the activation signal, a co-stimulatory domain was added in the second-generation CARs; and third generation CARs include two or more co-stimulatory domain. Said co-stimulatory domains may be selected from the group consisting of CD28, OX40 and/or 4-1BB. Apart from CD3-zeta, other ITAM-containing domains have been explored including the Fc receptor for IgE-γ domain.

Thus, in one embodiment, the invention provides a chimeric antigen receptor (CAR) that specifically recognizes HLA-A*02:01/MAGE-A4²³⁰⁻²³⁹, comprising: i) an ectodomain that comprises an antigen binding domain which binds to, or specifically binds to, HLA-A*02:01/MAGE-A4²³⁰⁻²³⁹, as defined anywhere elsewhere herein; ii) a transmembrane domain; and iii) an intracellular signalling domain.

In certain embodiments, the transmembrane domain is selected from the group consisting of an artificial hydrophobic sequence and transmembrane domains of a type I transmembrane protein, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the intracellular signalling domain is selected from the group consisting of cytoplasmic signalling domains of a human CD3 zeta chain, FcγRIII, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

Other suitable components, e.g. domains, of CARs, and means for their inclusion are well-known in the field, and any suitable component or domain may be included in the CARs of the present invention.

In a further aspect, the invention provides immune cells engineered to express CARs comprising the antigen binding proteins described herein.

Suitable immune cells include, without being limited to, T cells, Natural Killer T (NKT) cells, natural killer (NK) cells, human embryonic stem cells, hematopoietic stem cells (HSC) or induced pluripotent stem cells (iPS). Such T cells may be a cytotoxic T lymphocyte (CTL), a regulatory T lymphocyte, an inflammatory T-lymphocytes, or a helper T-lymphocyte or a gamma-delta T cell. The T cell may be a CD4+ or CD8+ or a mixed population of CD4+ and CD8+ cells. T cells expressing a CAR are termed CAR-T cells and in embodiments, immune cells engineered to express CARs comprising the antigen binding proteins described herein are CAR-T cells.

### Footprint/Binding Specificity

Example 1 herein describes the examination of the fine-specificity of an antibody of the invention, 1-H02, towards the HLA-A*02:01 presented MAGE-A4²³⁰⁻²³⁹ peptide. Positional alanine (Ala) scanning was performed. An array of MAGE-A4²³⁰⁻²³⁹ variant peptides were produced, in each of which one residue of the MAGE-A4²³⁰⁻²³⁹ sequence was mutated to alanine. The variant peptides sequences are shown in Table C. The binding of 1-H02 to the Ala-mutated peptides was assessed using pHLA phage capture ELISA, as described in Example 1, relative to the binding to HLA-A*02:01 presented MAGE-A4²³⁰⁻²³⁹.

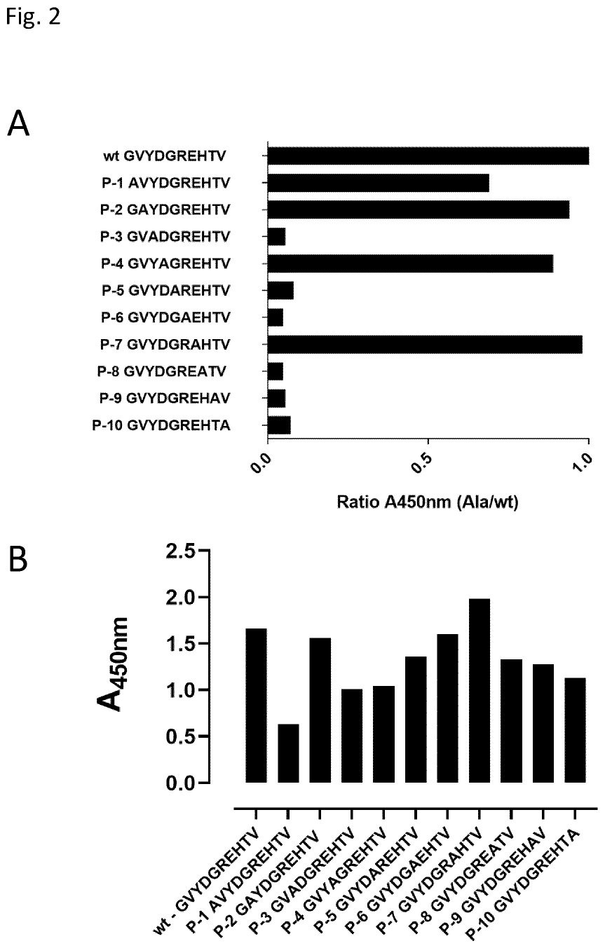

As shown in FIG. 2, 1-H02 exhibited peptide-dependent binding. Specific single Ala mutations in the MAGE-A4²³⁰⁻²³⁹ peptide affected the extent of binding. Specifically, the antibody-pHLA binding was abrogated with peptide variants P3, P5, P6, P8, P9 and P10, indicating that amino acids at positions 3, 5, 6, 8, 9 and 10 of WT MAGE-A4²³⁰⁻²³⁹ (SEQ ID NO: 19) were important for antibody binding. Canonically, HLA restricted peptides are 9mers, whereas WT MAGE-A4²³⁰⁻²³⁹ is a 10mer. For this reasons, amino acid positions 1 to 10 of WT MAGE-A4²³⁰⁻²³⁹ (SEQ ID NO: 19) may be referred to as positions −1 to 9 (i.e. −1, 1, 2, 3, 4, 5, 6, 7, 8 and 9). Using such a canonical numbering system, the amino acids important for binding would be at positions termed 2, 4, 5, 7, 8 and 9, though for the avoidance of doubt, these are actually positions 3, 5, 6, 8, 9 and 10 of WT MAGE-A4²³⁰⁻²³⁹ (SEQ ID NO: 19).

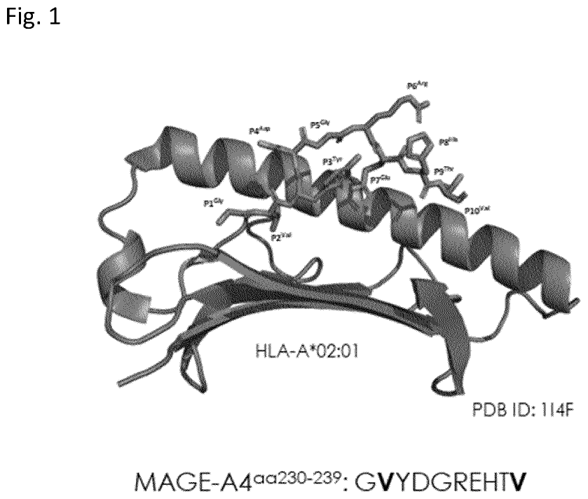

Positions 3, 4, 5, 6, and 8 of the MAGE-A4²³⁰⁻²³⁹ peptide sequence (SEQ ID NO: 19) are solvent-exposed when presented in the HLA-A*02:01 complex (termed positions 2, 3, 4, 5 and 7 in the canonical numbering system, see FIG. 1). Thus, the results showed that clone 1-H02 exhibited restricted positional binding dependency towards 4 of the 5 solvent exposed residues—namely the amino acids at positions 3, 5, 6 and 8 of MAGE-A4²³⁰⁻²³⁹ (SEQ ID NO: 19). This indicates a central binding mode and high degree of specificity for 1-H02 since a binding footprint covering the N- or C-terminal ends of the presented peptide is more likely to result in increased potential for binding promiscuity, i.e. confer a binding specificity less dependent on the particular sequence of the presented peptide. The central binding mode of 1-H02 indicates a high specificity for the particular peptide sequence of MAGE-A4²³⁰⁻²³⁹ in the HLA-A*02:01/MAGE-A4²³⁰⁻²³⁹ complex. The same analysis was performed using many other of the antibodies disclosed in Table A, including but not limited to AM2, AM6, and AM15, with the peptide-dependent binding observed being essentially the same as 1-H02 (data not shown).

Thus, in an embodiment, the antigen binding proteins (e.g. antibodies) of the invention bind to (i.e. are capable of binding to) any one or more of, preferably all of, the amino acids at positions 3, 5, 6 and 8 of the HLA-A*02:01 restricted peptide MAGE-A4²³⁰⁻²³⁹ (SEQ ID NO: 19).

In an embodiment, the antigen binding proteins (e.g. antibodies) of the invention preferentially bind to (or selectively bind to) HLA-A*02:01/MAGE-A4²³⁰⁻²³⁹ as compared to a HLA-A*02:01 restricted peptide comprising (or consisting of) the amino acid sequence of any one, preferably each, of SEQ ID NOs: 33, 35, 36 and 38 (P3, P5, P6, P8 of Table C).

In an embodiment, the antigen binding proteins (e.g. antibodies) of the invention exhibit binding to a HLA-A*02:01 restricted peptide comprising (or consisting of) the amino acid sequence of any one, preferably each, of SEQ ID NOs: 33, 35, 36 and 38 (P3, P5, P6, P8 of Table C), which is at most 15%, e.g. at most 10%, of the exhibited binding of the same antigen binding protein (e.g. antibody) to HLA-A*02:01/MAGE-A4²³⁰⁻²³⁹.

Alternatively viewed, in an embodiment the antigen binding proteins (e.g. antibodies) of the invention exhibit a decrease of at least 85%, e.g. at least 90%, in binding to a HLA-A*02:01 restricted peptide comprising (or consisting of) the amino acid sequence of any one, preferably each, of SEQ ID NOs: 33, 35, 36 and 38 (P3, P5, P6, P8 of Table C), as compared to binding to HLA-A*02:01/MAGE-A4²³⁰⁻²³⁹.

Any substantially homologous antigen binding protein (e.g. antibody) of the invention preferably exhibits such binding.

Preferably such binding is exhibited when the antigen binding protein is an antibody in the scFv format, preferably wherein the scFV is present on the surface of a phage particle.

Example 1 herein describes the assessment of the specificity of an antibody of the invention, 1-H02, AM2 and AM15, for HLA-A*02:01/MAGE-A4²³⁰⁻²³⁹ over HLA-A*02:01 restricted peptides derived from MAGE-A4 homologues with high levels of sequence similarity to MAGE-A4²³⁰⁻²³⁹.

A panel of MAGE-A4²³⁰⁻²³⁹ homologue peptides was prepared (Table D). The binding of 1-H02, AM2 and AM15 to these HLA-A*02:01 presented MAGE-A4²³⁰⁻²³⁹ homologue peptides was assessed using pHLA phage capture ELISA, as described in Example 1, relative to the binding to HLA-A*02:01 presented MAGE-A4²³⁰⁻²³⁹.

Significant binding to any of these homologues, with the exception of the MAGE-A8 derived peptide (SEQ ID NO: 44), could represent a potentially dangerous cross-reactivity risk, should antigen binding proteins (e.g. antibodies) directed to HLA-A*02:01/MAGE-A4²³⁰⁻²³⁹ also bind to them and induce T-cell activation and redirection against the cells displaying them. The peptide derived from MAGE-A8 does not represent a dangerous cross-reactivity risk because MAGE-A8, like MAGE-A4, is a clinically validated cancer specific target antigen.

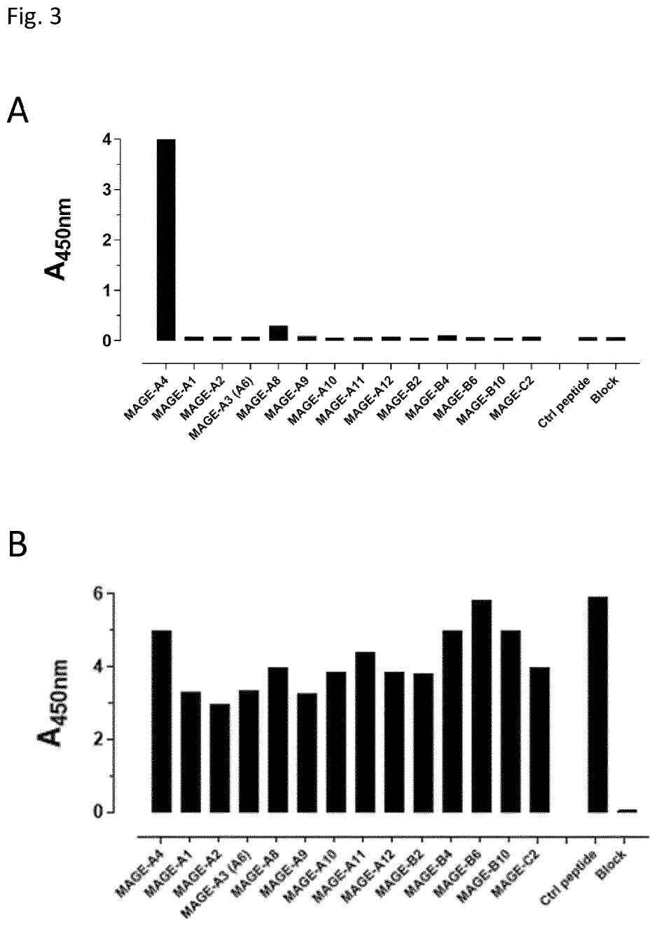

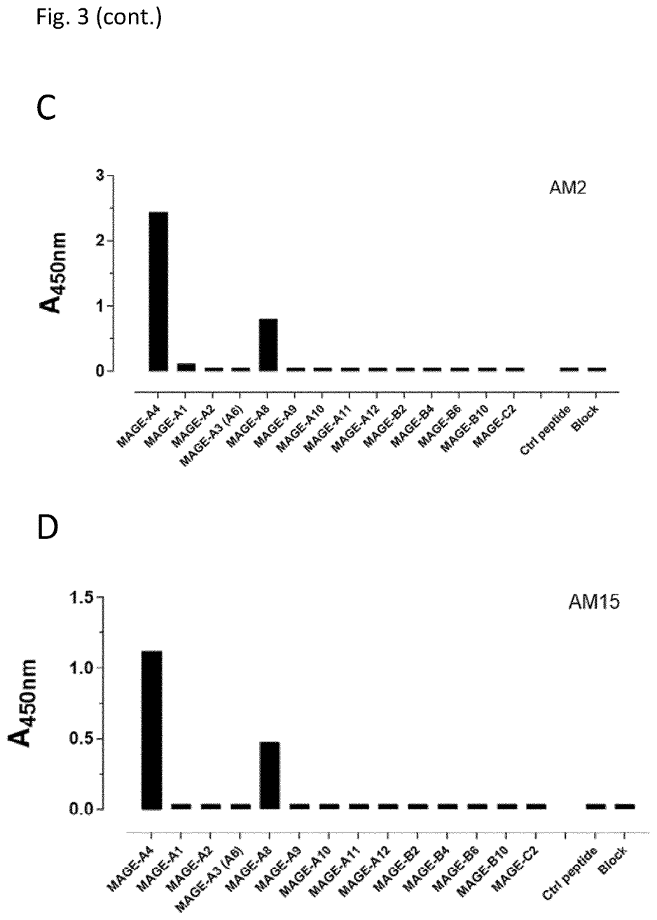

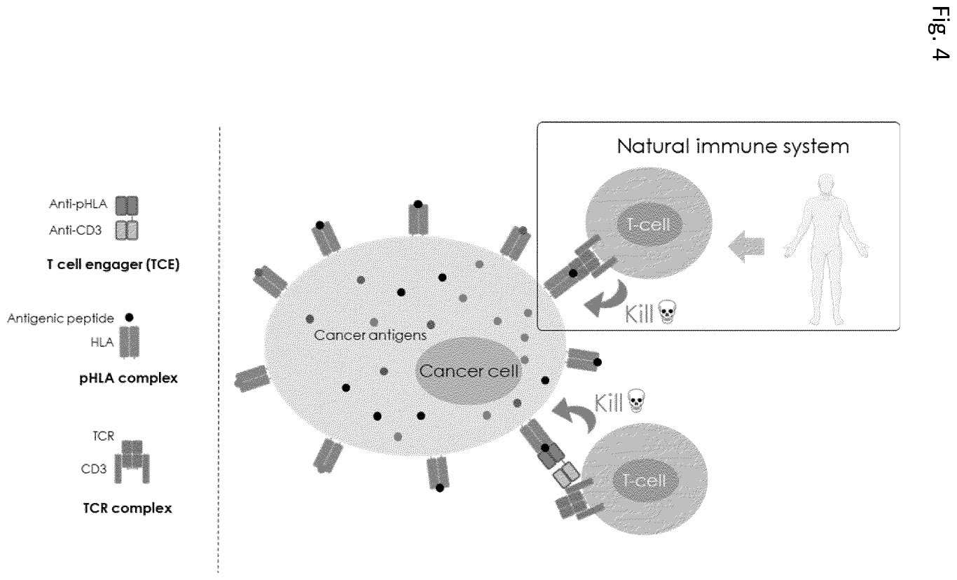

As shown in FIGS. 3A, C and D, 1-H02, AM2 and AM15 exhibited remarkable binding specificity for HLA-A*02:01/MAGE-A4²³⁰⁻²³⁹ over HLA-A*02:01 restricted peptides from all of the MAGE-A4 homologues representing a potentially dangerous cross-reactivity risk (i.e. peptides of SEQ ID NOs: 41 to 43 and 45 to 53), despite the peptides sharing high sequence similarity with MAGE-A4²³⁰⁻²³⁹. This demonstrates the remarkable specificity of the antibodies of the invention.

The only HLA-restricted peptide to which the antibodies of the invention bound to any extent was the MAGE-A8 derived peptide, and this binding was reduced at least 2-fold (AM2 and AM15), and approximately 10-fold (1-H02) as compared to binding to HLA-A*02:01/MAGE-A4²³⁰⁻²³⁹. This specificity of binding is notable, given that the MAGE-A4 and MAGE-A8 peptides only differ in two positions (position 2 and position 9) of SEQ ID NOs: 19 and 44, and the different amino acids at this position in the two sequences are of a similar nature (threonine and serine).

The same analysis was performed using many other of the antibodies disclosed in Table A, with the same binding pattern observed as for 1-H02, AM2 and AM15, i.e. specific binding to HLA-A*02:01/MAGE-A4²³⁰⁻²³⁹ over HLA-A*02:01 restricted peptides from MAGE-A4 homologues (data not shown).

Thus, in an embodiment, the antigen binding proteins (e.g. antibodies) of the invention preferentially bind to (or selectively bind to) (i.e. are capable of binding to (or selectively binding to) HLA-A*02:01/MAGE-A4²³⁰⁻²³⁹ as compared to a HLA-A*02:01 restricted peptide comprising (or consisting of) the amino acid sequence of any one, preferably each, of SEQ ID NOs: 41 to 43 and 45 to 53 (MAGE-A4 homologue peptides).

Thus, in an embodiment, the antigen binding proteins (e.g. antibodies) of the invention exhibit binding to a HLA-A*02:01 restricted peptide comprising (or consisting of) the amino acid sequence of any one, preferably each, of SEQ ID NOs: 41 to 43 and 45 to 53 (MAGE-A4 homologue peptides), which is at most 10%, e.g. at most 5%, of the exhibited binding of the same antigen binding protein (e.g. antibody) to HLA-A*02:01/MAGE-A4²³⁰⁻²³⁹.

Alternatively viewed, in an embodiment the antigen binding proteins (e.g. antibodies) of the invention exhibit a decrease of at least 90%, e.g. at least 95%, in binding to a HLA-A*02:01 restricted peptide comprising (or consisting of) the amino acid sequence of any one, preferably each, of SEQ ID NOs: 41 to 43 and 45 to 53 (MAGE-A4 homologue peptides) as compared to binding to HLA-A*02:01/MAGE-A4²³⁰⁻²³⁹.

Any substantially homologous antigen binding protein (e.g. antibody) of the invention preferably exhibits such binding.

Preferably such binding is exhibited when the antigen binding protein is an antibody in the scFv format, preferably wherein the scFV is present on the surface of a phage particle.

### Assay 1: Assay for Binding Specificity (ELISA)

Binding of an antigen binding protein (e.g. antibody) to a HLA-restricted peptide (pHLA) can be assessed by any appropriate means and the skilled person is familiar with suitable methods (e.g. an ELISA assay such as an ELISA assay in which the pHLA is coated on ELISA plates/wells).

In some embodiments, binding of an antigen binding protein (e.g. antibody) of the invention to HLA-restricted peptide (pHLA) may be as determined by (or as assessed by) an ELISA assay, e.g. an ELISA assay that comprises:
(a) Coating an ELISA plate (i.e. wells of the plate) with neutravidin (e.g. 5 μg/mL) in PBS (phosphate-buffered saline) and incubating for about 16 hours e.g. at 4° C.;
(b) Washing the coated plate, for example with PBS containing 0.05% Tween (from hereon “PBST” means “PBS containing 0.05% Tween”), at least once, e.g. three times;
(c) Incubating the avidin coated plate (e.g. for about 1 hour), e.g. at room (or ambient) temperature (e.g. 25° C.) with a blocking buffer (e.g. 5% skimmed milk powder in PBST (from hereon “PBSTM”);
(d) Washing the blocked plate, for example with PBST, at least once, e.g. three times;
(e) Incubating the ELISA plate with biotinylated pHLA (e.g. in blocking buffer, e.g. PBSTM) and incubating for about 1 h at room (or ambient) temperature (e.g. 25° C.);
(f) Washing the coated plate (wells of the coated plate), for example with PBST, at least once, e.g. three times
(g) Incubating the antigen binding protein (e.g. antibody) to be tested (e.g. in scDb, IgG, or scFv format wherein scFv is present on the surface of phage particles) diluted in blocking buffer (e.g. PBSTM), e.g. over a dilution series, e.g. starting at 385 nM for scDb, 250 nM for IgG, or 10⁹ phage/ml for scFv-phage, in wells of the ELISA plate, e.g. for 1 h at for example room (or ambient) temperature (e.g. 25° C.) for scDb or IgG, or 37° C. for scFv-phage;
(h) Washing the plate (wells of the plate), for example with PBST, at least once, e.g. three times;
(i) Incubating in the wells of the plate, e.g. for 1 h, for example at room (or ambient) temperature (e.g. 25° C.), a secondary antibody having (e.g. conjugated to) a detectable label (e.g. horse radish peroxidase, “HRP”), e.g. anti-M13-HRP (e.g. stock solution diluted 1:5000 in 100 μl blocking buffer (e.g. PBSTM)/well) for scFv-phage, or protL (e.g. diluted 1:10,000 in 100 μl blocking buffer (e.g. PBSTM)/well) for scDb or IgG;
(j) Washing the plate (wells of the plate), for example with PBST, at least once, e.g. three times;
(k) Detecting (and quantifying) the detectable label. For example, if HRP (horseradish peroxidase) is used as the detectable label, then a HRP substrate (e.g. TMB substrate (3,3′,5,5′-Tetramethylbenzidine)) may be added to the wells and incubated (e.g. for 10 min at 37° C.), and the reaction may then be stopped (e.g. with 100 μl/well of 1M HCl) and absorbance measured, e.g. at 450 nm, e.g. using a microplate reader.

A preferred ELISA assay for assessing the ability of an antigen binding protein (e.g. antibody) to bind to a HLA-restricted peptide (pHLA) is described in the Example section herein.

In such an ELISA assay used to determine binding, the antigen binding protein may be an antibody in any format. In a preferred assay, the antibody is in the scDb, IgG or scFv format. If the scFv format is used, then the scFv is present on the surface of a phage particle. If phage particles are used, then preferably 10⁹ phages/well are used in step (g) of the above method.

### Specificity of T-Cell Redirection

Example 3 herein describes the assessment of the specificity with which an antibody of the invention, 1-H02 in the scDb format, redirects T-cell activity against cells displaying the target antigen HLA-A*02:01/MAGE-A4²³⁰⁻²³⁹ as compared to against cells displaying HLA-A*02:01 restricted peptides comprising (or consisting of) sequences of human genomic origin identified as having high levels of sequence similarity to MAGE-A4²³⁰⁻²³⁹ (termed “risk peptides”). The identified human peptides are shown in Table G. Such HLA-A2 restricted peptides represent a potential cross-reactivity risk, should antigen binding proteins (e.g. antibodies) directed to HLA-A*02:01/MAGE-A4²³⁰⁻²³⁹ also bind to them and induce T-cell activation and redirection against the cells displaying them.

A panel of genomic risk peptides was prepared (Table G), and exogenously added to a culture of T2 cells which were pulsed to induce HLA-A*02:01 presentation of said peptides. A Jurkat-NFAT activation assay was performed as described in Example 3. This is a well-known and widely used assay in the field, which comprises the use of Jurkat T cell line (TCR/CD3 “Effector Cells”) that expresses a luciferase reporter driven by a Nuclear factor of activated T-cells-response element (NFAT-RE). When the Jurkat T cells are engaged with an anti-CD3 antibody, their T-cell receptor (TCR) transduces intracellular signals resulting in NFAT-RE-mediated luminescence. Thus, the assay indicates the ability of a T-cell engaging antigen binding protein (e.g. antibody) with a first antigen binding domain specific for a target antigen, and a second antigen binding domain specific to a T-cell surface antigen (e.g. CD3), to redirect T cell activity in a specific manner against cells displaying the target antigen.

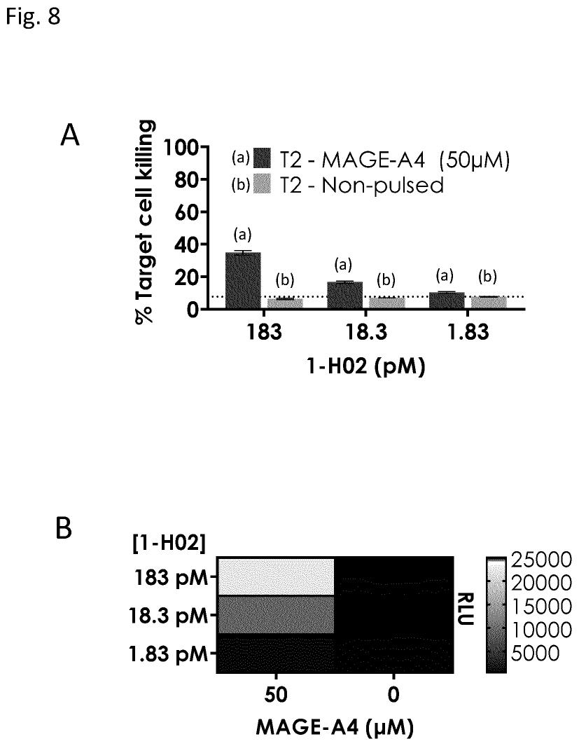

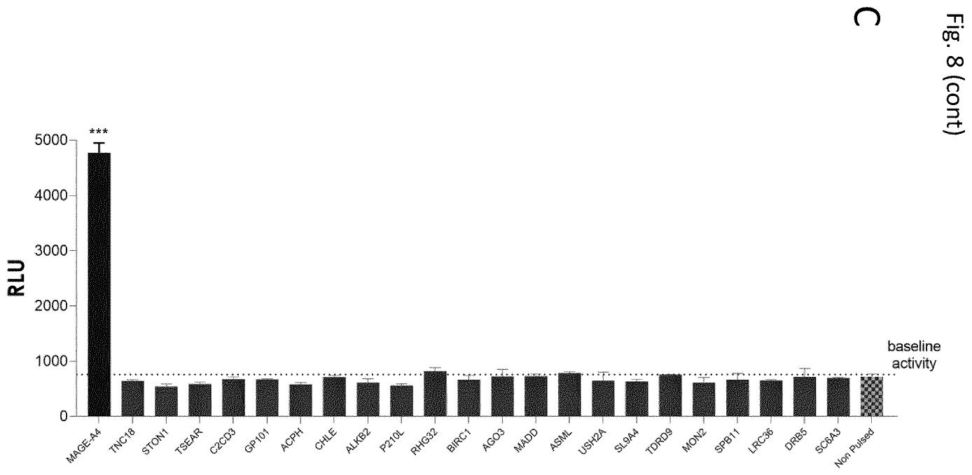

FIGS. 8A and 8B demonstrate that the assay is a valid proxy for T-cell mediated killing, i.e. the level of T-cell activation seen via NFAT-RE-mediated luminescence, correlates with the level of target cell killing. FIG. 8C shows the extent to which 1-H02 induced Jurkat T-cell activation/redirection against pHLA restricted risk peptides relative to HLA-A*02:01/MAGE-A4²³⁰⁻²³⁹ and relative to non-pulsed T2 cells (i.e. cells lacking any pHLA restricted exogenous peptide). FIGS. 16A to 16F show the extent to which AM2, AM6, AM15, AMC8, AMC9 and AMC11 induced Jurkat T-cell activation/redirection against pHLA restricted risk peptides relative to HLA-A*02:01/MAGE-A4²³⁰⁻²³⁹ and relative to non-pulsed T2 cells (i.e. cells lacking any pHLA restricted exogenous peptide).

As shown in FIGS. 8C and 16A to 16F, the antibodies of the invention induced T-cell activation (redirected T-cell activity) with remarkable specificity against cells displaying HLA-A*02:01/MAGE-A4²³⁰⁻²³⁹ over cells displaying all of the HLA-A*02:01 restricted risk peptides. Jurkat activation was only observed when T2 cells were pulsed with MAGE-A4²³⁰⁻²³⁹ peptide; the level of activation observed with all (each) of the risk peptides was comparable to non-pulsed T2 cells, despite these risk peptides sharing high sequence similarity with MAGE-A4²³⁰⁻²³⁹. This further demonstrates the remarkable specificity of the antibodies, which is not only in terms of antigen binding (demonstrated above), but is here demonstrated also in terms of redirecting T-cell activity specifically against HLA-A*02:01/MAGE-A4²³⁰⁻²³⁹ positive cells.

The antigen binding proteins (e.g. antibodies) of the invention, when in a T-cell engager format (e.g. in the scDb format), preferentially (or selectively) redirect (i.e. are capable of preferentially (or selectively) redirecting) T-cell activity against cells displaying HLA-A*02:01/MAGE-A4²³⁰⁻²³⁹ (i.e. HLA-A*02:01/MAGE-A4²³⁰⁻²³⁹ positive cells).

In an embodiment, the antigen binding proteins (e.g. antibodies) of the invention, when in a T-cell engager format (e.g. in the scDb format), preferentially (or selectively) redirect T-cell activity against cells displaying HLA-A*02:01/MAGE-A4²³⁰⁻²³⁹ as compared to against cells displaying a HLA-A*02:01 restricted peptide that is not MAGE-A4²³⁰⁻²³⁹.

Thus, in an embodiment, the antigen binding proteins (e.g. antibodies) of the invention, when in a T-cell engager format (e.g. in the scDb format), preferentially (or selectively) redirect T-cell activity against cells displaying HLA-A*02:01/MAGE-A4²³⁰⁻²³⁹ as compared to against cells displaying a HLA-A*02:01 restricted peptide comprising (or consisting of) the amino acid sequence of any one, preferably each, of SEQ ID NOs: 54 to 75 (genomic risk peptides).

In this context, the term “redirect T-cell activity” against cells displaying a given (target) antigen is meant that the T-cell engaging antigen binding protein (e.g. antibody) forms an immunological synapse by binding to the T-cell antigen (e.g. CD3) and to the given target antigen displayed on target cells, leading to recruitment of T cells to the target cells and activation of T-cell function against said target cells. In other words, the term means induce (i.e., promote, enhance or increase) T-cell activity against the cells displaying the target antigen. Induce need not mean that the starting activity level is zero, i.e. it may mean “induce further”.

Thus, in an embodiment, the antigen binding proteins (e.g. antibodies) of the invention when in a T-cell engager format (e.g. in the scDb format), redirect T-cell activity against pulsed T2 cells displaying a HLA-A*02:01 restricted peptide comprising (or consisting of) the amino acid sequence of any one, preferably each, of SEQ ID NOs: 54 to 75 (genomic risk peptides) to an extent that is not different, e.g. is not significantly different, from the extent of T-cell activity redirected by the same antigen binding protein (e.g. antibody) against negative control cells. Suitable negative control cells may be non-pulsed T2 cells, i.e. T2 cells lacking any pHLA-restricted exogenous peptide.

Alternatively viewed, in an embodiment, the antigen binding proteins (e.g. antibodies) of the invention when in a T-cell engager format (e.g. in the scDb format), redirect T-cell activity against cells displaying a HLA-A*02:01 restricted peptide comprising (or consisting of) the amino acid sequence of any one, preferably each, of SEQ ID NOs: 54 to 75 (genomic risk peptides) to an extent that is not different, e.g. is not significantly different, from the extent of T-cell activity redirected by the same antigen binding protein (e.g. antibody) against negative control cells. Suitable negative control cell cells may be cells displaying neither said HLA-A*02:01 restricted genomic risk peptides, nor HLA-A*02:01/MAGE-A4²³⁰⁻²³⁹, i.e. cells against which merely background levels of redirected T-cell activity are observed.

Thus, in an embodiment, the antigen binding proteins (e.g. antibodies) of the invention, when in a T-cell engager format (e.g. in the scDb format), redirect T-cell activity against cells displaying a HLA-A*02:01 restricted peptide comprising (or consisting of) the amino acid sequence of any one, preferably each, of SEQ ID NOs: 54 to 75 (genomic risk peptides) to an extent that is at most 25%, e.g. at most 20% of the T-cell activity redirected by the same antigen binding protein (e.g. antibody) against cells displaying HLA-A*02:01/MAGE-A4²³⁰⁻²³⁹.

Alternatively viewed, in an embodiment, the antigen binding proteins (e.g. antibodies) of the invention, when in a T-cell engager format, exhibit a decrease of at least 75%, e.g. at least 80%, in T-cell activity redirected against cells displaying a HLA-A*02:01 restricted peptide comprising (or consisting of) the amino acid sequence of any one, preferably each, of SEQ ID NOs: 54 to 75 (genomic risk peptides) as compared to the T-cell activity redirected by the same antigen binding protein (e.g. antibody) against cells displaying HLA-A*02:01/MAGE-A4²³⁰⁻²³⁹.

Any substantially homologous antigen binding protein (e.g. antibody) of the invention preferably exhibits such redirection of T-cell activity.

Preferably such redirection of T-cell activity is exhibited when the antigen binding protein is any T-cell engager format, preferably in a bispecific format. Preferably, the antibody is in the scDb format.

Preferably such redirection of T cell activity is exhibited when the ratio of T cells (or effector cells comprising T cells) to cells displaying HLA-A*02:01/MAGE-A4²³⁰⁻²³⁹ (“target cells”) is about 1:1. Thus, preferably such redirection of T cell activity is exhibited in an assay comprising the co-culturing of HLA-A*02:01/MAGE-A4²³⁰⁻²³⁹ positive target cells (“T”) and effector cells comprising T cells (“E”) at an E:T ratio of about 1:1 (e.g. 1:1), together with the antigen binding protein (e.g. antibody), preferably tested over a concentration range, with subsequent detection and quantification of T cell activity. Preferably, such detection and quantification is as described below.

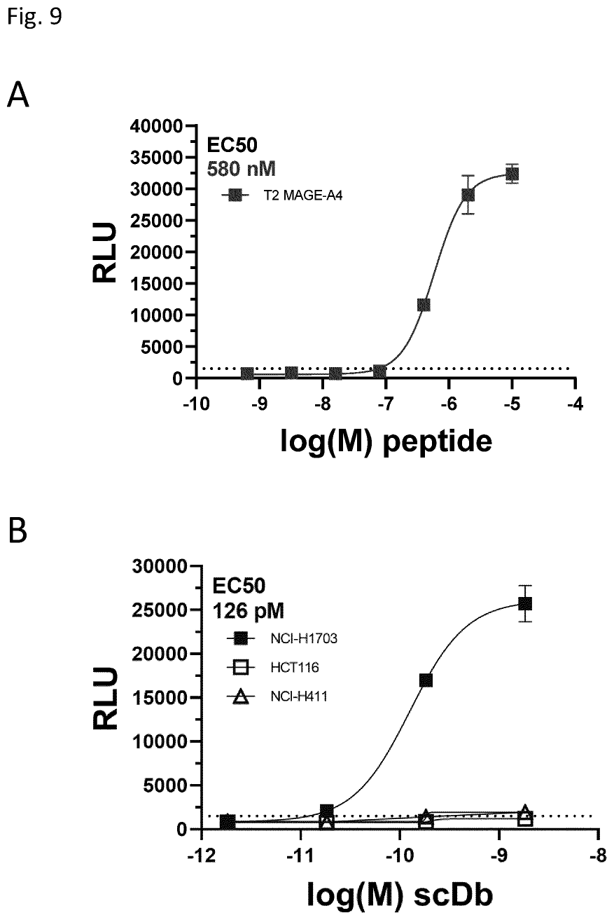

Example 3 herein also describes, again using a Jurkat-NFAT activation assay, that 1-H02 effectively redirects T-cell activity against cancer cells displaying HLA-A*02:01/MAGE-A4²³⁰⁻²³⁹ but not against cancer cells displaying HLA-A*02:01 lacking the MAGE-A4²³⁰⁻²³⁹ peptide (FIG. 9B).

Thus, in an embodiment, the antigen binding proteins (e.g. antibodies) of the invention, when in a T-cell engager format (e.g. in the scDb format), preferentially (or selectively) redirect (i.e. are capable of preferentially (or selectively) redirecting) T-cell activity against cells displaying HLA-A*02:01/MAGE-A4²³⁰⁻²³⁹ as compared to against cells displaying HLA-A*02:01 without the MAGE-A4²³⁰⁻²³⁹ peptide complexed thereto.

In an embodiment, the antigen binding proteins (e.g. antibodies) of the invention, when in a T-cell engager format (e.g. in the scDb format), preferentially (or selectively) redirect T-cell activity against cells displaying HLA-A*02:01/MAGE-A4²³⁰⁻²³⁹ as compared to against cells displaying HLA-A*02:01 without the MAGE-A4²³⁰⁻²³⁹ peptide complexed thereto and/or (preferably “and”) against cells displaying a HLA-A*02:01 restricted peptide that is not MAGE-A4²³⁰⁻²³⁹.

Alternatively viewed, in an embodiment, the antigen binding proteins (e.g. antibodies) of the invention, when in a T-cell engager format (e.g. in the scDb format), preferentially (or selectively) redirect T-cell activity against HLA-A*02:01/MAGE-A4²³⁰⁻²³⁹ positive cells as compared to against HLA-A*02:01 positive, MAGE-A4²³⁰⁻²³⁹ negative cells.

Any substantially homologous antigen binding protein (e.g. antibody) of the invention preferably exhibits such redirection of T-cell activity.

Preferably such redirection of T-cell activity is exhibited when the antigen binding protein is any T-cell engager format, preferably a bispecific format. Preferably, the antibody is in the scDb format.

Preferably such redirection of T cell activity is exhibited when the ratio of T cells (or effector cells comprising T cells) to cells displaying HLA-A*02:01/MAGE-A4²³⁰⁻²³⁹ (“target cells”) is about 1:1. Thus, preferably such redirection of T cell activity is exhibited in an assay comprising the co-culturing of HLA-A*02:01/MAGE-A4²³⁰⁻²³⁹ positive target cells (“T”) and effector cells comprising T cells (“E”) at an E:T ratio of about 1:1 (e.g. 1:1), together with the antigen binding protein (e.g. antibody), preferably tested over a concentration range, with subsequent detection and quantification of T cell activity. Preferably, such detection and quantification is as described below.

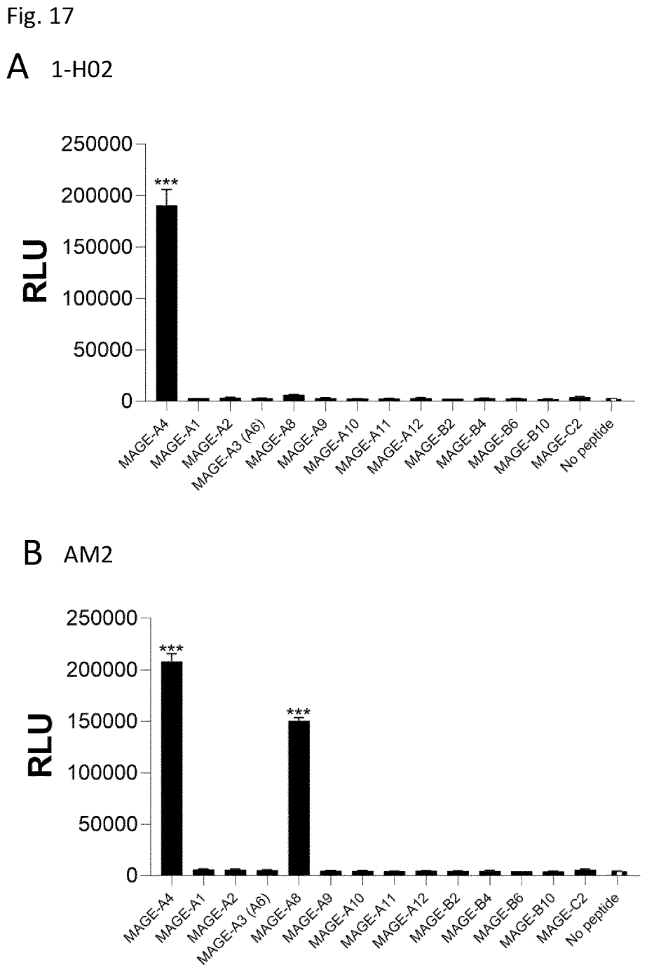

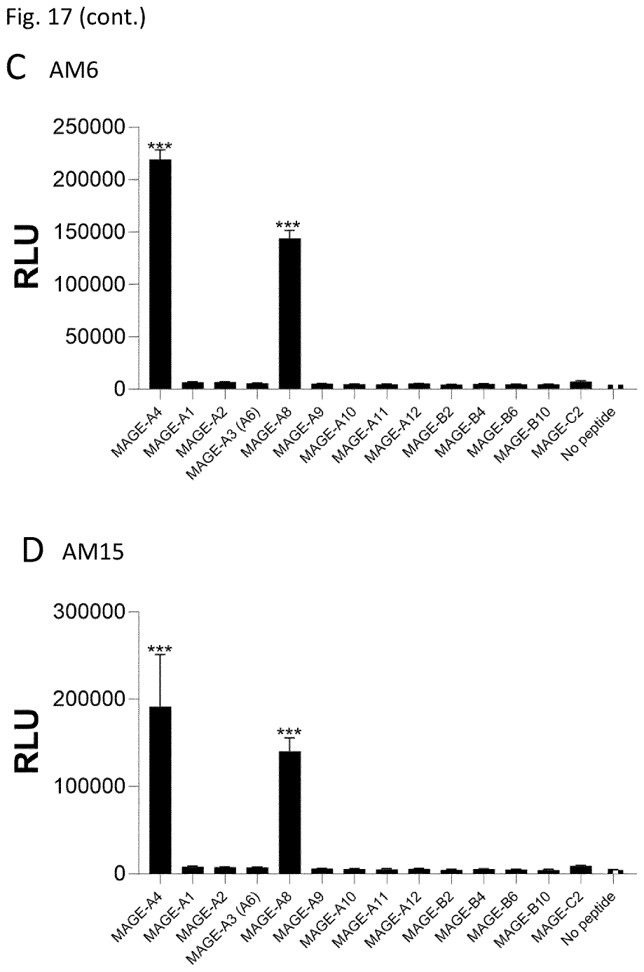

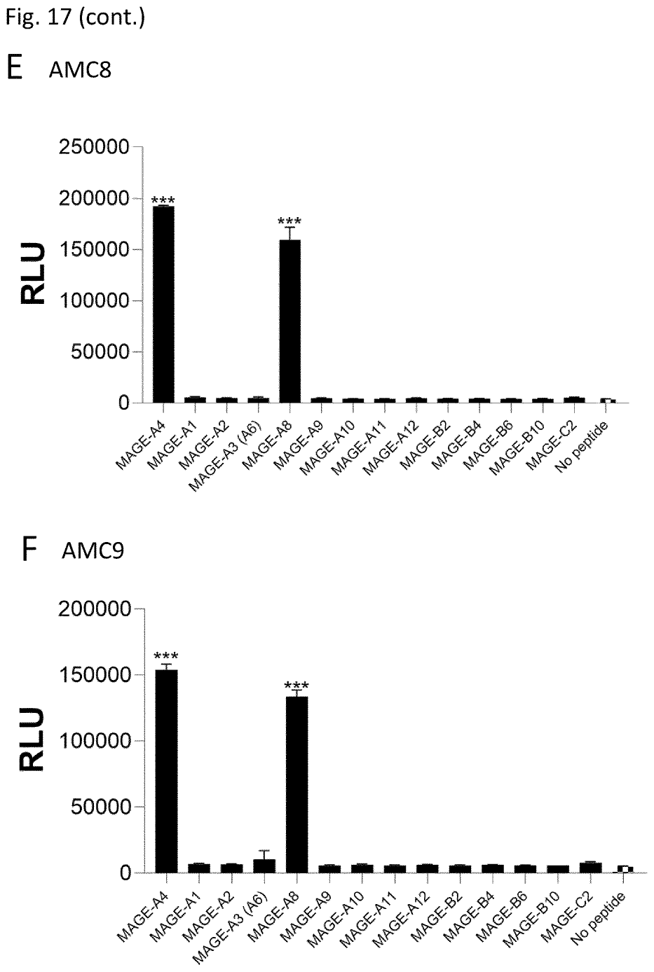

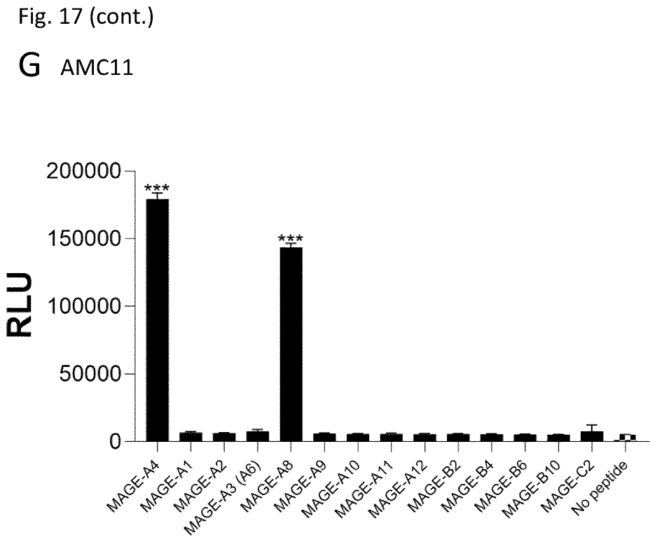

Example 3 herein also describes, as shown in FIG. 17A to G again using a Jurkat-NFAT activation assay, that the antibodies of the invention induced T-cell activation (redirected T-cell activity) with remarkable specificity against cells displaying HLA-A*02:01/MAGE-A4²³⁰⁻²³⁹ over cells displaying all of the HLA-A*02:01 restricted MAGE-A4 homologues representing a potentially dangerous cross-reactivity risk (i.e. peptides of SEQ ID NOs: 41 to 43 and 45 to 53), despite the peptides sharing high sequence similarity with MAGE-A4²³⁰⁻²³⁹. This further demonstrates the remarkable specificity of the antibodies of the invention.

Jurkat activation was only observed when T2 cells were pulsed with MAGE-A4²³⁰⁻²³⁹ peptide; the level of activation observed with all (each) of the homologue risk peptides was comparable to non-pulsed T2 cells, despite these risk peptides sharing high sequence similarity with MAGE-A4²³⁰⁻²³⁹. This further demonstrates the remarkable specificity of the antibodies, which is not only in terms of antigen binding (demonstrated above), but is here demonstrated also in terms of redirecting T-cell activity specifically against HLA-A*02:01/MAGE-A4²³⁰⁻²³⁹ positive cells.

Again, the only HLA-restricted peptide to which the antibodies of the invention redirected T-cell activity to any extent was the MAGE-A8 derived peptide, which as explained above does not represent a cross-reactivity risk.

Thus, in an embodiment, the antigen binding proteins (e.g. antibodies) of the invention, when in a T-cell engager format (e.g. in the scDb format), preferentially (or selectively) redirect T-cell activity against cells displaying HLA-A*02:01/MAGE-A4²³⁰⁻²³⁹ as compared to against cells displaying a HLA-A*02:01 restricted peptide comprising (or consisting of) the amino acid sequence of any one, preferably each, of SEQ ID NOs: 41 to 43 and 45 to 53 (MAGE-A4 homologues peptides).

Thus, in an embodiment, the antigen binding proteins (e.g. antibodies) of the invention when in a T-cell engager format (e.g. in the scDb format), redirect T-cell activity against pulsed T2 cells displaying a HLA-A*02:01 restricted peptide comprising (or consisting of) the amino acid sequence of any one, preferably each, of SEQ ID NOs: 41 to 43 and 45 to 53 (MAGE-A4 homologues peptides) to an extent that is not different, e.g. is not significantly different, from the extent of T-cell activity redirected by the same antigen binding protein (e.g. antibody) against negative control cells. Suitable negative control cells may be non-pulsed T2 cells, i.e. T2 cells lacking any pHLA-restricted exogenous peptide.

Alternatively viewed, in an embodiment, the antigen binding proteins (e.g. antibodies) of the invention when in a T-cell engager format (e.g. in the scDb format), redirect T-cell activity against cells displaying a HLA-A*02:01 restricted peptide comprising (or consisting of) the amino acid sequence of any one, preferably each, of SEQ ID NOs: 41 to 43 and 45 to 53 (MAGE-A4 homologues peptides) to an extent that is not different, e.g. is not significantly different, from the extent of T-cell activity redirected by the same antigen binding protein (e.g. antibody) against negative control cells. Suitable negative control cell cells may be cells displaying neither said HLA-A*02:01 restricted genomic risk peptides, nor HLA-A*02:01/MAGE-A4²³⁰⁻²³⁹ i.e. cells against which merely background levels of redirected T-cell activity are observed.

Thus, in an embodiment, the antigen binding proteins (e.g. antibodies) of the invention, when in a T-cell engager format (e.g. in the scDb format), redirect T-cell activity against cells displaying a HLA-A*02:01 restricted peptide comprising (or consisting of) the amino acid sequence of any one, preferably each, of SEQ ID NOs: 41 to 43 and 45 to 53 (MAGE-A4 homologues peptides) to an extent that is at most 10%, e.g. at most 5% of the T-cell activity redirected by the same antigen binding protein (e.g. antibody) against cells displaying HLA-A*02:01/MAGE-A4²³⁰⁻²³⁹.

Alternatively viewed, in an embodiment, the antigen binding proteins (e.g. antibodies) of the invention, when in a T-cell engager format, exhibit a decrease of at least 90%, e.g. at least 95%, in T-cell activity redirected against cells displaying a HLA-A*02:01 restricted peptide comprising (or consisting of) the amino acid sequence of any one, preferably each, of SEQ ID NOs: 41 to 43 and 45 to 53 (MAGE-A4 homologues peptides) as compared to the T-cell activity redirected by the same antigen binding protein (e.g. antibody) against cells displaying HLA-A*02:01/MAGE-A4²³⁰⁻²³⁹.

Any substantially homologous antigen binding protein (e.g. antibody) of the invention preferably exhibits such redirection of T-cell activity.

Preferably such redirection of T-cell activity is exhibited when the antigen binding protein is any T-cell engager format, preferably in a bispecific format. Preferably, the antibody is in the scDb format.

Preferably such redirection of T cell activity is exhibited when the ratio of T cells (or effector cells comprising T cells) to cells displaying HLA-A*02:01/MAGE-A4²³⁰⁻²³⁹ (“target cells”) is about 1:1. Thus, preferably such redirection of T cell activity is exhibited in an assay comprising the co-culturing of HLA-A*02:01/MAGE-A4²³⁰⁻²³⁹ positive target cells (“T”) and effector cells comprising T cells (“E”) at an E:T ratio of about 1:1 (e.g. 1:1), together with the antigen binding protein (e.g. antibody), preferably tested over a concentration range, with subsequent detection and quantification of T cell activity. Preferably, such detection and quantification is as described below.

### Assay 2: Assay for Redirection of T-Cell Activity (Jurkat)

The ability of an antigen binding protein (e.g. antibody) in a T-cell engager format to redirect T-cell activity against cells displaying a HLA-restricted peptide (pHLA) can be assessed by any appropriate means and the skilled person is familiar with suitable methods, e.g. a Jurkat activation assay in which the pHLA is displayed on the surface of a target cell (e.g. a T2 cell or a cancer cell).

Jurkat assays may comprise the co-culturing of Jurkat NFAT reporter cells representing effector cells (“E”), target cells (“T”) (e.g. peptide pulsed T2 cells (described elsewhere herein) or cancer cells e.g. cancer cell line cells), preferably at an E:T ratio of about 1:1, together with the antigen binding protein (e.g. antibody) to be tested, and subsequently applying the cultured cells to wells comprising a luciferase substrate and detecting luminescence.

In some embodiments, the ability of an antigen binding protein (e.g. antibody) in a T-cell engager format to redirect T-cell activity against cells displaying a HLA-restricted peptide (pHLA) may be as determined by (or as assessed by) a Jurkat activation, e.g. a Jurkat activation assay that comprises:
(a) Culturing target cells (e.g. T2 cells, or cancer cells e.g. cancer cell line cells) in cell culture medium (e.g. RPMI1640 supplemented with 10% FBS (Fetal Bovine Serum) and 5 U/ml PS (Penicillin-Streptomycin);
(b) Harvesting said target cells, e.g. by transferring suspension cells to a Falcon tube (e.g. of 50 ml volume), or by removing cell culture supernatant from adherent cells (cancer cells may be adherent cells), washing the adherent cells once with sterile 1×PBS, incubating with an appropriate amount of Trypsin (e.g. 1-2 ml) at about 37° C. in a humidified incubator until the cells detach from the cell culture vessel, adding an appropriate amount of cell culture medium (e.g. 10 ml) and transferring the suspension of adherent cells to a new vessel, e.g. to a 50 ml Falcon tube;
(c) Washing said harvested target cells, e.g. by pelleting the cells (e.g. at 300 g for 5 minutes), resuspending in cell culture medium (e.g. 10 ml), pelleting a second time (e.g. at 300 g for 5 minutes), then resuspending pelleted cells to an appropriate volume (e.g. 5-10 ml);
(d) Counting said target cells and diluting said target cells to an appropriate density (e.g. 500000/ml);
(e) Seeding said target cells in the wells of cell culture plates (e.g. white 96 well plates*) in cell culture medium (e.g. at 50000 cancer cells/well the day before co-culturing (step (q)) to allow for monolayer formation of adherent cells, or 25000 T2 cells/well on the day of co-culture);
(f) If cancer cells are seeded in step (e), then incubating said seeded adherent cancer cells e.g. for 18 h at 37° C. in a humidified incubator to allow for monolayer formation;
(g) Culturing Jurkat NFAT reporter cells e.g. in cell culture medium (e.g. RPMI1640 supplemented with 10% FBS and 5 U/ml PS);
(h) Harvesting said Jurkat NFAT reporter cells e.g. by transferring cells to a Falcon tube (e.g. of 50 ml volume);
(i) Washing said Jurkat NFAT reporter cells, e.g. by pelleting the cells (e.g. at 300 g for 5 minutes), resuspending in cell culture medium (e.g. 10 ml), pelleting a second time (e.g. at 300 g for 5 minutes), then resuspending pelleted cells to an appropriate volume (e.g. 5-10 ml);
(j) Counting said Jurkat NFAT reporter cells and diluting to an appropriate density (e.g. 500000/ml);
(k) Adding said re-suspended Jurkat NFAT reporter cells to the target cells at a ratio of about 1:1 Jurkat NFAT reporter cells to target cells;
(l) Diluting the antigen binding protein (e.g. antibody) to be tested in assay medium (e.g. RPMI1640 supplemented with 10% FBS and 5 U/ml PS), for example at a concentration range of 0.2 nM to 200 nM;
(m) Adding the antigen binding protein (e.g. antibody) to be tested to the co-culture of cells of step (k) (e.g. at a final concentration range from 0.02 nM to 20 nM);
(n) Diluting HLA-restricted peptides (e.g. MAGE-A4²³⁰⁻²³⁹ or other peptides) in assay medium (e.g. RPMI1640 supplemented with 10% FBS and 5 U/ml PS), for example at a concentration range of 2.4 nM to 200 μM (if cancer cells are used as target cells, this step is not performed);
(o) If T2 cells are used as target cells, transfer (e.g. about 50 μl) of peptide dilutions (e.g. in duplicates or triplicates) to a 96-well cell culture plate (i.e. wells of the plate) e.g. at final concentration in the range of 600 pM to 50 μM (if cancer cells are used as target cells, this step is not performed)
(p) Adding culture medium (e.g. to a final volume of 150 μl per well);
(q) Co-culturing by incubating the co-culture e.g. for about 16 h at about 37° C. in a humidified incubator;
(r) Diluting luciferase substrate (e.g. D-Luciferin, Monopotassium Salt (Promega)) e.g. at 1:10 in Milli-Q water;
(s) Adding diluted luciferase substrate (e.g. D-Luciferin Monopotassium Salt (Promega)) from step (r), e.g. at a ratio of 1:3 luciferase substrate to cell co-culture (e.g. by adding 50 μl of luciferase substrate to the 150 μl of co-culture from step (q)); and
(t) Recording luminescence, e.g. using Varioscan LUX (Thermo Fisher).
* Step (t) typically requires the co-culture and D-Luciferin to be present in the wells of a white cell culture plate in order for the luminescence to be recorded, so it is preferable that cells are seeded in white cell culture plates in step (e). However, this is not essential; step (e) may alternatively comprise seeding said target cells in the wells of cell culture plates that are not white. If so, then step (q) further comprises pelleting the co-culture plates (e.g. at 300 g for 5 min), discarding excess cell culture supernatant, e.g. 100 μl; and re-suspending pelleted cells in remaining cell culture supernatant (e.g 100 μl). Subsequently, in step (s), an aliquot of the diluted luciferase substrate from step (r) and an aliquot of the resuspended cells would be added at a ratio of 1:3 luciferase substrate to cell co-culture in the wells of a white cell culture plate (e.g. a white 96-well plate), e.g. 25 μl per well of luciferase substrate and 75 μl per well of cell co-culture, prior to the performance of the luminescence recordal step (t).

A preferred Jurkat activation assay is described in the Example section herein.

In such a Jurkat activation assay, the antigen binding protein may be an antibody in any T-cell engager format, preferably a bispecific format. In a preferred assay, the antibody is in the scDb format.

### T-Cell Activation, Differentiation, Proliferation

Example 4 herein describes the further assessment of the ability of an antibody of the invention, 1-H02 in the scDb format, to specifically induce activation, differentiation and proliferation of both CD4+ and CD8+ T cells directed against cells displaying the HLA-A*02:01/MAGE-A4²³⁰⁻²³⁹ antigen, as compared to T cells directed against cells displaying HLA-A*02:01 absent the MAGE-A4²³⁰⁻²³⁹ peptide complexed thereto and as compared to T cells directed cells displaying different peptides complexed thereto.

The present inventors performed assays in which target (“T”) cells (positive or negative for HLA-A*02:01/MAGE-A4²³⁰⁻²³⁹), effector cells (“E”) comprising T cells, and 1-H02, were co-incubated, with subsequent analysis by flow cytometry comprising detection of T-cell activation, differentiation and proliferation markers, and assessment of killing of target cells.

In some assays the target cells were T2 cells which were pulsed with exogenously applied MAGE-A4²³⁰⁻²³⁹ peptide. In these assays, control cells were non-pulsed T2 cells, and T2 cells pulsed with an unrelated peptide. In other assays, the target cells were HLA-A*02:01/MAGE-A4²³⁰⁻²³⁹ positive cancer cells. In these assays control cells were HLA-A*02:01/MAGE-A4²³⁰⁻²³⁹ negative cancer cells.

In assays of the ability of a T-cell engager to induce T-cell activity, proliferation, differentiation and/or cytotoxicity, it is possible to use T cells, or a subset of T cells as the effector cells (e.g. PBMCs or pan T-cells isolated from PBMCs).

In such assays, the apparent efficacy of an antibody can be artificially inflated through use of a high E:T ratio in the co-culturing steps. The E:T ratio is simply the ratio of the number of effector cells to the number of target cells. The use of a greater number of effector cells relative to target cells is more likely to lead to a higher level of observed effector cell activity against the target cells. E:T ratios of 10:1 are seen in the art, but this is considered a high E:T ratio that can mask poor performance of the antibodies being tested.

Therefore, the present inventors have performed the relevant assays using an E:T ratio of 1:1. Despite an E:T ratio of only 1:1, remarkable results were achieved with the present antigen binding proteins (e.g. antibodies).

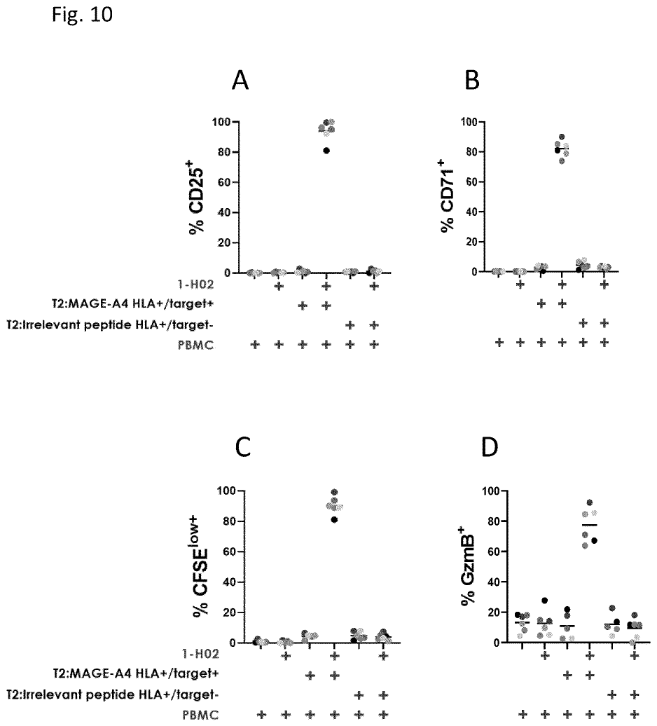

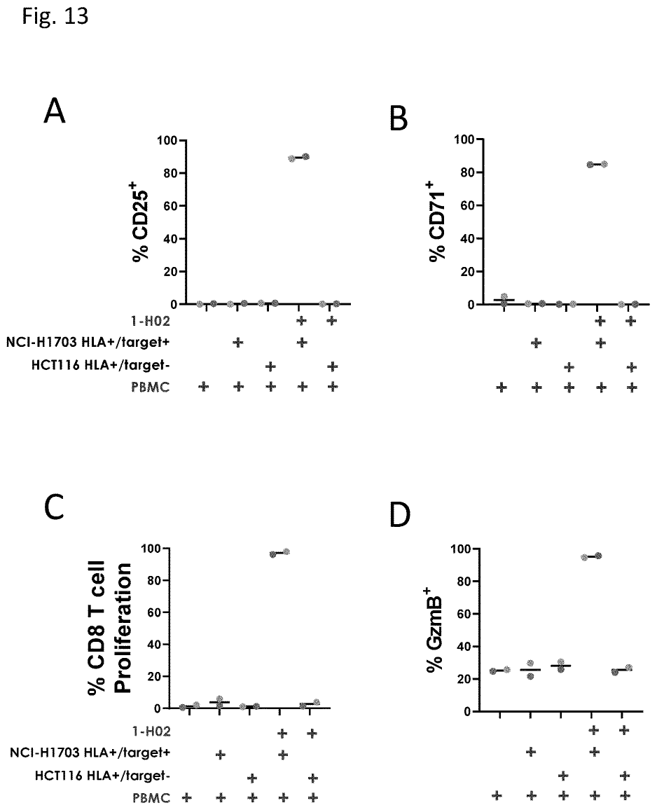

As shown in FIGS. 10A, B & D, and 11A, B & D, the capacity of 1-H02 to induce CD4+ and CD8+ T cell activation and differentiation (measured by upregulation of CD25 and CD71 surface expression on said T cells, and intracellular granzyme B expression), was only observed when 1-H02 was co-cultured with HLA-A*02:01/MAGE-A4²³⁰⁻²³⁹ positive T2 cells, and not with non-pulsed T2 cells (i.e. cells lacking HLA-A*02:01/MAGE-A4²³⁰⁻²³⁹), nor with T2 cells pulsed with a different exogenous peptide (i.e. cells lacking HLA-A*02:01/MAGE-A4²³⁰⁻²³⁹ and displaying a different HLA-A*02:01 restricted exogenous peptide). As shown in FIGS. 13A, B & D, and 14A, B & D, CD4+ and CD8+ T-cell activation and differentiation (measured in the same manner) was only observed when 1-H02 was co-cultured with HLA-A*02:01/MAGE-A4²³⁰⁻²³⁹ positive cancer cells, and not with HLA-A*02:01/MAGE-A4²³⁰⁻²³⁹ negative cancer cells.

The capacity of 1-H02 to induce proliferation of T cells directed against HLA-A*02:01/MAGE-A4²³⁰⁻²³⁹ positive cells was also assessed. CD4+ and CD8+ T-cell proliferation (measured by detection of CFSE-labelled PBMCs), was only observed when 1-H02 was co-cultured with HLA-A*02:01/MAGE-A4²³⁰⁻²³⁹ positive cells (both T2 cells pulsed with MAGE-A4²³⁰⁻²³⁹ peptide, and HLA-A*02:01/MAGE-A4²³⁰⁻²³⁹ positive cancer cells) and not with non-pulsed T2 cells, T2 cells pulsed with a different exogenous peptide, or HLA-A*02:01/MAGE-A4²³⁰⁻²³⁹ negative cancer cells (FIGS. 10C, 11C, 13C & 14C).

Absence of the MAGE-A4²³⁰⁻²³⁹ peptide, i) in non-pulsed T2 cells, ii) in T2 cells pulsed with an peptide structurally unrelated to HLA-A*02:01/MAGE-A4²³⁰⁻²³⁹ and iii) in HLA-A*02:01/MAGE-A4²³⁰⁻²³⁹ negative cancer cells, led to no CD4+ or CD8+ T-cell activation, differentiation, or proliferation beyond base levels.

Together, these data demonstrate the advantageous specificity and safety profile of the antigen binding proteins (e.g. antibodies) of the invention.

The antigen binding proteins (e.g. antibodies) of the invention, when in a T-cell engager format (e.g. in the scDb format), preferentially (or selectively) induce activation and/or (preferably “and”) differentiation and/or (preferably “and”) proliferation of T cells directed against cells displaying HLA-A*02:01/MAGE-A4²³⁰⁻²³⁹.

Alternatively viewed, the antigen binding proteins (e.g. antibodies) of the invention, when in a T-cell engager format (e.g. in the scDb format), are capable of preferentially (or selectively) inducing activation and/or (preferably “and”) differentiation and/or (preferably “and”) proliferation of T cells directed against cells displaying HLA-A*02:01/MAGE-A4²³⁰⁻²³⁹.

Thus, in an embodiment, the antigen binding proteins (e.g. antibodies) of the invention, when in a T-cell engager format (e.g. in the scDb format), preferentially (or selectively) induce activation and/or (preferably “and”) differentiation and/or (preferably “and”) proliferation of T cells directed against cells displaying HLA-A*02:01/MAGE-A4²³⁰⁻²³⁹ as compared to against cells displaying HLA-A*02:01 without the MAGE-A4²³⁰⁻²³⁹ peptide complexed thereto.

In an embodiment, the antigen binding proteins (e.g. antibodies) of the invention, when in a T-cell engager format (e.g. in the scDb format), preferentially (or selectively) induce activation and/or (preferably “and”) differentiation and/or (preferably “and”) proliferation of T cells directed against cells displaying HLA-A*02:01/MAGE-A4²³⁰⁻²³⁹ as compared to against cells displaying a HLA-A*02:01 restricted peptide that is not MAGE-A4²³⁰⁻²³⁹.

In an embodiment, the antigen binding proteins (e.g. antibodies) of the invention, when in a T-cell engager format (e.g. in the scDb format), preferentially (or selectively) induce activation and/or (preferably “and”) differentiation and/or (preferably “and”) proliferation of T cells directed against cells displaying HLA-A*02:01/MAGE-A4²³⁰⁻²³⁹ as compared to against cells displaying HLA-A*02:01 without the MAGE-A4²³⁰⁻²³⁹ peptide complexed thereto and/or (preferably “and”) against cells displaying a HLA-A*02:01 restricted peptide that is not MAGE-A4²³⁰⁻²³⁹.

Alternatively viewed, in an embodiment, the antigen binding proteins (e.g. antibodies) of the invention, when in a T-cell engager format (e.g. in the scDb format), preferentially (or selectively) induce activation and/or (preferably “and”) differentiation and/or (preferably “and”) proliferation of T cells directed against HLA-A*02:01/MAGE-A4²³⁰⁻²³⁹ positive cells as compared to against HLA-A*02:01 positive, MAGE-A4²³⁰⁻²³⁹ negative cells.

The HLA-A*02:01/MAGE-A4²³⁰⁻²³⁹ displaying, or positive or negative cells referred to herein, may be any cell type including the preferred target and cancer cell types disclosed elsewhere herein.

The T-cells may be as defined anywhere else herein, and are preferably CD4⁺ T cells or CD8⁺ T cells.

Any substantially homologous antigen binding protein (e.g. antibody) of the invention preferably exhibits such inducement of T-cell activation, differentiation, or proliferation.

The term “induce” as used herein means promote, i.e. enhance or increase the stated property or activity. Induce need not mean that the starting level of the property or activity is zero, i.e. it may mean “induce further”.

Preferably such induction of activation, differentiation or proliferation of T cells is exhibited when the antigen binding protein is any T-cell engager format, preferably a bispecific format. Preferably, the antibody is in the scDb format.

Preferably such induction of activation, differentiation or proliferation of T cells is exhibited when the ratio of T cells (or effector cells comprising T cells) to cells displaying HLA-A*02:01/MAGE-A4²³⁰⁻²³⁹ (“target cells”) is about 1:1. Thus, preferably such induction of activation, differentiation or proliferation of T cells is exhibited in an assay comprising the co-culturing of HLA-A*02:01/MAGE-A4²³⁰⁻²³⁹ positive target cells (“T”) and effector cells comprising T cells (“E”) at an E:T ratio of about 1:1 (e.g. 1:1), together with the antigen binding protein (e.g. antibody), preferably tested over a concentration range, with subsequent detection and quantification of T cell activation, differentiation or proliferation. Preferably, such detection and quantification is as described below.

### Assay 3: Assay for Induced T-Cell Activation/Differentiation

The ability of an antigen binding protein (e.g. antibody) in a T-cell engager format to induce activation or differentiation of T cells directed against cells displaying a HLA-restricted peptide (pHLA) (“target cells”) can be assessed by any appropriate means, and the skilled person is familiar with suitable methods, for example co-culturing the target cells (“T”), and effector cells (“E”) comprising T cells, such as PBMCs, preferably at an E:T ratio of about 1:1, together with the antigen binding protein (e.g. antibody) to be tested, and subsequently detecting and quantifying markers of T-cell activation or differentiation.

Suitable T-cell activation/differentiation markers are well-known, and include CD25 and CD71 (both markers of T-cell activation), and Granzyme B (a marker of T-cell differentiation). Markers can be detected by any suitable means, including using fluorescently-labelled antibodies against said markers, with subsequent quantification thereof, e.g. by flow cytometry techniques, which are well-known. If desired, signal specifically from T cells, or specifically from CD8⁺ or specifically from CD4⁺ T cells in particular can be assessed by also performing detection using fluorescently labelled antibodies against CD3, CD8⁺ and CD4⁺, respectively.

Thus, a suitable assay may comprise
(a) Co-culturing in wells of an assay plate target cells (“T”) (e.g. cancer cells (e.g. cancer cell line cells), or peptide pulsed T2 cells), e.g. as discussed above, and effector cells (“E”) comprising T cells (e.g. peripheral blood mononuclear cells (PBMCs)), e.g. as discussed above, preferably at an E:T ratio of about 1:1, and the antigen binding protein (e.g. antibody) to be tested (e.g. 100 ng/ml), e.g. for about 72 h at about 37° C.; and
(b) Performing (multi-colour) flow cytometry staining and analysis, wherein
   (i) staining may be performed with anti-human fluorescently-labelled antibodies against one or more biomarkers of T-cell activity, e.g. CD25 (e.g. APC-CD25 (clone BC96)) or CD71 (e.g. PercpCy5.5-CD71 (clone CY1G4)); and/or
   (ii) staining may be performed with anti-human fluorescently-labelled antibodies against one or more biomarkers of T-cell differentiation, e.g. Granzyme B (e.g. PE-Granzyme B (clone QA18A28)); and
(c) Quantifying the signal of the fluorescent label, e.g. using Zombie Yellow fixable viability kit, using appropriate fluorescence minus one (FMO) controls to exclude background staining.

If peptide pulsed T2 cells are used as target cells, then T2 cells are pulsed with the relevant peptide prior to step (a), e.g. by resuspension in assay buffer containing the peptide (e.g. about 50 μM of peptide) and incubating e.g. for about 4.5 hrs at about 37° C., and subsequently centrifuging the resulting peptide pulsed T2 cells and resuspending them in fresh assay buffer to remove excess peptide.

A suitable assay for assessing the ability of an antigen binding protein (e.g. antibody) in a T-cell engager format to induce activation or differentiation of T cells directed against a HLA-restricted peptide (pHLA) is described in the Example section herein.

In some embodiments, such an assay comprises the use of peptide pulsed T2 cells or cancer cells (e.g. cancer cell line cells) as target cells, and PBMCs as effector cells, and the assay comprises:
(a) If peptide pulsed T2 cells are used as target cells, then T2 cells are resuspended in assay buffer (e.g. RPMI/10% FBS/1% P/S) containing peptide (e.g. about 50 μM of peptide), which may be MAGE-A4²³⁰⁻²³⁹ or a comparator peptide, incubating e.g. for about 4.5 hrs at about 37° C., and subsequently centrifuging the resulting peptide pulsed T2 cells and resuspending them in fresh assay buffer to remove excess peptide (if cancer cells are used as target cells, this step is not performed);
(b) Diluting the antigen binding protein (e.g. antibody) to be tested e.g. to 10× the final concentrations required in assay buffer;
(c) Preparing PBMCs, e.g. by thawing frozen PBMCs obtained from liquid nitrogen storage (e.g. in a 37° C. water bath), and transferring said PBMCs drop wise into pre-warmed assay buffer, centrifuging and resuspending the PBMCs in fresh assay buffer, counting and resuspending the PBMCs at a desired concentration;
(d) Preparing a co-culture of PMBCs (effector cells “E”) and target cells (“T”) in wells of a culture plate at an E:T ratio of about 1:1 (e.g. 10,000 PBMCs and 10,000 target cells) e.g. in a total volume of about 180 μl;
(e) Adding e.g. about 20 μl of the antigen binding protein (e.g. antibody) to be tested to the wells (e.g. about 100 ng/ml) e.g. to make a final volume of about 200 μl;
(f) Incubating the co-culture at about 37° C. e.g. for about 72 hrs; and
(g) Combining contents of a number (e.g. 4) wells of identical setup, and centrifuging and washing cells therein (e.g. by discarding supernatant and resuspending cells in PBS and centrifuging cells);
(h) Discarding supernatant, and resuspending cells in flow cytometry staining buffer (e.g. PBS/2% FBS) containing Fc block, to prevent any non-specific binding, and incubating e.g. for about 15 minutes, at about 4° C. in the dark;
(i) Centrifuging, and washing cells, and discarding supernatant, then resuspending cells in PBS containing a viability marker e.g. for about 15 minutes in the dark at about 25° C.;
(j) Centrifuging and washing cells and discarding the supernatant; and

### For Assaying T Cell Activation:

(k) Resuspending cells in staining buffer e.g. for about 30 minutes in the dark at about 4° C., said staining buffer containing fluorescently labelled antibodies against T cell antigens (e.g. CD3, CD4, CD8), and against markers of T-cell activation (e.g. CD25, CD71);
(l) Centrifuging, washing and resuspending cells in fixation buffer, e.g. BD cytofix, for e.g. about 20 mins, at about 4° C. in the dark (the fixation buffer preserves the light-scattering characteristics and fluorescence intensities of the anti-bound T cells);
(m) Centrifuging cells, twice washing cells and resuspending cells in staining buffer (and optionally storing cells at about 4° C.); and
(n) Analysing fluorescence (of cells) using a flow cytometer (e.g. a BD Canto II); or

### For T Cell Differentiation

after steps (a) to (j) above
(k) Resuspending cells in staining buffer e.g. for about 30 minutes, in the dark at about 4° C., said staining buffer containing fluorescently labelled antibodies against T cell antigens (e.g. CD3, CD4, CD8);
(l) Centrifuging, washing and resuspending cells in fixation buffer, e.g. BD cytofix, for e.g. about 20 mins, at about 4° C. in the dark;
(m) Centrifuging cells, twice washing cells and resuspending cells in staining buffer;
(n) Permeabilizing cells by
   i) Centrifuging cells and discarding supernatant, and resuspending cells in a permeabilization buffer (e.g. eBiosciences Perm/Wash (1×)) e.g. for about 10 minutes at about 4° C. in the dark; and
   ii) centrifuging cells, washing and discarding supernatant, and resuspending cells in permeabilization buffer (e.g. eBiosciences Fixation/permeabilization buffer) e.g. for about 30 minutes, at about 4° C. in the dark;
(o) Centrifuging cells, washing cells with a permeabilization/wash buffer (e.g. eBioscience Perm/Wash (1×)) and discarding the supernatant;
(p) Resuspending cells in a permeabilization/wash buffer (e.g. eBioscience Perm/Wash (1×)) containing fluorescently labelled antibodies against markers of T-cell differentiation, e.g. anti-human Granzyme B antibody, e.g. for about 45 minutes at about 4° C. in the dark;
(q) Centrifuging cells, twice washing cells with a permeabilization/wash buffer (e.g. eBioscience Perm/Wash (1×)) and resuspending cells in staining buffer; and
(r) Analysing fluorescence (of cells) using a flow cytometer (e.g. a BD Canto II).

In such assays, fluorescence (of cells) is from differently labelled antibodies against different T cell antigens and activation/differentiation markers. The different fluorescence signals can be distinguished using standard flow cytometry techniques, thereby permitting detection of particular activation/differentiation markers of interest, either from all T cells and/or from particular T cell types of interest.

In such an assay, the antigen binding protein may be an antibody in any bispecific antibody format. In a preferred assay, the antibody is in the scDb format.

A preferred assay for assessing the ability of an antigen binding protein (e.g. antibody) in a T-cell engager format to induce activation and/or differentiation of T cells directed against a HLA-restricted peptide (pHLA) is described in the Example section herein.

### Assay 4: Assay for T-Cell Proliferation

The ability of an antigen binding protein (e.g. antibody) in a T-cell engager format to induce proliferation of T cells directed against cells displaying a HLA-restricted peptide (pHLA) (“target cells”) can be assessed by any appropriate means and the skilled person is familiar with suitable methods, for example co-culturing the target cells (“T”) (e.g. cancer cells (e.g. cancer cell line cells), or peptide pulsed T2 cells), e.g. as discussed above, and fluorescently-labelled effector cells (“E”) comprising T cells, such as CFSE-labelled PBMCs, preferably at an E:T ratio of about 1:1, together with the antigen binding protein (e.g. antibody) to be tested, and subsequently detecting the fluorescent-label via flow cytometry. If desired, signal from specifically T cells, or CD8⁺ or CD4⁺ T cells in particular can be assessed by also performing detection using fluorescently labelled antibodies against CD3, CD8⁺ and CD4⁺, respectively.

Thus, a suitable assay may comprise:
(a) Co-culturing in wells of an assay plate target cells (“T”) (e.g. cancer cells (e.g. cancer cell line cells), or peptide pulsed T2 cells), e.g. as discussed above, and effector cells comprising T cells (“E”) (e.g. PBMCs), e.g. as discussed above, labelled with a fluorescent label (e.g. Carboxyfluorescein succinimidyl ester (CFSE)), at an E:T ratio of about 1:1, and the antigen binding protein (e.g. antibody) to be tested (e.g. about 100 ng/ml), e.g. for about 72 h at about 37° C.;
(b) Detecting the fluorescent-label (e.g. CFSE) e.g. using (multi-colour) flow cytometry; and
(c) Quantifying the signal of the fluorescent label, e.g. using Zombie Yellow fixable viability kit, using appropriate fluorescence minus one (FMO) controls to exclude background staining.

If peptide pulsed T2 cells are used as target cells, then T2 cells are pulsed with the relevant peptide prior to step (a), e.g. by resuspension in assay buffer containing the peptide (e.g. about 50 μM of peptide) and incubating e.g. for about 4.5 hrs at about 37° C., and subsequently centrifuging the resulting peptide pulsed T2 cells and resuspending them in fresh assay buffer to remove excess peptide.

In some embodiments, such an assay comprises the use of peptide pulsed T2 cells or cancer cells (e.g. cancer cell line cells) as target cells, and PBMCs as effector cells, and the assay comprises
(a) If peptide pulsed T2 cells are used as target cells, then T2 cells are resuspended in assay buffer (e.g. RPMI/10% FBS/1% P/S) containing peptide (e.g. about 50 μM of peptide), which may be MAGE-A4²³⁰⁻²³⁹ or a comparator peptide, incubating e.g. for about 4.5 hrs at about 37° C., and subsequently centrifuging the resulting peptide pulsed T2 cells and resuspending them in fresh assay buffer to remove excess peptide (if cancer cells are used as target cells, then this step is not performed);
(b) Diluting the antigen binding protein (e.g. antibody) to be tested, e.g. to 10× the final concentrations required in assay buffer;
(c) Preparing PBMCs, e.g. by thawing frozen PBMCs obtained from liquid nitrogen storage (e.g. in a 37° C. water bath), and transferring said PBMCs drop wise into pre-warmed assay buffer, centrifuging and resuspending the PBMCs in fresh assay buffer;
(d) Pelleting and resuspending the PBMCs in PBS (e.g. 1 ml Dulbecco's phosphate-buffered saline (DPBS)) containing CFSE (e.g. about 0.5 μM CFSE), incubating for e.g. about 8 minutes at room (or ambient) temperature (e.g. about 25° C.), and adding assay buffer (e.g. about 9 ml of assay buffer);
(e) Pelleting the PBMCs, washing the PBMCs once in assay buffer, counting and resuspending the PBMCs at a desired concentration;
(f) Preparing a co-culture of PMBCs (effector cells “E”) and target cells (“T”) in wells of a culture plate at an E:T ratio of about 1:1 (e.g. 10,000 PBMCs and 10,000 target cells) e.g. in a total volume of about 180 μl;
(g) Adding e.g. about 20 μl of the antigen binding protein (e.g. antibody) to be tested to the wells e.g. to make a final volume of about 200 μl;
(h) Incubating the co-culture at about 37° C. e.g. for about 72 hrs;
(i) Combining the contents of a number (e.g. 4) wells of identical setup, and centrifuging cells and washing cells (e.g. by discarding supernatant and resuspending cells in PBS), discarding supernatant;
(j) Resuspending cells in flow cytometry staining buffer (e.g. PBS/2% FBS) containing Fc block, to prevent any non-specific binding, and incubating e.g. for about 15 minutes, at about 4° C. in the dark;
(k) Centrifuging cells, washing cells, and discarding supernatant, then resuspending cells in PBS containing a viability marker, e.g. for about 15 minutes in the dark, at about 25° C.;
(l) Centrifuging cells, washing cells and discarding the supernatant;
(m) Resuspending cells in staining buffer, e.g. for about 30 minutes in the dark at about 4° C., said staining buffer containing fluorescently labelled antibodies against T cell antigens (e.g. CD3, CD4, CD8);
(n) Centrifuging cells, washing cells and resuspending cells in fixation buffer, e.g. BD cytofix, e.g. for about 20 mins at about 4° C. in the dark (the fixation buffer preserves the light-scattering characteristics and fluorescence intensities of the anti-bound T cells);
(o) Centrifuging cells, twice washing cells and resuspending cells in staining buffer (and optionally storing cells at about 4° C.); and
(p) Analysing fluorescence (of cells) using a flow cytometer (e.g. a BD Canto II).

In such assays, fluorescence (of cells) is from differently labelled antibodies against different T cell antigens, and from CFSE. The different fluorescence can be distinguished using standard flow cytometry techniques, thereby permitting detection of proliferation signal either from all T cells and/or from particular T cell types of interest.

In such assays, fluorescence (from cells) is from differently labelled antibodies against different T cell antigens and activation/differentiation markers. The different fluorescence signals can be distinguished using standard flow cytometry techniques, thereby permitting detection of proliferation either from all T cells and/or from particular T cell types of interest.

In such an assay, the antigen binding protein may be an antibody in any T-cell engager format. In a preferred assay, the antibody is in the scDb format.

A preferred assay for assessing the ability of an antigen binding protein (e.g. antibody) in a T-cell engager format to induce proliferation of T cells directed against a HLA-restricted peptide (pHLA) is described in the Example section herein.

### T-Mediated Cytotoxicity

Example 4 herein also describes that a 7-Aminoactinomycin D (7-AAD) viability staining assay was used to assess the ability of antibodies of the invention to specifically induce T-cell mediated cytotoxicity of cells displaying the HLA-A*02:01/MAGE-A4²³⁰⁻²³⁹ antigen, as compared to cells displaying HLA-A*02:01 absent the MAGE-A4²³⁰⁻²³⁹ peptide complexed thereto, and as compared to cells displaying a HLA-A*02:01 restricted peptide having a sequence unrelated to MAGE-A4²³⁰⁻²³⁹.

The present inventors performed assays in which target (“T”) cells (positive or negative for HLA-A*02:01/MAGE-A4²³⁰⁻²³⁹), CFSE-labelled effector cells (“E”) comprising T cells (e.g. PBMCs or pan-T cells isolated therefrom), and 1-H02, were co-incubated, as described above, with subsequent staining with the non-viable cell stain 7-AAD, detection thereof via flow cytometry and quantification of the signal.

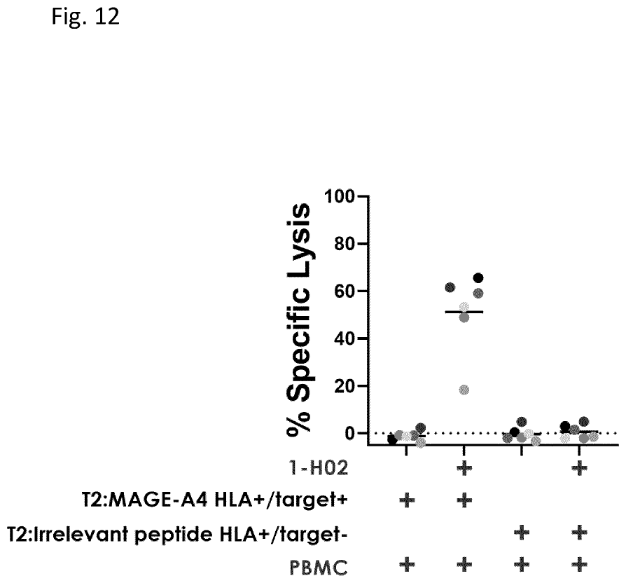

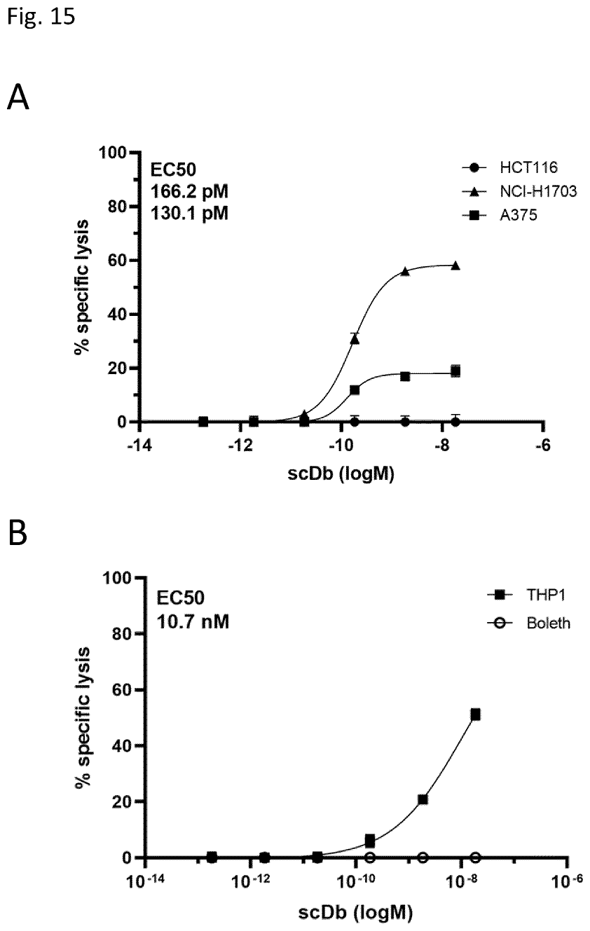

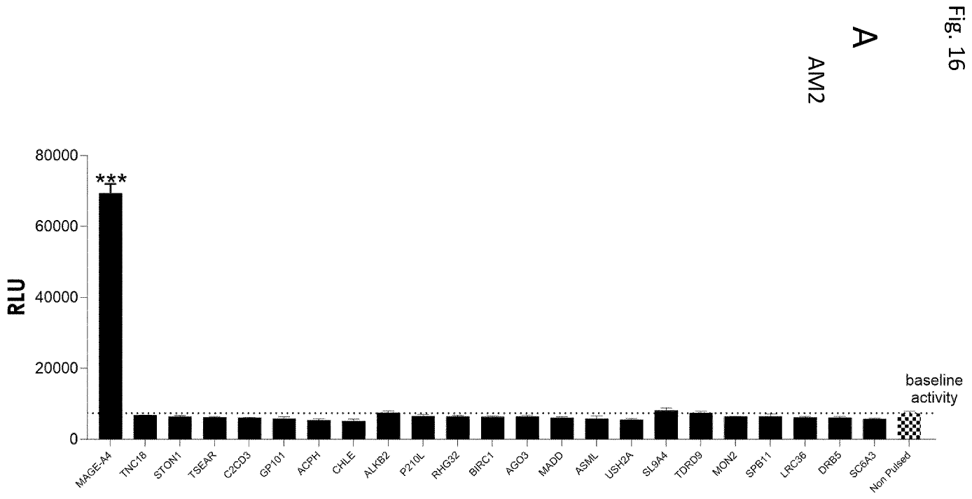

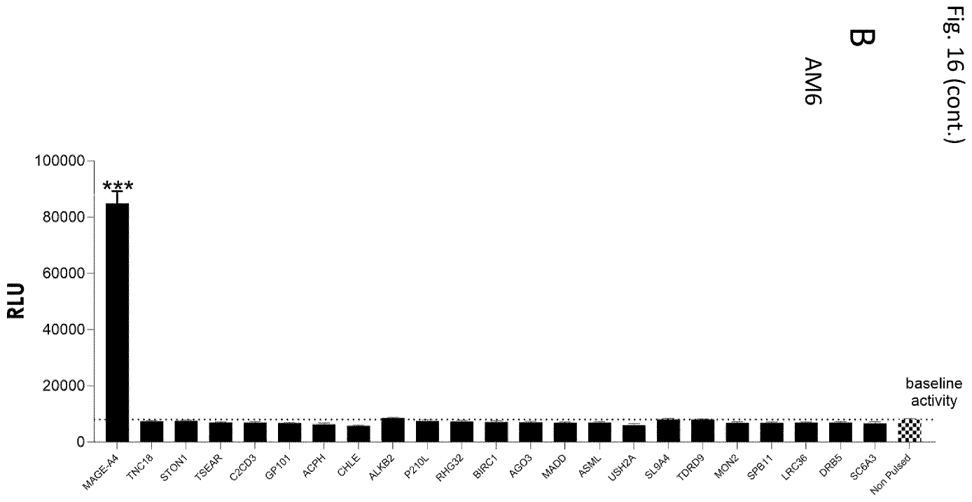

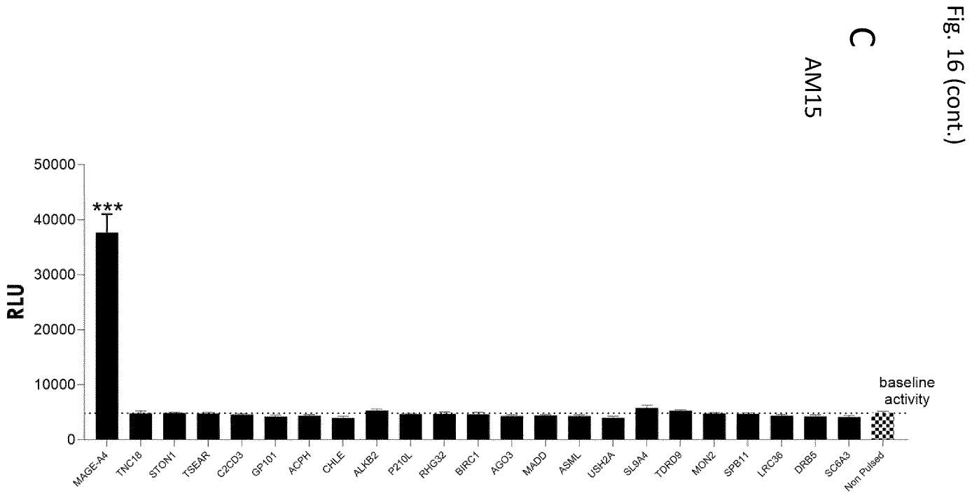

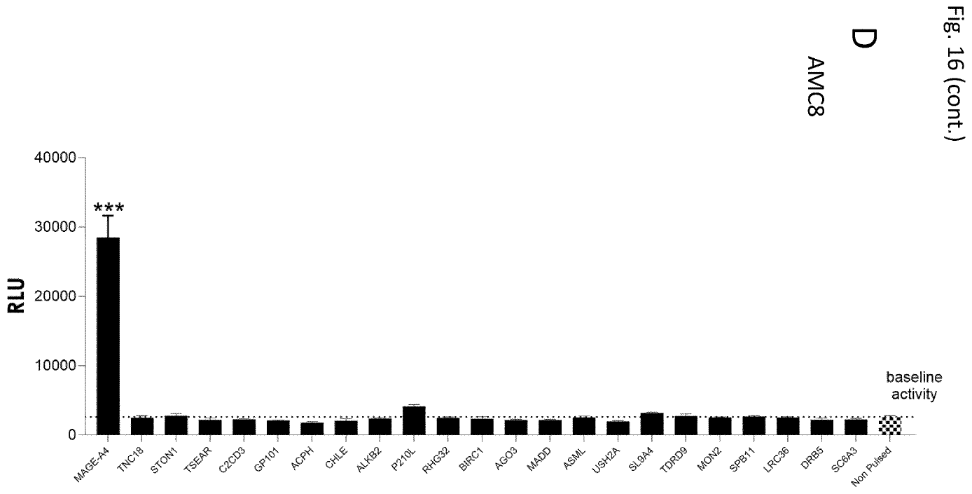

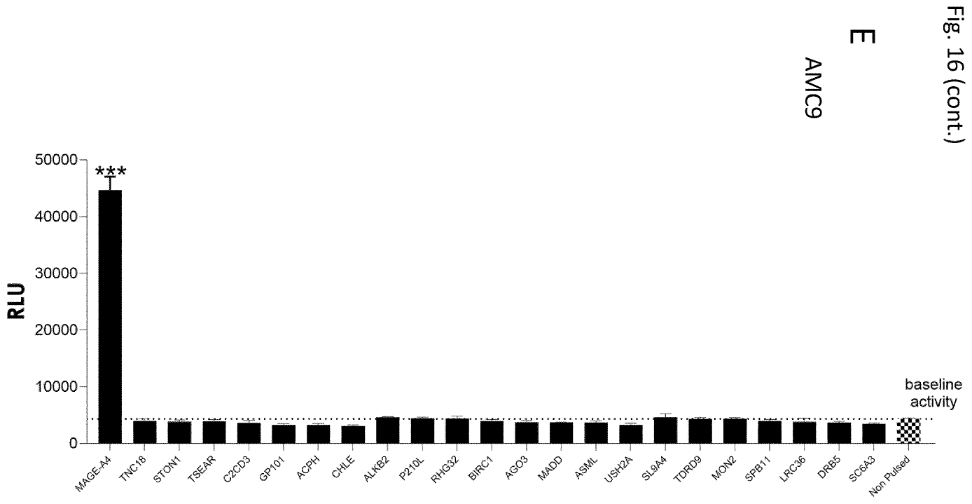

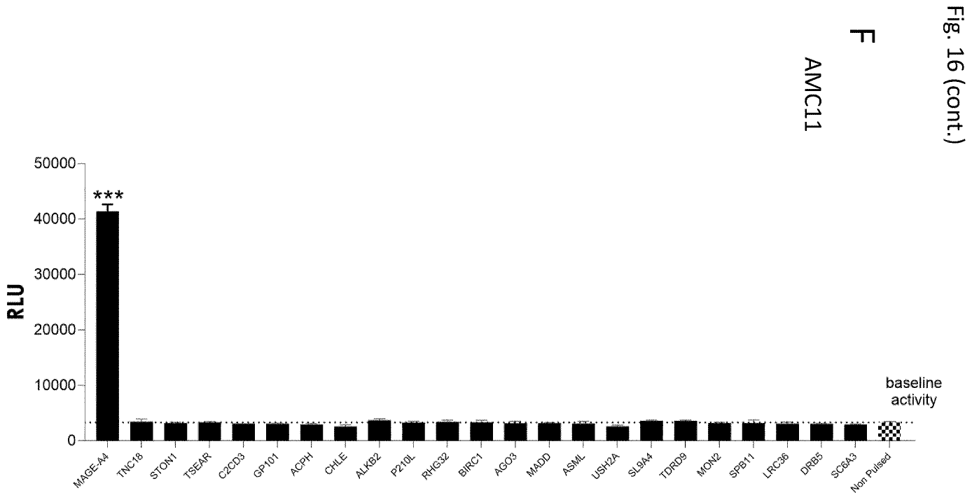

Specifically, the assay was used to assess the capacity of 1-H02 to induce T-cell mediated cytotoxicity of HLA-A*02:01/MAGE-A4²³⁰⁻²³⁹ positive and negative cancer cells (FIGS. 15A and 15B), and MAGE-A4²³⁰⁻²³⁹ peptide pulsed T2 cells (FIG. 12). As above, in some assays the target cells were T2 cells which were pulsed with exogenously applied MAGE-A4²³⁰⁻²³⁹ peptide. In these assays, control cells were non-pulsed T2 cells, and T2 cells pulsed with an unrelated peptide. In other assays, the target cells were HLA-A*02:01/MAGE-A4²³⁰⁻²³⁹ positive cancer cells. In these assays control cells were HLA-A*02:01/MAGE-A4²³⁰⁻²³⁹ negative cancer cells.

In these assays, a 1:1 ratio of effector cells to target cells was again used. As shown in FIGS. 12, 15A and 15B, 1-H02 induced T-cell mediated target cell killing only when co-cultured with HLA-A*02:01/MAGE-A4²³⁰⁻²³⁹ positive cells, i.e. T2 cells pulsed with MAGE-A4²³⁰⁻²³⁹ peptide, and with HLA-A*02:01/MAGE-A4²³⁰⁻²³⁹ positive cancer cell lines. No T-cell mediated cytotoxicity resulted against cells lacking HLA-A*02:01/MAGE-A4²³⁰⁻²³⁹, i.e. in non-pulsed T2 cells, T2 cells pulsed with a different peptide, and HLA-A*02:01/MAGE-A4²³⁰⁻²³⁹ negative cancer cells. Cytotoxicity against HLA-A*02:01/MAGE-A4²³⁰⁻²³⁹ positive tumor cell lines representing both solid tumors (15A) and liquid tumors (15B) was demonstrated, and with different T-cell comprising effector cell populations; PBMCs (15A) and pan T-cells isolated therefrom (15B).

Similar analyses were performed using many other of the antibodies disclosed in Table A, including but not limited to AM2, AM6, AM15, AMC8, AMC9 and AMC11, with similarly specific cytotoxicity effects being observed as for 1-H02 (data not shown).

The antigen binding proteins (e.g. antibodies) of the invention, when in a T-cell engager format (e.g. in the scDb format), are capable of preferentially (or selectively) inducing T-cell mediated cytotoxicity against cells displaying HLA-A*02:01/MAGE-A4²³⁰⁻²³⁹.

Thus, in an embodiment, the antigen binding proteins (e.g. antibodies) of the invention, when in a T-cell engager format (e.g. in the scDb format), preferentially (or selectively) induce T-cell mediated cytotoxicity against cells displaying HLA-A*02:01/MAGE-A4²³⁰⁻²³⁹ as compared to against cells displaying HLA-A*02:01 without the MAGE-A4²³⁰⁻²³⁹ peptide complexed thereto.

In an embodiment, the antigen binding proteins (e.g. antibodies) of the invention, when in a T-cell engager format (e.g. in the scDb format), preferentially (or selectively) induce T-cell mediated cytotoxicity against cells displaying HLA-A*02:01/MAGE-A4²³⁰⁻²³⁹ as compared to against cells displaying a HLA-A*02:01 restricted peptide that is not MAGE-A4²³⁰⁻²³⁹.

In an embodiment, the antigen binding proteins (e.g. antibodies) of the invention, when in a T-cell engager format (e.g. in the scDb format), preferentially (or selectively) induce T-cell mediated cytotoxicity against cells displaying HLA-A*02:01/MAGE-A4²³⁰⁻²³⁹ as compared to against cells displaying HLA-A*02:01 without the MAGE-A4²³⁰⁻²³⁹ peptide complexed thereto and/or (preferably “and”) against cells displaying a HLA-A*02:01 restricted peptide that is not MAGE-A4²³⁰⁻²³⁹.

Alternatively viewed, in an embodiment, the antigen binding proteins (e.g. antibodies) of the invention, when in a T-cell engager format (e.g. in the scDb format), preferentially (or selectively) induce T-cell mediated cytotoxicity against HLA-A*02:01/MAGE-A4²³⁰⁻²³⁹ positive cells as compared to against HLA-A*02:01 positive, MAGE-A4²³⁰⁻²³⁹ negative cells.

The HLA-A*02:01/MAGE-A4²³⁰⁻²³⁹ displaying, or positive or negative cells referred to herein, may be any cell type including the preferred target and cancer cell types disclosed elsewhere herein.

The T-cells may be as defined anywhere else herein, and are preferably CD4⁺ T cells or CD8⁺ T cells.

Any substantially homologous antigen binding protein (e.g. antibody) of the invention preferably exhibits such inducement of T-cell mediated cytotoxicity.

The term “induce” as used herein means promote, i.e. enhance or increase the stated property or activity. Induce need not mean that the starting level of the property or activity is zero, i.e. it may mean “induce further”.

Preferably such T-cell mediated cytotoxicity is exhibited when the antigen binding protein is an antibody in any T-cell engager format, preferably a bispecific format. Preferably, the antibody is in the scDb format.

Preferably such T-cell mediated cytotoxicity is exhibited when the ratio of T-cells (or effector cells comprising T cells) to cells displaying HLA-A*02:01/MAGE-A4²³⁰⁻²³⁹ (“target cells”) is about 1:1. Thus, preferably such T-cell mediated cytotoxicity is exhibited in an assay comprising the co-culturing of HLA-A*02:01/MAGE-A4²³⁰⁻²³⁹ positive target cells (“T”) and effector cells comprising T cells (“E”) at an E:T ratio of about 1:1 (e.g. 1:1), together with the antigen binding protein (e.g. antibody), preferably tested over a concentration range, with subsequent detection and quantification of a marker of cell death. Preferably, such detection and quantification is as described below.

### Assay 5: Assay for T-Cell Mediated Cytotoxicity

The ability of an antigen binding protein (e.g. antibody) in a T-cell engager format to induce T-cell mediated cytotoxicity against cells displaying a HLA-restricted peptide (pHLA) (“target cells”) can be assessed by any appropriate means and the skilled person is familiar with suitable methods, e.g. co-culturing CFSE-labelled target cells (“T”), and effector cells (“E”) comprising T cells, such as PBMCs or pan-T-cells derived therefrom, preferably at an E:T ratio of about 1:1, together with the antigen binding protein (e.g. antibody) to be tested, and subsequently detecting and quantifying a marker of cytotoxicity. Such detection and quantification may be performed by staining for non-viable cells (e.g. with 7-amino actinomycin D (7-AAD)) and detecting said stain, e.g. via flow cytometry.

Thus, a suitable assay may comprise:
(a) Co-culturing in wells of an assay plate CFSE-labelled target cells (“T”), e.g. cancer cells (e.g. cancer cell line cells), or peptide pulsed T2 cells, e.g. as described above, and effector cells comprising T cells (“E”), e.g. PBMCs or pan-T-cells derived therefrom, e.g. as described above, at an E:T ratio of about 1:1, and the antigen binding protein (e.g. antibody) to be tested, e.g. at a single concentration (e.g. about 100 ng/ml), or over a concentration range (e.g. of about 0.01-1000 ng/ml), and co-culturing e.g. for about 72 h at about 37° C.;
(b) Harvesting assay supernatants and cells from the assay plates;
(c) Adding a fluorescent stain for non-viable cells (e.g. 7-AAD);
(d) Detecting the fluorescent-stain (e.g. 7-AAD) using multi-colour flow cytometry; and
(e) Quantifying the signal of the stain, e.g. using Zombie Yellow fixable viability kit, using appropriate fluorescence minus one (FMO) controls to exclude background staining.

If peptide pulsed T2 cells are used as target cells, then T2 cells are pulsed with the relevant peptide prior to step (a), e.g. by resuspension in assay buffer containing the peptide (e.g. 50 μM of peptide) and incubating e.g. for about 4.5 hrs at about 37° C., and subsequently centrifuging the resulting peptide pulsed T2 cells and resuspending them in fresh assay buffer to remove excess peptide.

In some embodiments, such an assay comprises the use of peptide pulsed T2 cells or cancer cells (e.g. cancer cell line cells) as target cells, and PBMCs or pan-T cells as effector cells, and the assay comprises:
(a) If peptide pulsed T2 cells are used as target cells, then T2 cells are resuspended in assay buffer (e.g. RPMI/10% FBS/1% P/S) containing peptide (e.g. about 50 μM of peptide), which may be MAGE-A4²³⁰⁻²³⁹ or a comparator peptide, incubating e.g. for about 4.5 hrs at about 37° C., and subsequently centrifuging the resulting peptide pulsed T2 cells and resuspending them in fresh assay buffer to remove excess peptide (if cancer cells are used as target cells then this step is not performed);
(b) Pelleting and resuspending target cells (peptide pulsed T2 cells or cancer cells) in PBS (e.g. 1 ml Dulbecco's phosphate-buffered saline (DPBS)) containing CFSE (e.g. about 0.5 μM CFSE), incubating for e.g. about 8 minutes at room (or ambient) temperature (e.g. about 25° C.), and adding assay buffer (e.g. about 9 ml of assay buffer);
(c) Pelleting the target cells, washing the target cells once in assay buffer, counting and resuspending the target cells at a desired concentration;
(d) Diluting the antigen binding protein (e.g. antibody) to be tested e.g. to 10× the final concentrations required in assay buffer;
(e) If PBMCs are used as effector cells, preparing PBMCs, e.g. by thawing frozen PBMCs obtained from liquid nitrogen storage (e.g. in a 37° C. water bath), and transferring said PBMCs drop wise into pre-warmed assay buffer, centrifuging and resuspending the PBMCs in fresh assay buffer, counting and resuspending the PBMCs at a desired concentration; or
If pan-T cells are used as effector cells, preparing pan-T cells from PBMCs using any known protocol, e.g. using the EasySep™ Human T cell isolation kit/protocol, e.g. said kit/protocol described in the Examples herein;
(f) Preparing a co-culture of PMBCs or pan-T cells (effector cells “E”) and target cells (“T”) (i.e. peptide pulsed T2 or cancer cells (e.g. cancer cell line cells)) in wells of a culture plate at an E:T ratio of about 1:1 (e.g. 10,000 PBMCs and 10,000 target cells) e.g. in a total volume of about 180 μl;
(g) Adding e.g. about 20 μl of the antigen binding protein (e.g. antibody) to be tested to the wells e.g. to make a final volume of about 200 μl;
(h) Incubating the co-culture at about 37° C. e.g. for about 72 hrs;
(i) If adherent cancer cells were used as target cells, then detaching said adherent cells by incubation with Trypsin/EDTA, and adding cell culture supernatant back to detached cells (this step is not performed if the target cells are T2 cells or non-adherent cancer cells);
(j) Transferring target cells to a fresh culture plate (e.g. a 96-well plate);
(k) Staining dead cells using 7-AAD (e.g. about 1 μl 7-AAD/well); and
(l) Analysing fluorescence (of cells) using a flow cytometer (e.g. a BD Canto II).

In such assays, fluorescence (from cells) is from CFSE and 7-AAD labelled cells. The different fluorescence signals can be distinguished using standard flow cytometry techniques, thereby permitting detection of proliferation either from all T cells and/or from particular T cell types of interest detection of dead cell signal (via 7-AAD fluorescence) specifically from target cells (via CFSE fluorescence).

In such an assay, the antigen binding protein may be an antibody in any T-cell engager antibody format, preferably a bispecific format. In a preferred assay, the antibody is in the scDb format.

A preferred assay for assessing the ability of an antigen binding protein (e.g. antibody) to induce T-cell mediated cytotoxicity against a target cell is described in the Example section herein.

### EC50

The T-cell mediated cytotoxicity assay described above (Assay 5) was performed in Example 4 (results shown in FIG. 15), which allowed for the determination of the EC₅₀ value for many of the antibodies of the invention disclosed in Table A, which were assayed over a concentration range of 0.01-1000 ng/ml.

The parameter “EC₅₀” is the concentration of the antigen binding protein (e.g. antibody) of the invention that is necessary to achieve half of the maximum possible effect, which in the present case is half of the observed T-cell mediated cytotoxicity of target cells.

For example, the exemplified 1-H02 antibody of the invention in the scDb format shows (FIGS. 15A & 15B):
an EC₅₀ (for cytotoxicity) of 166.2 pM as measured in the cancer cell line NCI-H1703;
an EC₅₀ (for cytotoxicity) of 130.1 pM as measured in the cancer cell line A375; and
an EC₅₀ (for cytotoxicity) of 10.7 nM (10700 pM) as measured in the cancer cell line THP1.

Example 4 (Tables I to K) further demonstrates EC₅₀ values for many of the antibodies of the invention disclosed in Table A as measured in the cancer cell lines NCI-H1703, A375, C-33-A and HuTu80, with EC₅₀ values ranging from 0.72 pM to 11 pM, and most below 5 pM. Such cytotoxicity values in the low (single digit or low double digit) picomolar range are markedly advantageous. The present inventors have demonstrated that the antibodies of the invention, advantageously, have marked cytotoxicity against a wide and varied range of different cancer types/cell lines.

As mentioned above, in assays of the capability of an antigen binding protein (e.g. antibody) in a T-cell engager format to induce T-cell activity, including cytotoxicity, against target cells, the apparent activity/efficacy of the antigen binding protein (e.g. antibody) can be artificially inflated through use of a high E:T ratio in the step(s) of co-culturing the effector cells comprising T cells (“E”) and the target cells (“T”). The E:T ratio is simply the ratio of the number of effector cells to the number of target cells. The use of a greater number of effector cells relative to target cells is more likely to lead to a higher level of observed effector cell activity against the target cells. E:T ratios of 10:1 are seen in the art, but this is considered a high E:T ratio that can mask poor performance of the antibodies being tested.

The presently described EC₅₀ values exhibited by the antigen binding proteins (e.g. antibodies) of the invention are exhibited in assays in which an E:T ratio of 1:1 is used. Lower (i.e. better) EC₅₀ values could be achieved if a higher E:T ratio was used, e.g. 10:1.

Using the same assay protocol in each case, i.e. in which an E:T ratio of 1:1 was used, the present inventors have demonstrated that the antigen binding proteins (e.g. antibodies) of the invention have marked cytotoxicity, exhibiting EC₅₀ values in the picomolar (or low nanomolar) range as measured in disparate cancer cell types. Preferably the antigen binding proteins (e.g. antibodies) of the invention exhibit EC₅₀ values in the picomolar of femtomolar range as measured in cancer cells.

Thus, preferably, antigen binding proteins (e.g. antibodies) of the present invention, for example when in a T-cell engager format, preferably a bispecific format, e.g. the scDb format, exhibit an EC₅₀ of less than 1.1×10⁻⁸ M as measured in cancer cells, e.g. less than 10⁻⁸ M, more preferably less than 10⁻⁹ M, 10⁻¹⁰ M, 10⁻¹¹ M, 10⁻¹² M, 10⁻¹³ M, or 10⁻¹⁴ M as measured in cancer cells.

Preferably, antigen binding proteins (e.g. antibodies) of the present invention, for example when in a T-cell engager format, preferably a bispecific format, e.g. the scDb format, exhibit an EC₅₀ of less than 2×10⁻¹⁰, 5×10⁻¹¹, 2.5×10⁻¹¹, or 1×10⁻¹¹ M, preferably less than 8×10⁻¹² M, more preferably less than 7×10⁻¹² M, more preferably less than 6×10⁻¹² M, more preferably less than 5×10⁻¹² M, e.g. less than 4×10⁻¹² M, e.g. less than 3×10⁻¹² M, less than 2×10⁻¹² M or less than 1×10⁻¹² M, as measured in cancer cells.

Preferably the EC50 value is as measured in HLA-A*02:01/MAGE-A4²³⁰⁻²³⁹ positive cancer cells e.g. cancer cell line cells, preferably NCI-H1703 cells, A375 cells, THP1 cells, C-33-A cells or HuTu80 cells, more preferably NCI-H1703 cells.

Preferably, such EC₅₀ values are as determined in an assay comprising the co-culturing of cancer cells (target cells “T”) and effector cells comprising T cells (“E”) at an E:T ratio of about 1:1 (e.g. 1:1), together with the antigen binding protein (e.g. antibody), preferably tested over a concentration range, with subsequent detection and quantification of a marker of cell death. Preferably, such detection and quantification is performed by staining for non-viable cells (e.g. with 7-amino actinomycin D (7-AAD)) and detecting said stain, e.g. via flow cytometry.

In such assays, the effector cells may be as defined elsewhere herein. Preferred effector cells are PBMCs, or pan-T cells derived therefrom.

In such assays, preferably the step of co-culturing the target and effector cells is performed for about 72 hours, at about 37° C.

In such an assay, the antigen binding protein may be an antibody in any T-cell engager format, preferably a bispecific format. In a preferred assay, the antibody is in the scDb format.

A preferred assay for determining EC₅₀ values is set out in Assay 5 above, and in the Examples.

Any substantially homologous antigen binding protein (e.g. antibody) of the invention preferably exhibits such EC₅₀ values.

The antigen binding proteins (e.g. antibodies) of the invention exhibit cytotoxicity against HLA-A*02:01/MAGE-A4²³⁰⁻²³⁹ cancer cells, preferably with the above-mentioned EC₅₀ values. Preferably, the antigen binding proteins (e.g. antibodies) of the invention exhibit cytotoxicity against one or more of NCI-H1703 cells, A375 cells, THP1 cells, C-33-A cells and HuTu80 cells, preferably with the above-mentioned EC₅₀ values. Preferably, the antigen binding proteins (e.g. antibodies) of the invention exhibit cytotoxicity against NCI-H1703 cells, preferably with the above-mentioned EC₅₀ values. Preferably, the antigen binding proteins (e.g. antibodies) of the invention exhibit cytotoxicity against NCI-H1703 cells, and one or more of A375 cells, THP1 cells, C-33-A cells and HuTu80 cells, preferably with the above-mentioned EC₅₀ values. Preferably, the antigen binding proteins (e.g. antibodies) of the invention exhibit cytotoxicity against NCI-H1703 cells, and A375 cells, preferably with the above-mentioned EC₅₀ values. Preferably, the antigen binding proteins (e.g. antibodies) of the invention exhibit cytotoxicity against NCI-H1703 cells, A375 cells, C-33-A cells and HuTu80 cells, preferably with the above-mentioned EC₅₀ values. Preferably, the antigen binding proteins (e.g. antibodies) of the invention exhibit cytotoxicity against NCI-H1703 cells, A375 cells, and THP1 cells, preferably with the above-mentioned EC₅₀ values.

Preferably, such cytotoxicity is determined in an assay comprising the co-culturing of cancer cells (target cells “T”) and effector cells comprising T cells (“E”) at an E:T ratio of about 1:1 (e.g. 1:1), together with the antigen binding protein (e.g. antibody), preferably tested over a concentration range, with subsequent detection and quantification of a marker of cell death. Preferably, such detection and quantification is be performed by staining for non-viable cells (e.g. with 7-amino actinomycin D (7-AAD)) and detecting said stain, e.g. via flow cytometry.

In such assays, the effector cells may be as defined elsewhere herein. Preferred effector cells are PBMCs, or pan-T cells derived therefrom.

In such assays, preferably the step of co-culturing the target and effector cells is performed for about 72 hours, at about 37° C.

In such an assay, the antigen binding protein may be an antibody in any T-cell engager format, preferably a bispecific format. In a preferred assay, the antibody is in the scDb format.

A preferred assay for determining cytotoxicity is set out in Assay 5 above, and in the Examples.

Any substantially homologous antigen binding protein (e.g. antibody) of the invention preferably exhibits such cytotoxicity.

### Binding Affinity

Preferably, antigen binding proteins (e.g. antibodies) of the present invention, for example when in an scDb format, have a binding affinity for HLA-A*02:01/MAGE-A4²³⁰⁻²³⁹), e.g. have a K_{D} (equilibrium dissociation constant) in the range of 500 nM or less, e.g. 400 nM or less, e.g. 370 nM or less, for example when determined in an SPR assay.

Thus, antigen binding proteins (e.g. antibodies) of the invention, for example when in a T-cell engager format, preferably in the scDb format, may have a binding affinity for HLA-A*02:01/MAGE-A4²³⁰⁻²³⁹ that is or corresponds to a K_{D} of less than 500 nM, less than 400 nM, less than 375 nM, less than 350 nM, or less than 300 nM, 250 nM, 200 nM, 150 nM, 100 nM, 50 nM, 10, 5, 4, 3, 2, or 1 nM, or less than 500 pM, less than 400 pM, less than 300 pM, 200 pM, 100 pM, 50 pM, 20 pM, 10 pM, or 5 pM.

In embodiments, the antigen binding proteins (e.g. antibodies) of the invention when in a T-cell engager format, preferably in the scDb format, have a binding affinity for HLA-A*02:01/MAGE-A4²³⁰⁻²³⁹ that is or corresponds to a K_{D} of about 400 nM, 375 nM, 350 nM, 300 nM, 250 nM, 200 nM, 150 nM, 100 nM, 50 nM, 10, 5, 4, 3, 2, or 1 nM, 500 pM, 400 pM, 300 pM, 200 pM, 100 pM, 50 pM, 20 pM, 10 pM, or 5 pM.

Preferably, the antigen binding proteins (e.g. antibodies) of the invention when in a T-cell engager format, preferably in the scDb format, have a binding affinity for HLA-A*02:01/MAGE-A4²³⁰⁻²³⁹ that is or corresponds to a K_{D} of 0.1 to 450 nM, e.g. 0.1 to 200 nM, 0.1 to 100 nM, 0.1 to 50 nM, 0.1 to 25 nM, or 0.1 to 20 nM.

In embodiments, the antigen binding proteins (e.g. antibodies) of the invention when in the Fab format, have a binding affinity for HLA-A*02:01/MAGE-A4²³⁰⁻²³⁹ that is or corresponds to a KD of 1 to 1500 nM, e.g. 1 to 500 nM, 1 to 400 nM, or 1 to 350 nM, or preferably 10 to 1500 nM, e.g. 10 to 500 nM, 10 to 400 nM, or 10 to 350 nM.

The present antibodies demonstrate advantageous cytotoxicity against target-positive cancer cells, despite having relatively weak target binding affinity (in the nanomolar or high picomolar range, rather than in the mid- or low picomolar range). Far from being a disadvantage of the present antibodies, this relatively weak target binding affinity is advantageous. When a plurality of different antibodies that achieve the same or similar levels of efficacy/activity/cytotoxicity, antibodies that display a weaker binding affinity are actually advantageous in terms of their safety profile, since an antibody with a weaker affinity for the target is less likely to bind strongly to off-target molecules of a similar structure. Highly cytotoxic antibodies often bind to their targets with high affinity, but antibodies that display similar cytotoxicity with a lower affinity would be desirable given the balance of efficacy and safety.

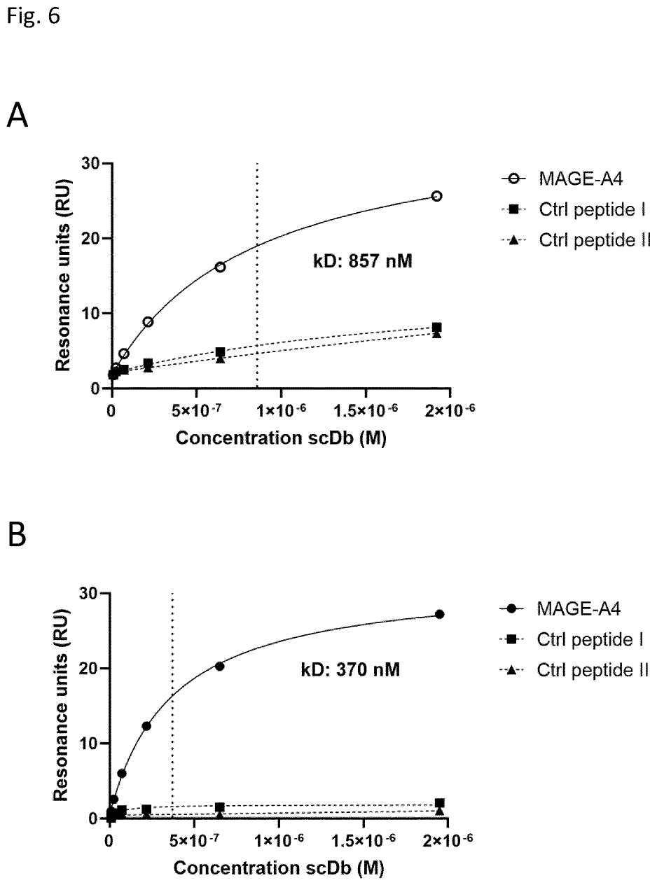

For example, an exemplified 1-H02 antibody of the invention shows a binding affinity of 370 nM in the scDb format (Example 2, FIG. 6B), and other exemplified antibodies in this format show binding affinities of 3.2 nM (AM11), 7.0 nM (AM13), 0.44 nM (AM14), and 10.8 nM (AM17) (Example 2).

For example, exemplified antibodies of the invention in the Fab format show the following binding affinities: 15.8 nM (AMC9), 86.3 nM (AM2), 148 nM (AM15), 325 nM (AM10) and 1410 nM (1-H02) (Example 2).

Any substantially homologous antigen binding protein (e.g. antibody) of the invention preferably exhibits such binding affinity.

### Assay 6: Binding Affinity

Any suitable method of determining the binding affinity (K_{D}) may be used and the skilled person is familiar with suitable methods. Preferably the K_{D} is determined in a Surface Plasmon Resonance assay (e.g. a BIAcore assay), preferably in which kinetic parameters are determined.

Suitable SPR assays are known in the art and for example may involve immobilising HLA-A*02:01/MAGE-A4²³⁰⁻²³⁹ on a solid support and applying the antigen binding protein (e.g. antibody) to be tested. Suitable assays are discussed elsewhere herein. Particularly preferred SPR assays are described in the Examples section herein.

In certain preferred SPR assays, HLA-A*02:01/MAGE-A4²³⁰⁻²³⁹ is captured (or immobilised) on a solid support (e.g. a sensor chip) and various concentrations (e.g. a dilution series, e.g. a doubling dilution series) of the antigen binding protein (e.g. antibody) to be tested is then injected. Antigen binding protein (e.g. antibody) concentrations and response units (RU) are generally selected at a range and level, respectively, such that the chip is not saturated and which allow robust fitting, e.g. robust 1:1 fitting, by the SPR/Biacore software. Preferred concentrations and flow-rates for injection, together with appropriate RU Units are described in the Examples section.

Suitable association periods and dissociation periods to be used in an SPR assay are known to a skilled person, for example, a preferred association period in the SPR assay is 2 minutes and a preferred dissociation period in the SPR assay is 2 minutes (in a single cycle analysis). In certain embodiments, all measurements may be performed at 25° C. in 20 mM PBS, pH7.4, 2.7 mM KCl, 137 mM NaCl. Kinetic parameters may be determined or calculated by any suitable model or software, for example by fitting the sensorgram experimental data assuming a 1:1 interaction, in other words using a 1:1 binding model, for example using Single Cycle Kinetics software. Particularly preferred SPR assays are described in the Examples section herein. Preferably a single cycle analysis is used.

In some embodiments, binding affinity (K_{D}) of an antigen binding protein (e.g. antibody) of the invention to HLA-restricted peptide (pHLA), e.g. HLA-A*02:01/MAGE-A4²³⁰⁻²³⁹, may be as determined by (or as assessed by) an SPR assay, e.g. an SPR assay that comprises the following (Assay 6-1):
(a) Coupling NeutrAvidin (e.g. 10 μg/mL in 10 mM sodium acetate, pH 5.0), to a solid support (e.g. a sensor chip, such as a CM3 series S sensor chip) via amine coupling, e.g. to 500 response units (RU);
(b) Adding (capturing), pHLA (e.g. soluble, recombinant, biotinylated pHLA) (e.g. 1 μg/mL) on said solid support e.g. to 40-100 RU,
(c) Applying the antigen binding protein (e.g. antibody) to be tested to the solid support;
(d) Using an association period of 2 min for injection of samples, in a concentration series of single cycle kinetic experiments;
(e) Using dissociation periods of 2 min between injection of samples in a concentration series of single cycle kinetics experiments, and a final dissociation time of 20 min; and
(f) Obtaining sensorgram data and determining binding affinity (K_{D}) using a suitable model or software, for example by using S200 Evaluation Software v1.1., e.g. by fitting the sensorgram data from Single Cycle Kinetics method to a 1:1 Langmuir binding model after buffer subtraction and NeutrAvidin-reference-cell subtraction,
wherein all measurements are performed at 25° C. in 20 mM PBS, pH7.4, 2.7 mM KCl, 137 mM NaCl.

In some embodiments, binding affinity (K_{D}) of an antigen binding protein (e.g. antibody) of the invention to HLA-restricted peptide (pHLA), e.g. HLA-A*02:01/MAGE-A4²³⁰⁻²³⁹, may be as determined by (or as assessed by) an SPR assay, e.g. an SPR assay that comprises the following (Assay 6-2):
(g) Coupling NeutrAvidin (e.g. 10 μg/mL in 10 mM sodium acetate, pH 5.0), to a solid support (e.g. a sensor chip, such as a CM3 series S sensor chip) via amine coupling, e.g. to 500 response units (RU);
(h) Adding (capturing), pHLA (e.g. soluble, recombinant, biotinylated pHLA) (e.g. 1 μg/mL) on said solid support e.g. to 130-150 RU,
(i) Applying the antigen binding protein (e.g. antibody) to be tested to the solid support;
(j) Using an association period of 2 min for injection of samples, in single cycle kinetic experiments;
(k) Using dissociation periods of 30 min between injection of samples; and
(l) Obtaining sensorgram data and determining binding affinity (K_{D}) using a suitable model or software, for example by using S200 Evaluation Software v1.1., e.g. by fitting the sensorgram data from Single Cycle Kinetics method to a 1:1 Langmuir binding model after buffer subtraction and NeutrAvidin-reference-cell subtraction,
wherein all measurements are performed at 25° C. in 20 mM PBS, pH7.4, 2.7 mM KCl, 137 mM NaCl.

### Thermostability

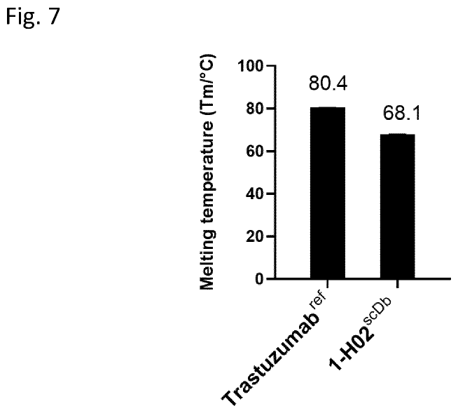

Example 2 herein describes the assessment of the thermal stability of an antibody of the invention, 1-H02, in the scDb format. As shown in FIG. 7, the melting temperature of 1-H02, measured by thermal unfolding and defined as the temperature at which 50% of the molecules are unfolded, was 68.1° C. This advantageous stability was surprising. The antigen binding Fab unit of the well-performing therapeutic antibody Trastuzumab was included as control, in a format (Fab) which normally has higher thermostability due to the CH1-CL pair, yet 1-H02 (in scDb) format had a similar melting temperature.

The same analysis was performed using all of the other specific antibodies disclosed in Table A, and similar thermal stabilities were observed as for 1-H02; all antibodies were determined to have a melting temperature in the range of 68.1° C. to 70.4° C. (data not shown).

Thus, in an embodiment, the antigen binding proteins (e.g. antibodies) of the invention, when in an scDb format, have a melting temperature of at least 60° C., preferably at least 62° C., more preferably at least 64° C., more preferably at least 66° C., more preferably at least 68° C., e.g. 60° C. to 80° C., 62° C. to 80° C., 64° C. to 80° C., 66° C. to 80° C., or 68° C. to 80° C., or 60° C. to 75° C., 62° C. to 75° C., 64° C. to 75° C., 66° C. to 75° C., or 68° C. to 75° C., more preferably 64° C. to 72° C., 65° C. to 72° C., 66° C. to 72° C. or 68 to 72° C.

For example, the exemplified 1-H02 antibody of the invention shows a melting temperature of 68.1° C. in the scDb format (Example 2, FIG. 7).

Any substantially homologous antigen binding protein (e.g. antibody) of the invention preferably exhibits such a melting temperature.

The melting temperature is the temperature at which 50% of the molecules are unfolded.

### Assay 7: Thermostability

Any suitable method of determining the melting temperature may be used and the skilled person is familiar with suitable methods. Preferably the melting temperature is determined in a nano differential scanning fluorimetry (nanoDSF) assay, which is a well-known assay in the field, in which tryptophan or tyrosine fluorescence is used to monitor protein unfolding, and the ratio of the fluorescence intensities at 350 nm and 330 nm is suitable to detect any changes in protein structure due to protein unfolding. Particularly preferred melting temperature assays are described in the Examples section herein.

In some embodiments, the melting temperature of an antigen binding protein (e.g. antibody) of the invention to HLA-restricted peptide (pHLA), e.g. HLA-A*02:01/MAGE-A4²³⁰⁻²³⁹, may be as determined by (or as assessed by) a nanoDSF method, e.g. a nanoDSF method that comprises:
(a) Preparing a solution of antigen binding protein (e.g. antibody) to be tested, e.g. in PBS, e.g. at a concentration of 0.04-0.46 mg/ml;
(b) Transferring about 10 μL of said solution to glass capillaries (e.g. NanoTemper capillaries), e.g. in triplicates;
(c) Performing a discovery scan to set a suitable instrument laser intensity (e.g. set to 40% of maximal intensity)
(d) Subjecting samples to a temperature scan, e.g. from about 20° C. to about 95° C., with 1° C./min increments, e.g. using a Prometheus nanoDSF (NanoTemper);
(e) Applying an excitation wavelength of 295 nm, and measuring emission at 330 nm and 350 nm;
(f) Analysing collected data, e.g. using AB-Protein PR.ThermControl V2.12, to determine melting temperatures.

A preferred assay is described in the Example section herein.

Induction of IFNγ Release from T-Cells

Cytotoxic T cells release cytokines such as IFN-γ, which induce the increased expression of MHC class I and other molecules involved in peptide loading in cancer cells, which in turn increases the chance that cancer cells will be recognized as target cells for cytotoxic attack. IFN-γ also activates macrophages, recruiting them to target sites both as effector cells and as antigen-presenting cells. The ability of an antigen binding protein, particularly at low concentrations, to induce the release of IFN-γ from T-cells when co-cultured with HLA-A*02:01/MAGE-A4²³⁰⁻²³⁹ positive target cells would therefore be advantageous, and demonstrative of the cytotoxic and therapeutic activity of the molecules.

The antigen binding proteins (e.g. antibodies) of the invention, when in a T-cell engager format (e.g. in the scDb format), preferentially (or selectively) induce release of interferon gamma (IFNγ) from T cells directed against cells displaying HLA-A*02:01/MAGE-A4²³⁰⁻²³⁹.

Alternatively viewed, the antigen binding proteins (e.g. antibodies) of the invention, when in a T-cell engager format (e.g. in the scDb format), are capable of preferentially (or selectively) inducing release of IFNγ from T cells directed against cells displaying HLA-A*02:01/MAGE-A4²³⁰⁻²³⁹.

Thus, in an embodiment, the antigen binding proteins (e.g. antibodies) of the invention, when in a T-cell engager format (e.g. in the scDb format), preferentially (or selectively) induce release of IFNγ from T cells directed against cells displaying HLA-A*02:01/MAGE-A4²³⁰⁻²³⁹ as compared to against cells displaying HLA-A*02:01 without the MAGE-A4²³⁰⁻²³⁹ peptide complexed thereto.

In an embodiment, the antigen binding proteins (e.g. antibodies) of the invention, when in a T-cell engager format (e.g. in the scDb format), preferentially (or selectively) induce release of IFNγ of T cells directed against cells displaying HLA-A*02:01/MAGE-A4²³⁰⁻²³⁹ as compared to against cells displaying a HLA-A*02:01 restricted peptide that is not MAGE-A4²³⁰⁻²³⁹.

In an embodiment, the antigen binding proteins (e.g. antibodies) of the invention, when in a T-cell engager format (e.g. in the scDb format), preferentially (or selectively) induce release of IFNγ from T cells directed against cells displaying HLA-A*02:01/MAGE-A4²³⁰⁻²³⁹ as compared to against cells displaying HLA-A*02:01 without the MAGE-A4²³⁰⁻²³⁹ peptide complexed thereto and/or (preferably “and”) against cells displaying a HLA-A*02:01 restricted peptide that is not MAGE-A4²³⁰⁻²³⁹.

Alternatively viewed, in an embodiment, the antigen binding proteins (e.g. antibodies) of the invention, when in a T-cell engager format (e.g. in the scDb format), preferentially (or selectively) induce release of IFNγ from T cells directed against HLA-A*02:01/MAGE-A4²³⁰⁻²³⁹ positive cells as compared to against HLA-A*02:01 positive, MAGE-A4²³⁰⁻²³⁹ negative cells.

The HLA-A*02:01/MAGE-A4²³⁰⁻²³⁹ displaying, or positive or negative cells referred to herein, may be any cell type including the preferred target and cancer cell types disclosed elsewhere herein.

The T-cells may be as defined anywhere else herein, and are preferably CD4⁺ T cells or CD8⁺ T cells.

Any substantially homologous antigen binding protein (e.g. antibody) of the invention preferably exhibits such inducement of T-cell activation, differentiation, or proliferation.

The term “induce” as used herein means promote, i.e. enhance or increase the stated property or activity. Induce need not mean that the starting level of the property or activity is zero, i.e. it may mean “induce further”.

Preferably such induction of release of IFNγ from T cells is exhibited when the antigen binding protein is any T-cell engager format, preferably a bispecific format. Preferably, the antibody is in the scDb format.

Preferably such induction of release of IFNγ from T cells is exhibited when the ratio of T cells (or effector cells comprising T cells) to cells displaying HLA-A*02:01/MAGE-A4²³⁰⁻²³⁹ (“target cells”) is about 1:1. Thus, preferably such induction of release of IFNγ from T cells is exhibited in an assay comprising the co-culturing of HLA-A*02:01/MAGE-A4²³⁰⁻²³⁹ positive target cells (“T”) and effector cells comprising T cells (“E”) at an E:T ratio of about 1:1 (e.g. 1:1), together with the antigen binding protein (e.g. antibody), preferably tested over a concentration range, with subsequent detection and quantification of IFNγ. Preferably, such detection and quantification is as described below.

### Assay 8: Assay for Induced T-Cell Release of IFNγ

The ability of an antigen binding protein (e.g. antibody) in a T-cell engager format to induce IFNγ release from PBMCs, more specifically T cells when co-cultured with cells displaying a HLA-restricted peptide (pHLA) (“target cells”) can be assessed by any appropriate means and the skilled person is familiar with suitable methods, e.g. co-culturing target cells (“T”), and effector cells (“E”) comprising T cells, such as PBMCs, preferably at an E:T ratio of about 1:1, together with the antigen binding protein (e.g. antibody) to be tested, and subsequently detecting and quantifying IFNγ release from PBMCs. Such detection and quantification may be performed by measuring the concentration of IFNγ in the cell culture medium following co-culture.

Target cells may be T2 cells pulsed with exogenously applied MAGE-A4²³⁰⁻²³⁹ peptide. In these assays, control cells are non-pulsed T2 cells, and T2 cells pulsed with an unrelated peptide. Alternatively, target cells may be HLA-A*02:01/MAGE-A4²³⁰⁻²³⁹ positive cancer cells. In these assays control cells are HLA-A*02:01/MAGE-A4²³⁰⁻²³⁹ negative cancer cells.

In assays of the ability of a T-cell engager to induce T-cell release of IFNγ, it is possible to use T cells, or a subset of T cells as the effector cells (e.g. PBMCs or pan T-cells isolated from PBMCs).

In such assays, the apparent efficacy of an antibody can be artificially inflated through use of a high E:T ratio in the co-culturing steps. The E:T ratio is simply the ratio of the number of effector cells to the number of target cells. The use of a greater number of effector cells relative to target cells is more likely to lead to a higher level of observed effector cell activity (e.g. IFNγ release) against the target cells. E:T ratios of 10:1 are seen in the art, but this is considered a high E:T ratio that can mask poor performance of the antibodies being tested. Therefore, it is preferred that an E:T ratio of 1:1 is used.

Assay kits for the detection and quantification of IFN-γ are well-known and widely available, for instance the ELISA Max Set from BioLegend®, and any suitable assay or kit may be used.

Thus, a suitable assay may comprise
(a) Co-culturing in wells of an assay plate HLA-A*02:01/MAGE-A4²³⁰⁻²³⁹ positive target cells (“T”), e.g. cancer cells (e.g. cancer cell line cells), as described above, and effector cells comprising T cells (“E”), e.g. PBMCs as described above, at an E:T ratio of about 1:1, and the antigen binding protein (e.g. antibody) to be tested, e.g. at a single concentration (e.g. about 1 nM), or over a concentration range (e.g. of about 0.1-10,000 pM), and co-culturing e.g. for about 24 h at about 37° C.;
(b) Harvesting assay supernatants from the assay plates;
(c) Assessing the concentration of IFNγ and the EC50 of the response, e.g. via ELISA, e.g. using a commercially available human IFNγ ELISA kit.

It is within the competencies of the person of ordinary skill in the art to prepare and make use of suitable controls and standard curves to facilitate correct running of assays and the determination of accurate concentrations.

In some embodiments, such an assay comprises the use of HLA-A*02:01/MAGE-A4²³⁰⁻²³⁹ positive cancer cells (e.g. cancer cell line cells) as target cells, and PBMCs as effector cells, and the assay comprises:
(a) Co-culturing (e.g. in a total volume of about 200 μl) the HLA-A*02:01/MAGE-A4²³⁰⁻²³⁹ positive target cells (“T”) and the PBMC effector cells at an E:T ratio of about 1:1 (e.g. 50,000 PBMCs and 50,000 target cells), and the antigen binding protein (e.g. antibody) to be tested, e.g. at a single concentration (e.g. about 1 nM), or over a concentration range (e.g. of about 0.1-10,000 pM), and co-culturing e.g. for about 24 h at about 37° C., and harvesting supernatant;
(b) Coating an ELISA plate (i.e. wells of the plate) with IFNγ capture antibody, as per manufacturer's instructions, e.g. for about 16 hrs at e.g. at 4° C.;
(c) Washing the plate, for example with wash buffer (e.g. PBS with 0.05% Tween 20) at least once (e.g. 4 times);
(d) Incubating the plate (e.g. for about 1 hr), e.g. at room (or ambient) temperature (e.g. 25° C.) with assay diluent);
(e) Washing the blocked plates, for example with wash buffer (e.g. PBS with 0.05% Tween 20), at least once e.g. 4 times;
(f) Adding 100 μl of supernatant obtained in step (a) (e.g. for about 2 hrs) at room (or ambient) temperature (e.g. 25° C.);
(g) Washing the ELISA plate, for example with wash buffer (e.g. PBS with 0.05% Tween 20) at least once e.g. 4 times;
(h) Incubating in the wells of the plate, e.g. for about 1 hour, for example at room (or ambient) temperature (e.g. 25° C.), with about 100 μl of a secondary antibody, e.g. a biotin labelled “detection” antibody;
(i) Washing plates, for example with wash buffer (e.g. PBS with 0.05% Tween 20) at least once (e.g. 4 times);
(j) Incubating in the wells of the plate, e.g. for about 30 minutes, for example at room (or ambient) temperature (e.g. 25° C.) with about 100 μl of 1:1000 diluted avidin-HRP;
(k) Washing plates, for example with wash buffer (e.g. PBS with 0.05% Tween 20) at least once (e.g. 4 times);
(l) Incubating in the wells of the plate, e.g. for up to 5 minutes, in the dark, about 100 μl of TMB solution (3,3′,5,5′-Tetramethylbenzidine);
(m) Immediately after incubation step (1), adding about 100 μl of 1M HCl (i.e. stop solution);
(n) Measuring absorbance e.g. at 450 nM, e.g. using a plate reader;
(o) Determining the concentration of IFNγ, e.g. by utilising a standard curve.

The T-cell IFNγ release assay described above (Assay 8) was performed in Example 6 (results shown in Table L), which allowed for the determination of an EC₅₀ value for the antibodies of the invention disclosed in Table A.

The parameter “EC₅₀” is the concentration of the antigen binding protein (e.g. antibody) of the invention that is necessary to achieve half of the maximum possible effect, which in the present case is half of the observed maximal amount of IFNγ released from T-cells when co-cultured with HLA-A*02:01/MAGE-A4²³⁰⁻²³⁹ positive target cells.

Example 6 (Table L) demonstrates EC₅₀ values (for IFNγ release from T-cells in the presence of HLA-A*02:01/MAGE-A4²³⁰⁻²³⁹ positive cancer cells) for each of the antibodies of the invention disclosed in Table A, with EC₅₀ values ranging from 1.7 pM to 83.1 pM, and most below 10 pM. Such values are markedly advantageous.

The present inventors have demonstrated that the antibodies of the invention, advantageously, have marked ability to induce release of IFNγ from T-cells when co-cultured with HLA-A*02:01/MAGE-A4²³⁰⁻²³⁹ positive target (cancer) cells.

As mentioned above, in assays of the capability of an antigen binding protein (e.g. antibody) in a T-cell engager format to induce T-cell activity, including IFNγ release, the apparent activity/efficacy of the antigen binding protein (e.g. antibody) can be artificially inflated through use of a high E:T ratio in the step(s) of co-culturing the effector cells comprising T cells (“E”) and the target cells (“T”). The E:T ratio is simply the ratio of the number of effector cells to the number of target cells. The use of a greater number of effector cells relative to target cells is more likely to lead to a higher level of observed effector cell activity against the target cells. E:T ratios of 10:1 are seen in the art, but this is considered a high E:T ratio that can mask poor performance of the antibodies being tested.

The presently described EC₅₀ values for IFNγ release from T-cells exhibited by the antigen binding proteins (e.g. antibodies) of the invention are exhibited in assays in which an E:T ratio of 1:1 is used. Lower (i.e. better) EC₅₀ values could be achieved if a higher E:T ratio was used, e.g. 10:1.

Using the same assay protocol in each case, i.e. in which an E:T ratio of 1:1 was used, the present inventors have demonstrated that the antigen binding proteins (e.g. antibodies) of the invention achieve marked IFNγ release, exhibiting EC₅₀ values in the picomolar range. Preferably the antigen binding proteins (e.g. antibodies) of the invention exhibit EC₅₀ values for IFNγ release from T-cells in the presence of HLA-A*02:01/MAGE-A4²³⁰⁻²³⁹ positive target (cancer) cells in the picomolar range.

Preferably, antigen binding proteins (e.g. antibodies) of the present invention, for example when in a T-cell engager format, preferably a bispecific format, e.g. the scDb format, exhibit an EC₅₀ for IFNγ release from T-cells in the presence of HLA-A*02:01/MAGE-A4²³⁰⁻²³⁹ positive target (cancer) cells of less than 5×10⁻¹⁰ M, e.g. less than 1×10⁻¹⁰, 5×10⁻¹¹ M, 2.5×10⁻¹¹ M, 10⁻¹¹ M, 5×10⁻¹² M, 10⁻¹² M, 10⁻¹³ M, or 10⁻¹⁴ M.

Preferably, antigen binding proteins (e.g. antibodies) of the present invention, for example when in a T-cell engager format, preferably a bispecific format, e.g. the scDb format, exhibit an EC₅₀ for IFNγ release from T-cells in the presence of HLA-A*02:01/MAGE-A4²³⁰⁻²³⁹ positive target (cancer) cells of less than 5×10⁻¹⁰ M, less than 1×10⁻¹⁰ M, less than 5×10⁻¹¹ M, less than 2.5×10⁻¹¹ M, less than 1×10⁻¹¹ M or less than 5×10⁻¹² M.

Preferably the EC50 value is as measured in HLA-A*02:01/MAGE-A4²³⁰⁻²³⁹ positive cancer cells e.g. cancer cell line cells, preferably A375 cells or NCI-H1703 cells, preferably A375 cells.

Preferably, such EC₅₀ values are as determined in an assay comprising the co-culturing of cancer cells (target cells “T”) and effector cells comprising T cells (“E”) at an E:T ratio of about 1:1 (e.g. 1:1), together with the antigen binding protein (e.g. antibody) to be tested over a concentration range, and subsequent detection and quantification of IFNγ. Preferably, such detection and quantification is performed by ELISA.

In such assays, the effector cells may be as defined elsewhere herein. Preferred effector cells are PBMCs, or pan-T cells derived therefrom.

In such assays, preferably the step of co-culturing the target and effector cells is performed for about 72 hours, at about 37° C.

In such an assay, the antigen binding protein may be an antibody in any T-cell engager format, preferably a bispecific format. In a preferred assay, the antibody is in the scDb format.

A preferred assay for determining said EC₅₀ values is set out in Assay 8 above, and in the Examples.

Any substantially homologous antigen binding protein (e.g. antibody) of the invention preferably exhibits such EC₅₀ values for IFNγ release from T-cells in the presence of HLA-A*02:01/MAGE-A4²³⁰⁻²³⁹ positive target (cancer) cells.

As discussed above, several of the above assays comprise the use of effector cells (“E”) and target cells (“T”). Preferably, the E:T ratio used in such assays is no greater than about 1:1, i.e. no more than about 1:1. Preferably the number of effector cells is not greater than, or is not significantly greater than, the number of target cells. E:T ratios lower than 1:1 may be used, however ratios of about 1:1, e.g. 1:1 are preferred. It is within the competencies of the person of ordinary skill in the art to count cell densities in cell cultures, dilute cells to a desired density and provide a suitable volume of cells to provide a given number of cells for an assay. In this way, desired E:T ratios can be straightforwardly provided.

In the above discussions of antigen binding protein (e.g. antibody) properties and assays for their assessment, there are disclosed parameters/conditions with the term “about” applied to a specific value, e.g. “about 72 hours”, “about 20° C.”, “about 100 ng/ml”, “about 1:1”, etc. The person of ordinary skill in the art will appreciate that minor modifications of parameters/conditions can be tolerated, and particular parameters/conditions can be readily selected by the skilled person for their particular purposes. For the avoidance of doubt, such disclosures are also disclosures of said specific value in particular, i.e. without the term “about” applied thereto.

Preferably, any substantially homologous antigen binding protein (e.g. antibody) should retain the ability to bind to, or specifically bind to, the same epitope of the antigen as recognized by the antigen binding protein (e.g. antibody) in question, for example, the same epitope recognized by the CDR domains of the invention, or the antigen binding domains of the invention, or the VH and VL domains of the invention, as described herein, e.g. bind to the same epitope as an antibody of the invention shown in Table A, e.g. the 1-H02 or AM15 antibody.

Thus, preferably any substantially homologous antigen binding protein (e.g. antibody) should retain the ability to bind to any one or more of, preferably all of, amino acid positions 3, 5, 6 and 8 of the HLA-A*02:01 restricted peptide MAGE-A4²³⁰⁻²³⁹ (SEQ ID NO: 19), which binding and assessment thereof is described elsewhere herein and in the Examples.

Thus, preferably, any substantially homologous antigen binding protein (e.g. antibody) should retain the ability to compete with one or more of the antigen binding proteins of the invention (e.g. shown in Table A) for binding to the relevant antigen. Binding to the same epitope/antigen can be readily tested by methods well known and described in the art, e.g. using binding assays, e.g. a competition assay. Suitable binding assays are discussed elsewhere herein.

Binding to the same epitope can, for example be tested, e.g. by using epitope mapping assays, e.g. by analysis of the crystal structure of the antigen-antibody complex, or by mutational studies of individual residues (e.g. using alanine scanning and/or deep mutational scanning, DMS, for example yeast display in combination with DMS, see for example as described in Sierocki et al., 2021, PLoS Negl Trop Dis., 15(3):e0009231; see also Van Blarcom et al., 2015, JMB, 427:6(B):1513-1534 and Medina-Cucurella and Whitehead, 2018, Methods Mol. Biol., 1764:101-121) and any of the above such analyses to determine the epitope can be used.

Thus, antibodies which bind to the same epitope as one or more of the various antibodies of the invention, for example as assessed or determined by one or more of the methods outlines above, form yet further aspects of the invention.

Retention of other functional properties, in particular binding affinity, can also readily be tested by methods well known and described in the art or herein.

Thus, a person skilled in the art will appreciate that binding assays can be used to test whether “substantially homologous” antigen binding proteins (e.g. antibodies) have the same binding specificities as the antigen binding proteins of the invention, for example, binding assays such as competition assays or ELISA assays, e.g. as described elsewhere herein. Surface Plasmon Resonance (e.g. BIAcore) assays could also readily be used to establish whether “substantially homologous” antigen binding proteins can bind to the relevant antigen. The skilled person will be aware of other suitable methods and variations.

As outlined below, a competition binding assay can be used to test whether “substantially homologous” antigen binding proteins (e.g. antibodies) retain the ability to bind to, or specifically bind to, substantially the same epitope of the relevant antigen as recognized by the antigen binding proteins (e.g. antibodies) of the invention (e.g. the 1-H02 or AM15 antibody), or have the ability to compete with one or more of the various antigen binding proteins (e.g. antibodies) of the invention. The method described below is only one example of a suitable competition assay. The skilled person will be aware of other suitable methods and variations.

An exemplary competition assay involves assessing the binding of various effective concentrations of an antigen binding protein (e.g. antibody) of the invention to the relevant antigen in the presence of varying concentrations of a test antigen binding protein (e.g. a substantially homologous antigen binding protein e.g. antibody). The amount of inhibition of binding induced by the test antigen binding protein can then be assessed. A test antigen binding protein that shows increased competition with an antigen binding protein of the invention at increasing concentrations (i.e. increasing concentrations of the test antigen binding protein result in a corresponding reduction in the amount of antigen binding protein of the invention binding to the relevant antigen) is evidence of binding to the same or substantially the same epitope. Preferably, the test antigen binding protein significantly reduces the amount of antigen binding protein of the invention that binds to the relevant antigen. Preferably, the test antigen binding protein reduces the amount of antigen binding protein of the invention that binds to the relevant antigen by at least about 95%. ELISA assays may be used for assessing inhibition of binding in such a competition assay but other suitable techniques would be well known to a person skilled in the art.

In some embodiments, “substantially homologous” antigen binding proteins (e.g. antibodies) which retain the ability to bind to, or specifically bind to, substantially the same (or the same) epitope of the relevant antigen as recognized by antigen binding proteins (e.g. antibodies) of the invention (e.g. the 1-H02 or AM15 antibody) or which have the ability to compete with one or more of the various antigen binding proteins (e.g. antibodies) of the invention (e.g. the 1-H02 or AM15 antibody) are preferred.

Such antigen binding proteins (e.g. antibodies) which have the ability to bind (or specifically bind) to the same (or substantially the same) epitope of HLA-A*02:01/MAGE-A4²³⁰⁻²³⁹ as recognized by the antibody of the invention (e.g. 1-H02 or AM15 of Table A) are further embodiments of the present invention. Such antigen binding proteins (e.g. antibodies) which have the ability to compete with one or more of the antibodies of the invention (e.g. with 1-H02 or AM15 shown in Table A) for binding to HLA-A*02:01/MAGE-A4²³⁰⁻²³⁹ are further embodiments of the present invention.

Thus, a yet further aspect of the invention provides an antigen binding protein (e.g. antibody), which binds to (or specifically binds to) HLA-A*02:01/MAGE-A4²³⁰⁻²³⁹ and which has the ability to bind to the same (or substantially the same) epitope as the 1-H02 or AM15 antibody (Table A), i.e. an antibody comprising the VL of SEQ ID NO:4 or 114 and the VH of SEQ ID NO:3, as described herein, or the ability to bind to the same (or substantially the same) epitope as an antibody comprising the same CDRs as the 1-H02 or AM15 antibody (Table A), i.e. an antibody comprising VL CDR sequences of SEQ ID NOs: 8, 9 and 10/96 and VH CDR sequences of SEQ ID NOs: 5, 6 and 7, for binding to HLA-A*02:01/MAGE-A4²³⁰⁻²³⁹. In a further aspect, such antigen binding proteins (e.g. antibodies) have the ability to compete with one or more of the antibodies of the invention (e.g. 1-H02 or AM15 (Table A)) for binding to HLA-A*02:01/MAGE-A4²³⁰⁻²³⁹. Other features and properties of other aspects of the invention apply, mutatis mutandis, to this aspect of the invention.

The term “competing antigen binding protein”, as used herein, refers to antigen binding proteins (e.g. antibodies) that bind to about, substantially or essentially the same, or even the same, epitope as a “reference antigen binding protein” (e.g. antibody). Competing antigen binding proteins are thus able to effectively compete with a reference antigen binding protein for binding to the relevant antigen. Preferably, the competing antigen binding protein (e.g. antibody) can bind to the same epitope as the reference antigen binding protein (e.g. antibody). Alternatively viewed, the competing antigen binding protein (e.g. antibody) preferably has the same epitope specificity as the reference antigen binding protein (e.g. antibody).

“Reference antigen binding proteins” as used herein are antigen binding proteins which can bind to the relevant antigen in accordance with the invention. Preferably, reference antigen binding proteins (e.g. antibodies) have a VH and a VL domain as defined herein. For example, a reference antigen binding protein which binds to, or specifically binds to, HLA-A*02:01/MAGE-A4²³⁰⁻²³⁹ may comprise a VL domain and a VH domain of the 1-H02 or AM15 antibody (i.e. comprise a VL domain of SEQ ID NO:4 or 114 and a VH domain of SEQ ID NO:3). Reference antigen binding proteins are preferably antibodies. Thus a preferred reference antigen binding protein may be a specific antibody of the invention as defined herein (Table A, e.g. the 1-H02 or AM15 antibody).

The identification of one or more competing antigen binding proteins (e.g. antibodies) is a straightforward technical matter now that reference antibodies (such as the 1-H02 and AM15 antibodies) have been provided (Table A). As the identification of competing antigen binding proteins (e.g. antibodies) is determined in comparison to a reference antigen binding protein (e.g. antibody), it will be understood that actually determining the epitope to which either or both antigen binding proteins bind is not in any way required in order to identify a competing antigen binding protein. However, epitope mapping can be performed using standard techniques, if desired.

The CDRs of the antigen binding proteins (e.g. antibodies) of the invention are preferably separated by appropriate framework regions such as those found in naturally occurring antibodies and/or effective engineered antibodies. Thus, the V_{H}, V_{L} and individual CDR sequences of the invention are preferably provided within or incorporated into an appropriate framework or scaffold to enable antigen binding, herein HLA-A*02:01/MAGE-A4²³⁰⁻²³⁹ binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions.

Appropriate sequences that can be used for framework regions are well known and documented in the art and any of these may be used. Exemplary sequences for framework regions are one or more of the framework regions making up the V_{H}, and/or V_{L} domains of the antibodies of the invention, e.g. one or more of the framework regions of the specific antigen binding proteins (e.g. antibodies) as disclosed in Table A (e.g. 1-H02 or AM15), or framework regions substantially homologous thereto, and in particular framework regions that allow the maintenance of antigen specificity, for example framework regions that result in substantially the same or the same 3D structure of the antibody.

Preferably, the antigen binding protein (e.g. antibody) comprises a VH domain that comprises a VH FR1, a VH FR2, a VH FR3 and a VH FR4 comprising the following amino acid sequences or sequences substantially homologous thereto:

| | VH FR1 | VH FR2 | VH FR3 | VH FR4 | |
|---|---|---|---|---|---|
| a) | SEQ ID NO: 11 | SEQ ID NO: 12 | SEQ ID NO: 13 | SEQ ID NO: 14 | ; or |
| b) | SEQ ID NO: 99 | SEQ ID NO: 12 | SEQ ID NO: 13 | SEQ ID NO: 14 | |

and/or (preferably “and”) a VL domain that comprises a VL FR1, a VL FR2, a VL FR3 and a VL FR4 comprising the following amino acid sequences or sequences substantially homologous thereto:

| | VL FR1 | VL FR2 | VL FR3 | VL FR4 | |
|---|---|---|---|---|---|
| a) | SEQ ID NO: | SEQ ID NO: | SEQ ID NO: | SEQ ID NO: | ; or |
| | 15 | 16 | 17 | 18 | |
| b) | SEQ ID NO: | SEQ ID NO: | SEQ ID NO: | SEQ ID NO: | ; or |
| | 102 | 16 | 109 | 112 | |
| c) | SEQ ID NO: | SEQ ID NO: | SEQ ID NO: | SEQ ID NO: | ; or |
| | 103 | 108 | 110 | 18 | |
| d) | SEQ ID NO: | SEQ ID NO: | SEQ ID NO: | SEQ ID NO: | ; or |
| | 105 | 16 | 17 | 18 | |
| e) | SEQ ID NO: | SEQ ID NO: | SEQ ID NO: | SEQ ID NO: | ; or |
| | 101 | 16 | 109 | 112 | |
| f) | SEQ ID NO: | SEQ ID NO: | SEQ ID NO: | SEQ ID NO: | ; or |
| | 100 | 106 | 111 | 112 | |
| g) | SEQ ID NO: | SEQ ID NO: | SEQ ID NO: | SEQ ID NO: | . |
| | 104 | 107 | 17 | 18 | |

Preferably, the antigen binding protein (e.g. antibody) comprises a VH domain that comprises a VH FR1, a VH FR2, a VH FR3 and a VH FR4 comprising the following amino acid sequences or sequences substantially homologous thereto:

| | VH FR1 | VH FR2 | VH FR3 | VH FR4 |
|---|---|---|---|---|
| a) | SEQ ID NO: 11 | SEQ ID NO: 12 | SEQ ID NO: 13 | SEQ ID NO: 14 |

and/or (preferably “and”) a VL domain that comprises a VL FR1, a VL FR2, a VL FR3 and a VL FR4 comprising the following amino acid sequences or sequences substantially homologous thereto:

| | VL FR1 | VL FR2 | VL FR3 | VL FR4 | |
|---|---|---|---|---|---|
| a) | SEQ ID NO: | SEQ ID NO: | SEQ ID NO: | SEQ ID NO: | ; or |
| | 15 | 16 | 17 | 18 | |
| b) | SEQ ID NO: | SEQ ID NO: | SEQ ID NO: | SEQ ID NO: | ; or |
| | 102 | 16 | 109 | 112 | |
| c) | SEQ ID NO: | SEQ ID NO: | SEQ ID NO: | SEQ ID NO: | . |
| | 103 | 108 | 110 | 18 | |

Preferably, the antigen binding protein (e.g. antibody) comprises one or more. preferably all, of the framework regions making up the V_{H}, and/or (preferably “and”) V_{L} domains of the 1-H02 antibody, the AM15 antibody, or the AMC8 antibody as disclosed in Table A, or framework regions substantially homologous thereto. Preferably, all four of the variable heavy chain (SEQ ID NOs:11, 12, 13 and 14) framework regions and/or (preferably “and”) variable light chain (a) SEQ ID NOs:15, 16, 17 and 18 or b) SEQ ID Nos: 102, 16, 109 and 112) framework regions (FR), as appropriate, or FR regions substantially homologous thereto, are found in the antibodies (or binding proteins) of the invention.

The antigen binding protein (e.g. antibody) and nucleic acid molecules of the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to nucleic acid molecules or sequences and proteins or polypeptides, e.g. antigen binding proteins (e.g. antibodies), refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

Thus, when used in connection with a protein or polypeptide molecule such as light chain CDRs 1, 2 and 3, heavy chain CDRs 1, 2 and 3, light chain variable regions (domains), heavy chain variable regions (domains), and antigen binding proteins (e.g. antibodies) of the invention, including full length antibodies, the term “isolated” or “purified” typically refers to a protein substantially free of cellular material or other proteins from the source from which it is derived. In some embodiments, particularly where the protein is to be administered to humans or animals, such isolated or purified proteins are substantially free of culture medium when produced by recombinant techniques, or chemical precursors or other chemicals when chemically synthesized.

It can be noted that in embodiments the antigen binding proteins (e.g. antibodies) etc., of the invention do not occur in nature and are, in that respect, man-made constructs in that they do not correspond to molecules that occur naturally. For example, preferred antigen binding proteins (e.g. antibodies) can be engineered or recombinantly produced. In other words in embodiments the antigen binding proteins (e.g. antibodies) etc, of the invention are non-native or not naturally occurring.

A person skilled in the art will appreciate that the proteins and polypeptides of the invention, such as the heavy and light chain CDRs, the heavy and light chain variable regions (domains), antigen binding proteins, antibodies and antibody fragments, may be prepared in any of several ways well known and described in the art, but are most preferably prepared using recombinant methods.

In preferred embodiments the antibodies of the invention are human antibodies, more preferably fully human antibodies. In this regard, human antibodies generally have at least two potential advantages for use in human therapy. First, the human immune system should not recognize the antibody as foreign. Second, the half-life in the human circulation will be similar to naturally occurring human antibodies, allowing smaller and less frequent doses to be given.

The term “human” as used herein in connection with antibody molecules and binding proteins first refers to antibodies and binding proteins having variable regions (e.g., V_{H}, V_{L}, CDR or FR regions) and, optionally, constant antibody regions, isolated or derived from a human repertoire or derived from or corresponding to sequences found in humans or a human repertoire, e.g., in the human germline or somatic cells.

The “human” antibodies and binding proteins of the invention further include amino acid residues not encoded by human sequences, e.g., mutations introduced by random or site directed mutations in vitro, for example mutations introduced by in vitro cloning or PCR, for example in an affinity maturation method. Particular examples of such mutations are mutations that involve conservative substitutions or other mutations in a small number of residues of the antibody or binding protein, e.g., in up to 6, 5, 4, 3, 2 or 1 of the residues of the antibody or binding protein, preferably e.g., in up to 4, 3, 2 or 1 of the residues making up one or more of the CDRs of the antibody or binding protein. Certain examples of such “human” antibodies include antibodies and variable regions that have been subjected to standard modification techniques to reduce the amount of potentially immunogenic sites.

Thus, the “human” antibodies of the invention include sequences derived from and related to sequences found in humans, but which may not naturally exist within the human antibody germline repertoire in vivo. In addition, the human antibodies and binding proteins of the present invention include proteins comprising human consensus sequences identified from human sequences, or sequences substantially homologous to human sequences.

In addition, the human antibodies and binding proteins of the present invention are not limited to combinations of V_{H}, V_{L}, CDR or FR regions that are themselves found in combination in human antibody molecules. Thus, the human antibodies and binding proteins of the invention can include or correspond to combinations of such regions that do not necessarily exist naturally in humans (e.g. are not naturally occurring antibodies).

In preferred embodiments, the human antibodies will be fully human antibodies. “Fully human” antibodies, as used herein, are antibodies comprising “human” variable region domains and/or CDRs, as defined above, without substantial non-human antibody sequences or without any non-human antibody sequences. For example, antibodies comprising human variable region domains “without substantial non-human antibody sequences” are antibodies and/or domains in which only up to 6, 5, 4, 3, 2 or 1 amino acids are amino acids that are not encoded by human antibody sequences. Antibodies comprising human CDRs “without substantial non-human antibody sequences” are antibodies, and/or CDRs in which only up to 4, 3, 2 or 1 amino acids are amino acids that are not encoded by human antibody sequences. Thus, “fully human” antibodies are distinguished from “humanized” antibodies, which are based on substantially non-human variable region domains, e.g., mouse variable region domains, in which certain amino acids have been changed to better correspond with the amino acids typically present in human antibodies.

The “fully human” antibodies of the invention may be human variable region domains and/or CDRs without any other substantial antibody sequences, such as being single chain antibodies. Alternatively, the “fully human” antibodies of the invention may be human variable region domains and/or CDRs integral with or operatively attached to one or more human antibody constant regions. Certain preferred fully human antibodies are IgG antibodies with the full complement of IgG constant regions.

In other embodiments, “human” antibodies of the invention will be part-human chimeric antibodies. “Part-human chimeric” antibodies, as used herein, are antibodies comprising “human” variable region domains and/or CDRs operatively attached to, or grafted onto, a constant region of a non-human species, such as rat or mouse. Such part-human chimeric antibodies may be used, for example, in pre-clinical studies, wherein the constant region will preferably be of the same species of animal used in the pre-clinical testing. These part-human chimeric antibodies may also be used, for example, in ex vivo diagnostics, wherein the constant region of the non-human species may provide additional options for antibody detection.

Antibodies of the present invention may also be CDR grafted antibodies. Such antibodies are antibodies comprising the CDR sequences (e.g. 3 VH CDRs and/or 3 VL CDRs) of an antibody of the invention grafted into a framework region that is different from the framework region with which the CDRs are associated in the VL and/or VH domains described herein.

Preferred heavy and light chain variable region framework sequences are set forth in the sequence tables herein.

Nucleic acid molecules (e.g. one or more nucleic acid molecules) comprising nucleotide sequences that encode the antigen binding proteins (e.g. antibodies) or immunoconjugates of the present invention as defined herein or parts or fragments thereof, or nucleic acid molecules substantially homologous thereto, form yet further aspects of the invention.

Preferred nucleic acid molecules are those encoding an antigen binding protein (e.g. antibody) of the present invention as described elsewhere herein which can bind to HLA-A*02:01/MAGE-A4²³⁰⁻²³⁹, e.g. an antigen binding protein (e.g. antibody) of the invention with CDR and optionally FR and other regions as defined in Table A, or antibodies with sequences substantially homologous thereto.

Preferred nucleic acid molecules are those encoding a VH region of an antigen binding protein (e.g. antibody) of the present invention. Other preferred nucleic acid molecules are those encoding a VL region of an antigen binding protein (e.g. antibody) of the present invention. Other preferred nucleic acid molecules are those encoding a VH region of an antigen binding protein (e.g. antibody) of the present invention and a VL region of an antigen binding protein (e.g. antibody) of the present invention.

In some embodiments, preferred nucleic acid molecules are those encoding a VL region of SEQ ID NO:4 (such as SEQ ID NO:2 or 135), of SEQ ID NO: 114 (such as SEQ ID NO: 113), of SEQ ID NO: 116 (such as SEQ ID NO: 115), of SEQ ID NO: 120 (such as SEQ ID NO: 119), of SEQ ID NO: 122 (such as SEQ ID NO: 121), of SEQ ID NO: 124 (such as SEQ ID NO: 123), of SEQ ID NO: 128 (such as SEQ ID NO: 127), of SEQ ID NO:140 (such as SEQ ID NO:139), of SEQ ID NO: 142 (such as SEQ ID NO: 141), or of SEQ ID NO: 144 (such as SEQ ID NO: 143) and/or those encoding a VH region of SEQ ID NO:3 (such as SEQ ID NO:1), of SEQ ID NO: 118 (such as SEQ ID NO: 117), of SEQ ID NO: 126 (such as SEQ ID NO: 125), of SEQ ID NO: 130 (such as SEQ ID NO: 129), of SEQ ID NO: 132 (such as SEQ ID NO: 131), or of SEQ ID NO: 134 (such as SEQ ID NO: 133).

Preferred nucleic acid molecules are those encoding an antigen binding protein (e.g. antibody) of the present invention which can bind to HLA-A*02:01/MAGE-A4²³⁰⁻²³⁹ (e.g. as defined in Table A, e.g. comprising nucleic acid sequences encoding SEQ ID NO:3 and/or SEQ ID NO:4, 114, or 116, such as SEQ ID NO:1 and/or SEQ ID NO:2, 113 and 115, respectively, or comprising nucleic acid sequences encoding SEQ ID NO:118 and/or SEQ ID NO: 116, such as SEQ ID NO:117 and/or SEQ ID NO:115, respectively).

In some embodiments nucleic acid molecules are those having a nucleic acid sequence that is substantially homologous to the specific nucleic acid sequences defined herein, for example having at least 80% sequence identity to specific nucleic acid sequences defined herein.

The term “nucleic acid sequence” or “nucleic acid molecule” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

A person skilled in the art will appreciate that the antigen binding proteins (e.g. antibodies) and polypeptides of the invention, such as the light and heavy CDRs, the light and heavy chain variable regions (domains), antibodies, antibody fragments, and immunoconjugates, may be prepared in any of several ways well known and described in the art, but are most preferably prepared using recombinant methods.

Nucleic acid fragments encoding the light and heavy chain variable regions (domains) of the antigen binding proteins (e.g. antibodies) of the invention can be derived or produced by any appropriate method, e.g. by cloning or synthesis.

Once nucleic acid fragments encoding the light and heavy chain variable regions (domains) of the antigen binding proteins (e.g. antibodies) of the invention have been obtained, these fragments can be further manipulated by standard recombinant DNA techniques, for example to convert the variable region fragments into full length antigen binding protein (e.g. antibody) molecules with appropriate constant region domains, or into particular formats of antibody fragment discussed elsewhere herein, e.g. Fab fragments, scFv fragments, scDb formats, etc. Typically, or as part of this further manipulation procedure, the nucleic acid fragments encoding the antigen binding protein (e.g. antibody) molecules of the invention are generally incorporated into one or more appropriate expression vectors in order to facilitate production of the antigen binding proteins (e.g. antibodies) of the invention.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates a recombinant expression vector containing a nucleic acid molecule of the invention, or a fragment thereof, and the necessary regulatory sequences for the transcription and translation of the protein sequence encoded by the nucleic acid molecule of the invention.

Suitable regulatory sequences may be derived from a variety of sources, including bacterial, fungal, viral, mammalian, or insect genes and are well known in the art. Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the invention may also contain a selectable marker gene that facilitates the selection of host cells transformed or transfected with a recombinant molecule of the invention.

The recombinant expression vectors may also contain genes that encode a fusion moiety that provides increased expression of the recombinant protein; increased solubility of the recombinant protein; and aid in the purification of the target recombinant protein by acting as a ligand in affinity purification (for example appropriate C-terminal or N-terminal “tags” to enable purification and/or identification may be present, e.g., His tags or myc tags). In some embodiments, however, the antigen binding proteins (e.g. antibodies) of the invention do not comprise a C-terminal or N-terminal tag, e.g. a His-tag.

Recombinant expression vectors can be introduced into host cells to produce a transformed host cell. The terms “transformed with”, “transfected with”, “transformation” and “transfection” are intended to encompass introduction of nucleic acid (e.g., a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells, as will be well known to a person skilled in the art. For example, the proteins of the invention may be expressed in yeast cells or mammalian cells, for example HEK or CHO cells. Cell-free expression systems might also be used.

Given the teachings provided herein, promoters, terminators, and methods for introducing expression vectors of an appropriate type into plant, avian, and insect cells may also be readily accomplished.

Alternatively, the proteins of the invention may also be expressed in non-human transgenic animals such as, rats, rabbits, sheep and pigs.

The proteins of the invention may also be prepared by chemical synthesis using techniques well known in the chemistry of proteins such as solid phase synthesis.

N-terminal or C-terminal fusion proteins comprising the antigen binding proteins (e.g. antibodies) of the invention conjugated to other molecules, such as proteins, may be prepared by fusing through recombinant techniques. The resultant fusion proteins contain an antigen binding protein (e.g. antibody) of the invention fused to the selected protein or marker protein, or tag protein as described herein. The antigen binding proteins (e.g. antibodies) of the invention may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate.

A yet further aspect provides an expression construct or expression vector or expression system (e.g. a viral or bacterial or other expression construct, vector or system), e.g. one or more expression constructs or expression vectors, comprising one or more of the nucleic acid fragments or segments or molecules of the invention. Preferably the expression constructs or vectors or systems are recombinant. Preferably said constructs or vectors or systems further comprise the necessary regulatory sequences for the transcription and translation of the protein sequence encoded by the nucleic acid molecule of the invention.

A yet further aspect provides a host cell (e.g. a mammalian or bacterial or yeast host cell) or virus, e.g. one or more host cells or viruses, comprising one or more expression constructs or expression vectors of the invention. Also provided are host cells (e.g. a mammalian or bacterial or yeast host cell) or viruses, e.g. one or more host cells or viruses, comprising one or more of the nucleic acid molecules of the invention. A host cell (e.g. a mammalian host cell or bacterial host cell, or yeast host cell) or virus expressing an antigen binding protein (e.g. antibody) of the invention forms a yet further aspect.

A yet further aspect of the invention provides a method of producing (or manufacturing) a protein (e.g. antibody) of the present invention comprising a step of culturing the host cells of the invention. Preferred methods comprise the steps of (i) culturing a host cell comprising one or more of the recombinant expression vectors or one or more of the nucleic acid sequences of the invention under conditions suitable for the expression of the encoded antigen binding protein (e.g. antibody); and optionally (ii) isolating or obtaining the antigen binding protein (e.g. antibody) from the host cell or from the growth medium/supernatant. Such methods of production (or manufacture) may also comprise a step of purification of the antigen binding protein (e.g. antibody) product and/or formulating the antigen binding protein (e.g. antibody) into a composition including at least one additional component, such as a pharmaceutically acceptable carrier or excipient.

In embodiments when the antigen binding protein (e.g. antibody) of the invention is made up of more than one polypeptide chain (e.g. whole antibodies), then all the polypeptides are preferably expressed in the host cell, either from the same or a different expression vector, so that the complete proteins, e.g. antibody proteins of the invention, can assemble in the host cell and be isolated or purified therefrom.

One therapeutic (or diagnostic) approach is the use of antibodies that can target specific antigens expressed on cancer cells, that are not expressed or are expressed at a lower level on normal cells. These target antigens, of which HLA-A*02:01/MAGE-A4²³⁰⁻²³⁹ is an example, can be exploited using antibodies to specifically kill antigen-bearing cancer cells by a variety of mechanisms including by delivering immuno- or radio labelled conjugates that, when delivered to the antigen-bearing cell, specifically kill the target cell. Such targeting can also be used for diagnosis.

The invention thus also provides a range of conjugated antigen binding proteins (e.g. antibodies), i.e. “immunoconjugates”, in which the antigen binding protein (e.g. antibody) of the invention is operatively attached to at least one other therapeutic or diagnostic agent. The term “immunoconjugate” is broadly used to define the operative association of the antibody (or binding protein) with another effective agent (e.g. therapeutic or diagnostic agent) and is not intended to refer solely to any type of operative association, and is particularly not limited to chemical “conjugation”. Fusion proteins, e.g. recombinant fusion proteins, are particularly contemplated. So long as the delivery or targeting agent (the anti-HLA-A*02:01/MAGE-A4²³⁰⁻²³⁹ component) is able to bind to the target and the therapeutic or diagnostic agent is sufficiently functional upon delivery, the mode of attachment will be suitable.

For example, in some preferred embodiments, antibodies (or binding proteins) of the invention are part of an immunotoxin or are used (e.g. used therapeutically) as part of immunotoxins wherein the antibody is itself operatively associated or combined with a toxic agent (e.g. a chemotherapeutic agent or toxin or a radioactive material such as a radiotracer, e.g. for use in radioimmunotherapy). The operative attachment includes all forms of direct and indirect attachment as described herein and known in the art.

Suitable chemotherapeutic agents or toxins are well known and described in the art, for example cytotoxic proteins derived from bacteria or plants can be used. The toxin should be capable of killing target cells once it has been taken up into said cells. Thus, preferred immunoconjugates of the invention are immunotoxins comprising an antibody of the invention linked or otherwise conjugated to a toxin (also sometimes referred to as antibody drug conjugates, ADCs). In preferred immunoconjugates, active ingredients such as radionuclides, toxins (for example, the diphtheria toxin), or other cytostatic agents can be bonded (conjugated) or otherwise linked to the corresponding antibodies.

In some embodiments, antibodies of the invention are used (e.g. used therapeutically) in their “naked” unconjugated form.

An immunoconjugate comprising a therapeutic agent, or a carrier comprising a therapeutic agent, may be used in therapy. An immunoconjugate comprising a diagnostic agent, or a carrier comprising a diagnostic agent, may be used in in vivo and/or in vitro diagnostic methods.

In some embodiments, antigen binding proteins (e.g. antibodies) of the invention are used (e.g. used therapeutically) in their “naked” unconjugated form.

Compositions comprising at least a first antigen binding protein (e.g. antibody) or immunoconjugate of the invention, or at least a first nucleic acid molecule or expression vector of the invention, or at least a first host cell of the invention, constitute further aspects of the present invention. Formulations (compositions) comprising one or more antigen binding proteins (e.g. antibodies), etc., of the invention, optionally in admixture with a suitable diluent, carrier or excipient constitute a preferred embodiment of the present invention. Such formulations may be for pharmaceutical use, and thus compositions of the invention are preferably pharmaceutically acceptable or otherwise acceptable for administration to human or non-human animals, but in particular humans. Suitable diluents, excipients and carriers are known to the skilled man.

The pharmaceutical compositions may additionally comprise further active ingredients (e.g. as described elsewhere herein) in the context of co-administration regimens or combined regimens.

Any appropriate mode of administration can be used for administering/delivering the compositions according to the invention. The compositions according to the invention may be presented, for example, in a form suitable for oral, nasal, parenteral (e.g. intravenous, intraperitoneal, subcutaneous, intradermal, intramuscular), topical or rectal administration, or for mucosal delivery, and any of these modes of administration, or indeed any other appropriate mode of administration, can be used. In a preferred embodiment, compositions according to the invention are presented in a form suitable for intravenous administration. In some embodiments, compositions according to the invention are presented in a form suitable for intraperitoneal (i.p.) administration. In some embodiments, compositions according to the invention are presented in a form suitable for injection directly into a tumour (intratumoural).

The active compounds (e.g. the antigen binding proteins (e.g. antibodies) of the invention) as defined herein may be presented in the conventional pharmacological forms of administration, such as tablets, coated tablets, nasal sprays, solutions, emulsions, liposomes, exosomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Nasal sprays may be formulated similarly in aqueous solution and packed into spray containers, either with an aerosol propellant or provided with means for manual compression.

The pharmaceutical compositions (formulations) of the present invention are preferably administered parenterally. Intravenous administration is preferred. In some embodiments, administration is intraperitoneal (i.p.) administration. In some embodiments, administration is by injection into a tumour. Parenteral administration may be performed by subcutaneous, intramuscular, intraperitoneal or intravenous injection by means of a syringe. Alternatively, parenteral administration can be performed by means of an infusion pump. A further option is a composition which may be a powder or a liquid for the administration of the antibody in the form of a nasal or pulmonal spray. As a still further option, the antigen binding proteins (e.g. antibodies) of the invention can also be administered transdermally, e.g. from a patch, optionally an iontophoretic patch, or transmucosally, e.g. bucally.

Suitable dosage units can be determined by a person skilled in the art.

In another aspect, the invention provides a method of binding HLA-A*02:01/MAGE-A4²³⁰⁻²³⁹, comprising contacting a composition comprising HLA-A*02:01/MAGE-A4²³⁰⁻²³⁹ with an antigen binding protein (e.g. antibody) of the invention, or an immunoconjugate thereof.

In yet another aspect, the invention provides a method of detecting HLA-A*02:01/MAGE-A4²³⁰⁻²³⁹, comprising contacting a composition suspected of containing HLA-A*02:01/MAGE-A4²³⁰⁻²³⁹ with an antigen binding protein (e.g. antibody) of the invention, or an immunoconjugate thereof, under conditions effective to allow the formation of HLA-A*02:01/MAGE-A4²³⁰⁻²³⁹/antigen binding protein complexes (e.g. HLA-A*02:01/MAGE-A4²³⁰⁻²³⁹/antibody complexes) and detecting the complexes so formed.

In embodiments, the composition suspected of containing HLA-A*02:01/MAGE-A4²³⁰⁻²³⁹ may have been obtained from a subject, e.g. may be a subject sample e.g. blood sample or biopsy.

Yet further aspects are methods of diagnosis or imaging of a subject comprising the administration of an appropriate amount of an antigen binding protein (e.g. antibody) of the invention as defined herein to the subject and detecting the presence and/or amount and/or the location of the antigen binding protein (e.g. antibody) of the invention in the subject.

Appropriate diseases to be imaged or diagnosed in accordance with the present invention are cancers, e.g. as described elsewhere herein in connection with disease treatments.

In vitro methods of diagnosis are provided. Thus, in a further aspect, the invention provides a method of diagnosing cancer in a subject comprising the step of:
(a) contacting a test sample taken from said subject with one or more of the antigen binding proteins (e.g. antibodies) of the invention.

In an embodiment, the invention provides a method of diagnosing cancer in a subject comprising the steps of:
(a) contacting a test sample taken from said subject with one or more of the antigen binding proteins (e.g. antibodies) of the invention;
(b) measuring the presence and/or amount and/or location of antigen binding protein-antigen complex (e.g. antibody-antigen complex) in the test sample; and, optionally
(c) comparing the presence and/or amount and/or location of antigen binding protein-antigen complex (e.g. antibody-antigen complex) in the test sample to a control.

In the above methods, said contacting step is carried out under conditions that permit the formation of an antigen binding protein-antigen complex (e.g. antibody-antigen complex). Appropriate conditions can readily be determined by a person skilled in the art.

In the above methods any appropriate test sample may be used, for example biopsy cells, tissues or organs suspected of being affected by disease, or histological sections.

The subject is as defined elsewhere herein, and is preferably a human subject. The subject may be a subject at risk of developing cancer or at risk of the occurrence of cancer, e.g. a healthy subject or a subject not displaying any symptoms of cancer or any other appropriate “at risk” subject. In another embodiment the subject is a subject having, or suspected of having (or developing), or potentially having (or developing) cancer.

In certain of the above methods, the presence of any amount of antigen binding protein-antigen complex (e.g. antibody-antigen complex) in the test sample would be indicative of the presence of disease. Preferably, for a positive diagnosis to be made, the amount of antigen binding protein-antigen complex (e.g. antibody-antigen complex) in the test sample is greater than, preferably significantly greater than, the amount found in an appropriate control e.g. control sample. More preferably, the significantly greater levels are statistically significant, preferably with a probability value of <0.1, preferably <0.05. Appropriate methods of determining statistical significance are well known and documented in the art and any of these may be used.

Appropriate control controls or samples could be readily chosen by a person skilled in the art, for example, in the case of diagnosis of a particular disease, an appropriate control would be a sample from a subject that did not have that disease. Appropriate control “values” could also be readily determined without running a control “sample” in every test, e.g. by reference to the range for normal subjects known in the art.

For use in the diagnostic or imaging applications, the antigen binding proteins (e.g. antibodies) of the invention may be labeled with a detectable marker such as a radioisotope, such as ³H, ¹⁴C, ³²P, ³⁵S, ¹²³I, ¹²⁵I, ¹³¹I; a radioactive emitter (e.g. α, β or γ emitters); a fluorescent (fluorophore) or chemiluminescent (chromophore) compound, such as fluorescein isothiocyanate, rhodamine, luciferin or Europium; an enzyme, such as alkaline phosphatase, beta-galactosidase or horseradish peroxidase; an imaging agent; or a metal ion; or a chemical moiety such as biotin which may be detected by binding to a specific cognate detectable moiety, e.g. labelled avidin/streptavidin. Methods of attaching a label to an antigen binding protein (e.g. an antibody) are known in the art. Such detectable markers allow the presence, amount or location of antigen binding protein-antigen complexes (e.g. antibody-antigen complexes) in the test sample to be examined.

Preferred detectable markers for in vivo use include an X-ray detectable compound, such as bismuth (III), gold (III), lanthanum (III) or lead (II); a radioactive ion, such as copper⁶⁷, gallium⁶⁷, gallium⁶⁸, indium¹¹¹, indium¹¹³, iodine¹²³, iodine¹²⁵, iodine¹³¹, mercury¹⁹⁷, mercury²⁰³, rhenium¹⁸⁶, rhenium¹⁸⁸, rubidium⁹⁷, rubidium¹⁰³, technetium^{99m} or yttrium⁹⁰; a nuclear magnetic spin-resonance isotope, such as cobalt (II), copper (II), chromium (III), dysprosium (III), erbium (III), gadolinium (III), holmium (III), iron (II), iron (III), manganese (II), neodymium (III), nickel (II), samarium (III), terbium (III), vanadium (II) or ytterbium (III); or rhodamine or fluorescein.

The invention also includes diagnostic or imaging agents comprising the antigen binding proteins (e.g. antibodies) of the invention attached to a label or detectable marker that produces a detectable signal, directly or indirectly. Appropriate labels or detectable markers are described elsewhere herein.

In one embodiment the method of diagnosing cancer is an in vitro method.

In one embodiment the method of diagnosing cancer is an in vivo method.

Alternatively viewed, the present invention provides a method for screening for cancer in a subject.

In some embodiments, antigen binding proteins (e.g. antibodies) of the present invention can be used as companion diagnostics.

In some aspects, diagnostic methods of the invention are provided which further comprise a step of treating cancer by therapy, e.g. using an antigen binding protein (e.g. antibody) of the present invention. For example, if the result of a method of the invention is indicative of cancer in the subject (e.g. a positive diagnosis of cancer is made), then an additional step of treating the cancer by therapy or surgery can be performed.

A further aspect of the present invention provides the antigen binding proteins (e.g. antibodies) or immunoconjugates of the invention for use in therapy, in particular for use in the treatment or prevention of cancer. In other embodiments, the nucleic acid molecules, expression vectors, host cells or viruses of the invention can also be used in the therapeutic methods described herein.

By “therapy” as used herein is meant the treatment of any medical condition. Such treatment may be prophylactic (i.e. preventative), curative (or treatment intended to be curative), or palliative (i.e. treatment designed merely to limit, relieve or improve the symptoms of a condition).

The cancer is typically in a subject, i.e. a subject suffering therefrom. The cancer is typically HLA-A*02:01/MAGE-A4²³⁰⁻²³⁹ positive. The term “cancer” encompasses cancerous cells of any type, including both tumours (e.g. solid tumours) and hematological or blood cancers. Thus, “cancer cells” may be tumour cells, or may be blood cancer cells.

In another aspect, the present invention provides immunoconjugates of the invention for use in therapy, in particular for use in the treatment or prevention of cancer.

In another aspect, the present invention provides antigen binding proteins (e.g. antibodies) or immunoconjugates of the invention, for use in killing cancer cells presenting/displaying HLA-A*02:01/MAGE-A4²³⁰⁻²³⁹, i.e. HLA-A*02:01/MAGE-A4²³⁰⁻²³⁹ positive cancer cells. Said killing is preferably T-cell mediated killing. Such killing may also play a role in the therapeutic treatments as discussed herein.

In another aspect, the present invention provides an antigen binding protein (e.g. antibody) of the invention in a T-cell engager format, for use in redirecting T-cell activity against a cancer cell. Preferably, the T-cell activity is cytotoxic activity. The use typically comprises the step of contacting said cancer cell with said T-cell engager antigen binding protein (e.g. antibody).

Alternatively viewed, the present invention provides an antigen binding protein (e.g. antibody) of the invention in a T-cell engager format, for use in redirecting T-cell activity against a tumour. Preferably, the T cell activity is cytotoxic activity. The use typically comprises the step of contacting said tumour with said T-cell engager antigen binding protein (e.g. antibody).

In another aspect, the present invention provides an antigen binding protein (e.g. antibody) of the invention in a T-cell engager format, for use in inducing T-cell mediated cytotoxicity against a cancer cell or tumour. The use typically comprises the step of contacting said cancer cell with said T-cell engager antigen binding protein (e.g. antibody).

Preferred antigen binding proteins (e.g. antibodies) of the invention for use in the therapies disclosed herein, including in the treatment or prevention of cancer are in a T-cell engager format, preferably a bispecific format, preferably the scDb format (as defined herein).

Preferred T cells are as described elsewhere herein.

In accordance with the present invention the antigen binding proteins (e.g. antibodies) may target HLA-A*02:01/MAGE-A4²³⁰⁻²³⁹ positive cells, e.g. cancer cells.

In one embodiment, solid tumours are treated.

In one embodiment, hematological or blood cancers are treated.

In some embodiments, a tumour or cancer that is characterized by expressing or over-expressing HLA-A*02:01/MAGE-A4²³⁰⁻²³⁹ is treated.

Thus, a further aspect of the present invention provides antigen binding proteins (e.g. antibodies) or immunoconjugates as defined herein for use in the treatment or prevention of a cancer (e.g. a tumour or blood cancer) that is HLA-A*02:01/MAGE-A4²³⁰⁻²³⁹ positive.

Preferred cancers are as described elsewhere herein. In embodiments, the cancer cells are cancer cells in or from a subject suffering from cancer, preferably wherein the cancer is lung cancer, skin cancer or leukaemia. Preferred cancers include non-small cell lung cancer (NSCLC, e.g. squamous NSCLC), small cell lung cancer (e.g extensive stage disease small cell lung cancer), melanoma (e.g. metastatic melanoma such as BRAF negative metastatic melanoma, or multiple melanoma), lymphoma (e.g. acute T-cell lymphoma, Hodgkin lymphoma, non-Hodgkin lymphoma or chronic lymphocytic lymphoma), leukemia (e.g acute myeloid leukemia, acute lymphoblastic leukemia or myelodysplastic syndrome), renal cell cancer (RCC, e.g clear cell renal cancer), colorectal cancer, urothelial bladder cancer, urethral cancer, head and neck cancer (e.g. recurrent or metastatic head and neck squamous cell cancer), breast cancer (e.g. metastatic HER-2 negative breast cancer) advanced liver cancer, brain cancer (e.g. glioblastoma or astrocytoma), stomach cancer, oesophageal cancer, pancreatic carcinoma, adenocarcinomas, mesothelioma, peritoneal cancer, fallopian tube cancer, cervical cancer, ovarian cancer, sarcomas, e.g. metastatic sarcoma, hematological neoplasms, thyroid cancer, salivary cancer, laryngeal (larynx) cancer, neuroblastoma, retinoblastoma, and testis (testicular) cancer.

Preferred “cancer cells” are described elsewhere herein, and include cells from the above-mentioned preferred cancers. Cancer cells include cells selected from the group consisting of lung cancer cells, melanoma cells, and monocytes.

Without wishing to be bound by theory, it is believed that antigen binding proteins (e.g. antibodies) of the present invention may be superior to prior art antibodies in terms of their therapeutic efficacy (via T-cell mediated cytotoxicity) in particular in terms of the concentration of antigen binding protein (e.g. antibody) required to achieve marked T-cell mediated cytotoxicity. Low concentrations/doses of the antigen binding proteins (e.g. antibodies) of the invention have been shown to induce significant T-cell mediated cancer cell killing, notably in assays comprising a T-cell effector cell to target cell ratio (E:T) of only 1:1, and notably in disparate cancer types. Efficacy of antigen binding proteins (e.g. antibodies) of the present invention in initiating T-cell mediated cytotoxicity in both solid tumour and blood cancer cells has been demonstrated at low concentrations/doses after only a single application.

The antigen binding proteins (e.g. antibodies) of the present invention may also be superior to prior art antibodies in terms of their specificity, in particular in terms of their demonstrated lack of binding to/cross-reactivity with many HLA-A*02:01 restricted peptides with high and very high sequence identity to the target peptide MAGE-A4²³⁰⁻²³⁹; and in terms of their demonstrated thermal stability.

The antigen binding proteins (e.g. antibodies) of the present invention may also be superior to prior art antibodies in terms of their preferentially redirection of T-cell activity against HLA-A*02:01/MAGE-A4²³⁰⁻²³⁹ positive cells as compared to HLA-A*02:01 positive, MAGE-A4²³⁰⁻²³⁹ negative cells, and as compared to cells displaying HLA-A*02:01 restricted genomic risk peptides with high sequence identity to the target peptide MAGE-A4²³⁰⁻²³⁹.

The antigen binding proteins (e.g. antibodies) of the present invention may also be superior to prior art antibodies in terms of their preferential induction of activation, differentiation and proliferation of T cells directed against HLA-A*02:01/MAGE-A4²³⁰⁻²³⁹ positive cells as compared to against HLA-A*02:01 positive, MAGE-A4²³⁰⁻²³⁹ negative cells.

The antigen binding proteins (e.g. antibodies) of the present invention may also be superior to prior art antibodies in terms of their preferential induction of T-cell mediated cytotoxicity against HLA-A*02:01/MAGE-A4²³⁰⁻²³⁹ positive cells as compared to against HLA-A*02:01 positive, MAGE-A4²³⁰⁻²³⁹ negative cells.

The antigen binding proteins (e.g. antibodies) of the present invention thus represent an exciting development in the field of anti-cancer therapeutic agents.

The administration of the antigen binding proteins (e.g. antibodies) in the therapeutic methods and uses of the invention is carried out in pharmaceutically, therapeutically, or physiologically effective amounts, to subjects (e.g. animals, e.g. human or non-human mammals) in need of treatment. Thus, said methods and uses may involve the additional step of identifying a subject in need of treatment.

Appropriate and effective concentrations/doses to be administered can readily be determined by a person skilled in the art.

Treatment of diseases or conditions in accordance with the present invention (for example treatment of pre-existing disease) includes cure of said disease or condition, or any reduction or alleviation of disease, e.g. reduction in disease severity, or symptoms of disease.

The therapeutic methods and uses of the prevent invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis, or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

In some aspects, a therapeutic method or therapeutic use of the invention may further comprise an initial step of selecting a subject (e.g. a human subject) at risk of developing cancer, or at risk of the occurrence of cancer, or suspected of having (or developing) cancer, or potentially having (or developing) cancer. Subjects may be selected on the basis that, for example, the subject (or sample, e.g. tissue biopsy, from the subject) is positive for one or more cancer markers or risk factors.

Suitable subjects for treatment in accordance with the present invention thus include any type of animal that can suffer from cancer, and more specifically cancers comprising cells that express HLA-A*02:01/MAGE-A4²³⁰⁻²³⁹.

Thus, the in vivo methods and uses as described herein are generally carried out in a mammal. Any mammal may be treated, for example humans and any livestock, domestic or laboratory animal. Specific examples include mice, rats, pigs, cats, dogs, sheep, rabbits, horses, cows and monkeys. Preferably, however, the mammal is a human.

Thus, the term “animal” or “patient” or “subject” as used herein includes any mammal, for example humans and any livestock, domestic or laboratory animal. Specific examples include mice, rats, pigs, cats, dogs, sheep, rabbits, horses, cows and monkeys. Preferably, however, the animal or patient or subject is a human. Thus, subjects or patients treated in accordance with the present invention will preferably be humans.

In another embodiment the subject is a subject having, or suspected of having (or developing), or potentially having (or developing) the disease or condition in question as described above.

In some embodiments, the antibodies (or binding proteins) of the invention can be used in monotherapy. In other embodiments they can be used in combination with other standard cancer therapeutics.

Alternatively viewed, the present invention provides a method of treating or preventing cancer, which method comprises administering to a patient in need thereof a therapeutically effective amount of an antigen binding protein (e.g. antibody) or immunoconjugate of the invention.

Embodiments of the therapeutic uses of the invention described herein apply, mutatis mutandis, to the methods of treatment/re-directing T-cell activity of the invention.

In another aspect, the present invention provides a method of treating or preventing cancer which method comprises administering to a patient in need thereof a therapeutically effective amount of an immunoconjugate of the invention.

In another aspect, the present invention provides a method of killing cancer cells presenting/displaying HLA-A*02:01/MAGE-A4²³⁰⁻²³⁹, i.e. HLA-A*02:01/MAGE-A4²³⁰⁻²³⁹ positive cancer cells, which method comprises administering to a patient in need thereof a therapeutically effective amount of an antigen binding protein (e.g. antibody) or immunoconjugate of the invention. Said killing is preferably T-cell mediated killing. Such killing may also play a role in the therapeutic treatments as discussed herein.

In another aspect, the present invention provides a method of redirecting T-cell activity against a cancer cell, which method comprises the step of contacting said cancer cell with an antigen binding protein (e.g. antibody) of the invention in a T-cell engager format. Preferably, the T-cell activity is cytotoxic activity. The cancer cell is typically in a patient and said method typically comprises administering to said patient the T-cell engager antigen binding protein (e.g. antibody).

Alternatively viewed, the present invention provides a method of redirecting T-cell activity against a tumour, comprising the step of contacting said tumour with an antigen binding protein (e.g. antibody) of the invention in a T-cell engager format. Preferably, the T-cell activity is cytotoxic activity. The tumour is typically in a patient and said method typically comprises administering to said patient the T-cell engager antigen binding protein (e.g. antibody).

In another aspect, the present invention provides a method of inducing T-cell mediated cytotoxicity against a cancer cell or tumour comprising the step of contacting said cancer cell or tumour with an antigen binding protein (e.g. antibody) of the invention in a T-cell engager format. The cancer cell or tumour is typically in a patient and said method typically comprises administering to said patient the T-cell engager antigen binding protein (e.g. antibody).

Preferred T cells are as described elsewhere herein.

A further aspect of the present invention provides a method of treating or preventing of a cancer (e.g. a tumour or blood cancer) that is HLA-A*02:01/MAGE-A4²³⁰⁻²³⁹ positive.

By “therapeutically effective amount” is meant an amount sufficient to show benefit to the condition of the subject. Whether an amount is sufficient to show benefit to the condition of the subject may be determined by the subject him/herself or a physician; preferably it is determined by clinical assessment and can be readily monitored. Preferred cancer therapies are as described elsewhere herein.

Further alternatively viewed, the present invention provides the use of an antigen binding protein (e.g. antibody) of the invention as defined herein in the manufacture of a medicament for use in therapy. Preferred therapy is the treatment or prevention of cancer as described elsewhere herein. Embodiments of the therapeutic uses of the invention described herein apply, mutatis mutandis, to this aspect of the invention.

Further alternatively viewed, the present invention provides the use of an antigen binding protein (e.g. antibody) of the invention as defined herein for the treatment of cancer. Embodiments of the therapeutic uses of the invention described herein apply, mutatis mutandis, to this aspect of the invention.

The invention further includes kits comprising one or more of the antigen binding proteins (e.g. antibodies), or compositions of the invention, or one or more of the nucleic acid molecules encoding the antigen binding proteins (e.g. antibodies) of the invention, or one or more expression vectors comprising the nucleic acid sequences of the invention, or one or more host cells or viruses comprising the expression vectors or nucleic acid sequences of the invention. Preferably said kits are for use in the methods and uses as described herein, e.g. the therapeutic, diagnostic or imaging methods as described herein, or are for use in the in vitro assays or methods as described herein. The antigen binding protein (e.g. antibody) in such kits may be a conjugate as described elsewhere herein, e.g. may be conjugated to a detectable moiety or may be an immunoconjugate. Preferably said kits comprise instructions for use of the kit components. Preferably said kits are for diagnosis or imaging, or treating or preventing diseases or conditions as described elsewhere herein, e.g. cancer, and optionally comprise instructions for use of the kit components to diagnose, image, treat or prevent such diseases or conditions.

The antigen binding proteins (e.g. antibodies) of the invention as defined herein may also be used as molecular tools for in vitro or in vivo applications and assays, for example binding assays or diagnostic assays. As the antigen binding proteins (e.g. antibodies) comprise at least one antigen binding site that binds to HLA-A*02:01/MAGE-A4²³⁰⁻²³⁹ these can be used in any assay where a HLA-A*02:01/MAGE-A4²³⁰⁻²³⁹ binding member is required.

Thus, yet further aspects of the invention provide a reagent that comprises an antigen binding protein (e.g. antibody) of the invention as defined herein and the use of such antigen binding proteins (e.g. antibodies) as molecular tools, for example in in vitro or in vivo assays, for example for the detection of HLA-A*02:01/MAGE-A4²³⁰⁻²³⁹, e.g. in a sample of interest.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”.

In addition, where the terms “comprise”, “comprises”, “has” or “having”, or other equivalent terms are used herein, then in some more specific embodiments, for example in the definition of the CDR or FR sequences herein, these terms include the term “consists of” or “consists essentially of”, or other equivalent terms.

The term “decrease” or “reduce” (or equivalent terms) as described herein includes any measurable decrease or reduction when compared with an appropriate control. Appropriate controls would readily be identified by a person skilled in the art and might include non-treated or placebo treated subjects or healthy subjects, or samples or assays where no antibody (or binding protein) of the invention is present, or where a control antibody (or binding protein), for example an isotype control antibody (or binding protein), is present. Preferably the decrease or reduction will be significant, for example clinically or statistically significant.

The term “increase” (or equivalent terms) as described herein includes any measurable increase or elevation when compared with an appropriate control. Appropriate controls would readily be identified by a person skilled in the art and might include non-treated or placebo treated subjects or healthy subjects, or samples or assays where no antibody (or binding protein) of the invention is present, or where a control antibody (or binding protein), for example an isotype control antibody (or binding protein), is present. Preferably the increase will be significant, for example clinically or statistically significant.

Preferably such increases (and indeed other increases, improvements or positive effects as mentioned elsewhere herein) or such decreases (and indeed other decreases, reductions or negative effects as mentioned elsewhere herein) are measurable increases, decreases, etc., (as appropriate), more preferably they are significant increases, decreases, etc., preferably clinically significant or statistically significant increases, for example with a probability value of 50.05 or <0.05, when compared to an appropriate control level or value.

Methods of determining the statistical significance of differences between test groups of subjects or differences in levels of a particular parameter are well known and documented in the art. For example herein a decrease or increase in level of a particular parameter or a difference between test groups of subjects is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of 50.05 or <0.05.

Tables of Amino Acid Sequences Disclosed Herein and their Sequence Identifiers (SEQ ID NOs)

All amino acid sequences are recited herein from the N-terminus to the C-terminus in line with convention in this technical field.

All nucleotide sequences are recited herein 5′ to 3′ in line with convention in this technical field.

TABLE A
sequences of specific antibodies of the invention that bind to, or specifically
bind to, HLA-A*02:01/MAGE-A4230-239

| | | |
|---|---|---|
| SEQ ID | | |
| NO: | Description | Sequence |

1-H02

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 2 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAGCATTAGCAGCT |
| | | ATTTAAATTGGTATCAGCAGAAACCAGGGAAA |
| | | GCCCCTAAGCTCCTGATCTATGCTGCATCCAG |
| | | TTTGCAAAGTGGGGTCCCATCAAGGTTCAGTG |
| | | GCAGTGGATCTGGGACAGATTTCACTCTCACC |
| | | ATCAGCAGTCTGCAACCTGAAGATTTTGCAAC |
| | | TTACTACTGTCAACAGAGTTACAGTACCCCGT |
| | | ACACTTTTGGCCAGGGGACCAAGCTGGAGAT |
| | | CAAA |
| 3 | VH domain (aa) | QVQLVQSGAEVKKPGASVKVSCKVSGYTLTELS |
| | | MHWVRQAPGKGLEWMGGFDPEDGETIYAQKF |
| | | QGRVTMTEDTSTDTAYMELSSLRSEDTAVYYC |
| | | ATDQGSSWGFYWGQGTLVTVSS |
| 4 | VL domain (aa) | DIRVTQSPSSLSASVGDRVTITCRASQSISSYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 6 | Heavy CDR2 | FDPEDGET |
| 7 | Heavy CDR3 | ATDQGSSWGFY |
| 8 | Light CDR1 | QSISSY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |
| 29 | scFv 1-H02 (full | QVQLVQSGAEVKKPGASVKVSCKVSGYTLTELS |
| | sequence) | MHWVRQAPGKGLEWMGGFDPEDGETIYAQKF |
| | | QGRVTMTEDTSTDTAYMELSSLRSEDTAVYYC |
| | | ATDQGSSWGFYWGQGTLVTVSSKLSGSASAPK |
| | | LEEGEFSEARVDIRVTQSPSSLSASVGDRVTITC |
| | | RASQSISSYLNWYQQKPGKAPKLLIYAASSLQS |
| | | GVPSRFSGSGSGTDFTLTISSLQPEDFATYYCQ |
| | | QSYSTPYTFGQGTKLEIK |
| 30 | scDb 1-H02 (full | DIRVTQSPSSLSASVGDRVTITCRASQSISSYLN |
| | sequence) | WYQQKPGKAPKLLIYAASSLQSGVPSRFSGSG |
| | | SGTDFTLTISSLQPEDFATYYCQQSYSTPYTFG |
| | | QGTKLEIKGGGGSEVQLQQSGPELVKPGASMKI |
| | | SCKASGYSFTGYTMNWVKQSHGKNLEWMGLIN |
| | | PYKGVSTYNQKFKDKATLTVDKSSSTAYMELLS |
| | | LTSEDSAVYYCARSGYYGDSDWYFDVWGAGT |
| | | TVTVSSGGGGSGGGGSGGGGSDIQMTQTTSS |
| | | LSASLGDRVTISCRASQDIRNYLNWYQQKPDGT |
| | | VKLLIYYTSRLHSGVPSKFSGSGSGTDYSLTISN |
| | | LEQEDIATYFCQQGNTLPWTFAGGTKLEIKGGG |
| | | GSQVQLVQSGAEVKKPGASVKVSCKVSGYTLT |
| | | ELSMHWVRQAPGKGLEWMGGFDPEDGETIYA |
| | | QKFQGRVTMTEDTSTDTAYMELSSLRSEDTAV |
| | | YYCATDQGSSWGFYWGQGTLVTVSS |
| 77 | 1-H02 full length | QVQLVQSGAEVKKPGASVKVSCKVSGYTLTELS |
| | heavy chain | MHWVRQAPGKGLEWMGGFDPEDGETIYAQKF |
| | | QGRVTMTEDTSTDTAYMELSSLRSEDTAVYYC |
| | | ATDQGSSWGFYWGQGTLVTVSSASTKGPSVFP |
| | | LAPSSKSTSGGTAALGCLVKDYFPEPVTVSWNS |
| | | GALTSGVHTFPAVLQSSGLYSLSSVVTVPSSSL |
| | | GTQTYICNVNHKPSNTKVDKKVEPKSCDKTHTC |
| | | PPCPAPELLGGPSVFLFPPKPKDTLMISRTPEVT |
| | | CVVVDVSHEDPEVKFNWYVDGVEVHNAKTKPR |
| | | EEQYNSTYRVVSVLTVLHQDWLNGKEYKCKVS |
| | | NKALPAPIEKTISKAKGQPREPQVYTLPPSRDEL |
| | | TKNQVSLTCLVKGFYPSDIAVEWESNGQPENNY |
| | | KTTPPVLDSDGSFFLYSKLTVDKSRWQQGNVF |
| | | SCSVMHEALHNHYTQKSLSLSPGK |
| 78 | 1-H02 full length | DIRVTQSPSSLSASVGDRVTITCRASQSISSYLN |
| | light chain | WYQQKPGKAPKLLIYAASSLQSGVPSRFSGSG |
| | | SGTDFTLTISSLQPEDFATYYCQQSYSTPYTFG |
| | | QGTKLEIKRTVAAPSVFIFPPSDEQLKSGTASVV |
| | | CLLNNFYPREAKVQWKVDNALQSGNSQESVTE |
| | | QDSKDSTYSLSSTLTLSKADYEKHKVYACEVTH |
| | | QGLSSPVTKSFNRGEC |

AM15

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 113 | VL domain (nt) | GACATCCAGTTGACCCAGTCTCCATCCTCCCT |
| | | GTCTGCATCTGTAGGAGACAGAGTCACCATC |
| | | ACTTGCCGGGCAAGTCAGAGCATTAGCAGCT |
| | | ATTTAAATTGGTATCAGCAGAAACCAGGGAAA |
| | | GCCCCTAAGCTCCTGATCTATGCTGCATCCG |
| | | GTTTGCAAAGTGGGGTCCCATCAAGGTTCAG |
| | | TGGCAGTGGATCTGGGACAGATTTCACTCTCA |
| | | CCATCAGCAGTCTGCAACCTGAAGATTTTGCA |
| | | ACTTACTACTGTCAACAGAGTTACAGTACCCC |
| | | CTTCACTTTCGGCCCTGGGACCAAAGTGGAT |
| | | ATCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 114 | VL domain (aa) | DIQLTQSPSSLSASVGDRVTITCRASQSISSYLN |
| | | WYQQKPGKAPKLLIYAASGLQSGVPSRFSGSG |
| | | SGTDFTLTISSLQPEDFATYYCQQSYSTPFTFGP |
| | | GTKVDIK |
| 5 | Heavy CDR1 | GYTLTELS |
| 6 | Heavy CDR2 | FDPEDGET |
| 7 | Heavy CDR3 | ATDQGSSWGFY |
| 8 | Light CDR1 | QSISSY |
| 9 | Light CDR2 | AAS |
| 96 | Light CDR3 | QQSYSTPFT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 102 | Light FR1 | DIQLTQSPSSLSASVGDRVTITCRAS |
| 16 | Light FR2 | LNWYQQKPGKAPKLLIY |
| 109 | Light FR3 | GLQSGVPSRFSGSGSGTDFTLTISSLQPEDFAT |
| | | YYC |
| 112 | Light FR4 | FGPGTKVDIK |
| 148 | scDb AM15 full | DIQLTQSPSSLSASVGDRVTITCRASQSISSYLN |
| | sequence | WYQQKPGKAPKLLIYAASGLQSGVPSRFSGSG |
| | | SGTDFTLTISSLQPEDFATYYCQQSYSTPFTFGP |
| | | GTKVDIKGGGGSEVQLQQSGPELVKPGASMKI |
| | | SCKASGYSFTGYTMNWVKQSHGKNLEWMGLI |
| | | NPYKGVSTYNQKFKDKATLTVDKSSSTAYMELL |
| | | SLTSEDSAVYYCARSGYYGDSDWYFDVWGAG |
| | | TTVTVSSGGGGSGGGGSGGGGSDIQMTQTTS |
| | | SLSASLGDRVTISCRASQDIRNYLNWYQQKPDG |
| | | TVKLLIYYTSRLHSGVPSKFSGSGSGTDYSLTIS |
| | | NLEQEDIATYFCQQGNTLPWTFAGGTKLEIKGG |
| | | GGSQVQLVQSGAEVKKPGASVKVSCKVSGYTL |
| | | TELSMHWVRQAPGKGLEWMGGFDPEDGETIY |
| | | AQKFQGRVTMTEDTSTDTAYMELSSLRSEDTA |
| | | VYYCATDQGSSWGFYWGQGTLVTVSS |
| 149 | scFv AM 15 full | QVQLVQSGAEVKKPGASVKVSCKVSGYTLTEL |
| | sequence | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSSKLSGSASA |
| | | PKLEEGEFSEARVDIQLTQSPSSLSASVGDRVTI |
| | | TCRASQSISSYLNWYQQKPGKAPKLLIYAASGL |
| | | QSGVPSRFSGSGSGTDFTLTISSLQPEDFATYY |
| | | CQQSYSTPFTFGPGTKVDIK |
| 150 | AM 15 full length | QVQLVQSGAEVKKPGASVKVSCKVSGYTLTEL |
| | heavy chain | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKTH |
| | | TCPPCPAPELLGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALPAPIEKTISKAKGQPREPQVYTLPPSRD |
| | | ELTKNQVSLTCLVKGFYPSDIAVEWESNGQPEN |
| | | NYKTTPPVLDSDGSFFLYSKLTVDKSRWQQGN |
| | | VFSCSVMHEALHNHYTQKSLSLSPGK |
| 151 | AM15 full length | DIQLTQSPSSLSASVGDRVTITCRASQSISSYLN |
| | light chain | WYQQKPGKAPKLLIYAASGLQSGVPSRFSGSG |
| | | SGTDFTLTISSLQPEDFATYYCQQSYSTPFTFGP |
| | | GTKVDIKRTVAAPSVFIFPPSDEQLKSGTASVVC |
| | | LLNNFYPREAKVQWKVDNALQSGNSQESVTEQ |
| | | DSKDSTYSLSSTLTLSKADYEKHKVYACEVTHQ |
| | | GLSSPVTKSFNRGEC |
| 160 | AM15 Fab VH/CH1 | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKTH |
| | | T |
| 161 | AM15 Fab VL/CL | DIQLTQSPSSLSASVGDRVTITCRASQSISSYLN |
| | | WYQQKPGKAPKLLIYAASGLQSGVPSRFSGSG |
| | | SGTDFTLTISSLQPEDFATYYCQQSYSTPFTFGP |
| | | GTKVDIKRTVAAPSVFIFPPSDEQLKSGTASVVC |
| | | LLNNFYPREAKVQWKVDNALQSGNSQESVTEQ |
| | | DSKDSTYSLSSTLTLSKADYEKHKVYACEVTHQ |
| | | GLSSPVTKSFNRGEC |

AM2

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 115 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAATATTATGTGGTA |
| | | TTTAAATTGGTATCAGCAGAAACCAGGGAAAG |
| | | CCCCTAAGCTCCTGATCTATGCTGCATCCAGT |
| | | TTGCAAAGTGGGGTCCCATCAAGGTTCAGTG |
| | | GCAGTGGATCTGGGACAGATTTCACTCTCAC |
| | | CATCAGCAGTCTGCAACCTGAAGATTTTGCAA |
| | | CTTACTACTGTCAACAGAGTTACAGTACCCCG |
| | | TACACTTTTGGCCAGGGGACCAAGCTGGAGA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 116 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIK |
| 5 | Heavy CDR1 | GYTLTELS |
| 6 | Heavy CDR2 | FDPEDGET |
| 7 | Heavy CDR3 | ATDQGSSWGFY |
| 87 | Light CDR1 | QNIMWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |
| 152 | scDb AM2 full | DIRVTQSPSSLSASVGDRVTITCRASQNIMWYL |
| | sequence | NWYQQKPGKAPKLLIYAASSLQSGVPSRFSGS |
| | | GSGTDFTLTISSLQPEDFATYYCQQSYSTPYTF |
| | | GQGTKLEIKGGGGSEVQLQQSGPELVKPGASM |
| | | KISCKASGYSFTGYTMNWVKQSHGKNLEWMGL |
| | | INPYKGVSTYNQKFKDKATLTVDKSSSTAYMEL |
| | | LSLTSEDSAVYYCARSGYYGDSDWYFDVWGA |
| | | GTTVTVSSGGGGSGGGGSGGGGSDIQMTQTT |
| | | SSLSASLGDRVTISCRASQDIRNYLNWYQQKPD |
| | | GTVKLLIYYTSRLHSGVPSKFSGSGSGTDYSLTI |
| | | SNLEQEDIATYFCQQGNTLPWTFAGGTKLEIKG |
| | | GGGSQVQLVQSGAEVKKPGASVKVSCKVSGYT |
| | | LTELSMHWVRQAPGKGLEWMGGFDPEDGETIY |
| | | AQKFQGRVTMTEDTSTDTAYMELSSLRSEDTA |
| | | VYYCATDQGSSWGFYWGQGTLVTVSS |
| 153 | scFv AM2 full | QVQLVQSGAEVKKPGASVKVSCKVSGYTLTEL |
| | sequence | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSSKLSGSASA |
| | | PKLEEGEFSEARVDIRVTQSPSSLSASVGDRVTI |
| | | TCRASQNIMWYLNWYQQKPGKAPKLLIYAASSL |
| | | QSGVPSRFSGSGSGTDFTLTISSLQPEDFATYY |
| | | CQQSYSTPYTFGQGTKLEIK |
| 154 | AM2 full length | QVQLVQSGAEVKKPGASVKVSCKVSGYTLTEL |
| | heavy chain | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKTH |
| | | TCPPCPAPELLGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALPAPIEKTISKAKGQPREPQVYTLPPSRD |
| | | ELTKNQVSLTCLVKGFYPSDIAVEWESNGQPEN |
| | | NYKTTPPVLDSDGSFFLYSKLTVDKSRWQQGN |
| | | VFSCSVMHEALHNHYTQKSLSLSPGK |
| 155 | AM2 full length light | DIRVTQSPSSLSASVGDRVTITCRASQNIMWYL |
| | chain | NWYQQKPGKAPKLLIYAASSLQSGVPSRFSGS |
| | | GSGTDFTLTISSLQPEDFATYYCQQSYSTPYTF |
| | | GQGTKLEIKRTVAAPSVFIFPPSDEQLKSGTASV |
| | | VCLLNNFYPREAKVQWKVDNALQSGNSQESVT |
| | | EQDSKDSTYSLSSTLTLSKADYEKHKVYACEVT |
| | | HQGLSSPVTKSFNRGEC |
| 162 | AM2 Fab VH/CH1 | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKTH |
| | | T |
| 163 | AM2 Fab VL/CL | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIKRTVAAPSVFIFPPSDEQLKSGTASV |
| | | VCLLNNFYPREAKVQWKVDNALQSGNSQESVT |
| | | EQDSKDSTYSLSSTLTLSKADYEKHKVYACEVT |
| | | HQGLSSPVTKSFNRGEC |

AMC9

| | | |
|---|---|---|
| 117 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTGCTAGCTGG |
| | | GGATTCTACTGGGGCCAGGGAACCCTGGTCA |
| | | CCGTCTCCTCA |
| 115 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAATATTATGTGGTA |
| | | TTTAAATTGGTATCAGCAGAAACCAGGGAAAG |
| | | CCCCTAAGCTCCTGATCTATGCTGCATCCAGT |
| | | TTGCAAAGTGGGGTCCCATCAAGGTTCAGTG |
| | | GCAGTGGATCTGGGACAGATTTCACTCTCAC |
| | | CATCAGCAGTCTGCAACCTGAAGATTTTGCAA |
| | | CTTACTACTGTCAACAGAGTTACAGTACCCCG |
| | | TACACTTTTGGCCAGGGGACCAAGCTGGAGA |
| | | TCAAA |
| 118 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 116 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIK |
| 5 | Heavy CDR1 | GYTLTELS |
| 6 | Heavy CDR2 | FDPEDGET |
| 85 | Heavy CDR3 | ATDQGASWGFY |
| 87 | Light CDR1 | QNIMWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |
| 156 | scDb AMC9 full | DIRVTQSPSSLSASVGDRVTITCRASQNIMWYL |
| | sequence | NWYQQKPGKAPKLLIYAASSLQSGVPSRFSGS |
| | | GSGTDFTLTISSLQPEDFATYYCQQSYSTPYTF |
| | | GQGTKLEIKGGGGSEVQLQQSGPELVKPGASM |
| | | KISCKASGYSFTGYTMNWVKQSHGKNLEWMGL |
| | | INPYKGVSTYNQKFKDKATLTVDKSSSTAYMEL |
| | | LSLTSEDSAVYYCARSGYYGDSDWYFDVWGA |
| | | GTTVTVSSGGGGSGGGGSGGGGSDIQMTQTT |
| | | SSLSASLGDRVTISCRASQDIRNYLNWYQQKPD |
| | | GTVKLLIYYTSRLHSGVPSKFSGSGSGTDYSLTI |
| | | SNLEQEDIATYFCQQGNTLPWTFAGGTKLEIKG |
| | | GGGSQVQLVQSGAEVKKPGASVKVSCKVSGYT |
| | | LTELSMHWVRQAPGKGLEWMGGFDPEDGETIY |
| | | AQKFQGRVTMTEDTSTDTAYMELSSLRSEDTA |
| | | VYYCATDQGASWGFYWGQGTLVTVSS |
| 157 | scFv AMC9 full | QVQLVQSGAEVKKPGASVKVSCKVSGYTLTEL |
| | sequence | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSSKLSGSASA |
| | | PKLEEGEFSEARVDIRVTQSPSSLSASVGDRVTI |
| | | TCRASQNIMWYLNWYQQKPGKAPKLLIYAASSL |
| | | QSGVPSRFSGSGSGTDFTLTISSLQPEDFATYY |
| | | CQQSYSTPYTFGQGTKLEIK |
| 158 | AMC9 full length | QVQLVQSGAEVKKPGASVKVSCKVSGYTLTEL |
| | heavy chain | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKTH |
| | | TCPPCPAPELLGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALPAPIEKTISKAKGQPREPQVYTLPPSRD |
| | | ELTKNQVSLTCLVKGFYPSDIAVEWESNGQPEN |
| | | NYKTTPPVLDSDGSFFLYSKLTVDKSRWQQGN |
| | | VFSCSVMHEALHNHYTQKSLSLSPGK |
| 159 | AMC9 full length | DIRVTQSPSSLSASVGDRVTITCRASQNIMWYL |
| | light chain | NWYQQKPGKAPKLLIYAASSLQSGVPSRFSGS |
| | | GSGTDFTLTISSLQPEDFATYYCQQSYSTPYTF |
| | | GQGTKLEIKRTVAAPSVFIFPPSDEQLKSGTASV |
| | | VCLLNNFYPREAKVQWKVDNALQSGNSQESVT |
| | | EQDSKDSTYSLSSTLTLSKADYEKHKVYACEVT |
| | | HQGLSSPVTKSFNRGEC |
| 164 | AMC9 Fab VH/CH1 | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKTH |
| | | T |
| 165 | AMC9 Fab VL/CL | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIKRTVAAPSVFIFPPSDEQLKSGTASV |
| | | VCLLNNFYPREAKVQWKVDNALQSGNSQESVT |
| | | EQDSKDSTYSLSSTLTLSKADYEKHKVYACEVT |
| | | HQGLSSPVTKSFNRGEC |

AM1

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 119 | VL domain (nt) | GACATCCGGGTGACCCAGTCTCCATCCTCCC |
| | | TGTCTGCATCTGTAGGAGACAGAGTCACCATC |
| | | ACTTGCCGGGCAGATGTGCATATTTATTGGTA |
| | | TTTAAATTGGTATCAGCAGAAACCAGGGAAAG |
| | | CCCCTAAGCTCCTGATCTATGCTGCATCCAGT |
| | | TTGCAAAGTGGGGTCCCATCAAGGTTCAGTG |
| | | GCAGTGGATCTGGGACAGATTTCACTCTCAC |
| | | CATCAGCAGTCTGCAACCTGAAGATTTTGCAA |
| | | CTTACTACTGTCAACAGAGTTACAGTACCCCG |
| | | TACACTTTTGGCCAGGGGACCAAGCTGGAGA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 120 | VL domain (aa) | DIRVTQSPSSLSASVGDRVTITCRADVHIYWYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 6 | Heavy CDR2 | FDPEDGET |
| 7 | Heavy CDR3 | ATDQGSSWGFY |
| 88 | Light CDR1 | VHIYWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 105 | Light FR1 | DIRVTQSPSSLSASVGDRVTITCRAD |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM3

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 121 | VL domain (nt) | GACATCCGGGTGACCCAGTCTCCATCCTCCC |
| | | TGTCTGCATCTGTAGGAGACAGAGTCACCATC |
| | | ACTTGCCGGGCAGATCATCATATTTTTTGGTA |
| | | TTTAAATTGGTATCAGCAGAAACCAGGGAAAG |
| | | CCCCTAAGCTCCTGATCTATGCTGCATCCAGT |
| | | TTGCAAAGTGGGGTCCCATCAAGGTTCAGTG |
| | | GCAGTGGATCTGGGACAGATTTCACTCTCAC |
| | | CATCAGCAGTCTGCAACCTGAAGATTTTGCAA |
| | | CTTACTACTGTCAACAGAGTTACAGTACCCCG |
| | | TACACTTTTGGCCAGGGGACCAAGCTGGAGA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 122 | VL domain (aa) | DIRVTQSPSSLSASVGDRVTITCRADHHIFWYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 6 | Heavy CDR2 | FDPEDGET |
| 7 | Heavy CDR3 | ATDQGSSWGFY |
| 89 | Light CDR1 | HHIFWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 105 | Light FR1 | DIRVTQSPSSLSASVGDRVTITCRAD |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM4

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 123 | VL domain (nt) | GACATCCGGGTGACCCAGTCTCCATCCTCCC |
| | | TGTCTGCATCTGTAGGAGACAGAGTCACCATC |
| | | ACTTGCCGGGCAGATATTGATATTCGGTGGTA |
| | | TTTAAATTGGTATCAGCAGAAACCAGGGAAAG |
| | | CCCCTAAGCTCCTGATCTATGCTGCATCCAGT |
| | | TTGCAAAGTGGGGTCCCATCAAGGTTCAGTG |
| | | GCAGTGGATCTGGGACAGATTTCACTCTCAC |
| | | CATCAGCAGTCTGCAACCTGAAGATTTTGCAA |
| | | CTTACTACTGTCAACAGAGTTACAGTACCCCG |
| | | TACACTTTTGGCCAGGGGACCAAGCTGGAGA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 124 | VL domain (aa) | DIRVTQSPSSLSASVGDRVTITCRADIDIRWYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 6 | Heavy CDR2 | FDPEDGET |
| 7 | Heavy CDR3 | ATDQGSSWGFY |
| 90 | Light CDR1 | IDIRWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 105 | Light FR1 | DIRVTQSPSSLSASVGDRVTITCRAD |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM5

| | | |
|---|---|---|
| 125 | VH domain (nt) | CAGGTCCAGCTTGTGCAGTCTGTGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAGCAGATCAGGGTAGTAGCTGG |
| | | GGATTCTACTGGGGCCAGGGAACCCTGGTCA |
| | | CCGTCTCCTCA |
| 127 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAGCATTAGCAGCT |
| | | ATTTAAATTGGTATCAGCAGAAACCAGGGAAA |
| | | GCCCCTAAGCTCCTGATCTATGCTGCATCCAG |
| | | TTTGCAAAGTGGGGTCCCATCAAGGTTCAGT |
| | | GGCAGTGGATCTGGGACAGATTTCACTCTCA |
| | | CCATCAGCAGTCTGCAACCTGAAGATTTTGCA |
| | | ACTTACTACTGTCAACAGGCCTACCGCATCCC |
| | | CTACACTTTTGGCCAGGGGACCAAGCTGGAG |
| | | ATCAAA |
| 126 | VH domain (aa) | QVQLVQSVAEVKKPGASVKVSCKVSGYTLTELS |
| | | MHWVRQAPGKGLEWMGGFDPEDGETIYAQKF |
| | | QGRVTMTEDTSTDTAYMELSSLRSEDTAVYYC |
| | | AADQGSSWGFYWGQGTLVTVSS |
| 128 | VL domain (aa) | DIRVTQSPSSLSASVGDRVTITCRASQSISSYLN |
| | | WYQQKPGKAPKLLIYAASSLQSGVPSRFSGSG |
| | | SGTDFTLTISSLQPEDFATYYCQQAYRIPYTFGQ |
| | | GTKLEIK |
| 5 | Heavy CDR1 | GYTLTELS |
| 6 | Heavy CDR2 | FDPEDGET |
| 86 | Heavy CDR3 | AADQGSSWGFY |
| 8 | Light CDR1 | QSISSY |
| 9 | Light CDR2 | AAS |
| 97 | Light CDR3 | QQAYRIPYT |
| 99 | Heavy FR1 | QVQLVQSVAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM6

| | | |
|---|---|---|
| 129 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGACCGAAGCTGTATGAG |
| | | TATCCATGCACTGGGTGCGACAGGCTCCTGG |
| | | GTTTCCATGCACTGGGTGCGACAGGCTCCTG |
| | | GAAAAGGGCTTGAGTGGATGGGAGGTTTTGA |
| | | TCCTGAAGATGGTGAAACAATCTACGCACAGA |
| | | AGTTCCAGGGCAGAGTCACCATGACCGAGGA |
| | | CACATCTACAGACACAGCCTACATGGAGCTG |
| | | AGCAGCCTGAGATCTGAGGACACGGCCGTGT |
| | | ATTACTGTGCAACAGATCAGGGTAGTAGCTG |
| | | GGGATTCTACTGGGGCCAGGGAACCCTGGTC |
| | | ACCGTCTCCTCA |
| 2 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAGCATTAGCAGCT |
| | | ATTTAAATTGGTATCAGCAGAAACCAGGGAAA |
| | | GCCCCTAAGCTCCTGATCTATGCTGCATCCAG |
| | | TTTGCAAAGTGGGGTCCCATCAAGGTTCAGT |
| | | GGCAGTGGATCTGGGACAGATTTCACTCTCA |
| | | CCATCAGCAGTCTGCAACCTGAAGATTTTGCA |
| | | ACTTACTACTGTCAACAGAGTTACAGTACCCC |
| | | GTACACTTTTGGCCAGGGGACCAAGCTGGAG |
| | | ATCAAA |
| 130 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 4 | VL domain (aa) | DIRVTQSPSSLSASVGDRVTITCRASQSISSYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 81 | Heavy CDR1 | GPKLYEVS |
| 6 | Heavy CDR2 | FDPEDGET |
| 7 | Heavy CDR3 | ATDQGSSWGFY |
| 8 | Light CDR1 | QSISSY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM7

| | | |
|---|---|---|
| 131 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTTATATGTCTCGGACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 2 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAGCATTAGCAGCT |
| | | ATTTAAATTGGTATCAGCAGAAACCAGGGAAA |
| | | GCCCCTAAGCTCCTGATCTATGCTGCATCCAG |
| | | TTTGCAAAGTGGGGTCCCATCAAGGTTCAGT |
| | | GGCAGTGGATCTGGGACAGATTTCACTCTCA |
| | | CCATCAGCAGTCTGCAACCTGAAGATTTTGCA |
| | | ACTTACTACTGTCAACAGAGTTACAGTACCCC |
| | | GTACACTTTTGGCCAGGGGACCAAGCTGGAG |
| | | ATCAAA |
| 132 | VH domain (aa) | QVQLVQSGAEVKKPGASVKVSCKVSGYTLTEL |
| | | SMHWVRQAPGKGLEWMGGFDPYMSRTIYAQK |
| | | FQGRVTMTEDTSTDTAYMELSSLRSEDTAVYY |
| | | CATDQGSSWGFYWGQGTLVTVSS |
| 4 | VL domain (aa) | DIRVTQSPSSLSASVGDRVTITCRASQSISSYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 82 | Heavy CDR2 | FDPYMSRT |
| 7 | Heavy CDR3 | ATDQGSSWGFY |
| 8 | Light CDR1 | QSISSY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM9

| | | |
|---|---|---|
| 133 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTTATCTTGCGCGTACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 2 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAGCATTAGCAGCT |
| | | ATTTAAATTGGTATCAGCAGAAACCAGGGAAA |
| | | GCCCCTAAGCTCCTGATCTATGCTGCATCCAG |
| | | TTTGCAAAGTGGGGTCCCATCAAGGTTCAGT |
| | | GGCAGTGGATCTGGGACAGATTTCACTCTCA |
| | | CCATCAGCAGTCTGCAACCTGAAGATTTTGCA |
| | | ACTTACTACTGTCAACAGAGTTACAGTACCCC |
| | | GTACACTTTTGGCCAGGGGACCAAGCTGGAG |
| | | ATCAAA |
| 134 | VH domain (aa) | QVQLVQSGAEVKKPGASVKVSCKVSGYTLTEL |
| | | SMHWVRQAPGKGLEWMGGFDPYLARTIYAQK |
| | | FQGRVTMTEDTSTDTAYMELSSLRSEDTAVYY |
| | | CATDQGSSWGFYWGQGTLVTVSS |
| 4 | VL domain (aa) | DIRVTQSPSSLSASVGDRVTITCRASQSISSYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 83 | Heavy CDR2 | FDPYLART |
| 7 | Heavy CDR3 | ATDQGSSWGFY |
| 8 | Light CDR1 | QSISSY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM10

| | | |
|---|---|---|
| 117 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTGCTAGCTGG |
| | | GGATTCTACTGGGGCCAGGGAACCCTGGTCA |
| | | CCGTCTCCTCA |
| 135 | VL domain (nt) | GACATCCGGGTGACCCAGTCTCCATCCTCCC |
| | | TGTCTGCATCTGTAGGAGACAGAGTCACCATC |
| | | ACTTGCCGGGCAAGTCAGAGCATTAGCAGCT |
| | | ATTTAAATTGGTATCAACAGAAACCAGGGAAA |
| | | GCCCCTAAGCTCCTGATCTATGCTGCATCCAG |
| | | TTTGCAAAGTGGGGTCCCATCAAGGTTCAGT |
| | | GGCAGTGGATCTGGGACAGATTTCACTCTCA |
| | | CCATCAGCAGTCTGCAACCTGAAGATTTTGCA |
| | | ACTTACTACTGTCAACAGAGTTACAGTACCCC |
| | | GTACACTTTTGGCCAGGGGACCAAGCTGGAG |
| | | ATCAAA |
| 118 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 4 | VL domain (aa) | DIRVTQSPSSLSASVGDRVTITCRASQSISSYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 6 | Heavy CDR2 | FDPEDGET |
| 85 | Heavy CDR3 | ATDQGASWGFY |
| 8 | Light CDR1 | QSISSY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM11

| | | |
|---|---|---|
| 134 | VH domain (aa) | QVQLVQSGAEVKKPGASVKVSCKVSGYTLTEL |
| | | SMHWVRQAPGKGLEWMGGFDPYLARTIYAQK |
| | | FQGRVTMTEDTSTDTAYMELSSLRSEDTAVYY |
| | | CATDQGSSWGFYWGQGTLVTVSS |
| 124 | VL domain (aa) | DIRVTQSPSSLSASVGDRVTITCRADIDIRWYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 83 | Heavy CDR2 | FDPYLART |
| 7 | Heavy CDR3 | ATDQGSSWGFY |
| 90 | Light CDR1 | IDIRWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 105 | Light FR1 | DIRVTQSPSSLSASVGDRVTITCRAD |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM12

| | | |
|---|---|---|
| 136 | VH domain (aa) | QVQLVQSGAEVKKPGASVKVSCKVSGYTLTEL |
| | | SMHWVRQAPGKGLEWMGGFDPEQGETIYAQK |
| | | FQGRVTMTEDTSTDTAYMELSSLRSEDTAVYY |
| | | CATDQGASWGFYWGQGTLVTVSS |
| 116 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIK |
| 5 | Heavy CDR1 | GYTLTELS |
| 84 | Heavy CDR2 | FDPEQGET |
| 85 | Heavy CDR3 | ATDQGASWGFY |
| 87 | Light CDR1 | QNIMWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AM13

| | | |
|---|---|---|
| 137 | VH domain (aa) | QVQLVQSGAEVKKPGASVKVSCKVSGYTLTEL |
| | | SMHWVRQAPGKGLEWMGGFDPYLARTIYAQK |
| | | FQGRVTMTEDTSTDTAYMELSSLRSEDTAVYY |
| | | CATDQGASWGFYWGQGTLVTVSS |
| 114 | VL domain (aa) | DIQLTQSPSSLSASVGDRVTITCRASQSISSYLN |
| | | WYQQKPGKAPKLLIYAASGLQSGVPSRFSGSG |
| | | SGTDFTLTISSLQPEDFATYYCQQSYSTPFTFGP |
| | | GTKVDIK |
| 5 | Heavy CDR1 | GYTLTELS |
| 83 | Heavy CDR2 | FDPYLART |
| 85 | Heavy CDR3 | ATDQGASWGFY |
| 8 | Light CDR1 | QSISSY |
| 9 | Light CDR2 | AAS |
| 96 | Light CDR3 | QQSYSTPFT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 102 | Light FR1 | DIQLTQSPSSLSASVGDRVTITCRAS |
| 16 | Light FR2 | LNWYQQKPGKAPKLLIY |
| 109 | Light FR3 | GLQSGVPSRFSGSGSGTDFTLTISSLQPEDFAT |
| | | YYC |
| 112 | Light FR4 | FGPGTKVDIK |

AM14

| | | |
|---|---|---|
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 138 | VL domain (aa) | DIQLTQSPSSLSASVGDRVTITCRADIDIRWYLN |
| | | WYQQKPGKAPKLLIYAASGLQSGVPSRFSGSG |
| | | SGTDFTLTISSLQPEDFATYYCQQSYSTPFTFGP |
| | | GTKVDIK |
| 5 | Heavy CDR1 | GYTLTELS |
| 6 | Heavy CDR2 | FDPEDGET |
| 7 | Heavy CDR3 | ATDQGSSWGFY |
| 90 | Light CDR1 | IDIRWY |
| 9 | Light CDR2 | AAS |
| 96 | Light CDR3 | QQSYSTPFT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 101 | Light FR1 | DIQLTQSPSSLSASVGDRVTITCRAD |
| 16 | Light FR2 | LNWYQQKPGKAPKLLIY |
| 109 | Light FR3 | GLQSGVPSRFSGSGSGTDFTLTISSLQPEDFAT |
| | | YYC |
| 112 | Light FR4 | FGPGTKVDIK |

AM16

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 139 | VL domain (nt) | GACATCCAGTTGACCCAGTCTCCATCCTCCCT |
| | | GTCTGCATCTGTAGGAGACAGAGTCACCATC |
| | | AGTTGCCGGGCAAGTCAGAGCATTATGACCT |
| | | ATTTAAATTGGTATCAGCAGAGACCAGGGAAA |
| | | GCCCCTAAGCTCCTGATCTATTCTGCATCCAG |
| | | GTTGCAAAGTGGGGTCCCATCCAGGTTCAGT |
| | | GGCAGTGGATATGGGACAGAATTCACTCTCA |
| | | CCATCAGCAGCCTGCAACCTGAAGATTTTGCA |
| | | ACTTACTACTGTCAACAGAGTTACAGTACCCC |
| | | TTTCACTTTCGGCCCTGGGACCAAAGTGGATA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 140 | VL domain (aa) | DIQLTQSPSSLSASVGDRVTISCRASQSIMTYLN |
| | | WYQQRPGKAPKLLIYSASRLQSGVPSRFSGSG |
| | | YGTEFTLTISSLQPEDFATYYCQQSYSTPFTFGP |
| | | GTKVDIK |
| 5 | Heavy CDR1 | GYTLTELS |
| 6 | Heavy CDR2 | FDPEDGET |
| 7 | Heavy CDR3 | ATDQGSSWGFY |
| 91 | Light CDR1 | QSIMTY |
| 94 | Light CDR2 | SAS |
| 96 | Light CDR3 | QQSYSTPFT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 100 | Light FR1 | DIQLTQSPSSLSASVGDRVTISCRAS |
| 106 | Light FR2 | LNWYQQRPGKAPKLLIY |
| 111 | Light FR3 | RLQSGVPSRFSGSGYGTEFTLTISSLQPEDFAT |
| | | YYC |
| 112 | Light FR4 | FGPGTKVDIK |

AM17

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 141 | VL domain (nt) | GACATCCGGTTGACCCAGTCTCCATCCTCCCT |
| | | GTCTGCTTCTGTAGGCGACAGAGTCACCATG |
| | | ACTTGCCGGGCAAGTCAGACCGTTGCCACTT |
| | | ATGTAAATTGGTATCAACAAAAACCAGGGAGA |
| | | GCCCCTAAACTCCTAATGTATGTCACGTCCAT |
| | | TCGACAAAGTGAGGTCCCATCAAGGTTCAGT |
| | | GGTGATGGATTTGGCACAGACTTCACTCTCAC |
| | | CATCAATCCTCTGCAGCCTGACGATTTCGCAA |
| | | CTTACTACTGTCAACAGGCCTACAGTACTCCT |
| | | GTGACGTTCGGCCAAGGGACCAAGCTGGAGA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 142 | VL domain (aa) | DIRLTQSPSSLSASVGDRVTMTCRASQTVATYV |
| | | NWYQQKPGRAPKLLMYVTSIRQSEVPSRFSGD |
| | | GFGTDFTLTINPLQPDDFATYYCQQAYSTPVTF |
| | | GQGTKLEIK |
| 5 | Heavy CDR1 | GYTLTELS |
| 6 | Heavy CDR2 | FDPEDGET |
| 7 | Heavy CDR3 | ATDQGSSWGFY |
| 92 | Light CDR1 | QTVATY |
| 95 | Light CDR2 | VTS |
| 98 | Light CDR3 | QQAYSTPVT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 103 | Light FR1 | DIRLTQSPSSLSASVGDRVTMTCRAS |
| 108 | Light FR2 | VNWYQQKPGRAPKLLMY |
| 110 | Light FR3 | IRQSEVPSRFSGDGFGTDFTLTINPLQPDDFATY |
| | | YC |
| 18 | Light FR4 | FGQGTKLEIK |

AM18

| | | |
|---|---|---|
| 1 | VH domain (nt) | CAGGTCCAGCTTGTGCAGTCTGGGGCTGAGG |
| | | TGAAGAAGCCTGGGGCCTCAGTGAAGGTCTC |
| | | CTGCAAGGTTTCCGGATACACCCTCACTGAAT |
| | | TATCCATGCACTGGGTGCGACAGGCTCCTGG |
| | | AAAAGGGCTTGAGTGGATGGGAGGTTTTGAT |
| | | CCTGAAGATGGTGAAACAATCTACGCACAGAA |
| | | GTTCCAGGGCAGAGTCACCATGACCGAGGAC |
| | | ACATCTACAGACACAGCCTACATGGAGCTGA |
| | | GCAGCCTGAGATCTGAGGACACGGCCGTGTA |
| | | TTACTGTGCAACAGATCAGGGTAGTAGCTGG |
| | | GGATTCTACTGGGGCCAGGGAACCCTGGTCA |
| | | CCGTCTCCTCA |
| 143 | VL domain (nt) | GACATCCGGTTGACCCAGTCTCCATCCTCCCT |
| | | GTCTGTATCTGTAGGAGACAGAGTCACCATCA |
| | | CTTGCCGGGCGAGTGAGGACATTAGATACTA |
| | | TGTAAATTGGTATCAGCAGAAACCAGGGAAAG |
| | | CCCCTAAGCTCCTGATCTATGCTGCATCCAGT |
| | | TTGCAAAGTGGGGTCCCATCAAGGTTCAGTG |
| | | GCAGTGGATCTGGGACAGATTTCACTCTCAC |
| | | CATCAGCAGTCTGCAACCTGAAGATTTTGCAA |
| | | CTTACTACTGTCAACAGAGTTACAGTACCCCG |
| | | TACACTTTTGGCCAGGGGACCAAGCTGGAGA |
| | | TCAAA |
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 144 | VL domain (aa) | DIRLTQSPSSLSVSVGDRVTITCRASEDIRYYVN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 5 | Heavy CDR1 | GYTLTELS |
| 6 | Heavy CDR2 | FDPEDGET |
| 7 | Heavy CDR3 | ATDQGSSWGFY |
| 93 | Light CDR1 | EDIRYY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 104 | Light FR1 | DIRLTQSPSSLSVSVGDRVTITCRAS |
| 107 | Light FR2 | VNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC1

| | | |
|---|---|---|
| 145 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 142 | VL domain (aa) | DIRLTQSPSSLSASVGDRVTMTCRASQTVATYV |
| | | NWYQQKPGRAPKLLMYVTSIRQSEVPSRFSGD |
| | | GFGTDFTLTINPLQPDDFATYYCQQAYSTPVTF |
| | | GQGTKLEIK |
| 81 | Heavy CDR1 | GPKLYEVS |
| 6 | Heavy CDR2 | FDPEDGET |
| 85 | Heavy CDR3 | ATDQGASWGFY |
| 92 | Light CDR1 | QTVATY |
| 95 | Light CDR2 | VTS |
| 98 | Light CDR3 | QQAYSTPVT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 103 | Light FR1 | DIRLTQSPSSLSASVGDRVTMTCRAS |
| 108 | Light FR2 | VNWYQQKPGRAPKLLMY |
| 110 | Light FR3 | IRQSEVPSRFSGDGFGTDFTLTINPLQPDDFATY |
| | | YC |
| 18 | Light FR4 | FGQGTKLEIK |

AMC3

| | | |
|---|---|---|
| 145 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 116 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIK |
| 81 | Heavy CDR1 | GPKLYEVS |
| 6 | Heavy CDR2 | FDPEDGET |
| 85 | Heavy CDR3 | ATDQGASWGFY |
| 87 | Light CDR1 | QNIMWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC4

| | | |
|---|---|---|
| 145 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 122 | VL domain (aa) | DIRVTQSPSSLSASVGDRVTITCRADHHIFWYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 81 | Heavy CDR1 | GPKLYEVS |
| 6 | Heavy CDR2 | FDPEDGET |
| 85 | Heavy CDR3 | ATDQGASWGFY |
| 89 | Light CDR1 | HHIFWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 105 | Light FR1 | DIRVTQSPSSLSASVGDRVTITCRAD |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC5

| | | |
|---|---|---|
| 145 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 124 | VL domain (aa) | DIRVTQSPSSLSASVGDRVTITCRADIDIRWYLN |
| | | WYQQKPGKAPKLLIYAASSLQSGVPSRFSGSG |
| | | SGTDFTLTISSLQPEDFATYYCQQSYSTPYTFG |
| | | QGTKLEIK |
| 81 | Heavy CDR1 | GPKLYEVS |
| 6 | Heavy CDR2 | FDPEDGET |
| 85 | Heavy CDR3 | ATDQGASWGFY |
| 90 | Light CDR1 | IDIRWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 105 | Light FR1 | DIRVTQSPSSLSASVGDRVTITCRAD |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC6

| | | |
|---|---|---|
| 130 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 142 | VL domain (aa) | DIRLTQSPSSLSASVGDRVTMTCRASQTVATYV |
| | | NWYQQKPGRAPKLLMYVTSIRQSEVPSRFSGD |
| | | GFGTDFTLTINPLQPDDFATYYCQQAYSTPVTF |
| | | GQGTKLEIK |
| 81 | Heavy CDR1 | GPKLYEVS |
| 6 | Heavy CDR2 | FDPEDGET |
| 7 | Heavy CDR3 | ATDQGSSWGFY |
| 92 | Light CDR1 | QTVATY |
| 95 | Light CDR2 | VTS |
| 98 | Light CDR3 | QQAYSTPVT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 103 | Light FR1 | DIRLTQSPSSLSASVGDRVTMTCRAS |
| 108 | Light FR2 | VNWYQQKPGRAPKLLMY |
| 110 | Light FR3 | IRQSEVPSRFSGDGFGTDFTLTINPLQPDDFATY |
| | | YC |
| 18 | Light FR4 | FGQGTKLEIK |

AMC7

| | | |
|---|---|---|
| 130 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 116 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQSYSTPYTF |
| | | GQGTKLEIK |
| 81 | Heavy CDR1 | GPKLYEVS |
| 6 | Heavy CDR2 | FDPEDGET |
| 7 | Heavy CDR3 | ATDQGSSWGFY |
| 87 | Light CDR1 | QNIMWY |
| 9 | Light CDR2 | AAS |
| 10 | Light CDR3 | QQSYSTPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC8

| | | |
|---|---|---|
| 118 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 142 | VL domain (aa) | DIRLTQSPSSLSASVGDRVTMTCRASQTVATYV |
| | | NWYQQKPGRAPKLLMYVTSIRQSEVPSRFSGD |
| | | GFGTDFTLTINPLQPDDFATYYCQQAYSTPVTF |
| | | GQGTKLEIK |
| 5 | Heavy CDR1 | GYTLTELS |
| 6 | Heavy CDR2 | FDPEDGET |
| 85 | Heavy CDR3 | ATDQGASWGFY |
| 92 | Light CDR1 | QTVATY |
| 95 | Light CDR2 | VTS |
| 98 | Light CDR3 | QQAYSTPVT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 103 | Light FR1 | DIRLTQSPSSLSASVGDRVTMTCRAS |
| 108 | Light FR2 | VNWYQQKPGRAPKLLMY |
| 110 | Light FR3 | IRQSEVPSRFSGDGFGTDFTLTINPLQPDDFATY |
| | | YC |
| 18 | Light FR4 | FGQGTKLEIK |

AMC10

| | | |
|---|---|---|
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 146 | VL domain (aa) | DIQLTQSPSSLSASVGDRVTITCRASQNIMWYL |
| | | NWYQQKPGKAPKLLIYAASGLQSGVPSRFSGS |
| | | GSGTDFTLTISSLQPEDFATYYCQQSYSTPFTF |
| | | GPGTKVDIK |
| 5 | Heavy CDR1 | GYTLTELS |
| 6 | Heavy CDR2 | FDPEDGET |
| 7 | Heavy CDR3 | ATDQGSSWGFY |
| 87 | Light CDR1 | QNIMWY |
| 9 | Light CDR2 | AAS |
| 96 | Light CDR3 | QQSYSTPFT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 102 | Light FR1 | DIQLTQSPSSLSASVGDRVTITCRAS |
| 16 | Light FR2 | LNWYQQKPGKAPKLLIY |
| 109 | Light FR3 | GLQSGVPSRFSGSGSGTDFTLTISSLQPEDFAT |
| | | YYC |
| 112 | Light FR4 | FGPGTKVDIK |

AMC11

| | | |
|---|---|---|
| 3 | VH domain (aa) | QVQLVQSGAEVKKPGASVKVSCKVSGYTLTEL |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 147 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQAYRIPYTFG |
| | | QGTKLEIK |
| 5 | Heavy CDR1 | GYTLTELS |
| 6 | Heavy CDR2 | FDPEDGET |
| 7 | Heavy CDR3 | ATDQGSSWGFY |
| 87 | Light CDR1 | QNIMWY |
| 9 | Light CDR2 | AAS |
| 97 | Light CDR3 | QQAYRIPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC12

| | | |
|---|---|---|
| 145 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGASWGFYWGQGTLVTVSS |
| 147 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQAYRIPYTFG |
| | | QGTKLEIK |
| 81 | Heavy CDR1 | GPKLYEVS |
| 6 | Heavy CDR2 | FDPEDGET |
| 85 | Heavy CDR3 | ATDQGASWGFY |
| 87 | Light CDR1 | QNIMWY |
| 9 | Light CDR2 | AAS |
| 97 | Light CDR3 | QQAYRIPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

AMC14

| | | |
|---|---|---|
| 130 | VH domain (aa) | QVQLVQSGAEVKKPGASVKVSCKVSGPKLYEV |
| | | SMHWVRQAPGKGLEWMGGFDPEDGETIYAQK |
| | | FQGRVTMTEDTSTDTAYMELSSLRSEDTAVYY |
| | | CATDQGSSWGFYWGQGTLVTVSS |
| 147 | VL domain (aa) | DIRVTQSPSSLSASVGDRVTITCRASQNIMWYL |
| | | NWYQQKPGKAPKLLIYAASSLQSGVPSRFSGS |
| | | GSGTDFTLTISSLQPEDFATYYCQQAYRIPYTFG |
| | | QGTKLEIK |
| 81 | Heavy CDR1 | GPKLYEVS |
| 6 | Heavy CDR2 | FDPEDGET |
| 7 | Heavy CDR3 | ATDQGSSWGFY |
| 87 | Light CDR1 | QNIMWY |
| 9 | Light CDR2 | AAS |
| 97 | Light CDR3 | QQAYRIPYT |
| 11 | Heavy FR1 | QVQLVQSGAEVKKPGASVKVSCKVS |
| 12 | Heavy FR2 | MHWVRQAPGKGLEWMGG |
| 13 | Heavy FR3 | IYAQKFQGRVTMTEDTSTDTAYMELSSLRSEDT |
| | | AVYYC |
| 14 | Heavy FR4 | WGQGTLVTVSS |
| 15 | Light FR1 | DIRVTQSPSSLSASVGDRVTITCRAS |
| 16 | Light FR2 | LNWYQQKPGKAPKLLIY |
| 17 | Light FR3 | SLQSGVPSRFSGSGSGTDFTLTISSLQPEDFAT |
| | | YYC |
| 18 | Light FR4 | FGQGTKLEIK |

The constant regions of 1-H02, AM15, AM2 and AMC9 shown respectively within SEQ ID NOs: 77 and 78, within SEQ ID NOs: 150 and 151, within SEQ ID NOs: 154 and 155 and within SEQ ID NOs: 158 and 159 in Table A are human IgG1 antibody sequences well known and described in the art. Such light chain constant regions, and parts of such heavy chain constant regions, are also present in the AM15, AM2 and AMC9 antibodies in the Fab format, which comprise the Fab VH/CH1 and Fab VL/CL sequences shown respectively in SEQ ID NOs: 160 and 161, in SEQ ID NOs: 162 and 163, and in SEQ ID NOs: 164 and 165 in Table A.

TABLE B
Other sequences disclosed herein
Table B

| SEQ ID | | |
|---|---|---|
| NO: | Description | Sequence |
| 19 | WT MAGE-A4²³⁰⁻²³⁹ | GVYDGREHTV |
| 20 | WT MAGE A4 | MSSEQKSQHCKPEEGVEAQEEALGLVGAQAP |
| | | TTEEQEAAVSSSSPLVPGTLEEVPAAESAGPP |
| | | QSPQGASALPTTISFTCWRQPNEGSSSQEEE |
| | | GPSTSPDAESLFREALSNKVDELAHFLLRKYR |
| | | AKELVTKAEMLERVIKNYKRCFPVIFGKASESL |
| | | KMIFGIDVKEVDPASNTYTLVTCLGLSYDGLLG |
| | | NNQIFPKTGLLIIVLGTIAMEGDSASEEEIWEEL |
| | | GVMGVYDGREHTVYGEPRKLLTQDWVQENY |
| | | LEYRQVPGSNPARYEFLWGPRALAETSYVKVL |
| | | EHVVRVNARVRIAYPSLREAALLEEEEGV |
| 21 | Full length | MAVMAPRTLVLLLSGALALTQTWAGSHSMRY |
| | (prototypic) HLA | FFTSVSRPGRGEPRFIAVGYVDDTQFVRFDSD |
| | 02:01:01:01 allele | AASQRMEPRAPWIEQEGPEYWDGETRKVKA |
| | | HSQTHRVDLGTLRGYYNQSEAGSHTVQRMY |
| | | GCDVGSDWRFLRGYHQYAYDGKDYIALKEDL |
| | | RSWTAADMAAQTTKHKWEAAHVAEQLRAYLE |
| | | GTCVEWLRRYLENGKETLQRTDAPKTHMTHH |
| | | AVSDHEATLRCWALSFYPAEITLTWQRDGED |
| | | QTQDTELVETRPAGDGTFQKWAAVVVPSGQE |
| | | QRYTCHVQHEGLPKPLTLRWEPSSQPTIPIVGI |
| | | IAGLVLFGAVITGAVVAAVMWRRKSSDRKGGS |
| | | YSQAASSDSAQGSDVSLTACKV |
| 22 | ECD of HLA | GSHSMRYFFTSVSRPGRGEPRFIAVGYVDDT |
| | 02:01:01:01 allele | QFVRFDSDAASQRMEPRAPWIEQEGPEYWD |
| | | GETRKVKAHSQTHRVDLGTLRGYYNQSEAGS |
| | | HTVQRMYGCDVGSDWRFLRGYHQYAYDGKD |
| | | YIALKEDLRSWTAADMAAQTTKHKWEAAHVA |
| | | EQLRAYLEGTCVEWLRRYLENGKETLQRTDA |
| | | PKTHMTHHAVSDHEATLRCWALSFYPAEITLT |
| | | WQRDGEDQTQDTELVETRPAGDGTFQKWAA |
| | | VVVPSGQEQRYTCHVQHEGLPKPLTLRWE |
| 23 | β2M | MIQRTPKIQVYSRHPAENGKSNFLNCYVSGFH |
| | | PSDIEVDLLKNGERIEKVEHSDLSFSKDWSFYL |
| | | LYYTEFTPTEKDEYACRVNHVTLSQPKIVKWD |
| | | RDMT |
| 24 | UCHT1 VH domain | EVQLQQSGPELVKPGASMKISCKASGYSFTGY |
| | | TMNWVKQSHGKNLEWMGLINPYKGVSTYNQK |
| | | FKDKATLTVDKSSSTAYMELLSLTSEDSAVYY |
| | | CARSGYYGDSDWYFDVWGAGTTVTVSS |
| 25 | UCHT1 VL domain | DIQMTQTTSSLSASLGDRVTISCRASQDIRNYL |
| | | NWYQQKPDGTVKLLIYYTSRLHSGVPSKFSGS |
| | | GSGTDYSLTISNLEQEDIATYFCQQGNTLPWT |
| | | FAGGTKLEIK |
| 26 | scDb short linker | GGGGS |
| 27 | scDb long linker | GGGGSGGGGSGGGGS |
| 28 | VH-VL linker in scFv | KLSGSASAPKLEEGEFSEARV |
| | 1-H02 | |
| 76 | Human CD3E | MQSGTHWRVLGLCLLSVGVWGQDGNEEMGGI |
| | (Uniprot P07766) | TQTPYKVSISGTTVILTCPQYPGSEILWQHN |
| | | DKNIGGDEDDKNIGSDEDHLSLKEFSELEQS |
| | | GYYVCYPRGSKPEDANFYLYLRARVCENCME |
| | | MDVMSVATIVIVDICITGGLLLLVYYWSKNR |
| | | KAKAKPVTRGAGAGGRQRGQNKERPPPVPNP |
| | | DYEPIRKGQRDLYSGLNQRRI |
| 79 | Positional | xx[Y]x[G/A/V][R/K/Q/H]x |
| | degenerate peptide | [H/Q/R][T/S][V/I/L] |
| | search motif | |
| 80 | Positional | xx[Y/V/I/L]x[G][R/K]x[H][T/S] |
| | degenerate peptide | [V/I/L] |
| | search motif | |

The invention will now be further described in the following non-limiting Examples with reference to the following drawings:

FIG. 1. Structural representation of the MAGE-A4²³⁰⁻²³⁹ T-cell epitope. The figure was made using the crystallographic coordinates of The Protein Databank POE: 1I4F visualized using Pymol v2.4.3.

FIG. 2. (A) Clone 1-H02 epitope specificity was assessed by positional Ala scanning mutagenesis across MAGE-A4²³⁰⁻²³⁹. The individual peptide variants were loaded onto recombinant biotinylated HLA-A0201 (pHLAs). The pHLAs were captured on neutravidin coated ELISA plates and incubated with 1-H02 phages at 10⁹ phages/well before detection with an anti-Fd antibody. (B) pHLA capture levels were controlled with the HLA-A2 specific mAb BB7.2.

FIG. 3. MAGE homologue peptides were synthesized and loaded onto recombinant biotinylated HLA-A0201 (pHLAs). The pHLAs were captured on neutravidin coated ELISA plates and incubated with (A) 1-H02 (C) AM2 and (D) AM15 phages at 10⁹ phages/well before detection with an anti-M13 antibody. (B) pHLA capture levels were controlled with the HLA-A2 specific mAb BB7.2.

FIG. 4. Principle of T-cell engager function in cancer immunotherapy. T cells eradicate target cells, such as cancer cells, by virtue of specifically recognizing intracellularly expressed antigens found as pHLA complexes displaying antigenic peptides on the target cell surface using their antigen-specific T-cell receptor (TCR). Upon productive TCR-pHLA interactions, the CD3 signaling units of the TCR activates the T cells and induce target cell killing. TCR-Like antibodies in the form of T-cell engagers (TCEs), such as the scDb, are (at least) bi-specific molecules recognizing both specific-pHLA and the CD3 signaling unit of T cells. Thus, TCEs generically redirect T cells towards target cells expressing specific pHLA and mediate polyclonal T-cell mediated target cell killing in a TCR independent manner activating the T cells through their naturally occurring CD3 signaling machinery.

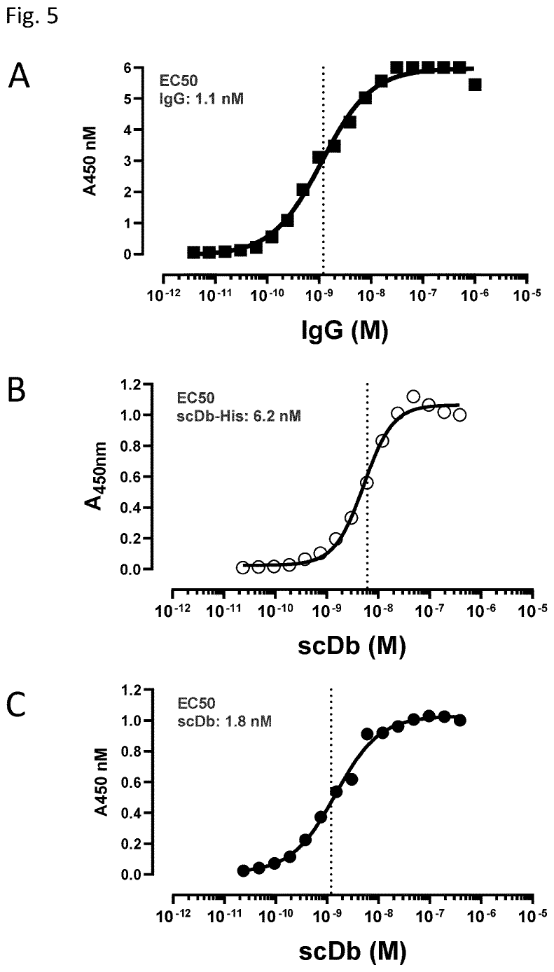

FIG. 5. 1-H02 was reformatted to full length hIgG1 (A) and scDb with (B) and without (C) a C-terminal linked His-tag. Soluble MAGE-A4-pHLA was captured on neutravidin coated ELISA wells and incubated with 1-H02 IgG or scDb at serial dilutions before detection with protL-HRP. Dotted lines indicate the EC₅₀ value (concentration at which half of maximal binding signal is achieved).

FIG. 6. Biotinylated pMHC was captured on neutravidin-coated sensor chips followed by injection of a 2-fold dilution series from 40 μM of 1-H02 scDb with (A) and without (B) a C-terminally linked His-tag. The equilibrium response was plotted against concentration to derive the equilibrium dissociation constant (kD) of the 1-H02. Dotted line indicates the kD based on fitting the response to saturation.

FIG. 7. Melting temperature of 1-H02 scDb was assessed by use of NanoDSF. The well-performing therapeutic antibody Trastuzumab (in the form of Fab) was included for comparison.

FIG. 8. TCE 1-H02 mediated T-cell redirection in co-culture of effector cells with 1-H02 and MAGE-A4 pulsed T2 cells assessed by (A) pan T-cell mediated cytotoxicity and (B) Jurkat NFAT mediated Luminescence. (C) TCE 1-H02 off-target profile assessed by reactivity towards MAGE-A4 peptide and a panel of peptides matching a regex motif based on the ala-scan in FIG. 2. Activation was measured by Luminescence after 16 h co-culture of Jurkat NFAT, 1-H02 and peptide pulsed T2 cells. Dotted line represents baseline activity as determined by Jurkat NFAT activation following co-culture non-pulsed T2s. Statistical analysis was completed using a one-way ANOVA (***p<0.001).

FIG. 9. 1-H02 was reformatted to scDb. (A) Jurkat activation assessed by Luminescence at serial dilutions of the target peptide was measured after 16 h co-culture of Jurkat NFAT, 1-H02 and peptide pulsed T2 cells. (B) Jurkat activation assessed by Luminescence at serial dilutions of TCE measured after 16 h co-culture of Jurkat NFAT, 1-H02 and MAGE-A4 positive (NCI-H1703) and MAGE-A4 negative (HCT116 and NCI-H441) cells.

FIG. 10. 1-H02 mediated re-direction of T cells was assessed by 72 h co-culture of PBMCs from six different donors, 1-H02, and peptide pulsed T2 cells. Following 72 h of co-culture, CD8+ T cells were assessed by flow cytometry analysis for surface expression of CD25 (A) and CD71 (B), proliferation (C), and intracellular expression of Granzyme (D). Different dots represent individual PBMC donors.

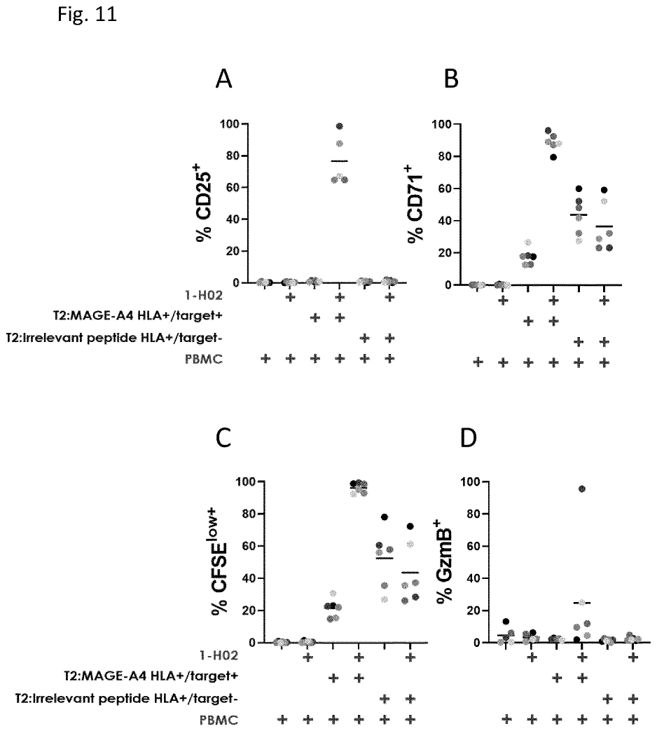

FIG. 11. 1-H02 mediated re-direction of T cells was assessed by 72 h co-culture of PBMCs from six different donors, 1-H02, and peptide pulsed T2 cells. Following 72 h of co-culture, CD4+ T cells were assessed by flow cytometry analysis for surface expression of CD25 (A) and CD71 (B), proliferation (C), and intracellular expression of Granzyme (D). Different dots represent individual PBMC donors.

FIG. 12. 1-H02 mediated re-direction of T cells was assessed by 72 h co-culture of PBMCs from six different donors, 1-H02, and peptide pulsed T2 cells. Following 72 h of co-culture, the specific lysis of target cells was assessed using 7-AAD by flow cytometry analysis. Different dots represent individual PBMC donors.

FIG. 13. 1-H02 mediated re-direction of T cells was assessed by 72 h co-culture of PBMCs from two different donors, 1-H02, and target positive (NCI-H1703 and A375) or target negative (HCT116) tumor cell lines. Following 72 h of co-culture, CD8⁺ T cells were assessed by flow cytometry analysis for surface expression of CD25 (A) and CD71 (B), proliferation (C) and intracellular expression of Granzyme B (D). Different dots represent individual PBMC donors.

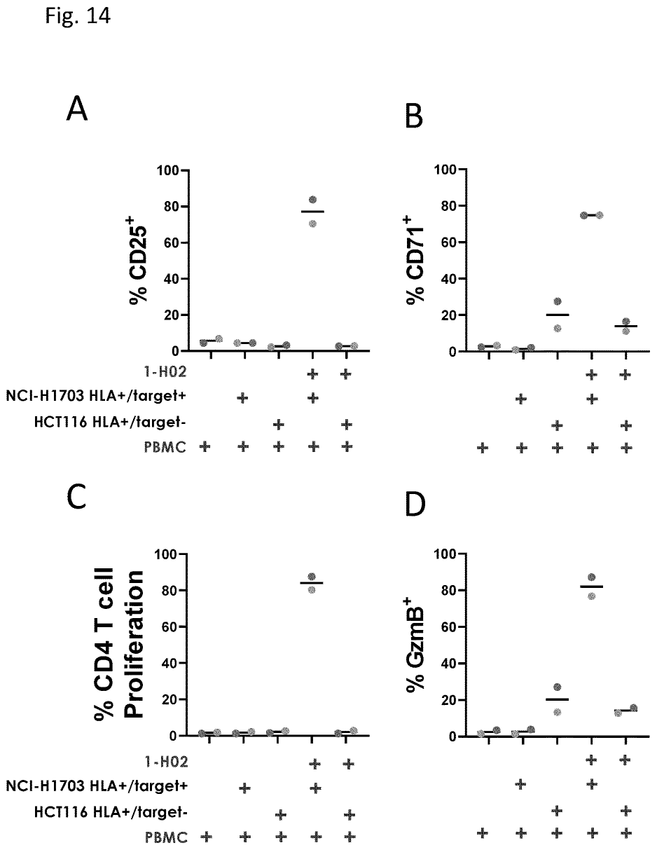

FIG. 14. 1-H02 mediated re-direction of T cells was assessed by 72 h co-culture of PBMCs from two different donors, 1-H02, and target positive (NCI-H1703 and A375) or target negative (HCT116) tumor cell lines. Following 72 h of co-culture, CD4⁺ T cells were assessed by flow cytometry analysis for surface expression of CD25 (A) and CD71 (B), proliferation (C) and intracellular expression of Granzyme B (D). Different dots represent individual PBMC donors.

FIG. 15. 1-H02 mediated re-direction of T cells was assessed by 72 h co-culture of PBMCs (A) or pan-T cells (B), 1-H02, and target positive (NCI-H1703, A375 or THP1) or target negative (HCT116 and Boleth) tumor cell lines. Following 72 h of co-culture the specific lysis of tumor cell lines representing solid tumor (A) or liquid tumor (B) was assessed using 7-AAD by flow cytometry analysis.

FIG. 16. TCE (scDb) AM2 (A), AM6 (B), AM15 (C), AMC8 (D), AMC9 (E) and AMC11 (F) off-target profile assessed by reactivity towards MAGE-A4 peptide and a panel of peptides matching a regex motif based on the ala-scan in FIG. 2. Activation was measured by Luminescence after 16 h co-culture of Jurkat NFAT, 1-H02 and peptide pulsed T2 cells. Dotted line represents baseline activity as determined by Jurkat NFAT activation following co-culture non-pulsed T2s. Statistical analysis was completed using a one-way ANOVA (***p<0.001).

FIG. 17. TCE (scDb) 1-H02 (A), AM2 (B), AM6 (C), AM15 (D), AMC8 (E), AMC9 (F) and AMC11 (G) off-target profile assessed by reactivity towards MAGE-A4 peptide and a panel of MAGE homologue peptides. Activation was measured by Luminescence after 16 h co-culture of Jurkat NFAT, 1-H02 and peptide pulsed T2 cells. Dotted line represents baseline activity as determined by Jurkat NFAT activation following co-culture non-pulsed T2s. Statistical analysis was completed using a one-way ANOVA with multiple comparisons controlled against no peptide (***p<0.001).

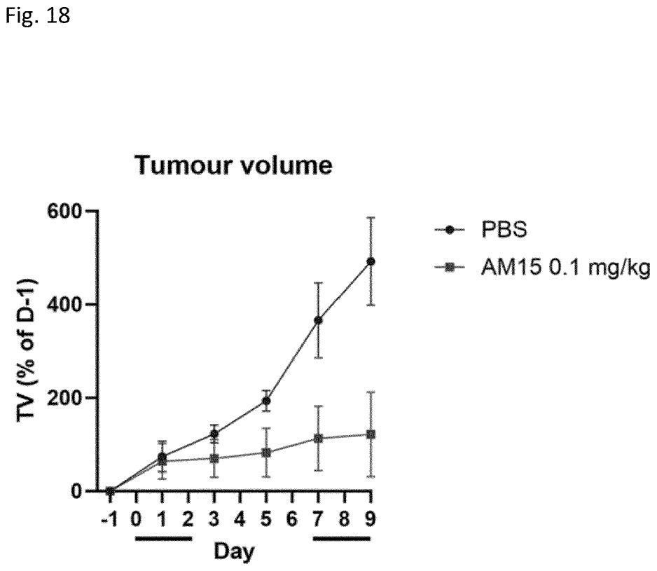

FIG. 18. In vivo tumour growth following administration of AM15 in CD34+ humanised mice. CD34+ humanised NSG mice (1 CD34+ HSC donor, 3 mice/group) were injected subcutaneously with 5×10⁶ A375 cells and tumour allowed to establish until an average of 89 mm³. Administration, via i.v. injection, on day 0-2 and day 7-9 with 0.1 mg/kg AM15 or PBS control.

### EXAMPLES

Throughout the Examples below, reference is made to Assays 1 to 8, which are described in detail above. The specific assay conditions and steps used in the Examples below are the specific conditions and steps disclosed in the above descriptions of Assays 1 to 8.

### Example 1: Binding Footprint and Specificity of HLA-A*02:01/MAGE-A4²³⁰⁻²³⁹ Antibodies

### Materials and Methods

### Antibodies

The nucleotide and amino acid sequences of the heavy and light variable domains of preferred HLA-A*02:01/MAGE-A4²³⁰⁻²³⁹ antibodies of the invention are shown in Table A. One such antibody is designated 1-H02, which was obtained via phage display.

An optimized version of a previously reported fully human naïve scFv library¹ was used for panning against specific soluble peptide-human leukocyte antigen (pHLA) complexes with the aim of isolating antibody (Ab) clones with specific binding towards the validated HLA-A*02:01/MAGE-A4²³⁰⁻²³⁹ T-cell epitope (FIG. 1)².

Three rounds of phage display selection were performed essentially as described³, before the libraries were screened for specific target binding hits. Screening was done using clonal phage capture ELISA against specific and unspecific pHLA targets. The response ratio of specific vs unspecific target binding was calculated to identify favourable primary clones, which were subsequently sequenced to reveal clonal identity.

These clones were subjected to further analysis and testing to look for appropriate and advantageous properties, and one clone, 1-H02, was identified as performing particularly well.

The CDR and framework regions of the light and heavy variable domains of 1-H02 are shown in Table A. This antibody, in the scFV format, comprised the VH and VL domains connected via a peptide linker (SEQ ID NO: 28).

Further preferred HLA-A*02:01/MAGE-A4²³⁰⁻²³⁹ antibodies of the invention shown in Table A are AM1, AM2, AM3, AM4, AM5, AM6, AM7, AM9, AM10, AM11, AM12, AM13, AM14, AM15, AM16, AM17, and AM18 (affinity matured clones). To increase target affinity of the 1-H02 parent clone, CDRs were targeted for diversification. NNK, NNC and NWW degenerate codons were used for amino acid mutagenesis of all six CDR loops, either by unbiased randomization of the whole loops or by rational randomization of particular residues informed by structure models. A total of 14 CDR libraries were constructed using custom designed genes containing degenerate codons. Libraries were transformed into electrocompetent cells and packaged as described (Høydahl et al, 2016). The selection and screening of the CDR libraries were performed essentially as described for the primary selection. These clones were subjected to further analysis and testing to look for appropriate and advantageous properties, and these clones were identified as performing particularly well.

Further preferred HLA-A*02:01/MAGE-A4²³⁰⁻²³⁹ antibodies of the invention shown in Table A are AMC1, AMC3, AMC4, AMC5, AMC6, AMC7, AMC8, AMC9, AMC10, AMC11, AMC12, and AMC14. Following the assessment of affinity matured clones for their ability to induce IFN-gamma release or T cell mediated killing, mutations from the previous affinity maturations were combined based on the location of the mutation (CDR) and ability to increase the ability to induce IFNg release or T cell mediated killing. These clones were subjected to further analysis and testing to look for appropriate and advantageous properties, and these clones were identified as performing particularly well.

The CDR and framework regions of the light and heavy variable domains of each of the antibodies of the invention are shown in Table A, together with the complete VH and VL domain sequences, and where appropriate the sequences of the antibody's full length heavy and light chains, scFv molecule, scDb molecule, and Fab VH/CH1 and VL/CL chains.

### Peptide Synthesis and HLA-A2 Peptide Loading

Peptides were synthesized at Genscript. Empty biotinylated HLA-A2 monomers (Tetramer Shop) were diluted in PBS to a concentration of 0.5 mg/ml and incubated with peptide (200 M) on ice for 30 min. The peptide loaded HLA molecules were further used in ELISA and SPR target binding assessments.

### Single Clone Phage ELISA

Assay 1 described above was performed. ELISA plates were coated with 5 μg/mL NeutrAvidin in PBS and incubated 16 h/4° C. (step (a)) and blocked with 5% skim milk powder in PBST for 1 h/room temperature (RT) (step (c)). Biotinylated pHLA variants were captured for 1 h/RT (step (e)), followed by addition and incubation of antibody, i.e. 10⁹ phages or dilution series starting at 385 nM (for scDb) or 250 nM (for IgG), as appropriate (step (g)). Bound phage particles were detected with an anti-Fd antibody (anti-M13-HRP (Amersham Biosciences)), whereas bound IgG1 and scDb was detected with HRP conjugated protein L (Genscript) (step (i)). Levels of immobilized pHLA were detected with the anti HLA-A2 specific mAb (Clone BB7.2 (AbCam)). pHLA, phage samples, IgG1, scDb, antibodies and protein L were all diluted in PBSTM. Plates were developed with TMB solution and read at 450 nm using a microplate reader (step (k)). Between each layer, the plates were washed 3× with PBST (steps (b), (d), (f), (h), and (j)).

### Results

### Specificity—Footprint

To examine the fine-specificity of clone 1-H02 towards the MAGE-A4²³⁰⁻²³⁹ peptide, positional alanine (Ala) scanning was performed using pHLA phage capture ELISA (as described above) towards the individual variants (Table C):

TABLE C
Positional alanine scanning
peptide sequences

| Peptide | Sequence | SEQ ID NO |
|---|---|---|
| WT MAGE- | GVYDGREHTV | 19 |
| A4²³⁰⁻²³⁹ | | |
| P1 | AVYDGREHTV | 31 |
| P2 | GAYDGREHTV | 32 |
| P3 | GVADGREHTV | 33 |
| P4 | GVYAGREHTV | 34 |
| P5 | GVYDAREHTV | 35 |
| P6 | GVYDGAEHTV | 36 |
| P7 | GVYDGRAHTV | 37 |
| P8 | GVYDGREATV | 38 |
| P9 | GVYDGREHAV | 39 |
| P10 | GVYDGREHTA | 40 |

As shown in FIG. 2, clone 1-H02 exhibited peptide dependent binding as specific mutations affected the binding sensitivity. Specifically, the Ab reactivity was abrogated with peptide variants P3, P5, P6, P8, P9 and P10, indicating that amino acids at positions 3, 5, 6, 8, 9 and 10 of WT MAGE-A4²³⁰⁻²³⁹ (SEQ ID NO: 19) were important for antibody binding. Canonically, HLA restricted peptides are 9mers, whereas WT MAGE-A4²³⁰⁻²³⁹ is a 10mer. For this reasons, amino acid positions 1 to 10 of WT MAGE-A4²³⁰⁻²³⁹ (SEQ ID NO: 19) may be referred to as positions −1 to 9. Using such an adjusted numbering system, the amino acids important for binding would be at positions termed 2, 4, 5, 7, 8 and 9, though for the avoidance of doubt, these are actually positions 3, 5, 6, 8, 9 and 10 of WT MAGE-A4²³⁰⁻²³⁹ (SEQ ID NO: 19).

Some peptide variants affect pHLA stability, the so-called anchor residues, whereas solvent exposed positions may be directly involved in the Ab reactivity. These exposed positions correspond to amino acid positions 3, 4, 5, 6, and 8 in the MAGE-A4²³⁰⁻²³⁹ peptide sequence (SEQ ID NO: 19). Thus, the overall, result showed that clone 1-H02 exhibited restricted positional binding dependency towards 4 of the 5 solvent exposed residues (those at positions 3, 5, 6, and 8 of SEQ ID NO: 19) suggesting a central binding mode and high degree of specificity.

The same analysis was performed using many other of the antibodies disclosed in Table A, including but not limited to AM2, AM6, and AM15, with the peptide-dependent binding observed being essentially the same as 1-H02 (data not shown).

### Specificity—vs MAGE-A4 Homologues

To further assess the specificity of 1-H02, a pHLA phage capture ELISA was conducted (as described above) using a panel of MAGE-A4²³⁰⁻²³⁹ homologue peptides (Table D) loaded onto soluble biotinylated HLA-A*02:01. The homologue peptides chosen were those with the closest sequence similarity to the MAGE-A4²³⁰⁻²³⁹ sequence (SEQ ID NO: 19). The amino acid positions indicated are the actual positions in the 10 mer (i.e. positions 1 to 10), which would equate to positions −1 to 9 using the canonical HLA restricted peptide numbering system.

TABLE D
MAGE-A4²³⁰⁻²³⁹ homologue peptides

| | | | | | | | | | | | SEQ |
|---|---|---|---|---|---|---|---|---|---|---|---|
| | POSITION | | | | | | | | | | ID |
| Peptide | 1 | 2 | 3 | 4 | 5 | 6 | 7 | 8 | 9 | 10 | NO |
| MAGE-A4 | G | V | Y | D | G | R | E | H | T | V | 19 |
| MAGE-A1 | E | V | Y | D | G | R | E | H | S | A | 41 |
| MAGE-A2 | E | V | F | E | G | R | E | D | S | V | 42 |
| MAGE-A3 | E | V | F | E | G | R | E | D | S | I | 43 |
| MAGE-A8 | G | L | Y | D | G | R | E | H | S | V | 44 |
| MAGE-A9 | G | V | Y | V | G | K | E | H | M | F | 45 |
| MAGE-A10 | G | L | Y | D | G | R | E | H | L | I | 46 |
| MAGE-A11 | G | V | Y | A | G | R | E | H | F | L | 47 |
| MAGE-A12 | E | A | S | D | G | R | E | D | S | V | 48 |
| MAGE-B2 | G | V | Y | D | G | E | E | H | S | V | 49 |
| MAGE-B4 | G | I | Y | D | G | K | R | H | L | I | 50 |
| MAGE-B6 | G | I | Y | D | G | I | L | H | S | I | 51 |
| MAGE-B10 | G | L | Y | D | G | I | E | H | F | M | 52 |
| MAGE-C2 | G | V | Y | A | G | R | E | H | F | V | 53 |

As shown in FIG. 3A, the only cross-reactive peptide was from MAGE-A8, which displayed a greatly reduced binding level as compared to the MAGE-A4²³⁰⁻²³⁹ peptide. The MAGE-A4 and MAGE-A8 peptides differ in two positions, of which only one is solvent exposed and thus accessible for putative ligand binding. The difference in binding strength can therefore likely be assigned to this position 9 T to S substitution.

Very low background binding was observed on an irrelevant control peptide, and the level of the various pHLA captured in the ELISA wells were validated by binding of mAb BB7.2 which binds all HLA-A2 peptide complexes (FIG. 3B). These results indicate that 1-H02 exhibits HLA-A2-restricted peptide binding with high specificity, as it bound with high binding sensitivity only to the MAGE-A4²³⁰⁻²³⁹ peptide.

The same analysis was performed using the AM2 and AM15 antibodies, with the results shown in FIGS. 3C and 3D, respectively. The same analysis was performed using many other antibodies disclosed in Table A, with the same binding pattern observed as for 1-H02, AM2 and AM15, i.e. specific binding to HLA-A*02:01/MAGE-A4²³⁰⁻²³⁹ over HLA-A*02:01 restricted peptides from MAGE-A4 homologues (data not shown).

### Example 2: Functional Characteristics of HLA-A*02:01/MAGE-A4²³⁰⁻²³⁹ Antibodies in T-Cell Engager (TCE) Format

### Materials and Methods

### Antibodies

For targeting of T cells to the HLA-A*02:01/MAGE-A4²³⁰⁻²³⁹ antigen, the present inventors designed bispecific T-cell engager antibodies in the form of a single chain diabody (scDb) based on each of the specific clones of Table A and which includes a CD3 binding domain (FIG. 4).

Briefly, the scDb comprises a first antigen binding domain with a first specificity for MAGE-A4²³⁰⁻²³⁹ pHLA comprising an antigen variable light (VLA) and a variable heavy (VHA) domain, and a second antigen binding domain with a second specificity for CD3 comprising an antigen variable light (VLB) and variable heavy (VHB) domain, wherein the domains are fused by peptide linkers and are in the following order: VLA-VHB-VLB-VHA-His-tag. The CD3 antigen binding domain possess the same specificity as the anti-human CD3 antibody UCHT1, as described^{4,5.}

Synthetic genes encoding the scDb were constructed and cloned into expression vector for transient expression in CHO cells. The recombinant scDb was expressed and purified by affinity chromatography on a HisTrap™ FF Crude column. After concentration and dialysis against PBS buffer, content and purity of the purified protein was assessed by SDS-PAGE and analytical size exclusion chromatography.

The scDb without the His-tag was also prepared (in the same manner, though purified by affinity chromatography on Protein L resin). The human IgG1 format was also constructed by linking the VH and VL domains to the IgG1 constant domains. Human IgG1 was expressed as described for the scDb and purified by affinity chromatography on Protein A.

By way of example, the amino acid sequence of the scDb 1-H02 molecule (without the His-tag) is shown in SEQ ID NO: 30, which consists of the following sequences reading in the N to C direction:

TABLE E
scDb 1-H02
TABLE E

| DOMAIN | | SEQUENCE | SEQ ID NO: |
|---|---|---|---|
| VLA | Light FR1 | DIRVTQSPSSLSASVGDRVTITCRAS | 15 |
| (vs. HLA- | Light CDR1 | QSISSY | 8 |
| A*02:01/MAGE- | Light FR2 | LNWYQQKPGKAPKLLIY | 16 |
| A4²³⁰⁻²³⁹) | Light CDR2 | AAS | 9 |
| | Light FR3 | SLQSGVPSRFSGSGSGTDFTLTISSLQP | 17 |
| | | EDFATYYC | |
| | Light CDR3 | QQSYSTPYT | 10 |
| | Light FR4 | FGQGTKLEIK | 18 |
| Short Linker | | GGGGS | 26 |
| VHB | VH | EVQLQQSGPELVKPGASMKISCKASGYS | 24 |
| (vs. CD3) | | FTGYTMNWVKQSHGKNLEWMGLINPYK | |
| | | GVSTYNQKFKDKATLTVDKSSSTAYMEL | |
| | | LSLTSEDSAVYYCARSGYYGDSDWYFD | |
| | | VWGAGTTVTVSS | |
| Long Linker | | GGGGSGGGGSGGGGS | 27 |
| VLB | VL | DIQMTQTTSSLSASLGDRVTISCRASQDI | 25 |
| (vs. CD3) | | RNYLNWYQQKPDGTVKLLIYYTSRLHSG | |
| | | VPSKFSGSGSGTDYSLTISNLEQEDIATY | |
| | | FCQQGNTLPWTFAGGTKLEIK | |
| Short Linker | | GGGGS | 26 |
| VHA | Heavy FR1 | QVQLVQSGAEVKKPGASVKVSCKVS | 11 |
| (vs. HLA- | Heavy CDR1 | GYTLTELS | 5 |
| A*02:01/MAGE- | Heavy FR2 | MHWVRQAPGKGLEWMGG | 12 |
| A4²³⁰⁻²³⁹) | Heavy CDR2 | FDPEDGET | 6 |
| | Heavy FR3 | IYAQKFQGRVTMTEDTSTDTAYMELSSL | 13 |
| | | RSEDTAVYYC | |
| | Heavy CDR3 | ATDQGSSWGFY | 7 |
| | Heavy FR4 | WGQGTLVTVSS | 14 |

In the scDb format (without the His-tag) of each of the other antibodies of the invention that were prepared (i.e. scDb AM15, scDb, AM2, scDb, AMC9, scDb AM6, scDb AMC8, scDb AMC11, scDb AM11, scDb AM3, and so on for each antibody of Table A), the same long and short linker sequences were present (those shown in Table E), and the same VHB and VLB sequences (vs. 003) were present (those shown in Table E). The VHA and VLA CDR and framework sequences that were present are those shown in Table A for the given antibody.

For instance the amino acid sequence of the scDb AM15 molecule (without the His-tag) is shown in SEQ ID NO: 148, which consists of the following sequences reading in the N to C direction:

TABLE E
TABLE E: scDb AM15

| DOMAIN | | SEQUENCE | SEQ ID NO: |
|---|---|---|---|
| VLA | Light FR1 | DIQLTQSPSSLSASVGDRVTITCRAS | 102 |
| (vs. HLA- | Light CDR1 | QSISSY | 8 |
| A*02:01/MAGE- | Light FR2 | LNWYQQKPGKAPKLLIY | 16 |
| A4²³⁰⁻²³⁹ | Light CDR2 | AAS | 9 |
| | Light FR3 | GLQSGVPSRFSGSGSGTDFTLTISSLQ | 109 |
| | | PEDFATYYC | |
| | Light CDR3 | QQSYSTPFT | 96 |
| | Light FR4 | FGPGTKVDIK | 112 |
| Short Linker | | GGGGS | 26 |
| VHB | VH | EVQLQQSGPELVKPGASMKISCKASGYS | 24 |
| (vs. CD3) | | FTGYTMNWVKQSHGKNLEWMGLINPYK | |
| | | GVSTYNQKFKDKATLTVDKSSSTAYMEL | |
| | | LSLTSEDSAVYYCARSGYYGDSDWYFD | |
| | | VWGAGTTVTVSS | |
| Long Linker | | GGGGSGGGGSGGGGS | 27 |
| VLB | VL | DIQMTQTTSSLSASLGDRVTISCRASQDI | 25 |
| (vs. CD3) | | RNYLNWYQQKPDGTVKLLIYYTSRLHSG | |
| | | VPSKFSGSGSGTDYSLTISNLEQEDIATY | |
| | | FCQQGNTLPWTFAGGTKLEIK | |
| Short Linker | | GGGGS | 26 |
| VHA | Heavy FR1 | QVQLVQSGAEVKKPGASVKVSCKVS | 11 |
| (vs. HLA- | Heavy CDR1 | GYTLTELS | 5 |
| A*02:01/MAGE- | Heavy FR2 | MHWVRQAPGKGLEWMGG | 12 |
| A4²³⁰⁻²³⁹ | Heavy CDR2 | FDPEDGET | 6 |
| | Heavy FR3 | IYAQKFQGRVTMTEDTSTDTAYMELSSL | 13 |
| | | RSEDTAVYYC | |
| | Heavy CDR3 | ATDQGSSWGFY | 7 |
| | Heavy FR4 | WGQGTLVTVSS | 14 |

### Binding Analysis by SPR

Assay 6 above was performed. SPR was conducted using a Biacore S200 (GE Healthcare). NeutrAvidin (10 μg/mL in 10 mM sodium acetate, pH 5.0) was coupled onto a CM3 series S sensor chip to 500 response units (RU) by amine coupling before capture of 40-100 RU of soluble, recombinant, biotinylated pHLA (1 μg/mL). Kinetics and affinity of scDb molecules was determined using single cycle kinetics method. All samples were diluted in PBS and run at 30 μL/min at 25° C. Data was fitted to a 1:1 Langmuir binding model after buffer subtraction and NeutrAvidin-reference-cell subtraction using the S200 Evaluation Software v1.0.

Thermostability Analysis by nanoDSF

Assay 7 above was performed. Purified Fab and scDb molecules were diluted to 0.04-0.46 mg/ml in PBS and 10 μL were transferred to glass capillaries (NanoTemper) in triplicates. Samples were subjected to a temperature from 20° C. to 95° C., with 1° C./min increments using a Prometheus nanoDSF (NanoTemper). An excitation wavelength of 295 nm was used, and emission was measured at 330 nm and 350 nm. Data was collected and analyzed using AB-Protein PR.ThermControl V2.12 to determine melting temperatures.

### Results

### Comparison of Ab Format Binding

For validation of the functional folding and integrity of the scDb antibodies, pHLA target binding was assessed by ELISA and compared with the 1-H02 hIgG1 (as described above in Example 1—Assay 1).

As shown in FIGS. 5A and 5B, the full length hIgG1 and scDb-His 1-H02 bound to the biotinylated HLA-A*02:01/MAGE-A4²³⁰⁻²³⁹ complex in a concentration dependent manner with a EC₅₀ value of 1.1 nM and 6.2 nM, respectively. As the His-tag, which was linked C-terminally to the VH domain of the 1-H02 molecule, potentially could interfere with the target binding activity, an untagged version of the scDb was made and compared head-to-head with the His-tagged molecule for target binding ability by ELISA. The scDb 1-H02 without the His-tag also bound to the biotinylated HLA-A*02:01/MAGE-A4²³⁰⁻²³⁹ complex in a concentration dependent manner with a EC₅₀ value of 1.8 nM (FIG. 5C). Thus, results showed that the IgG format and both 1-H02 scDb variants bound towards the HLA-A*02:01/MAGE-A4²³⁰⁻²³⁹ target with activities in the same range, but with slightly increased target sensitivity for the scDb molecule without the His-tag as compared to the His-tagged variant.

The assay was repeated with the full suite of antibodies shown in Table A, each in the scDb format described above. The results are shown in Table H. The data for the AM antibodies (and 1-H02) and AMC antibodies was obtained from two separate experiments where the intraexperimental difference was within an acceptable range. Likewise, the intraexperimental difference between these experiments and the above earlier assay of 1-H02 was within an acceptable range.

TABLE H
scDb antibody EC₅₀ value (concentration at which half of
maximal soluble target binding signal is achieved)

| | Binding to soluble target |
|---|---|
| Antibody | EC50 |
| 1-H02 | 2.79 nM |
| AM1 | 0.36 nM |
| AM2 | 0.32 nM |
| AM3 | 0.48 nM |
| AM4 | 0.28 nM |
| AM5 | 1.03 nM |
| AM6 | 2.32 nM |
| AM7 | 0.63 nM |
| AM9 | 0.77 nM |
| AM10 | 1.15 nM |
| AM11 | 0.17 nM |
| AM12 | 0.12 nM |
| AM13 | 0.43 nM |
| AM14 | 0.10 nM |
| AM15 | 1.36 nM |
| AM16 | 1.85 nM |
| AM17 | 0.30 nM |
| AM18 | 0.51nM |
| AMC1 | 0.45 nM |
| AMC3 | 0.35 nM |
| AMC4 | 0.36 nM |
| AMC5 | 0.32 nM |
| AMC6 | 0.61 nM |
| AMC7 | 0.41 nM |
| AMC8 | 0.88 nM |
| AMC9 | 0.62 nM |
| AMC10 | 0.46 nM |
| AMC11 | 0.81 nM |
| AMC12 | 0.25 nM |
| AMC14 | 0.47 nM |

### Affinity

Assay 6 above was performed. To determine the apparent affinity of the 1-H02 candidate for HLA-A*02:01/MAGE-A4²³⁰⁻²³⁹, surface plasmon resonance (SPR) binding analysis was used in which the biotinylated pHLA complex was captured on neutravidin-coated SPR sensor chips followed by injection of various dilutions of 1-H02 scDb (Assay 6-1 above). As shown in FIG. 6, the sensorgrams of SPR analysis revealed affinities in the higher nM range for both scDb 1-H02 molecules (i.e. with (A) and without (B) the C-terminal linked His-tag). Again, the molecule without the His-tag showed a slightly increased affinity of 370 nM as compared to the his-tagged molecules with an affinity of 857 nM. Very low to none binding was observed to HLA-A2 loaded with two irrelevant peptides.

Assay 6-1 above was performed with the following antibodies in the Fab format, with the following affinities determined: AMC9 (15.8 nM); AM2 (86.3 nM); AM15 (148 nM); AM10 (325 nM) and 1-H02 (1410 nM).

Assay 6-2 above was performed with the following antibodies in the scDb format (without a His-tag), with the following affinities determined: AM11 (3.2 nM); AM13 (7.0 nM); AM14 (0.44 nM); and AM17 (10.8 nM).

The results demonstrate that the antibodies binds relatively weakly to the target.

Thermostability Analysis by nanoDSF

Assay 7 above was performed. The thermostability of 1-H02 scDb was assessed by determining its melting temperature (Tₘ) using nanoDSF (FIG. 7). The melting temperature is measured by thermal unfolding and is defined as the temperature where 50% of the molecules is unfolded. The Tₘ value of 1-H02 scDb was determined to be 68.1° C. The antigen binding Fab unit of the well-performing therapeutic antibody Trastuzumab was included as control, in a format (Fab) which normally has higher thermostability due to the CH1-CL pair^{6,7}. The same analysis was performed using all of the other specific antibodies disclosed in Table A, and similar thermal stabilities were observed as for 1-H02; All antibodies were determined to have a melting temperature in the range of 68.1° C. to 70.4° C. (data not shown).

### Example 3: Specific Redirection of T-Cell Activity

### Materials and Methods

### Cancer Cell Lines and Culture Conditions

Cancer cell lines were purchased cell banks and cultured as per recommended conditions and media. Table F details the source of cancer cell lines and the media in which cells were cultured.

TABLE F
Details of cancer cell lines and culture media used in cell based assays

| Cell line | Source | Culture Media |
|---|---|---|
| T2 | ATCC (CRL-1992) | RPMI/10% FCS/1% Penicillin- |
| | | Streptomycin (P/S) |
| NCI-H1703 | ATCC (CRL-5889) | RPMI/10% FCS/1% P/S |
| HCT116 | ATCC (CCL-247) | McCoy's 5a Medium Modified/10% |
| | | FBS/1% P/S |
| NCI-H441 | ATCC (HTB-174) | RPMI/10% FCS/1% P/S |
| A375 | ATCC (CRL-1619) | DMEM/10% FBS/1% P/S |
| THP1 | ATCC (TIB-202) | RPMI/10% FBS/1% P/S/0.05 mM |
| | | Mercaptoethanol |
| Boleth | Oslo University | RPMI/10% FBS/1% P/S |
| | Hospital | |
| Jurkats | BPS Biosciences | RPMI/10% FBS/1% P/S |

PBMCs were isolated from Buffycoats from healthy donors. Briefly, the buffy coat was diluted 1:1 with PBS/2% FBS, layered onto Lymphoprep and centrifuged at 500 g for 30 minutes. Following centrifugation, PBMCs were harvested from the interface between Lymphoprep and plasma and washed with PBS. Stocks of PBMCs where generated and stored in liquid nitrogen.

Pan T cells were isolated from whole PBMCs using ‘EasySep™ Human T cell isolation kit’. Briefly, PBMCs were resuspended in StemCell buffer kit at a concentration of 50 million/ml and 50 μl of isolation cocktail was added per 1 ml of cell suspension. Cells were gently mixed and then incubated for 5 minutes. Following the incubation, 40 μl of pre-vortexed RapidSpheres™ were added per 1 ml of cell suspension and gently mixed. Stem cell buffer was then added to the cell suspension to ensure a final volume of 2.5 ml. The cell suspension was then docked to the magnetic stand and incubated at room temperature for 3 mins. Following the 3 minutes incubation, the supernatant was removed and transferred to a new tube containing 10 ml assay media (RPMI/10% FBS/1% P/S). The supernatant was when centrifuged to pellet the pan T cells contained within. The purity of the pan T cell population was assessed by flow cytometry using anti-human CD3 antibody.

The assay media in all assays containing PBMCs or pan T cells was RPMI/10% FBS/1% P/S

### Ala-Scan, Degenerate Search Motifs and Risk Panel Peptides

Peptides of human genomic origin having high levels of sequence similarity to MAGE-A4²³⁰⁻²³⁹ were identified via two routes. First, searches within human proteome databases were made to identify amino acid sequences with similarity to the MAGE-A4²³⁰⁻²³⁹ sequence. Secondly, based on the binding profile of 1-H02 in the MAGE-A4 peptide ala-scan (Table C, FIG. 2), positional degenerate peptide search motifs were constructed, which were based on the MAGE-A4²³⁰⁻²³⁹ sequence with positional allowed variation. All of the Ala substitutions were used as basis for the motifs. The first motif, xx[Y]x[G/A/V][R/K/Q/H]x[H/Q/R][T/S][V/I/L] allowed for any amino acid (represented by “x”) in position 1, 2, 4 and 7, amino acids with similar biophysical properties in positions 5, 6, 8, 9 and 10 (shown in brackets), and no change in position 3 [Y]. In the second motif, xx[YN/1/L]x[G][R/K]x[H][T/S][V/I/L], variation in position 3 was introduced, whereas position 5 and 8 were locked, again “x” represents any amino acid.

These motifs were used for scanning for amino acid sequence matches against the protein sequence data bases using ScanProsite (https://prosite.expasy.org/scanprosite/). Default settings were used, and taxonomy filters were restricted to Homo sapiens. All hits were further applied to The Immune Epitope Database (IEDB) epitope analysis by scanning the peptide sequences for amino acid patterns indicative of HLA-A*02:01 binding. Peptide hits with the best predicted affinities (rank 1-22), determined using https://tools.iedb.org/mhci/), shown in Table G below, were synthesized for use in risk assessment analysis. The amino acid positions indicated are the actual positions in the 10mer (i.e. positions 1 to 10), which would equate to positions −1 to 9 using the canonical HLA restricted peptide numbering system.

TABLE G
MAGE-A4²³⁰⁻²³⁹ related risk peptides

| | | | | | | | | | | | Predicted | SEQ |
|---|---|---|---|---|---|---|---|---|---|---|---|---|
| | POSITION | | | | | | | | | | Affinity | ID |
| Peptide | 1 | 2 | 3 | 4 | 5 | 6 | 7 | 8 | 9 | 10 | (nM) | NO |
| MAGE-A4 | G | V | Y | D | G | R | E | H | T | V | 560.08 | 19 |
| TNC18 | L | L | Y | A | G | H | V | Q | T | V | 16.77 | 54 |
| STON1 | F | S | V | A | G | K | I | H | T | V | 75.24 | 55 |
| TSEAR | A | L | V | D | G | R | W | H | T | L | 83.12 | 56 |
| C2CD3 | L | A | L | L | G | R | T | H | S | V | 116.93 | 57 |
| GP101 | L | L | Y | N | V | K | R | H | S | L | 332.14 | 58 |
| ACPH | T | Q | Y | G | G | Q | Y | R | T | V | 337.79 | 59 |
| CHLE | S | L | Y | E | A | R | N | R | T | L | 373.88 | 60 |
| ALKB2 | V | Q | V | F | G | K | W | H | S | V | 665.49 | 61 |
| P210L | K | L | Y | T | A | Q | G | R | T | L | 1063.87 | 62 |
| RHG32 | A | A | L | Q | G | K | F | H | T | I | 2025.88 | 63 |
| BIRC1 | M | V | Y | F | G | K | N | Q | S | L | 2620.95 | 64 |
| AGO3 | L | Q | Y | G | G | R | N | R | T | V | 3052.74 | 65 |
| MADD | S | T | I | F | G | K | A | H | S | L | 3199.03 | 66 |
| ASML | S | R | L | S | G | R | E | H | S | V | 3439.62 | 67 |
| USH2A | L | V | Y | Q | G | K | E | Q | S | V | 3554.52 | 68 |
| SL9A4 | N | M | Y | Q | V | R | Q | R | T | L | 4468.92 | 69 |
| TDRD9 | F | E | V | E | G | K | P | H | S | V | 6127.72 | 70 |
| MON2 | K | A | Y | S | V | Q | G | Q | S | V | 6826.16 | 71 |
| SPB11 | K | W | Y | Q | A | R | L | Q | T | V | 8278.19 | 72 |
| LRC36 | G | S | Y | A | G | K | I | H | S | I | 8628.35 | 73 |
| DRB5 | T | V | Y | P | A | R | T | Q | T | L | 9723.4 | 74 |
| SC6A3 | L | G | Y | M | A | Q | K | H | S | V | 9727.3 | 75 |

### TCE Mediated Cytotoxicity Assay

Assay 5 above was performed. The capacity of the 1-1H02 scDb to induce T-cell mediated cytotoxicity was assessed using co-cultures of target cells (“T”): CFSE (Biolegend) labelled target positive (NCI-H-1703, A375 and THP1) or negative (HCT116 and Boleth) cancer cell lines or peptide pulsed T2 cells (50 μM peptide overnight (16 h) pulsing, excess peptides washed away prior to co-culture) with effector cells (“E”): PBMCs, or pan T cells isolated from PBMCs, at an E:T ratio of 1:1. Solutions of 1-1H02 covering the concentration range of 0.01-1000 ng/ml were added to relevant wells,

Following 72 h co-culture, assay supernatants and cells were harvested from the assay plates and 7-AAD viability staining solution (BioLegend) was added. Samples were then analysed by flow cytometry.

### Jurkat NFAT Activation Assay

Assay 2 above was performed. The capacity of 1-H02, AM2, AM6, AM15, AMC8, AMC9 and AMC11 TCE antibody (i.e. scDb antibody) to induce CD3-mediated activation of Jurkat NFAT cells was assessed using co-cultures of Jurkat NFAT cells, TCE antibody and either target positive or negative cancer cell lines or peptide pulsed T2 cells. In brief, T2 cells or cancer (i.e. “target”) cells were cultured harvested, washed, re-suspended in cell culture medium, diluted and seeded at 50000 cells/well on day 0 (cancer cell lines) or 25000 cells/well on day 1 (T2) in cell culture medium in round bottom 96-well cell culture plates (steps (a) to (f)). Subsequently on day 1, Jurkat NFAT reporter cells were cultured harvested, washed, re-suspended in cell culture medium, diluted (steps (g) to (j)), then added to the target cells at a E:T ratio of 1:1 (step (k)). antibody was diluted in culture medium and added to the co-culture (0.2 nM to 200 nM)—controls comprised culture medium alone—to a final volume of 200 μl per well (steps (l) and (m). When Jurkat NFAT cells were added to T2 cells, the following diluted peptides were added to the co-culture: i) MAGE-A4²³⁰⁻²³⁹: 50 μM (FIGS. 8 A&B), ii) MAGE-A4²³⁰⁻²³⁹ and other peptides: 100 nM (FIGS. 8C and 16A to 16F), iii) MAGE-A4²³⁰⁻²³⁹: range 600 pM to 10 pM (FIG. 9A) (steps (n) to (p)), iv) of MAGE-A4²³⁰⁻²³⁹ homologue peptides of Table D (FIG. 17A to 17G). Cells were incubated for 16 h at 37° C. in a humidified incubator (step (q)). D-Luciferin, Monopotassium Salt (Promega) was diluted at 1:10 in Milli-Q water (step (r)).

In wells of a white 96-well plate, the D-Luciferin Monopotassium Salt (Promega) and cell co-culture were prepared at a ratio of 1:3 (step (s)). Where cells were seeded in step e) in white 96-well plates, this comprised adding 50 μl of luciferase substrate to the 150 μl of co-culture. Where non-white 96-well plates were used in step e) this comprised instead, in step (q), pelleting the co-culture plates at 300 g for 5 min, discarding excess cell culture supernatant, and re-suspending pelleted cells in remaining cell culture supernatant (100 μl) then in step (s), adding 25 μl per well of D-Luciferin Monopotassium Salt (Promega) and 75 μl per well of cell co-culture.

Luminescence was detected using Varioscan LUX (Thermo Fisher) (step (t)).

### Results

To initially explore the ability of 1-H02 scDb to mediate T-cell re-directed target cell killing and T-cell activation, 1-H02 scDb was co-cultured with MAGE-A4²³⁰⁻²³⁹ peptide pulsed T2 cells. Both dose-dependent killing of MAGE-A4²³⁰⁻²³⁹ pulsed T2 assessed via 7-AAD flow cytometry analysis (FIG. 8A) and activation of NFAT Jurkat cells (FIG. 8B) was observed. No response was observed when 1-H02 was co-cultured with non-pulsed T2 cells. Further, 1-H02 mediated cytotoxicity and Jurkat activation in co-culture with peptide pulsed T2 cells exhibit the same sensitivity as seen by the dose responses in FIGS. 9A and 9B, validating the Jurkat NFAT activation assay a good proxy of T-cell activity/killing.

Based on the Ala-scan in FIG. 2, regex motifs were created and used for scanning for matches against the prosite protein sequence data base (restricted to human proteins). Sequences matching the regex motifs may potentially represent risk peptides to the extent that they can be presented on HLA-A2 and induce TCE mediated T-cell activation. To further determine the specificity of the antibodies of the invention 1-H02, AM2, AM6, AM15, AMC8, AMC9 and AMC11, Assay 2 above was performed. Risk peptides matching the regex motif were ordered and pulsed onto T2 cells. Following 16 h co-culture of peptide pulsed T2 cells, 1-H02 and Jurkat NFAT cells, Jurkat activation was only observed when T2 cells were pulsed with MAGE-A4²³⁰⁻²³⁹ peptide (***p<0.001), with all risk peptides comparable to non-pulsed T2 cells (FIG. 8C). This analysis was also performed with antibodies AM2, AM6, AM15, AMC8, AMC9 and AMC11. Again, following 16 h co-culture of peptide pulsed T2 cells, antibody and Jurkat NFAT cells, Jurkat activation was only observed when T2 cells were pulsed with MAGE-A4²³⁰⁻²³⁹ peptide (***p<0.001), with all risk peptides comparable to non-pulsed T2 cells (FIG. 16A to 16F).

To further determine the specificity of the antibodies of the invention 1-H02, AM2, AM6, AM15, AMC8, AMC9 and AMC11, Assay 2 above was performed again, this time with the MAGE-A4²³⁰⁻²³⁹ homologue peptides of Table D being ordered and pulsed onto T2 cells. Following 16 h co-culture of peptide pulsed T2 cells, antibody and Jurkat NFAT cells, Jurkat activation was only observed when T2 cells were pulsed with i) MAGE-A4²³⁰⁻²³⁹ peptide, and to a lesser extent ii) MAGE-A8 peptide (FIG. 17A to 17G).

Jurkat NFAT cell activation was assessed following co-culture with peptide pulsed T2 cells or cancer cell lines and 1-H02 (Assay 2 above). Dose response curves were measured following 16 h co-culture of Jurkat cells, 1-H02 on T2 cells pulsed with different doses of MAGE-A4²³⁰⁻²³⁹ peptide (FIG. 9A). Similarly, activation was observed with co-culture with Jurkat NFAT, titrations of 1-H02, and target positive (NCI-H1703) cancer cell lines, but not on target negative (HCT116 and NCI-H441) cancer cell lines (FIG. 9B).

### Example 4: T-Cell Activation, Differentiation, Proliferation and Cytotoxicity

### Materials and Methods

### T-Cell Activation, Differentiation and Proliferation

Assay 3 and Assay 4 above were performed. The capacity of the 1-H02 scDb to induce CD3-mediated (i.e. T-cell mediated) activation, differentiation and proliferation of CD4+ and CD8+ T cells was assessed using co-cultures of target cells (“T”): HLA-A*02:01/MAGE-A4²³⁰⁻²³⁹ positive cancer cells (cell lines NCI-H1703 and A375) or HLA-A*02:01/MAGE-A4²³⁰⁻²³⁹ negative cancer cell lines (HCT116) or MAGE-A4²³⁰⁻²³⁹ peptide pulsed T2 cells (50 μM peptide overnight (16 h) pulsing, excess peptides washed away prior to co-culture) with effector cells (“E”) PBMCs at an E:T ratio of 1:1 for 72 h. A single 1-H02 concentration of 100 ng/ml was added for T-cell activation, differentiation and proliferation.

Following 72 h of co-culture (37° C.), multi-colour flow cytometry staining and analysis was performed.

For T-cell activation, extracellular staining was performed with anti-human antibodies from BioLegend: APC-CD25 (clone BC96), and PercpCy5.5-CD71 (clone CY1G4). For T-cell differentiation, intracellular staining was performed with anti-human antibodies from BioLegend: PE-Granzyme B (clone QA18A28). Live cells were assessed using Zombie Yellow fixable viability kit (BioLegend). Appropriate FMOs were used to exclude background staining.

For proliferation, PBMCs were labelled with CFSE (Carboxyfluorescein succinimidyl ester, BioLegend) prior to co-culture with target cells.

Extracellular staining was also performed with anti-human antibodies for CD3 (BV421-CD3 (clone OKT3)), CD4 APC-Cy7-CD4 (clone OKT4) and CD8 PECy7-CD8 (clone SK1) as previously described.

### T-Cell Mediated Cytotoxicity

Assay 5 above was performed to assess the capacity of the following antibodies of Table A (scDb format) to induce T-cell mediated cytotoxicity against target-positive cancer cells (NCI-H1703), i.e. target cells: 1-H02, AM1, AM2, AM3, AM4, AM5, AM6, AM7, AM9, AM10, AM11, AM12, AM13, AM14, AM15, AM16, AM17, AM18, AMC8, AMC9, and AMC11.

The capacity of the antibodies to induce T-cell mediated cytotoxicity was assessed using co-cultures of target cells (“T”): CFSE (Biolegend) labelled target positive (NCI-H1703 cell line) with effector cells (“E”): PBMCs, at an E:T ratio of 1:1. Solutions of antibody covering the concentration range of 0.01-1000 ng/ml were added to relevant wells, Following 72 h co-culture (37° C.), assay supernatants and cells were harvested from the assay plates and 7-AAD viability staining solution (BioLegend) was added. Samples were then analysed by flow cytometry.

The capacity of the certain scDb antibodies of the invention to induce T-cell mediated cytotoxicity was further assessed using co-cultures of target cells (“T”): CFSE (Biolegend) labelled target positive (A375, THP1, C-33-A or HuTu80) or negative (HCT116 and Boleth) cancer cell lines or peptide pulsed T2 cells (50 μM peptide overnight (16 h) pulsing, excess peptides washed away prior to co-culture) with effector cells (“E”): PBMCs, or pan T cells isolated from PBMCs, at an E:T ratio of 1:1. Solutions of 1-H02 covering the concentration range of 0.01-1000 ng/ml were added to relevant wells,

Following 72 h co-culture (37° C.), assay supernatants and cells were harvested from the assay plates and 7-AAD viability staining solution (BioLegend) was added. Samples were then analysed by flow cytometry.

### Results

### T-Cell Activation, Differentiation, Proliferation and T-Cell Mediated Cytotoxicity

To assess T-cell engager-mediated T-cell activation, differentiation and proliferation, 1-H02 was first co-cultured with PBMCs and MAGE-A4²³⁰⁻²³⁹ peptide pulsed T2 cells. Following 72 h co-culture, T-cell activation, as measured by upregulation of CD25 and CD71 surface expression on CD4+ and CD8+ T cells were only observed when 1-H02 was co-cultured with T2 cells pulsed with MAGE-A4²³⁰⁻²³⁹ peptide (FIGS. 10A, 10B, 11A and 11B). Similarly, intracellular granzyme B and proliferation of both CD4+ and CD8+ T cells were only induced following 1-H02 co-culture with PBMCs and HLA-A*02:01/MAGE-A4²³⁰⁻²³⁹ positive cells (T2 cells pulsed with MAGE-A4²³⁰⁻²³⁹ peptide) (FIGS. 10C, 10D, 11C and 11D). (Assays 3 and 4 above).

The ability of 1-H02 to induce re-directed T-cell specific target killing was further assessed via 7-AAD flow cytometry analysis (Assay 5 above). 1-H02 was able to induce re-directed T-cell specific target cell killing in a dose-dependent manner only when co-cultured with HLA-A*02:01/MAGE-A4²³⁰⁻²³⁹ positive T2 cells (FIG. 12). Notably, though the likely dominating effector cells mediating target cell cytotoxicity are the CD8+ and CD4+αβTCR expressing T cells (FIGS. 10 and 11), the target cell killing (FIG. 12) will be the integrated effect exerted by all CD3+ cells in the PBMC cell pool, including putative NKT cells and αβTCR expressing T cells.

Following the assessment of 1-H02 to mediate T-cell redirected killing and activation when incubated with cells pulsed with MAGE-A4²³⁰⁻²³⁹ peptide, 1-H02 was tested in a further relevant system with cancer cell lines (“target cells”) that naturally express the HLA-A*02:01/MAGE-A4²³⁰⁻²³⁹ target. To demonstrate engager mediated T-cell activation, differentiation, and proliferation with HLA-A*02:01/MAGE-A4²³⁰⁻²³⁹ positive target cell lines, 1-H02, PBMCs and HLA-A*02:01/MAGE-A4²³⁰⁻²³⁹ positive (NCI-H1703) or HLA-A*02:01/MAGE-A4²³⁰⁻²³⁹ negative (HCT116) target cells were co-cultured for 72 h. Following 72 h co-culture, T-cell activation, as measured by upregulation of CD25 and CD71 surface expression on CD8+ and CD4+ T cells was only observed when 1-H02 was co-cultured with HLA-A*02:01/MAGE-A4²³⁰⁻²³⁹ positive cells (FIGS. 13A, 13B, 14A and 14B). Similarly, increases in intracellular granzyme B and proliferation of CD8+ and CD4+ T cells were only induced following 1-H02 co-culture with PBMCs and HLA-A*02:01/MAGE-A4²³⁰⁻²³⁹ positive cells, NCI-H1703 (FIGS. 13C, 13D, 14C and 14D). (Assays 3 and 4 above).

The ability of 1-H02 to induce re-directed T-cell specific target killing was further assessed via 7-AAD flow cytometry analysis, using a range of HLA-A*02:01/MAGE-A4²³⁰⁻²³⁹ positive (NCI-H1703, A375 and THP1) and HLA-A*02:01/MAGE-A4²³⁰⁻²³⁹ negative (HCT116 and Boleth) cancer (“target”) cell lines (Assay 5 above). Following 72 h co-culture, 1-H02 was able to induce re-directed T-cell specific killing in a dose-dependent manner, only when cultured with HLA-A*02:01/MAGE-A4²³⁰⁻²³⁹ positive cancer cells (FIGS. 15A and B). This was the case both in tumor cell lines representing solid tumors (15A) and liquid tumors (15B), and for different T-cell comprising effector cell populations; PBMCs (15A) and pan T-cells isolated therefrom (15B).

Similar analyses were performed using many other of the antibodies disclosed in Table A, including but not limited to AM2, AM6, AM15, AMC8, AMC9 and AMC11, with similarly specific cytotoxicity effects being observed as for 1-H02 (data not shown).

EC₅₀ values were determined as follows for 1-H02: EC₅₀=166.2 pM as measured in the cancer cell line NCI-H1703; EC₅₀=130.1 pM as measured in the cancer cell line A375; and EC₅₀=10.7 nM (10700 pM) as measured in the cancer cell line THP1.

The ability of antibodies AM1, AM2, AM3, AM4, AM5, AM6, AM7, AM9, AM10, AM11, AM12, AM13, AM14, AM15, AM16, AM17, AM18, AMC8, AMC9, and AMC11 to induce re-directed T-cell specific target killing was further assessed via 7-AAD expression flow cytometry analysis, using HLA-A*02:01/MAGE-A4²³⁰⁻²³⁹ positive (NCI-H1703) cancer (“target”) cell lines (Assay 5 above). Following 72 h co-culture, each of the antibodies was able to induce re-directed T-cell specific killing in a dose-dependent manner, and at advantageously low concentrations (Table I, below).

TABLE I
Ability of antibodies of the invention to induce
T-cell specific killing vs. NCI-H1703

| | EC50 |
|---|---|
| Antibody | vs. NCI-H1703 (pM) |
| AM1 | 6.4 |
| AM2 | 2.2 |
| AM3 | 3.4 |
| AM4 | 3.1 |
| AM5 | 6.4 |
| AM6 | 4.8 |
| AM7 | 10.1 |
| AM9 | 7.6 |
| AM10 | 7.6 |
| AM11 | 3.1 |
| AM12 | 4.3 |
| AM13 | 3.9 |
| AM14 | 5.1 |
| AM15 | 2.2 |
| AM16 | 5.3 |
| AM17 | 2.9 |
| AM18 | 4.4 |
| AMC8 | 2.51 |
| AMC9 | 1.68 |
| AMC11 | 0.72 |

The ability of antibodies AM2, AM6, AM15, AMC8, AMC9, and AMC11 to induce re-directed T-cell specific target killing was further assessed via 7-AAD flow cytometry analysis, using HLA-A*02:01/MAGE-A4²³⁰⁻²³⁹ positive (A375) cancer (“target”) cell lines (Assay 5 above). Following 72 h co-culture, each of the antibodies was able to induce re-directed T-cell specific killing in a dose-dependent manner, and at advantageously low concentrations (Table J, below).

TABLE J
Ability of antibodies of the invention to induce
T-cell specific killing vs. A375

| | EC50 |
|---|---|
| Antibody | vs. A375 (pM) |
| AM2 | 3.7 |
| AM6 | 5.1 |
| AM15 | 4.2 |
| AMC8 | 1.6 |
| AMC9 | 2.4 |
| AMC11 | 2.3 |

The ability of antibodies AM2, AM15 and AMC9 to induce re-directed T-cell specific target killing against further HLA-A*02:01/MAGE-A4²³⁰⁻²³⁹ positive cancer cell types (C-33A and HuTu80 cell lines) was further assessed via 7-AAD flow cytometry analysis (Assay 5 above). Following 72 h co-culture, each of the antibodies was able to induce re-directed T-cell specific killing in a dose-dependent manner, and at advantageously low concentrations (Table K, below).

TABLE K
Ability of antibodies of the invention to induce
T-cell specific killing vs. C-33-A and HuTu80

| | EC50 | EC50 |
|---|---|---|
| Antibody | vs. C-33-A (pM) | vs. HuTu80 (pM) |
| AM2 | 7.2 | 7.8 |
| AM15 | 11 | 7.1 |
| AMC9 | 1.33 | 6.6 |

### Example 6: T-Cell Cytokine Release (IFNγ)

Cytotoxic T cells release cytokines such as IFN-γ, which induce the increased expression of MHC class I and other molecules involved in peptide loading in cancer cells, which in turn increases the chance that cancer cells will be recognized as target cells for cytotoxic attack. IFN-γ also activates macrophages, recruiting them to target sites both as effector cells and as antigen-presenting cells. The ability of an antigen binding protein, particularly at low concentrations, to induce the release of IFN-γ from T-cells when co-cultured with HLA-A*02:01/MAGE-A4²³⁰⁻²³⁹ positive target cells would therefore be advantageous, and demonstrative of the cytotoxic and therapeutic activity of the molecules.

Assay 8 above was performed to assess the capacity of each of the antibodies of Table A (scDb format) to induce CD3-mediated cytokine (IFNγ) release from T cells. Briefly, co-cultures of target positive cancer cell lines (target “T” cells) and effector cells (PBMCs comprising T cells, “E”), at an E:T ratio of about 1:1, were co-cultured in the presence of the antigen binding protein (e.g. antibody) to be tested, over a concentration range (of about 0.1-10,000 pM), for 24 h at about 37° C. Supernatant was harvested and 100 μl was added to ELISA plates previously coated with IFNγ capture antibody, washed, blocked and washed, then incubated for about 2 hours at room temperature. After washing, the plates were incubated with 100 μl of secondary antibody for 1 hour at room temperature, washed, then incubated for 30 minutes at room temperature with 100 μl of 1:1000 diluted avidin-HRP. After washing, wells of the plate were incubated for up to 5 minutes, in the dark, with 100 μl of TMB solution (3,3′,5,5′-Tetramethylbenzidine), with immediate addition of 100 μl of 1M HCl to stop the reaction. Absorbance was measured at 450 nM, and the concentration of IFNγ in each of the wells was determined using a standard curve.

Following 24 h co-culture, each of the antibodies was able to induce IFNγ release from PBMCs, more specifically T cells within the PBMCs, in a dose-dependent manner, and at advantageously low concentrations (Table L, below).

TABLE L
T-cell Cytokine release (IFNγ)

| | IFNγ EC50 vs. target |
|---|---|
| Antibody | positive cells (pM) |
| 1-H02 | 83.1 |
| AM1 | 11.8 |
| AM2 | 4.4 |
| AM3 | 5.8 |
| AM4 | 4.5 |
| AM5 | 9 |
| AM6 | 37.7 |
| AM7 | 13.4 |
| AM9 | 12.2 |
| AM10 | 31.1 |
| AM11 | 7.2 |
| AM12 | 6.4 |
| AM13 | 5.8 |
| AM14 | 4.1 |
| AM15 | 10.6 |
| AM16 | 12.3 |
| AM17 | 10.9 |
| AM18 | 9.5 |
| AMC1 | 3.6 |
| AMC3 | 7.3 |
| AMC4 | 9.2 |
| AMC5 | 8 |
| AMC6 | 8.6 |
| AMC7 | 8.5 |
| AMC8 | 7.7 |
| AMC9 | 7.6 |
| AMC10 | 1.7 |
| AMC11 | 8.5 |
| AMC12 | 6.9 |
| AMC14 | 8.2 |

### Example 7: In Vivo Efficacy of AM15

To generate humanised mice for an in vivo efficacy study, 3-week-old female NSG (NOD scid gamma) mice were irradiated and then engraftment, by i.v. injection, of human cord blood hematopoietic stem cells (HSCs) was performed. After 12 weeks, post-engraftment quality control (QC) was completed to ensure the engraftment was successful.

The humanised mice were then subcutaneously injected with 5×10⁶ A375 cells (discussed above) into the flank. The tumour was allowed to grow until the average tumour volume had reached 80-100 mm³.

Mice were treated, via i.v. injection, on days 0-2 and days 7-9 with 0.1 mg/kg AM15 (scDb) or PBS control. Tumour volume, health scores and body weight were assessed every other day until mice were euthanised on day 10.

The results are shown in FIG. 18, which demonstrates that administration of AM15 led to marked decrease in tumour growth as compared to control.

In summary, the present inventors have demonstrated the following:

The antibodies of the invention bind MAGE-A4²³⁰⁻²³⁹ presented on HLA-A2 with a centrally focused binding footprint. Binding of HLA-A2 presenting MAGE-A4²³⁰⁻²³⁹ homologues revealed no cross-reactivity, apart from with a greatly reduced binding to a non-risk homolog derived from MAGE-A8 (SEQ ID NO: 44) as compared with MAGE-A4²³⁰⁻²³⁹.

The TCE antibodies of the invention redirect T-cell activity in a specific manner, i.e. against HLA-A*02:01/MAGE-A4²³⁰⁻²³⁹ positive cells, and not against HLA-A*02:01 positive, MAGE-A4²³⁰⁻²³⁹ negative cells, nor cells displaying HLA-A*02:01 restricted genomic risk peptides with high sequence identity to MAGE-A4²³⁰⁻²³⁹.

TCE antibodies of the invention preferentially induce activation, differentiation and proliferation of T cells directed against HLA-A*02:01/MAGE-A4²³⁰⁻²³⁹ positive cells as compared to against HLA-A*02:01 positive, MAGE-A4²³⁰⁻²³⁹ negative cells.

TCE antibodies of the invention preferentially induce T-cell mediated cytotoxicity in a specific manner against HLA-A*02:01/MAGE-A4²³⁰⁻²³⁹ positive cells as compared to against HLA-A*02:01 positive, MAGE-A4²³⁰⁻²³⁹ negative cells.

TCE antibodies of the invention have demonstrated advantageous thermal stability.

TCE antibodies of the invention mediate T-cell re-directed target cell killing with surprisingly advantageous efficacy; cytotoxicity is demonstrated at very low concentrations (low EC₅₀ values), in assays with an effector cell: target cell ratio of only 1:1. This is surprising in light of the binding affinity. Further, it is well known that increasing Effector cell:Target cell ratios (e.g. from 1:1 to 10:1) greatly increases measured TCE potency^{8,9}. The present inventors have, surprisingly, shown advantageous levels of killing with a low effector to target ratio, i.e. a ratio of 1:1.

TCE antibodies of the invention induce release of IFN-gamma from T-cells in a targeted manner and with surprisingly advantageous efficacy; low EC₅₀ values are demonstrated, surprisingly and advantageously in assays with an effector cell: target cell ratio of only 1:1.

The antibodies of the invention achieve the above-mentioned effects, e.g. efficacy (cytotoxicity and induced IFN-gamma release), whilst having relatively low binding activities/strength, meaning that the antibodies of the invention have an advantageous cytotoxicity/safety profile.

### REFERENCES

1 Høydahl, L. S. et al. Multivalent pIX phage display selects for distinct and improved antibody properties. Sci. Rep. 6, 39066, doi:10.1038/srep39066 (2016).
2 Duffour, M.-T. et al. A MAGE-A4 peptide presented by HLA-A2 is recognized by cytolytic T lymphocytes. European journal of immunology 29, 3329-3337, doi:https://doi.org//10.1002/(SICI)1521-4141(199910)29:10<3329::AID-IMMU3329>3.0.CO; 2-7 (1999).
3 Frick, R. et al. Affinity maturation of TCR-like antibodies using phage display guided by structural modeling. Protein Engineering, Design and Selection 35, doi:10.1093/protein/gzac005 (2022).
4 Douglass, J. et al. Bispecific antibodies targeting mutant RAS neoantigens. Science Immunology 6, eabd5515, doi:10.1126/sciimmunol.abd5515 (2021).
Völkel, T., Korn, T., Bach, M., Müller, R. & Kontermann, R. E. Optimized linker sequences for the expression of monomeric and dimeric bispecific single-chain diabodies. Protein Engineering, Design and Selection 14, 815-823, doi:10.1093/protein/14.10.815 (2001).
6 Webber, K. O., Reiter, Y., Brinkmann, U., Kreitman, R. & Pastan, I. Preparation and characterization of a disulfide-stabilized Fv fragment of the anti-Tac antibody: comparison with its single-chain analog. Mol Immunol 32, 249-258, doi:10.1016/0161-5890(94)00150-y (1995).
7 Jager, M. & Pluckthun, A. Folding and assembly of an antibody Fv fragment, a heterodimer stabilized by antigen. J Mol Biol 285, 2005-2019, doi:10.1006/jmbi.1998.2425 (1999).
8 Dao, T. et al. Therapeutic bispecific T-cell engager antibody targeting the intracellular oncoprotein WT1. Nat Biotech 33, 1079-1086 (2015).
9 Qi, J. et al. Potent and selective antitumor activity of a T cell-engaging bispecific antibody targeting a membrane-proximal epitope of ROR1. Proceedings of the National Academy of Sciences 115, E5467-E5476, doi:doi:10.1073/pnas.1719905115 (2018).

1. An antigen binding protein comprising at least one antigen binding domain which binds to HLA-A*02:01/MAGE-A4²³⁰⁻²³⁹, said antigen binding domain comprising a heavy chain variable domain (VH domain) that comprises three complementarity determining regions (CDRs), and a light chain variable domain (VL domain) that comprises three CDRs, wherein
said heavy chain variable domain comprises:
(a) a variable heavy (VH) CDR1 that comprises the amino acid sequence of GYTLTELS (SEQ ID NO:5) or a sequence substantially homologous thereto,
(b) a VH CDR2 that comprises the amino acid sequence of FDPEDGET (SEQ ID NO:6) or a sequence substantially homologous thereto, and
(c) a VH CDR3 that comprises the amino acid sequence of ATDQGSSWGFY (SEQ ID NO:7) or a sequence substantially homologous thereto;
and/or wherein
said light chain variable domain comprises:
(d) a variable light (VL) CDR1 that comprises the amino acid sequence of QSISSY (SEQ ID NO:8) or a sequence substantially homologous thereto,
(e) a VL CDR2 that comprises the amino acid sequence of AAS (SEQ ID NO:9) or a sequence substantially homologous thereto, and
(f) a VL CDR3 that comprises the amino acid sequence of QQSYSTPYT (SEQ ID NO:10) or a sequence substantially homologous thereto.

2. The antigen binding protein of claim 1, wherein
said heavy chain variable domain comprises:
(a) a variable heavy (VH) CDR1 that comprises the amino acid sequence of
(SEQ ID NO: 5)
GYTLTELS;
or
(SEQ ID NO: 81)
GPKLYEVS,
or a sequence substantially homologous thereto,
(b) a VH CDR 2 that comprises the amino acid sequence of
(SEQ ID NO: 6)
FDPEDGET;
(SEQ ID NO: 82)
FDPYMSRT;
(SEQ ID NO: 83)
FDPYLART;
or
(SEQ ID NO: 84)
FDPEQGET,
or a sequence substantially homologous thereto, and
(c) a VH CDR 3 that comprises the amino acid sequence of
(SEQ ID NO: 7)
ATDQGSSWGFY;
(SEQ ID NO: 85)
ATDQGASWGFY;
or
(SEQ ID NO: 86)
AADQGSSWGFY,
or a sequence substantially homologous thereto;
and/or wherein
said light chain variable domain comprises:
(d) a variable light (VL) CDR1 that comprises the amino acid sequence of
(SEQ ID NO: 8)
QSISSY;
(SEQ ID NO: 87)
QNIMWY;
(SEQ ID NO: 88)
VHIYWY;
(SEQ ID NO: 89)
HHIFWY;
(SEQ ID NO: 90)
IDIRWY;
(SEQ ID NO: 91)
QSIMTY;
(SEQ ID NO: 92)
QTVATY;
or
(SEQ ID NO: 93)
EDIRYY,
or a sequence substantially homologous thereto,
(e) a variable light VL CDR2 that comprises the amino acid sequence of
(SEQ ID NO: 9)
AAS;
(SEQ ID NO: 94)
SAS;
or
(SEQ ID NO: 95)
VTS,
or a sequence substantially homologous thereto, and
(f) a VL CDR3 that comprises the amino acid sequence of
(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,
or a sequence substantially homologous thereto.

3. The antigen binding protein of claim 1 or claim 2, wherein
said heavy chain variable domain comprises:
(a) a variable heavy (VH) CDR1 that comprises the amino acid sequence of
(SEQ ID NO: 5)
GYTLTELS;
or
(SEQ ID NO: 81)
GPKLYEVS,
or a sequence substantially homologous thereto,
(b) a VH CDR 2 that comprises the amino acid sequence of FDPEDGET (SEQ ID NO:6),
(SEQ ID NO: 6)
FDPEDGET,
or a sequence substantially homologous thereto, and
(c) a VH CDR 3 that comprises the amino acid sequence of
(SEQ ID NO: 7)
ATDQGSSWGFY;
or
(SEQ ID NO: 85)
ATDQGASWGFY,
or a sequence substantially homologous thereto;
and/or wherein
said light chain variable domain comprises:
(d) a variable light (VL) CDR1 that comprises the amino acid sequence of
(SEQ ID NO: 8)
QSISSY;
(SEQ ID NO: 87)
QNIMWY;
or
(SEQ ID NO: 92)
QTVATY,
or a sequence substantially homologous thereto,
(e) a variable light VL CDR2 that comprises the amino acid sequence of
(SEQ ID NO: 9)
AAS;
or
(SEQ ID NO: 95)
VTS,
or a sequence substantially homologous thereto, and
(f) a VL CDR3 that comprises the amino acid sequence of
(SEQ ID NO: 10)
QQSYSTPYT;
(SEQ ID NO: 96)
QQSYSTPFT;
(SEQ ID NO: 97)
QQAYRIPYT;
or
(SEQ ID NO: 98)
QQAYSTPVT,
or a sequence substantially homologous thereto

4. The antigen binding protein of any one of claims 1 to 3, wherein
said heavy chain variable domain comprises:
(a) a variable heavy (VH) CDR1 that comprises the amino acid sequence of GYTLTELS (SEQ ID NO: 5) or a sequence substantially homologous thereto,
(b) a VH CDR 2 that comprises the amino acid sequence of FDPEDGET (SEQ ID NO:6) or a sequence substantially homologous thereto, and
(c) a VH CDR 3 that comprises the amino acid sequence of
(SEQ ID NO: 7)
ATDQGSSWGFY;
or
(SEQ ID NO: 85)
ATDQGASWGFY,
or a sequence substantially homologous thereto;
and/or wherein
said light chain variable domain comprises:
(d) a variable light (VL) CDR1 that comprises the amino acid sequence of
(SEQ ID NO: 8)
QSISSY;
or
(SEQ ID NO: 87)
QNIMWY,
or a sequence substantially homologous thereto,
(e) a variable light VL CDR2 that comprises the amino acid sequence of AAS (SEQ ID NO:9), or a sequence substantially homologous thereto, and
(f) a VL CDR3 that comprises the amino acid sequence of
(SEQ ID NO: 10)
QQSYSTPYT;
or
(SEQ ID NO: 96)
QQSYSTPFT,
or a sequence substantially homologous thereto.

5. The antigen binding protein of any one of claims 1 to 4, wherein
said heavy chain variable domain comprises:
(a) a variable heavy (VH) CDR1 that comprises the amino acid sequence of GYTLTELS (SEQ ID NO:5) or a sequence substantially homologous thereto,
(b) a VH CDR2 that comprises the amino acid sequence of FDPEDGET (SEQ ID NO:6) or a sequence substantially homologous thereto, and
(c) a VH CDR3 that comprises the amino acid sequence of ATDQGSSWGFY (SEQ ID NO:7) or a sequence substantially homologous thereto;
and/or wherein
said light chain variable domain comprises:
(d) a variable light (VL) CDR1 that comprises the amino acid sequence of QSISSY (SEQ ID NO:8) or a sequence substantially homologous thereto,
(e) a VL CDR2 that comprises the amino acid sequence of AAS (SEQ ID NO:9) or a sequence substantially homologous thereto, and
(f) a VL CDR3 that comprises the amino acid sequence of QQSYSTPFT (SEQ ID NO:96) or a sequence substantially homologous thereto.

6. The antigen binding protein of any one of claims 1 to 4, wherein
said heavy chain variable domain comprises:
(a) a variable heavy (VH) CDR1 that comprises the amino acid sequence of GYTLTELS (SEQ ID NO:5) or a sequence substantially homologous thereto,
(b) a VH CDR2 that comprises the amino acid sequence of FDPEDGET (SEQ ID NO:6) or a sequence substantially homologous thereto, and
(c) a VH CDR3 that comprises the amino acid sequence of ATDQGSSWGFY (SEQ ID NO:7) or ATDQGASWGFY (SEQ ID NO:85), or a sequence substantially homologous thereto;
and/or wherein
said light chain variable domain comprises:
(d) a variable light (VL) CDR1 that comprises the amino acid sequence of QNIMWY (SEQ ID NO:87) or a sequence substantially homologous thereto,
(e) a VL CDR2 that comprises the amino acid sequence of AAS (SEQ ID NO:9) or a sequence substantially homologous thereto, and
(f) a VL CDR3 that comprises the amino acid sequence of QQSYSTPYT (SEQ ID NO:10) or a sequence substantially homologous thereto.

7. The antigen binding protein of any one of claims 1 to 6, wherein:
the VH CDR1 comprises the amino acid sequence of the given SEQ ID NO or a sequence substantially homologous thereto that comprises up to 2 amino acid substitutions;
the VH CDR2 comprises the amino acid sequence of the given SEQ ID NO or a sequence substantially homologous thereto that comprises up to 2 amino acid substitutions;
the VH CDR3 comprises the amino acid sequence of the given SEQ ID NO or a sequence substantially homologous thereto that comprises up to 3 amino acid substitutions;
the VL CDR1 comprises the amino acid sequence of the given SEQ ID NO or a sequence substantially homologous thereto that comprises up to 2 amino acid substitutions;
the VL CDR2 comprises the amino acid sequence of the given SEQ ID NO or a sequence substantially homologous thereto that comprises 1 amino acid substitution; and
the VL CDR3 comprises the amino acid sequence of the given SEQ ID NO or a sequence substantially homologous thereto that comprises up to 3 amino acid substitutions.

8. The antigen binding protein of any one of claims 1 to 7, wherein said substantially homologous sequence is a sequence containing only 1 amino acid substitution as compared to the given CDR sequence.

9. The antigen binding protein of any one of claims 1 to 8, wherein the heavy chain variable domain comprises the amino acid sequence of SEQ ID NO:3, or a sequence having at least 80% sequence identity thereto, and/or wherein the light chain variable domain comprises the amino acid sequence of SEQ ID NO:4, or a sequence having at least 80% sequence identity thereto.

10. The antigen binding protein of claim 9, wherein the heavy chain variable domain and the light chain variable domain comprise, respectively, the following amino acid sequences:

| VH domain | VL domain | |
|---|---|---|
| SEQ ID NO: 3 | SEQ ID NO: 4 | ; |
| SEQ ID NO: 3 | SEQ ID NO: 114 | ; |
| SEQ ID NO: 3 | SEQ ID NO: 116 | ; |
| SEQ ID NO: 118 | SEQ ID NO: 116 | ; |
| SEQ ID NO: 3 | SEQ ID NO: 120 | ; |
| SEQ ID NO: 3 | SEQ ID NO: 122 | ; |
| SEQ ID NO: 3 | SEQ ID NO: 124 | ; |
| SEQ ID NO: 126 | SEQ ID NO: 128 | ; |
| SEQ ID NO: 130 | SEQ ID NO: 4 | ; |
| SEQ ID NO: 132 | SEQ ID NO: 4 | ; |
| SEQ ID NO: 134 | SEQ ID NO: 4 | ; |
| SEQ ID NO: 118 | SEQ ID NO: 4 | ; |
| SEQ ID NO: 134 | SEQ ID NO: 124 | ; |
| SEQ ID NO: 136 | SEQ ID NO: 116 | ; |
| SEQ ID NO: 137 | SEQ ID NO: 114 | ; |
| SEQ ID NO: 3 | SEQ ID NO: 138 | ; |
| SEQ ID NO: 3 | SEQ ID NO: 140 | ; |
| SEQ ID NO: 3 | SEQ ID NO: 142 | ; |
| SEQ ID NO: 3 | SEQ ID NO: 144 | ; |
| SEQ ID NO: 145 | SEQ ID NO: 142 | ; |
| SEQ ID NO: 145 | SEQ ID NO: 116 | ; |
| SEQ ID NO: 145 | SEQ ID NO: 122 | ; |
| SEQ ID NO: 145 | SEQ ID NO: 124 | ; |
| SEQ ID NO: 130 | SEQ ID NO: 142 | ; |
| SEQ ID NO: 130 | SEQ ID NO: 116 | ; |
| SEQ ID NO: 118 | SEQ ID NO: 142 | ; |
| SEQ ID NO: 3 | SEQ ID NO: 146 | ; |
| SEQ ID NO: 3 | SEQ ID NO: 147 | ; |
| SEQ ID NO: 145 | SEQ ID NO: 147 | ; or |
| SEQ ID NO: 130 | SEQ ID NO: 147 | . |

11. The antigen binding protein of any one of claims 1 to 10, wherein said antigen binding protein is an antibody.

12. The antigen binding protein of any one of claims 1 to 11, wherein said antigen binding protein is a bispecific antibody in which said antigen binding domain that binds to HLA-A*02:01/MAGE-A4²³⁰⁻²³⁹ is a first antigen binding domain, and wherein said antigen binding protein further comprises a second antigen binding domain that binds to a T-cell antigen present on the surface of T cells, preferably wherein said T-cell antigen is CD3.

13. The antigen binding protein of any one of claims 1 to 12, wherein said antigen binding protein is a single-chain bispecific diabody (scDb).

14. The antigen binding protein of claim 13, wherein said scDb comprises the amino acid sequence set out in SEQ ID NO: 30, 148, 152, or 156.

15. The antigen binding protein of any one of claims 1 to 14, wherein said antigen binding protein
i) preferentially binds to HLA-A*02:01/MAGE-A4²³⁰⁻²³⁹ as compared to any one, preferably each, of the HLA-A*02:01 restricted peptides comprising or consisting of SEQ ID NOs: 33, 35, 36 and 38; or
ii) preferentially binds to HLA-A*02:01/MAGE-A4²³⁰⁻²³⁹ as compared to any one, preferably each, of the HLA-A*02:01 restricted peptides comprising or consisting of SEQ ID NOs: 41 to 43 and 45 to 53.

16. The antigen binding protein of any one of claims 12 to 15, wherein said antigen binding protein is capable of redirecting T-cell activity against cells displaying HLA-A*02:01/MAGE-A4²³⁰⁻²³⁹.

17. The antigen binding protein of any one of claims 12 to 16, wherein said antigen binding protein preferentially redirects T-cell activity against cells displaying HLA-A*02:01/MAGE-A4²³⁰⁻²³⁹ as compared to against cells displaying a HLA-A*02:01 restricted peptide comprising or consisting of the amino acid sequence set out in any one of SEQ ID NOs: 54 to 75.

18. The antigen binding protein of any one of claims 6 to 15, wherein said antigen binding protein exhibits an EC₅₀ of less than 2×10⁻¹⁰ M as measured in cancer cells.

19. An immunoconjugate comprising the antigen binding protein of any one of claims 1 to 18, operatively attached to at least one other therapeutic or diagnostic agent.

20. One or more nucleic acid molecules comprising nucleotide sequences that encode the antigen binding protein or immunoconjugate of any one of claims 1 to 19.

21. One or more expression vectors comprising the one or more of the nucleic acid molecules of claim 20.

22. One or more host cells or viruses comprising said expression vectors of claim 21, or said nucleic acid molecules of claim 20, or expressing the antigen binding protein or immunoconjugate of any one of claims 1 to 19.

23. A method of producing the antigen binding protein or immunoconjugate of any one of claims 1 to 19, said method comprising the steps of (i) culturing a host cell comprising the expression vectors of claim 21 or the nucleic acid molecules of claim 20, under conditions suitable for the expression of the encoded antigen binding protein or immunoconjugate; and optionally (ii) isolating or obtaining the antigen binding protein or immunoconjugate from the host cell or from the growth medium/supernatant.

24. A composition comprising the antigen binding protein of any one of claims 1 to 18, the immunoconjugate of claim 19, the one or more nucleic acid molecules of claim 20, the one or more expression vectors of claim 21, or the one or more host cells or viruses of claim 22; and a diluent, carrier or excipient.

25. The antigen binding protein of any one of claims 1 to 18, the immunoconjugate of claim 19, the one or more nucleic acid molecules of claim 20, the one or more expression vectors of claim 21, or the one or more host cells or viruses of claim 22, for use in therapy.

26. The antigen binding protein of any one of claims 1 to 18, the immunoconjugate of claim 19, the one or more nucleic acid molecules of claim 20, the one or more expression vectors of claim 21, or the one or more host cells or viruses of claim 22, for use in the treatment or prevention of cancer.


SEQUENCE LISTING (165 sequences)

SEQ ID NO: 1 - DNA, 354 bp, synthetic construct
  source 1..354: mol_type = other DNA
caggtccagcttgtgcagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagatcagggtagtagctggggattctactggggccagggaaccctggtcaccgtctcctca

SEQ ID NO: 2 - DNA, 321 bp, Synthetic construct
  source 1..321: mol_type = other DNA
gacatccgggtgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 3 - protein, 118 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSS

SEQ ID NO: 4 - protein, 107 aa, Synthetic construct
DIRVTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 5 - protein, 8 aa, Synthetic construct
GYTLTELS

SEQ ID NO: 6 - protein, 8 aa, Synthetic construct
FDPEDGET

SEQ ID NO: 7 - protein, 11 aa, Synthetic construct
ATDQGSSWGFY

SEQ ID NO: 8 - protein, 6 aa, Synthetic construct
QSISSY

SEQ ID NO: 9 - skipped

SEQ ID NO: 10 - protein, 9 aa, Synthetic construct
QQSYSTPYT

SEQ ID NO: 11 - protein, 25 aa, Synthetic construct
QVQLVQSGAEVKKPGASVKVSCKVS

SEQ ID NO: 12 - protein, 17 aa, Synthetic construct
MHWVRQAPGKGLEWMGG

SEQ ID NO: 13 - protein, 38 aa, Synthetic construct
IYAQKFQGRVTMTEDTSTDTAYMELSSLRSEDTAVYYC

SEQ ID NO: 14 - protein, 11 aa, Synthetic construct
WGQGTLVTVSS

SEQ ID NO: 15 - protein, 26 aa, Synthetic construct
DIRVTQSPSSLSASVGDRVTITCRAS

SEQ ID NO: 16 - protein, 17 aa, Synthetic construct
LNWYQQKPGKAPKLLIY

SEQ ID NO: 17 - protein, 36 aa, Synthetic construct
SLQSGVPSRFSGSGSGTDFTLTISSLQPEDFATYYC

SEQ ID NO: 18 - protein, 10 aa, Synthetic construct
FGQGTKLEIK

SEQ ID NO: 19 - protein, 10 aa, Homo sapiens
GVYDGREHTV

SEQ ID NO: 20 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 21 - protein, 365 aa, Homo sapiens [sequence omitted]

SEQ ID NO: 22 - protein, 275 aa, Homo sapiens [sequence omitted]

SEQ ID NO: 23 - protein, 101 aa, Homo sapiens
MIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMT

SEQ ID NO: 24 - protein, 122 aa, Synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 25 - protein, 107 aa, Synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 26 - protein, 5 aa, Synthetic construct
GGGGS

SEQ ID NO: 27 - protein, 15 aa, Synthetic construct
GGGGSGGGGSGGGGS

SEQ ID NO: 28 - protein, 21 aa, Synthetic construct
KLSGSASAPKLEEGEFSEARV

SEQ ID NO: 29 - protein, 246 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSSKLSGSASAPKLEEGEFSEARVDIRVTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 30 - protein, 479 aa, Synthetic construct
DIRVTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSQVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSS

SEQ ID NO: 31 - protein, 10 aa, Synthetic construct
AVYDGREHTV

SEQ ID NO: 32 - protein, 10 aa, Synthetic construct
GAYDGREHTV

SEQ ID NO: 33 - protein, 10 aa, Synthetic construct
GVADGREHTV

SEQ ID NO: 34 - protein, 10 aa, Synthetic construct
GVYAGREHTV

SEQ ID NO: 35 - protein, 10 aa, Synthetic construct
GVYDAREHTV

SEQ ID NO: 36 - protein, 10 aa, Synthetic construct
GVYDGAEHTV

SEQ ID NO: 37 - protein, 10 aa, Synthetic construct
GVYDGRAHTV

SEQ ID NO: 38 - protein, 10 aa, Synthetic construct
GVYDGREATV

SEQ ID NO: 39 - protein, 10 aa, Synthetic construct
GVYDGREHAV

SEQ ID NO: 40 - protein, 10 aa, Synthetic construct
GVYDGREHTA

SEQ ID NO: 41 - protein, 10 aa, Homo sapiens
EVYDGREHSA

SEQ ID NO: 42 - protein, 10 aa, Homo sapiens
EVFEGREDSV

SEQ ID NO: 43 - protein, 10 aa, Homo sapiens
EVFEGREDSI

SEQ ID NO: 44 - protein, 10 aa, Homo sapiens
GLYDGREHSV

SEQ ID NO: 45 - protein, 10 aa, Homo sapiens
GVYVGKEHMF

SEQ ID NO: 46 - protein, 10 aa, Homo sapiens
GLYDGREHLI

SEQ ID NO: 47 - protein, 10 aa, Homo sapiens
GVYAGREHFL

SEQ ID NO: 48 - protein, 10 aa, Homo sapiens
EASDGREDSV

SEQ ID NO: 49 - protein, 10 aa, Homo sapiens
GVYDGEEHSV

SEQ ID NO: 50 - protein, 10 aa, Homo sapiens
GIYDGKRHLI

SEQ ID NO: 51 - protein, 10 aa, Homo sapiens
GIYDGILHSI

SEQ ID NO: 52 - protein, 10 aa, Homo sapiens
GLYDGIEHFM

SEQ ID NO: 53 - protein, 10 aa, Homo sapiens
GVYAGREHFV

SEQ ID NO: 54 - protein, 10 aa, Homo sapiens
LLYAGHVQTV

SEQ ID NO: 55 - protein, 10 aa, Homo sapiens
FSVAGKIHTV

SEQ ID NO: 56 - protein, 10 aa, Homo sapiens
ALVDGRWHTL

SEQ ID NO: 57 - protein, 10 aa, Homo sapiens
LALLGRTHSV

SEQ ID NO: 58 - protein, 10 aa, Homo sapiens
LLYNVKRHSL

SEQ ID NO: 59 - protein, 10 aa, Homo sapiens
TQYGGQYRTV

SEQ ID NO: 60 - protein, 10 aa, Homo sapiens
SLYEARNRTL

SEQ ID NO: 61 - protein, 10 aa, Homo sapiens
VQVFGKWHSV

SEQ ID NO: 62 - protein, 10 aa, Homo sapiens
KLYTAQGRTL

SEQ ID NO: 63 - protein, 10 aa, Homo sapiens
AALQGKFHTI

SEQ ID NO: 64 - protein, 10 aa, Homo sapiens
MVYFGKNQSL

SEQ ID NO: 65 - protein, 10 aa, Homo sapiens
LQYGGRNRTV

SEQ ID NO: 66 - protein, 10 aa, Homo sapiens
STIFGKAHSL

SEQ ID NO: 67 - protein, 10 aa, Homo sapiens
SRLSGREHSV

SEQ ID NO: 68 - protein, 10 aa, Homo sapiens
LVYQGKEQSV

SEQ ID NO: 69 - protein, 10 aa, Homo sapiens
NMYQVRQRTL

SEQ ID NO: 70 - protein, 10 aa, Homo sapiens
FEVEGKPHSV

SEQ ID NO: 71 - protein, 10 aa, Homo sapiens
KAYSVQGQSV

SEQ ID NO: 72 - protein, 10 aa, Homo sapiens
KWYQARLQTV

SEQ ID NO: 73 - protein, 10 aa, Homo sapiens
GSYAGKIHSI

SEQ ID NO: 74 - protein, 10 aa, Homo sapiens
TVYPARTQTL

SEQ ID NO: 75 - protein, 10 aa, Homo sapiens
LGYMAQKHSV

SEQ ID NO: 76 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 77 - protein, 448 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, Synthetic construct
DIRVTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 10 aa, Synthetic construct
  VARIANT 1..2: note = any amino acid
  VARIANT 5: note = can be replaced with A or V
  VARIANT 4: note = any amino acid
  VARIANT 6: note = can be replaced with K or Q or H
  VARIANT 8: note = can be replaced with Q or R
  VARIANT 9: note = can be replaced with S
  VARIANT 7: note = any amino acid
  VARIANT 10: note = can be replaced with I or L
XXYXGRXHTV

SEQ ID NO: 80 - protein, 10 aa, Synthetic construct
  VARIANT 1..2: note = any amino acid
  VARIANT 3: note = can be replaced with V or I or L
  VARIANT 4: note = any amino acid
  VARIANT 6: note = can be replaced with K
  VARIANT 7: note = any amino acid
  VARIANT 9: note = can be replaced with S
  VARIANT 10: note = can be replaced with I or L
XXYXGRXHTV

SEQ ID NO: 81 - protein, 8 aa, Synthetic construct
GPKLYEVS

SEQ ID NO: 82 - protein, 8 aa, Synthetic construct
FDPYMSRT

SEQ ID NO: 83 - protein, 8 aa, Synthetic construct
FDPYLART

SEQ ID NO: 84 - protein, 8 aa, Synthetic construct
FDPEQGET

SEQ ID NO: 85 - protein, 11 aa, Synthetic construct
ATDQGASWGFY

SEQ ID NO: 86 - protein, 11 aa, Synthetic construct
AADQGSSWGFY

SEQ ID NO: 87 - protein, 6 aa, Synthetic construct
QNIMWY

SEQ ID NO: 88 - protein, 6 aa, Synthetic construct
VHIYWY

SEQ ID NO: 89 - protein, 6 aa, Synthetic construct
HHIFWY

SEQ ID NO: 90 - protein, 6 aa, Synthetic construct
IDIRWY

SEQ ID NO: 91 - protein, 6 aa, Synthetic construct
QSIMTY

SEQ ID NO: 92 - protein, 6 aa, Synthetic construct
QTVATY

SEQ ID NO: 93 - protein, 6 aa, Synthetic construct
EDIRYY

SEQ ID NO: 94-95 - skipped (2 entries)

SEQ ID NO: 96 - protein, 9 aa, Synthetic construct
QQSYSTPFT

SEQ ID NO: 97 - protein, 9 aa, Synthetic construct
QQAYRIPYT

SEQ ID NO: 98 - protein, 9 aa, Synthetic construct
QQAYSTPVT

SEQ ID NO: 99 - protein, 25 aa, Synthetic construct
QVQLVQSVAEVKKPGASVKVSCKVS

SEQ ID NO: 100 - protein, 26 aa, Synthetic construct
DIQLTQSPSSLSASVGDRVTISCRAS

SEQ ID NO: 101 - protein, 26 aa, Synthetic construct
DIQLTQSPSSLSASVGDRVTITCRAD

SEQ ID NO: 102 - protein, 26 aa, Synthetic construct [sequence omitted]

SEQ ID NO: 103 - protein, 26 aa, Synthetic construct
DIRLTQSPSSLSASVGDRVTMTCRAS

SEQ ID NO: 104 - protein, 26 aa, Synthetic construct
DIRLTQSPSSLSVSVGDRVTITCRAS

SEQ ID NO: 105 - protein, 26 aa, Synthetic construct
DIRVTQSPSSLSASVGDRVTITCRAD

SEQ ID NO: 106 - protein, 17 aa, Synthetic construct
LNWYQQRPGKAPKLLIY

SEQ ID NO: 107 - protein, 17 aa, Synthetic construct
VNWYQQKPGKAPKLLIY

SEQ ID NO: 108 - protein, 17 aa, Synthetic construct
VNWYQQKPGRAPKLLMY

SEQ ID NO: 109 - protein, 36 aa, Synthetic construct
GLQSGVPSRFSGSGSGTDFTLTISSLQPEDFATYYC

SEQ ID NO: 110 - protein, 36 aa, Synthetic construct
IRQSEVPSRFSGDGFGTDFTLTINPLQPDDFATYYC

SEQ ID NO: 111 - protein, 36 aa, Synthetic construct
RLQSGVPSRFSGSGYGTEFTLTISSLQPEDFATYYC

SEQ ID NO: 112 - protein, 10 aa, Synthetic construct
FGPGTKVDIK

SEQ ID NO: 113 - DNA, 321 bp, Synthetic construct
  source 1..321: mol_type = other DNA
gacatccagttgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccggtttgcaaagtggggtcccatcaaggttcagtggcagtggatctgggacagatttcactctcaccatcagcagtctgcaacctgaagattttgcaacttactactgtcaacagagttacagtacccccttcactttcggccctgggaccaaagtggatatcaaa

SEQ ID NO: 114 - protein, 107 aa, Synthetic construct
DIQLTQSPSSLSASVGDRVTITCRASQSISSYLNWYQQKPGKAPKLLIYAASGLQSGVPSRFSGSGSGTDFTLTISSLQPEDFATYYCQQSYSTPFTFGPGTKVDIK

SEQ ID NO: 115 - DNA, 321 bp, Synthetic construct
  source 1..321: mol_type = other DNA
gacatccgggtgacccagtctccatcctccctgtctgcatctgtaggagacagagtcaccatcacttgccgggcaagtcagaatattatgtggtatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 116 - protein, 107 aa, Synthetic construct
DIRVTQSPSSLSASVGDRVTITCRASQNIMWYLNWYQQKPGKAPKLLIYAASSLQSGVPSRFSGSGSGTDFTLTISSLQPEDFATYYCQQSYSTPYTFGQGTKLEIK

SEQ ID NO: 117 - DNA, 354 bp, Synthetic construct
  source 1..354: mol_type = other DNA
caggtccagcttgtgcagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagatcagggtgctagctggggattctactggggccagggaaccctggtcaccgtctcctca

SEQ ID NO: 118 - protein, 118 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGASWGFYWGQGTLVTVSS

SEQ ID NO: 119 - DNA, 321 bp, Synthetic construct
  source 1..321: mol_type = other DNA
gacatccgggtgacccagtctccatcctccctgtctgcatctgtaggagacagagtcaccatcacttgccgggcagatgtgcatatttattggtatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 120 - protein, 107 aa, Synthetic construct
DIRVTQSPSSLSASVGDRVTITCRADVHIYWYLNWYQQKPGKAPKLLIYAASSLQSGVPSRFSGSGSGTDFTLTISSLQPEDFATYYCQQSYSTPYTFGQGTKLEIK

SEQ ID NO: 121 - DNA, 321 bp, Synthetic construct
  source 1..321: mol_type = other DNA
gacatccgggtgacccagtctccatcctccctgtctgcatctgtaggagacagagtcaccatcacttgccgggcagatcatcatattttttggtatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 122 - protein, 107 aa, Synthetic construct
DIRVTQSPSSLSASVGDRVTITCRADHHIFWYLNWYQQKPGKAPKLLIYAASSLQSGVPSRFSGSGSGTDFTLTISSLQPEDFATYYCQQSYSTPYTFGQGTKLEIK

SEQ ID NO: 123 - DNA, 321 bp, Synthetic construct
  source 1..321: mol_type = other DNA
gacatccgggtgacccagtctccatcctccctgtctgcatctgtaggagacagagtcaccatcacttgccgggcagatattgatattcggtggtatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 124 - protein, 107 aa, Synthetic construct
DIRVTQSPSSLSASVGDRVTITCRADIDIRWYLNWYQQKPGKAPKLLIYAASSLQSGVPSRFSGSGSGTDFTLTISSLQPEDFATYYCQQSYSTPYTFGQGTKLEIK

SEQ ID NO: 125 - DNA, 354 bp, Synthetic construct
  source 1..354: mol_type = other DNA
caggtccagcttgtgcagtctgtggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcagcagatcagggtagtagctggggattctactggggccagggaaccctggtcaccgtctcctca

SEQ ID NO: 126 - protein, 118 aa, Synthetic construct
QVQLVQSVAEVKKPGASVKVSCKVSGYTLTELSMHWVRQAPGKGLEWMGGFDPEDGETIYAQKFQGRVTMTEDTSTDTAYMELSSLRSEDTAVYYCAADQGSSWGFYWGQGTLVTVSS

SEQ ID NO: 127 - DNA, 321 bp, Synthetic construct
  source 1..321: mol_type = other DNA
gacatccgggtgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacaggcctaccgcatcccctacacttttggccaggggaccaagctggagatcaaa

SEQ ID NO: 128 - protein, 107 aa, Synthetic construct
DIRVTQSPSSLSASVGDRVTITCRASQSISSYLNWYQQKPGKAPKLLIYAASSLQSGVPSRFSGSGSGTDFTLTISSLQPEDFATYYCQQAYRIPYTFGQGTKLEIK

SEQ ID NO: 129 - DNA, 354 bp, Synthetic construct
  source 1..354: mol_type = other DNA
caggtccagcttgtgcagtctggggctgaggtgaagaagcctggggcctcagtgaaggtctcctgcaaggtttccggaccgaagctgtatgaggtttccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagatcagggtagtagctggggattctactggggccagggaaccctggtcaccgtctcctca

SEQ ID NO: 130 - protein, 118 aa, Synthetic construct
QVQLVQSGAEVKKPGASVKVSCKVSGPKLYEVSMHWVRQAPGKGLEWMGGFDPEDGETIYAQKFQGRVTMTEDTSTDTAYMELSSLRSEDTAVYYCATDQGSSWGFYWGQGTLVTVSS

SEQ ID NO: 131 - DNA, 354 bp, Synthetic construct
  source 1..354: mol_type = other DNA
caggtccagcttgtgcagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatccttatatgtctcggacaatctacgcacagaagttccagggcagagtcaccatgaccgaggacacatctacagacacagcctacatggagctgagcagcctgagatctgaggacacggccgtgtattactgtgcaacagatcagggtagtagctggggattctactggggccagggaaccctggtcaccgtctcctca

SEQ ID NO: 132 - protein, 118 aa, Synthetic construct
QVQLVQSGAEVKKPGASVKVSCKVSGYTLTELSMHWVRQAPGKGLEWMGGFDPYMSRTIYAQKFQGRVTMTEDTSTDTAYMELSSLRSEDTAVYYCATDQGSSWGFYWGQGTLVTVSS

SEQ ID NO: 133 - DNA, 354 bp, Synthetic construct
  source 1..354: mol_type = other DNA
caggtccagcttgtgcagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatccttatcttgcgcgtacaatctacgcacagaagttccagggcagagtcaccatgaccgaggacacatctacagacacagcctacatggagctgagcagcctgagatctgaggacacggccgtgtattactgtgcaacagatcagggtagtagctggggattctactggggccagggaaccctggtcaccgtctcctca

SEQ ID NO: 134 - protein, 118 aa, Synthetic construct
QVQLVQSGAEVKKPGASVKVSCKVSGYTLTELSMHWVRQAPGKGLEWMGGFDPYLARTIYAQKFQGRVTMTEDTSTDTAYMELSSLRSEDTAVYYCATDQGSSWGFYWGQGTLVTVSS

SEQ ID NO: 135 - DNA, 321 bp, Synthetic construct
  source 1..321: mol_type = other DNA
gacatccgggtgacccagtctccatcctccctgtctgcatctgtaggagacagagtcaccatcacttgccgggcaagtcagagcattagcagctatttaaattggtatcaacagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 136 - protein, 118 aa, Synthetic construct
QVQLVQSGAEVKKPGASVKVSCKVSGYTLTELSMHWVRQAPGKGLEWMGGFDPEQGETIYAQKFQGRVTMTEDTSTDTAYMELSSLRSEDTAVYYCATDQGASWGFYWGQGTLVTVSS

SEQ ID NO: 137 - protein, 118 aa, Synthetic construct
QVQLVQSGAEVKKPGASVKVSCKVSGYTLTELSMHWVRQAPGKGLEWMGGFDPYLARTIYAQKFQGRVTMTEDTSTDTAYMELSSLRSEDTAVYYCATDQGASWGFYWGQGTLVTVSS

SEQ ID NO: 138 - protein, 107 aa, Synthetic construct
DIQLTQSPSSLSASVGDRVTITCRADIDIRWYLNWYQQKPGKAPKLLIYAASGLQSGVPSRFSGSGSGTDFTLTISSLQPEDFATYYCQQSYSTPFTFGPGTKVDIK

SEQ ID NO: 139 - DNA, 321 bp, Synthetic construct
  source 1..321: mol_type = other DNA
gacatccagttgacccagtctccatcctccctgtctgcatctgtaggagacagagtcaccatcagttgccgggcaagtcagagcattatgacctatttaaattggtatcagcagagaccagggaaagcccctaagctcctgatctattctgcatccaggttgcaaagtggggtcccatccaggttcagtggcagtggatatgggacagaattcactctcaccatcagcagcctgcaacctgaagattttgcaacttactactgtcaacagagttacagtacccctttcactttcggccctgggaccaaagtggatatcaaa

SEQ ID NO: 140 - protein, 107 aa, Synthetic construct
DIQLTQSPSSLSASVGDRVTISCRASQSIMTYLNWYQQRPGKAPKLLIYSASRLQSGVPSRFSGSGYGTEFTLTISSLQPEDFATYYCQQSYSTPFTFGPGTKVDIK

SEQ ID NO: 141 - DNA, 321 bp, Synthetic construct
  source 1..321: mol_type = other DNA
gacatccggttgacccagtctccatcctccctgtctgcttctgtaggcgacagagtcaccatgacttgccgggcaagtcagaccgttgccacttatgtaaattggtatcaacaaaaaccagggagagcccctaaactcctaatgtatgtcacgtccattcgacaaagtgaggtcccatcaaggttcagtggtgatggatttggcacagacttcactctcaccatcaatcctctgcagcctgacgatttcgcaacttactactgtcaacaggcctacagtactcctgtgacgttcggccaagggaccaagctggagatcaaa

SEQ ID NO: 142 - protein, 107 aa, Synthetic construct
DIRLTQSPSSLSASVGDRVTMTCRASQTVATYVNWYQQKPGRAPKLLMYVTSIRQSEVPSRFSGDGFGTDFTLTINPLQPDDFATYYCQQAYSTPVTFGQGTKLEIK

SEQ ID NO: 143 - DNA, 321 bp, Synthetic construct
  source 1..321: mol_type = other DNA
gacatccggttgacccagtctccatcctccctgtctgtatctgtaggagacagagtcaccatcacttgccgggcgagtgaggacattagatactatgtaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 144 - protein, 107 aa, Synthetic construct
DIRLTQSPSSLSVSVGDRVTITCRASEDIRYYVNWYQQKPGKAPKLLIYAASSLQSGVPSRFSGSGSGTDFTLTISSLQPEDFATYYCQQSYSTPYTFGQGTKLEIK

SEQ ID NO: 145 - protein, 118 aa, Synthetic construct
QVQLVQSGAEVKKPGASVKVSCKVSGPKLYEVSMHWVRQAPGKGLEWMGGFDPEDGETIYAQKFQGRVTMTEDTSTDTAYMELSSLRSEDTAVYYCATDQGASWGFYWGQGTLVTVSS

SEQ ID NO: 146 - protein, 107 aa, Synthetic construct
DIQLTQSPSSLSASVGDRVTITCRASQNIMWYLNWYQQKPGKAPKLLIYAASGLQSGVPSRFSGSGSGTDFTLTISSLQPEDFATYYCQQSYSTPFTFGPGTKVDIK

SEQ ID NO: 147 - protein, 107 aa, Synthetic construct
DIRVTQSPSSLSASVGDRVTITCRASQNIMWYLNWYQQKPGKAPKLLIYAASSLQSGVPSRFSGSGSGTDFTLTISSLQPEDFATYYCQQAYRIPYTFGQGTKLEIK

SEQ ID NO: 148 - protein, 479 aa, Synthetic construct
DIQLTQSPSSLSASVGDRVTITCRASQSISSYLNWYQQKPGKAPKLLIYAASGLQSGVPSRFSGSGSGTDFTLTISSLQPEDFATYYCQQSYSTPFTFGPGTKVDIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSQVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSS

SEQ ID NO: 149 - protein, 246 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSSKLSGSASAPKLEEGEFSEARVDIQLTQSPSSLSASVGDRVTITCRASQSISSYLNWYQQKPGKAPKLLIYAASGLQSGVPSRFSGSGSGTDFTLTISSLQPEDFATYYCQQSYSTPFTFGPGTKVDIK

SEQ ID NO: 150 - protein, 448 aa, Synthetic construct; identical to SEQ ID NO: 77

SEQ ID NO: 151 - protein, 214 aa, Synthetic construct
DIQLTQSPSSLSASVGDRVTITCRASQSISSYLNWYQQKPGKAPKLLIYAASGLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 479 aa, Synthetic construct
DIRVTQSPSSLSASVGDRVTITCRASQNIMWYLNWYQQKPGKAPKLLIYAASSLQSGVPSRFSGSGSGTDFTLTISSLQPEDFATYYCQQSYSTPYTFGQGTKLEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSQVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSS

SEQ ID NO: 153 - protein, 246 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSSKLSGSASAPKLEEGEFSEARVDIRVTQSPSSLSASVGDRVTITCRASQNIMWYLNWYQQKPGKAPKLLIYAASSLQSGVPSRFSGSGSGTDFTLTISSLQPEDFATYYCQQSYSTPYTFGQGTKLEIK

SEQ ID NO: 154 - protein, 448 aa, Synthetic construct; identical to SEQ ID NO: 77

SEQ ID NO: 155 - protein, 214 aa, Synthetic construct
DIRVTQSPSSLSASVGDRVTITCRASQNIMWYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 479 aa, Synthetic construct
DIRVTQSPSSLSASVGDRVTITCRASQNIMWYLNWYQQKPGKAPKLLIYAASSLQSGVPSRFSGSGSGTDFTLTISSLQPEDFATYYCQQSYSTPYTFGQGTKLEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSQVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGASWGFYWGQGTLVTVSS

SEQ ID NO: 157 - protein, 246 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGASWGFYWGQGTLVTVSSKLSGSASAPKLEEGEFSEARVDIRVTQSPSSLSASVGDRVTITCRASQNIMWYLNWYQQKPGKAPKLLIYAASSLQSGVPSRFSGSGSGTDFTLTISSLQPEDFATYYCQQSYSTPYTFGQGTKLEIK

SEQ ID NO: 158 - protein, 448 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGASWGFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 214 aa, Synthetic construct; identical to SEQ ID NO: 155

SEQ ID NO: 160 - protein, 226 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGSSWGFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 161 - protein, 214 aa, Synthetic construct; identical to SEQ ID NO: 151

SEQ ID NO: 162 - protein, 226 aa, Synthetic construct; identical to SEQ ID NO: 160

SEQ ID NO: 163 - protein, 214 aa, Synthetic construct; identical to SEQ ID NO: 155

SEQ ID NO: 164 - protein, 226 aa, Synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDQGASWGFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 165 - protein, 214 aa, Synthetic construct; identical to SEQ ID NO: 155